(19) United States
(12) Patent Application Publication
(10) Pub. No.: US 2026/0150817 A1
(43) Pub. Date: Jun. 4, 2026

(54) ENGINEERED NON-HUMAN ANIMALS

(71) Applicant: LEVERAGEN, INC., Worcester, MA (US)

(72) Inventors: Weisheng Victor CHEN, Hopkinton, MA (US); Hugh CAM, Natick, MA (US); Frank WANG, Acton, MA (US); Shi LEI, Holden, MA (US)

(21) Appl. No.: 19/128,438

(22) PCT Filed: Nov. 9, 2023
(86) PCT No.: PCT/US2023/037114

§ 371 Date: May 8, 2025

Related U.S. Application Data
(60) Provisional application No. 63/424,123, filed on Nov. 9, 2022.
(60) Provisional application No. 63/424,126, filed on Nov. 9, 2022.
(60) Provisional application No. 63/424,130, filed on Nov. 9, 2022.
(60) Provisional application No. 63/424,124, filed on Nov. 9, 2022.
(60) Provisional application No. 63/424,128, filed on Nov. 9, 2022.
(60) Provisional application No. 63/424,119, filed on Nov. 9, 2022.

(51) Int. Cl. A01K 67/0275; C07K 16/00

(52) U.S. Cl. CPC A01K 67/0275; C07K 16/00; A01K 2217/052; A01K 2227/105; A01K 2267/01; C07K 2317/522; C07K 2317/565; C07K 2317/569; C07K 2317/92

(57) ABSTRACT

This document relates to methods and materials involved in producing antibodies (e.g., single domain antibody (sdAbs) and/or heavy chain only antibodies) having one or two chimeric heavy chains. For example, (A) genetically engineered non-human animals (e.g., genetically engineered mice) having the ability to produce an antibody having one or two chimeric heavy chains that include (1) a non-human Ig heavy chain constant domain (CH) 2 and/or a non-human CH3 domain and (2) a VH domain such as a VH domain set forth in any one of SEQ ID NOs:74-87. (B) genetically engineered non-human animals (e.g., genetically engineered mice) having the ability to produce antibody-like molecules that include (1) a non-human heavy chain constant (CH) 2 domain and/or a non-human CH3 domain (e.g., endogenous CH2 and/or CH3 domains) and (2) a TCR variable domain (e.g., a human TCR variable domain). (C) genetically engineered non-human animals (e.g., genetically engineered mice) having the ability to produce antibody-like molecules that include (a) a first amino acid sequence of a FN3 polypeptide (e.g., a ¹⁰FN3 polypeptide). (b) a human Ig variable D domain, (c) a second amino acid sequence of a FN3 polypeptide (e.g., a ¹⁰FN3 polypeptide), and (d) a non-human Ig heavy chain CH2 domain and/or a non-human CH3 domain (e.g., endogenous Ig CH2 and/or CH3 domains), (D) genetically engineered non-human animals (e.g., genetically engineered mice) having the ability to produce an antibody having one or two chimeric heavy chains that include (a) a non-human Ig heavy chain constant domain (CH) 2 and/or a non-human CH3 domain and (b) a variable region that includes a human JH domain (e.g., a human JH3 domain or a human JH4 domain) lacking all or at least one tryptophan amino acid residue(s), and (E) genetically engineered non-human animals (e.g., genetically engineered mice) having the ability to produce an antibody having a modified heavy chain including (a) a non-human CH2 domain and/or a non-human CH3 domain (e.g., endogenous CH2 and/or CH3 domains) and (b) a variable region that includes a human VH domain having a FR2 containing one, two, three, four, or more amino acid are provided. In addition, chimeric non-human animals (e.g., mice) generated from an embryo having (a) a first cell having one or more genomic modifications that prevent the first cell (and cells derived from the first cell) from producing immunoglobulins and (b) a second cell having an IgH locus that includes an exogenous nucleic acid sequence encoding a heavy chain variable region of an antibody of interest such that the chimeric non-human animal produces heavy chain antibodies containing the heavy chain variable region of the antibody of interest in addition to one or more variants of those heavy chain antibodies that underwent in vivo affinity maturation are provided.

### CROSS-REFERENCE TO RELATED APPLICATIONS

This application claims the benefit of U.S. Patent Application Ser. No. 63/424,123, filed on Nov. 9, 2022, U.S. Patent Application Ser. No. 63/424,126, filed on Nov. 9, 2022, U.S. Patent Application Ser. No. 63/424,130, filed on Nov. 9, 2022, U.S. Patent Application Ser. No. 63/424,124, filed on Nov. 9, 2022, U.S. Patent Application Ser. No. 63/424,128, filed on Nov. 9, 2022, and U.S. Patent Application Ser. No. 63/424,119, filed on Nov. 9, 2022. The disclosures of the prior applications are considered part of, and are incorporated by reference in, the disclosure of this application.

### TECHNICAL FIELD

This document relates to engineered non-human animals (e.g., engineered non-human animals having expanded variable heavy chain segments, engineered non-human animals for producing antibody-like molecules, engineered non-human animals for producing antibody-like molecules containing fibronectin sequences, and engineered non-human animals for producing antibodies with improved properties) as well as (1) methods and materials for producing antibodies (e.g., single domain antibody (sdAbs) and/or heavy chain only antibodies) having a chimeric heavy chain, (2) methods and materials involved in producing antibody-like molecules. (3) methods and materials involved in producing antibodies (e.g., single domain antibody (sdAbs) and/or heavy chain only antibodies) having a human JH domain engineered to lack all or at least one tryptophan amino acid residue(s), and (4) methods and materials involved in producing antibodies (e.g., single domain antibody (sdAbs) and/or heavy chain only antibodies) having one, two, three, four or more modified amino acids in the human framework region 2 (FR2). For example, genetically engineered non-human animals (e.g., genetically engineered mice) capable of producing an antibody having a chimeric heavy chain including (1) a non-human Ig heavy chain constant (CH) 2 domain and/or a non-human Ig CH3 domain (e.g., endogenous CH2 and/or CH3 domains) and (2) a heavy chain variable domain (e.g., a human heavy chain variable domain encoded by a heavy chain variable region (VH) gene segment) are provided. In another example, genetically engineered non-human animals (e.g., genetically engineered mice) having the ability to produce antibody-like molecules that include (1) a non-human Ig heavy chain constant (CH) 2 domain and/or a non-human Ig CH3 domain (e.g., endogenous Ig CH2 and/or CH3 domains) and (2) one or more TCR variable domains (e.g., one or more human TCR variable domains) are provided. In another example, genetically engineered non-human animals (e.g., genetically engineered mice) having the ability to produce antibody-like molecules that include (a) a first amino acid sequence from a fibronectin type III (FN3) polypeptide (e.g., a tenth FN3 (¹⁰FN3) polypeptide), (b) a human Ig variable D domain, (c) a second amino acid sequence from an FN3 polypeptide (e.g., a ¹⁰FN3 polypeptide), and (d) a non-human Ig heavy chain constant (CH) 2 domain and/or a non-human CH3 domain (e.g., endogenous Ig CH2 and/or CH3 domains) are provided. In another example, genetically engineered non-human animals (e.g., genetically engineered mice) having the ability to produce an antibody that includes (a) a non-human heavy chain constant (CH) 2 domain and/or a non-human CH3 domain (e.g., endogenous CH2 and/or CH3 domains) and (b) a variable region that includes a human JH domain (e.g., a human JH3 domain or a human JH4 domain) lacking all or at least one tryptophan amino acid residue(s) are provided. In another example, genetically engineered non-human animals (e.g., genetically engineered mice) having the ability to produce an antibody that includes (a) a non-human heavy chain constant (CH) 2 domain and/or a non-human CH3 domain (e.g., endogenous CH2 and/or CH3 domains) and (b) a variable region that includes a human VH domain wherein framework region 2 (FR2) contains one, two, three, four, or more amino acid modifications are provided.

This document also provides methods and materials for obtaining variants of antibodies that underwent affinity maturation in vivo (e.g., methods and materials involved in in vivo affinity maturation of antibodies). For example, this document describes the creation of chimeric non-human animals (e.g., mice) generated from an embryo having (a) a first cell having one or more genomic modifications that inhibit the production of immunoglobulins by the first cell and any cells derived from it and (b) a second cell having an IgH locus that contains an exogenous nucleic acid sequence encoding a heavy chain variable region of an antibody of interest such that the chimeric non-human animal produces heavy chain antibodies containing the heavy chain variable region of the antibody of interest in addition to one or more variants of those heavy chain antibodies that underwent in vivo affinity maturation. This document also provides methods and materials for obtaining such variants of heavy chain antibodies resulting from in vivo affinity maturation.

### BACKGROUND INFORMATION

The generation of antibodies starts with the expression of the B cell receptor (BCR) in pre-B cells through the assembly of V (variable), D (diversity), and J (joining) gene segments by VDJ recombination of the immunoglobulin heavy chain gene (IgH) to generate diverse VHs that are expressed as antibodies with heavy chain constant domains, such as an IgG constant domain.

The human IgH locus encodes more than 130 VH domains. Aside from the functional VH domains, most VH domains are either open reading frames or pseudogenes in various stages of degradation.

Similar to immunoglobulin-producing B-cells, T-cells can generate enormous diversity in their T-cell receptors (TCRs). A TCR is a membrane-bound heterodimeric protein consisting of pairs of either α and β subunits or γ and δ subunits. A TCR can associate with CD3 proteins and can recognize specific peptides presented within the MHC complex (pMHC) via the variable domains of either αβ or γδ. Analogous to VDJ recombination seen in the generation of the heavy chains and light chains of B-cell antibodies, the formation of distinct αβ and γδ TCRs is driven by RAG1/2-mediated recombination of discrete V, D, and J gene segments (VDJ recombination) to assemble the β or δ chains, or by the recombination of V and J gene segments to assemble the α or γ chains.

Disulfide bonds present in antibodies create considerable challenges for adapting antibodies to target antigens inside cells due to the reducing intracellular environment, which promotes the cleavage of disulfide-bonds and can potentially disrupt the antibody structure.

Fibronectin, a member of the immunoglobulin superfamily (IgSF), is a glycoprotein that consists of two nearly identical polypeptides. Fibronectin is a ubiquitous component of the extracellular matrix (ECM) and interacts with other ECM proteins including integrins, collagen, fibrin, and proteoglycans. Fibronectin has three types of modules, namely type I, II, and III. While type I and II modules possess intra-chain disulfide bonds, the type III module lacks these bonds, which renders it stable under redox conditions. A single fibronectin protomer may contain all three types of modules, along with different repeating modular units. The tenth type III module of fibronectin (¹⁰FN3) has been utilized as a framework on a phage display platform to introduce a diverse number of mutations into their ligand-binding loop regions. ¹⁰FN3 variants with measurable affinity for ubiquitin have been isolated (Koide et al., J. Mol. Biol., 284:1141-1151 (1998)). See, also, Gebauer et al., Ann. Rev. Pharmacol. Toxicol., 60:391-415 (2020)).

In addition to disulfide bonds, noncovalent interactions, such as hydrophobic forces between heavy and light chains in conventional antibodies, contribute to the structural stability of immunoglobulins. However, amino acids that normally promote interaction between the heavy and light chains can adversely affect solubility and thermostability of heavy chain only antibodies (HcAbs).

Heavy chain-only antibodies (HcAbs) produced in camelids often exhibit excellent solubility and thermostability. These features are attributed to the evolutionary selection of amino acid sequences that enable the heavy chain to function without pairing with a light chain. For instance, in conventional antibodies, the FR2 of the variable region of the heavy chain can interact with the light chain.

Therapeutic antibodies (TAbs) have proven successful for treating an increasing number of diseases, from inflammation-related conditions to a variety of cancers. Regulatory approval of therapeutic antibodies as biologics for human medications must undergo costly clinical trials and attain stringent criteria. These TAbs, particularly those derived from in vitro platforms, however, could benefit from further modifications to improve their epitope engagement or redirected binding to other epitopes of their ligands (Tian et al., Proc. Natl. Acad. Sci. USA. 118(10):e2025596118 (2021)).

In vitro affinity maturation using phage and yeast display libraries has several drawbacks. For instance, improved affinity binding of candidate antibodies for their target antigens typically requires multiple rounds of library panning and extensive manual intervention to hypothesize which critical residues must be modified in order to improve antigen binding without compromising antibody stability.

### SUMMARY

This document relates to non-human animals (e.g., mice) that produce (e.g., are designed to produce) antibodies (e.g., sdAbs, also referred to as nanobodies, and/or heavy chain only antibodies) having a chimeric heavy chain as well as methods of making and using such engineered non-human animals. For example, this document provides genetically engineered non-human animals (e.g., genetically engineered mice) that can be designed to produce chimeric antibody heavy chains that can be used to generate a sdAb having a chimeric heavy chain and/or heavy chain only antibodies. In some cases, the non-human animals (e.g., genetically engineered mice) provided herein can, when exposed to one or more antigens, produce antibodies (e.g., sdAbs and/or heavy chain only antibodies) having a chimeric heavy chain that includes (1) a non-human Ig CH2 domain and/or a non-human Ig CH3 domain (and optionally a non-human Ig hinge) and (2) a VH domain (e.g., a human VH domain). For example, the non-human animals (e.g., genetically engineered mice) provided herein can, when exposed to one or more antigens, produce antibodies (e.g., sdAbs and/or heavy chain only antibodies) having one or two chimeric heavy chains that include (1) a non-human CH2 encoded by a nucleic acid endogenous to the non-human animal and/or a non-human CH3 encoded by a nucleic acid endogenous to the non-human animal and (2) a VH domain (e.g., a human VH domain) encoded by a nucleic acid exogenous to the non-human animal. In another example, the non-human animals (e.g., genetically engineered mice) provided herein can, when exposed to one or more antigens, produce antibodies (e.g., sdAbs and/or heavy chain only antibodies) having one or two chimeric heavy chains that (1) include a non-human CH2 domain and/or a non-human CH3 domain (and optionally a non-human Ig hinge), (2) include a VH domain (e.g., a human VH domain), and (3) lack an endogenous CH1 domain. In another example, the non-human animals (e.g., genetically engineered mice) provided herein can, when exposed to one or more antigens, produce antibodies (e.g., sdAbs and/or heavy chain only antibodies) that (1) have one or two chimeric heavy chains that (1a) include a non-human CH2 domain and/or a non-human CH3 domain (and optionally a non-human Ig hinge) and (1b) include a VH domain (e.g., a human VH domain), and (2) lack endogenous light chains. In some cases, a genetically engineered non-human animal can be a genetically engineered mouse that can, when exposed to one or more antigens, produce (e.g., be designed to produce) antibodies (e.g., sdAbs and/or heavy chain only antibodies) having a mouse/human chimeric heavy chain that includes a mouse CH2 domain and/or a mouse CH3 domain (and optionally a non-human Ig hinge) and that includes a VH domain (e.g., a human VH domain) such as a human VH domain described herein.

As described herein, this document provides methods and materials for producing antibodies (e.g., sdAbs and/or heavy chain only antibodies) having one or two chimeric heavy chains that include (1) a non-human CH2 domain and/or a non-human CH3 domain (and optionally a non-human Ig hinge) and (2) a VH domain (e.g., a human VH domain). For example, a non-human animal (e.g., a mouse) can be engineered to have a genome where one or both endogenous IgH alleles include (1) an endogenous nucleic acid encoding a CH2 domain and/or an endogenous nucleic acid encoding a CH3, and (2) one or more exogenous Ig VH gene segments that encode an amino acid sequence selected from the group consisting of an IGHV1-8*01 encoded amino acid sequence (SEQ ID NO: 74), an IGHV3-9*01 encoded amino acid sequence (SEQ ID NO: 75), an IGHV4-31*03 encoded amino acid sequence (SEQ ID NO: 76), an IGHV4-30-4*01 encoded amino acid sequence (SEQ ID NO: 77), an IGHV4-38-2*02 encoded amino acid sequence (SEQ ID NO: 78), an IGHV3-43D*04 encoded amino acid sequence (SEQ ID NO: 79), an IGHV3-35*02 encoded amino acid sequence (SEQ ID NO: 80), an IGHV3-62*04 encoded amino acid sequence (SEQ ID NO: 81), an IGHV3-16*02 encoded amino acid sequence (SEQ ID NO: 82), an IGHV3-38*02 encoded amino acid sequence (SEQ ID NO: 83), an IGHV3-38-3*01 encoded amino acid sequence (SEQ ID NO: 84), an IGHV1-38-4*01 encoded amino acid sequence (SEQ ID NO: 85), an IGHV8-51-1*02 encoded amino acid sequence (SEQ ID NO: 86), and an IGHV7-81*01 encoded amino acid sequence (SEQ ID NO: 87), such that the non-human (e.g., mouse) can, when exposed to one or more antigens, produce an antibody having a chimeric heavy chain that includes (1) a non-human CH2 domain and/or a non-human CH3 and (2) a VH domain (e.g., a human VH domain). Also as described herein, non-human animals (e.g., mice) provided herein (e.g., non-human animals that can, when exposed to one or more antigens, produce an antibody having one or two chimeric heavy chains that include (1) a non-human CH2 domain and/or a non-human CH3 and (2) a VH domain such as a VH domain set forth in any one of SEQ ID NOs: 74-87) can be used to expand the immunoglobulin repertoire produced in vivo by producing antibodies having expanded VH diversity.

This document also relates to non-human animals (e.g., mice) that produce (e.g., are designed to produce) antibody-like molecules as well as the methods for making and using such engineered non-human animals. For example, this document provides genetically engineered non-human animals (e.g., genetically engineered mice) that can be designed to produce antibody-like molecules that can be used to express antibody-like molecules based on the TCR (e.g., TCRB) framework. In some cases, non-human animals (e.g., genetically engineered mice) provided herein can, when exposed to one or more antigens, produce an antibody-like molecule that includes (1) a non-human Ig CH2 domain and/or a non-human Ig CH3 domain (e.g., endogenous Ig CH2 and/or CH3 domains) (and optionally a non-human Ig hinge) and (2) a TCR variable domain (e.g., a human TCR variable domain). For example, the non-human animals (e.g., genetically engineered mice) provided herein can, when exposed to one or more antigens, produce an antibody-like molecule that includes (1) a non-human Ig CH2 encoded by a nucleic acid endogenous to the non-human animal and/or a non-human Ig CH3 encoded by a nucleic acid endogenous to the non-human animal (and optionally a non-human Ig hinge encoded by a nucleic acid endogenous to the non-human animal) and (2) a TCR variable domain (e.g., a human TCR variable domain) encoded by a nucleic acid exogenous to the non-human animal. In another example, non-human animals (e.g., genetically engineered mice) provided herein can, when exposed to one or more antigens, produce an antibody-like molecule that (1) includes a non-human CH2 domain and/or a non-human CH3 domain (e.g., endogenous CH2 and/or CH3 domains), and optionally a non-human Ig hinge, (2) includes a TCR variable domain (e.g., a human TCR variable domain), and (3) lacks an endogenous CH1 domain. In another example, non-human animals (e.g., genetically engineered mice) provided herein can, when exposed to one or more antigens, produce an antibody-like molecule that (1) includes a non-human CH2 domain and/or a non-human CH3 domain (e.g., endogenous CH2 and/or CH3 domains), and optionally a non-human Ig hinge (2) includes a TCR variable domain (e.g., a human TCR variable domain), and (3) lacks endogenous light chains. In some cases, a genetically engineered non-human animal can be a genetically engineered mouse that can, when exposed to one or more antigens, produce (e.g., be designed to produce) a mouse/human chimeric antibody-like molecule that includes (1) a mouse CH2 domain and/or a mouse CH3 domain, and optionally a non-human Ig hinge, and (2) a human TCR variable domain such as a TCR variable domain described herein.

As described herein, this document provides methods and materials for producing antibody-like molecules that include (1) a non-human CH2 domain and/or a non-human CH3 domain (e.g., endogenous CH2 and/or CH3 domains), and optionally a non-human Ig hinge, and (2) a TCR variable domain (e.g., a human TCR variable domain). For example, a non-human animal (e.g., a mouse) can be engineered to have a genome where one or both endogenous IgH alleles include (1) an endogenous nucleic acid encoding a CH2 domain and/or an endogenous nucleic acid encoding a CH3, and optionally a non-human Ig hinge, and (2) exogenous nucleic acid encoding one or more TCR variable domains such as nucleic acid including one or more TCR V gene segments (e.g., TRBV20-1. TRBV23-1, TRBV24-1. TRBV25-1, TRBV27, TRBV28, and TRBV29-1), such that the non-human animal (e.g., mouse) can, when exposed to one or more antigens, produce an antibody-like molecule that includes (1) a non-human CH2 domain and/or a non-human CH3 domain (e.g., endogenous CH2 and/or CH3 domains), and optionally a non-human Ig hinge, and (2) a TCR variable domain (e.g., a human TCR variable domain). In another example, a non-human animal (e.g., a mouse) can be engineered to have a genome where one or both endogenous IgH alleles include (1) an endogenous nucleic acid encoding a CH2 domain and/or an endogenous nucleic acid encoding a CH3, and optionally a non-human Ig hinge, and (2) exogenous nucleic acid encoding one or more TCR variable domains such as nucleic acid including seven or more V gene segments (e.g., TRBV20-1, TRBV23-1, TRBV24-1. TRBV25-1, TRBV27, TRBV28, and TRBV29-1) and optionally one or more TCR D gene segments (e.g., TRBD1) and optionally one or more TCR J gene segments (e.g., TRBJ1-1, TRBJ1-2. TRBJ1-3, TRBJ1-4. TRBJ1-5, and/or TRBJ1-6), such that the non-human (e.g., mouse) can, when exposed to one or more antigens, produce an antibody-like molecule that includes (1) a non-human CH2 domain and/or a non-human CH3 domain (e.g., endogenous CH2 and/or CH3 domains), and optionally a non-human Ig hinge, and (2) a TCR variable domain (e.g., a human TCR variable domain). Also as described herein, non-human animals (e.g., mice) provided herein (e.g., non-human animals that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can be used to expand the immune repertoire of binding molecules by enabling the non-human animals to produce antibody-like molecules based on a TCR (e.g., a TCRB) framework.

This document also relates to non-human animals (e.g., mice) that produce (e.g., are designed to produce) antibody-like molecules that include (a) a first amino acid sequence of a FN3 polypeptide (e.g., a ¹⁰FN3 polypeptide), (b) a human Ig variable D domain. (c) a second amino acid sequence of a FN3 polypeptide (e.g., a ¹⁰FN3 polypeptide), and (d) a non-human Ig heavy chain CH2 domain and/or a non-human CH3 domain (e.g., endogenous Ig CH2 and/or CH3 domains), and optionally a non-human Ig hinge, as well as methods for creating and utilizing such engineered non-human animals. For example, this document provides genetically engineered non-human animals (e.g., genetically engineered mice) that can be designed to produce antibody-like molecules that include (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig heavy chain CH2 domain and/or a non-human CH3 domain (e.g., endogenous Ig CH2 and/or CH3 domains), and optionally a non-human Ig hinge. In some cases, non-human animals (e.g., genetically engineered mice) provided herein can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous Ig CH2 and/or CH3 domains), and optionally a non-human Ig hinge. For example, non-human animals (e.g., genetically engineered mice) provided herein can, when exposed to one or more antigens, produce an antibody-like molecule that includes (a) a first amino acid sequence of a human FN3 polypeptide (e.g., a human ¹⁰FN3 polypeptide), (b) a human Ig variable D domain, (c) a second amino acid sequence of a human FN3 polypeptide (e.g., a human ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge, encoded by nucleic acids endogenous to the non-human animal. In another example, non-human animals (e.g., genetically engineered mice) provided herein can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), (d) a non-human Ig CH2 domain and/or a non-human Ig CH3 domain (e.g., endogenous IgG CH2 and/or IgG CH3 domains), and optionally a non-human Ig hinge, and (e) that lack an endogenous CH1 domain. In another example, non-human animals (e.g., genetically engineered mice) provided herein can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), (d) a non-human Ig CH2 domain and/or a non-human Ig CH3 domain (e.g., endogenous IgG CH2 and/or IgG CH3 domains), and optionally a non-human Ig hinge, and (e) that lack endogenous Ig light chains. In some cases, a genetically engineered non-human animal can be a genetically engineered mouse that can, when exposed to one or more antigens, produce (e.g., be designed to produce) a mouse/human chimeric antibody-like molecule that includes (a) a first amino acid sequence of a human FN3 polypeptide (e.g., a human ¹⁰FN3 polypeptide), (b) a human Ig variable D domain. (c) a second amino acid sequence of a human FN3 polypeptide (e.g., a human ¹⁰FN3 polypeptide), and (d) an endogenous mouse Ig CH2 domain and/or an endogenous mouse Ig CH3 domain (e.g., endogenous IgG CH2 and/or IgG CH3 domains), and optionally a non-human Ig hinge.

As described herein, this document provides methods and materials for producing an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge. For example, a non-human animal (e.g., a mouse) can be engineered to have a genome in which one or both endogenous IgH alleles include (a) an exogenous nucleic acid encoding a first amino acid sequence of a FN3 polypeptide (e.g., a ¹⁰FN3 polypeptide), (b) an exogenous nucleic acid encoding one or more human Ig variable D domains, (c) an exogenous nucleic acid encoding a second amino acid sequence of a FN3 polypeptide (e.g., a ¹⁰FN3 polypeptide), and (d) an endogenous nucleic acid encoding a non-human Ig CH2 domain and/or a non-human Ig CH3 domain (e.g., endogenous IgG CH2 and/or IgG CH3 domains), and optionally a non-human Ig hinge, such that the non-human animal (e.g., mouse) can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge. Antibody-like molecules produced by a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains) and optionally a non-human Ig hinge) can have improved solubility and/or stability (e.g., compared to antibodies produced by non-human animals that are not modified as described herein).

This document also relates to non-human animals (e.g., mice) that produce (e.g., are designed to produce) antibodies (e.g., sdAbs, also referred to as nanobodies, and/or heavy chain only antibodies) having a chimeric heavy chain, as well as methods of making and using such engineered non-human animals. For example, this document provides genetically engineered non-human animals (e.g., genetically engineered mice) that can be designed to produce chimeric antibody heavy chains that can be used to generate a sdAb having a chimeric heavy chain and/or used to generate heavy chain only antibodies. In some cases, non-human animals (e.g., genetically engineered mice) provided herein can, when exposed to one or more antigens, produce antibodies (e.g., sdAbs and/or heavy chain only antibodies) having a chimeric heavy chain that includes (a) a non-human CH2 domain and/or a non-human CH3 domain (and optionally a non-human Ig hinge) and (b) a variable region that includes a human JH domain (e.g., a human JH3 domain or a human JH4 domain) lacking all or at least one tryptophan amino acid residue(s). The human JH domains, human JH1, JH2, JH3, JH4, JH5, and JH6 domains, encoded by human JH gene segments, human IGHJ1, IGHJ2, IGHJ3, IGHJ4. IGHJ5, and IGHJ6 gene segments, respectively, each typically contain at least one tryptophan amino acid residue. As described herein, the genome of a non-human animal can be engineered to produce antibodies that include a variable region that includes a human JH domain (e.g., a human JH3 domain or a human JH4 domain) lacking all tryptophan amino acid residues or at least one tryptophan amino acid residue such as the one that immediately precedes the invariant residue glycine (e.g., the tryptophan amino acid residue at Kabat position 103: Kabat et al., Sequences of immunoglobulin chains; tabulation and analysis of amino acid sequences of precursors. V-regions. C-regions. J-chain and 2-microglobulins, National Institute of Health (1979); and Bélanger et al., Prot. Eng. Des. Select., 34: gzab012 (2021)). Such antibodies can possess improved biochemical properties, such as improved stability, compared to similar antibodies that contain a human JH domain which includes its naturally occurring tryptophan amino acid residue(s) at this position.

In some cases, this document describes non-human animals (e.g., genetically engineered mice) that can, when exposed to one or more antigens, produce antibodies (e.g., sdAbs and/or heavy chain only antibodies) having one or two chimeric heavy chains that include (a) a non-human CH2 encoded by a nucleic acid endogenous to the non-human animal and/or a non-human CH3 encoded by a nucleic acid endogenous to the non-human animal and (b) a variable region that (i) includes a human JH domain (e.g., a human JH3 domain or a human JH4 domain) lacking all or at least one tryptophan amino acid residue(s) and (ii) is encoded by a nucleic acid exogenous to the non-human animal. In another example, non-human animals (e.g., genetically engineered mice) provided herein can, when exposed to one or more antigens, produce antibodies (e.g., sdAbs and/or heavy chain only antibodies) having one or two chimeric heavy chains that include (a) a non-human CH2 domain and/or a non-human CH3 domain (and optionally a non-human Ig hinge). (b) a variable region that includes a human JH domain (e.g., a human JH3 domain or a human JH4 domain) lacking all or at least one tryptophan amino acid residue(s), and (c) lack an endogenous Ig CH1 domain. In another example, non-human animals (e.g., genetically engineered mice) provided herein can, when exposed to one or more antigens, produce antibodies (e.g., sdAbs and/or heavy chain only antibodies) that have one or two chimeric heavy chains that include (a) a non-human CH2 domain and/or a non-human CH3 domain (and optionally a non-human Ig hinge), (b) a variable region that includes a human JH domain (e.g., a human JH3 domain or a human JH4 domain) lacking all or at least one tryptophan amino acid residue(s), and (c) lack endogenous light chains. In some cases, a genetically engineered non-human animal can be a genetically engineered mouse that can, when exposed to one or more antigens, produce (e.g., be designed to produce) antibodies (e.g., sdAbs and/or heavy chain only antibodies) having a mouse/human chimeric heavy chain that includes (a) a mouse CH2 domain and/or a mouse CH3 domain and (b) includes a variable region that includes a human JH domain (e.g., a human JH3 domain or a human JH4 domain) lacking all or at least one tryptophan amino acid residue(s).

As described herein, this document provides methods and materials for producing antibodies (e.g., sdAbs and/or heavy chain only antibodies) having one or two chimeric heavy chains that include (a) a non-human CH2 domain and/or a non-human CH3 domain (and optionally a non-human Ig hinge) and (b) a variable region that includes a human JH domain (e.g., a human JH3 domain or a human JH4 domain) lacking all or at least one tryptophan amino acid residue(s). For example, a non-human animal (e.g., a mouse) can be engineered to have a genome where one or both endogenous IgH alleles include (a) an endogenous nucleic acid encoding a CH2 domain and/or an endogenous nucleic acid encoding a CH3, and (b) exogenous nucleic acid encoding one or more human JH domains (e.g., human JH3 and/or JH4 domains) lacking all or at least one tryptophan amino acid residue(s) such as exogenous nucleic acid that encodes one or more human JH domains modified as set forth in SEQ ID NOs:D18 and D19 where the X represents an amino acid substitution from tryptophan to any other amino acid that is not tryptophan. Also as described herein, non-human animals (e.g., mice) provided herein (e.g., non-human animals that can, when exposed to one or more antigens, produce an antibody having one or two chimeric heavy chains that include (a) a non-human CH2 domain and/or a non-human CH3 (and optionally a non-human Ig hinge) and (b) a variable region that includes a human JH domain (e.g., a human JH3 domain or a human JH4 domain) lacking all or at least one tryptophan amino acid residue(s) can be used for in vivo production of antibodies having improved biochemical properties (e.g., improved stability).

This document also relates to non-human animals (e.g., mice) that produce (e.g., are designed to produce) antibodies (e.g., sdAbs, also referred to as nanobodies, and/or heavy chain only antibodies) having a chimeric heavy chain as well as methods of making and using such engineered non-human animals. For example, this document provides genetically engineered non-human animals (e.g., genetically engineered mice) that can be designed to produce chimeric antibody heavy chains that can be used to generate a sdAb having a chimeric heavy chain and/or chimeric heavy chain only antibodies. In some cases, non-human animals (e.g., genetically engineered mice) provided herein can, when exposed to one or more antigens, produce antibodies (e.g., sdAbs and/or heavy chain only antibodies) having a chimeric heavy chain that includes (a) a non-human CH2 domain and/or a non-human CH3 domain (and optionally a non-human Ig hinge) and (b) a variable region that includes a human VH domain having a FR2 containing one, two, three, four, or more amino acid modifications (e.g., one, two, three, four, or more amino acid substitutions). For example, non-human animals (e.g., genetically engineered mice) provided herein can, when exposed to one or more antigens, produce antibodies (e.g., sdAbs and/or heavy chain only antibodies) having one or two chimeric heavy chains that include (a) a non-human CH2 encoded by a nucleic acid endogenous to the non-human animal and/or a non-human CH3 encoded by a nucleic acid endogenous to the non-human animal and (b) a variable region that includes a human VH domain having a FR2 containing one, two, three, four, or more amino acid modifications (e.g., one, two, three, four, or more amino acid substitutions) encoded by a nucleic acid exogenous to the non-human animal. In another example, non-human animals (e.g., genetically engineered mice) provided herein can, when exposed to one or more antigens, produce antibodies (e.g., sdAbs and/or heavy chain only antibodies) having one or two chimeric heavy chains that includes (a) a non-human CH2 domain and/or a non-human CH3 domain (and optionally a non-human Ig hinge), (b) a variable region that includes a human VH domain having a FR2 containing one, two, three, four, or more amino acid modifications (e.g., one, two, three, four, or more amino acid substitutions), and (c) lack an endogenous Ig CH1 domain. In another example, non-human animals (e.g., genetically engineered mice) provided herein can, when exposed to one or more antigens, produce antibodies (e.g., sdAbs and/or heavy chain only antibodies) that have one or two chimeric heavy chains that includes (a) a non-human CH2 domain and/or a non-human CH3 domain (and optionally a non-human Ig hinge), (b) a variable region that includes a human VH domain having a FR2 containing one, two, three, four, or more amino acid modifications (e.g., one, two, three, four, or more amino acid substitutions), and (c) lack endogenous light chains. In some cases, a genetically engineered non-human animal can be a genetically engineered mouse that can, when exposed to one or more antigens, produce (e.g., be designed to produce) antibodies (e.g., sdAbs and/or heavy chain only antibodies) having a mouse/human chimeric heavy chain that includes (a) a mouse CH2 domain and/or a mouse CH3 domain (and optionally a non-human Ig hinge) and t(b) a variable region that includes a human VH domain having a FR2 containing one, two, three, four, or more amino acid modifications (e.g., one, two, three, four, or more amino acid substitutions) as described herein.

As described herein, this document provides methods and materials for producing antibodies (e.g., sdAbs and/or heavy chain only antibodies) having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications (e.g., one, two, three, four, or more amino acid substitutions). For example, a non-human animal (e.g., a mouse) can be engineered to have a genome where one or both endogenous IgH alleles include (a) an endogenous nucleic acid encoding a CH2 domain and/or an endogenous nucleic acid encoding a CH3, and (b) exogenous nucleic acid encoding one or more human VH domains having FR2 containing one, two, three, four, or more amino acid modifications (e.g., one, two, three, four, or more amino acid substitutions) such as exogenous nucleic acid that encodes an IGHV3-11 VH domain modified to have the amino acid sequence set forth in SEQ ID NO: 119, an IGHV3-21 VH domain modified to have the amino acid sequence set forth in SEQ ID NO: 120, an IGHV3-23 VH domain modified to have the amino acid sequence set forth in SEQ ID NO: 121, an IGHV4-39 VH domain modified to have the amino acid sequence set forth in SEQ ID NO: 122, and/or an IGHV3-74 VH domain modified to have the amino acid sequence set forth in SEQ ID NO: 123. Also as described herein, non-human animals (e.g., mice or genetically engineered mice) provided herein (e.g., can, when exposed to one or more antigens, produce an antibody having one or two chimeric heavy chains that include (a) a non-human CH2 domain and/or a non-human CH3 (and optionally a non-human Ig hinge) and (b) a variable region that includes a human VH domain having a FR2 containing one, two, three, four, or more amino acid modifications (e.g., one, two, three, four, or more amino acid substitutions) be used for in vivo production of antibodies having improved biochemical properties (e.g., improved solubility).

This document also provides methods and materials involved in the in vivo affinity maturation of antibodies of interest. For example, this document provides chimeric non-human animals (e.g., mice) generated from an embryo having (a) a first cell having one or more genomic modifications that prevent the first cell (and cells derived from the first cell) from producing immunoglobulins and (b) a second cell having an IgH locus that includes an exogenous nucleic acid sequence encoding a heavy chain variable region of an antibody of interest such that the chimeric non-human animal produces heavy chain antibodies containing the heavy chain variable region of the antibody of interest in addition to one or more variants of those heavy chain antibodies that underwent in vivo affinity maturation.

As described herein, a chimeric non-human animal (e.g., mouse) can be generated from an embryo having at least two genetically different cells. The first cell can be that of a recipient embryo or blastocyst and can have one or more genomic modifications that prevent the first cell (and cells derived from the first cell) from producing immunoglobulins. Examples of genomic modifications that can prevent cells from producing immunoglobulins include, without limitation. Prkdc^{scid}, recombination activating gene 1 (Rag1) inactivation, Rag1 knock outs, Rag1^{tm1Mom}, recombination activating gene 2 (Rag2) inactivation, Rag2 knock outs. Rag2^{tm1}, IgH inactivation, IgH knock outs, and IgH VH/DH/JH knock outs.

The second cell can be a stem cell (e.g., an embryonic stem (ES) cell) having an IgH locus that includes an exogenous nucleic acid sequence encoding a heavy chain variable region of an antibody of interest. Examples of heavy chain variable regions of an antibody of interest include, without limitation, nanobodies themselves and the variable region of heavy chain only antibodies. In some cases, the heavy chain variable region of a full antibody of interest, the heavy chain variable region of a humanized antibody of interest, or the heavy chain variable region of a single-chain variable fragment (scFv) of interest can be used as a heavy chain variable region of an antibody of interest as described herein. In some cases, the heavy chain variable region of an antibody of interest described herein, whether a nanobody of interest itself or the heavy chain variable region of a heavy chain only antibody of interest, a full antibody of interest, a humanized antibody of interest, or a scFv of interest, can be referred to herein as the input nanobody domain (Nbx) for convenience.

In some cases, the IgH locus of the second cell can be designed to (i) include endogenous hinge, constant CH2, and/or CH3 gene segments of an Ig class (e.g., IgG) and (ii) exclude the endogenous constant CH1 gene segment of that Ig class and/or a regulatory element controlling expression of that endogenous constant CH1 gene segment of that Ig class such that the input nanobody domain is expressed by B cells derived from the second cell as the variable region of a heavy chain only antibody (e.g., a Nbx-IgΔCH1 such as a Nbx-IgG1-ΔCH1). See, e.g., FIG. 8. In some cases, the IgH locus of the second cell can be designed to exclude endogenous VH, DH, and/or JH gene segments. See. e.g., FIG. 8.

In some cases, a chimeric non-human animal (e.g., mouse) described herein is capable of expressing heavy chain only antibodies that include the input nanobody domain, while lacking the ability to express immunoglobulins endogenous to that non-human animal. For example, a chimeric mouse as described herein can express heavy chain only antibodies that include the input nanobody domain (e.g., Nbx-IgG1ΔCH1 heavy chain only antibodies), while lacking the ability to express endogenous mouse immunoglobulins.

This document also provides methods and materials for obtaining variants of heavy chain only antibodies that underwent in vivo affinity maturation within a chimeric non-human animal (e.g., mouse), as described herein. For example, a composition containing an antigen recognized by an antibody of interest can be administered to a chimeric non-human animal (e.g., mouse) described herein that is designed to express heavy chain only antibodies that include an input nanobody domain targeting that antigen, thereby inducing in vivo affinity maturation within the chimeric non-human animal and one or more variants of the expressed heavy chain only antibodies that include the input nanobody domain are produced. Such produced variants, or the B cells that produce such variants, can be obtained from the chimeric non-human animal, thereby providing enhanced versions of the antibody of interest.

In general, one aspect of this document features a non-human animal, wherein the genome of the non-human animal comprises an immunoglobulin heavy chain (IgH) allele, wherein the IgH allele comprises an endogenous nucleic acid encoding a CH2 or CH3 domain of an IgG subclass, wherein the IgH allele lacks nucleic acid encoding at least a portion of an endogenous CH1 domain of the IgG subclass or at least a portion of an endogenous regulatory element that drives expression of the endogenous CH1 domain, wherein the IgH allele comprises one or more Ig VH gene segments selected from the group consisting of an Ig VH gene segment that encodes the amino acid sequence set forth in SEQ ID NO:74, an Ig VH gene segment that encodes the amino acid sequence set forth in SEQ ID NO:75, an Ig VH gene segment that encodes the amino acid sequence set forth in SEQ ID NO:76, an Ig VH gene segment that encodes the amino acid sequence set forth in SEQ ID NO:77, an Ig VH gene segment that encodes the amino acid sequence set forth in SEQ ID NO:78, an Ig VH gene segment that encodes the amino acid sequence set forth in SEQ ID NO:79, an Ig VH gene segment that encodes the amino acid sequence set forth in SEQ ID NO:80, an Ig VH gene segment that encodes the amino acid sequence set forth in SEQ ID NO:81, an Ig VH gene segment that encodes the amino acid sequence set forth in SEQ ID NO:82, an Ig VH gene segment that encodes the amino acid sequence set forth in SEQ ID NO:83, an Ig VH gene segment that encodes the amino acid sequence set forth in SEQ ID NO:84, an Ig VH gene segment that encodes the amino acid sequence set forth in SEQ ID NO:85, an Ig VH gene segment that encodes the amino acid sequence set forth in SEQ ID NO:86, and an Ig VH gene segment that encodes the amino acid sequence set forth in SEQ ID NO:87, wherein the non-human animal is capable of producing an antibody, wherein the antibody comprises the CH2 or CH3 domain of the IgG subclass and a variable domain comprising the amino acid sequence encoded by one of the Ig VH gene segments and lacks the CH1 domain. The IgH allele can lack endogenous nucleic acid encoding at least a portion of an IgM constant domain, endogenous nucleic acid encoding at least a portion of an IgD constant domain, endogenous nucleic acid encoding at least a portion of an IgE constant domain, or endogenous nucleic acid encoding at least a portion of an IgA constant domain. The IgH allele of the non-human animal can include endogenous nucleic acid encoding the CH2 domain and the CH3 domain of the IgG subclass. The IgH allele of the non-human animal comprises endogenous nucleic acid encoding a hinge domain of the IgG subclass. The IgG subclass can be an IgG2 subclass. The IgG subclass can be an IgG2a. IgG2b. IgG2c, IgG3, or IgG4 subclass. The IgG subclass can be an IgG1 subclass. The IgH allele can lack endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain. The IgH allele can lack endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain. The IgH allele can lack endogenous nucleic acid encoding each of the IgG2 constant domains, endogenous nucleic acid encoding each of the IgG3 constant domains, or endogenous nucleic acid encoding each of the IgG4 constant domains. The IgH allele can lack endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains. The non IgH allele can lack endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain. The IgH allele can lack endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains. The IgH allele can lack endogenous nucleic acid encoding each of the IgM constant domains. The IgH allele can lack endogenous nucleic acid encoding each of the IgD constant domains. The IgH allele can lack endogenous nucleic acid encoding each of the IgE constant domains. The IgH allele can lack endogenous nucleic acid encoding IgA CH1 and CH2 constant domains. The IgH allele can lack nucleic acid encoding the endogenous CH1 domain. The IgH allele can lack the endogenous regulatory element. The IgH allele can include an endogenous Eμ. The first nucleic acid sequence encoding a full length CH2 domain downstream of the endogenous Eμ is nucleic acid encoding an IgG CH2 domain. The first nucleic acid sequence encoding a full length CH2 domain downstream of the endogenous Eμ can be nucleic acid encoding an IgG1 CH2 domain. The IgH allele can include an endogenous Sμ, an endogenous Iμ promoter, an endogenous Iμ exon, or a combination thereof. The first nucleic acid sequence encoding a full length CH2 domain downstream of the endogenous Sμ, the endogenous Iμ promoter, or the endogenous Iμ exon is nucleic acid encoding an IgG CH2 domain. The first nucleic acid sequence encoding a full length CH2 domain downstream of the endogenous Sμ, the endogenous Iμ promoter, or the endogenous Iμ exon is nucleic acid encoding an IgG1 CH2 domain. The IgH allele can include an endogenous 3′γ1E. The IgH allele can lack endogenous nucleic acid encoding a full length CH2 domain downstream of the endogenous 3′γ1E. The IgH allele can include an endogenous S′hsR1. The first nucleic acid sequence encoding a full length CH2 domain upstream of the endogenous 5′hsR1 can be nucleic acid encoding an IgG CH2 domain. The first nucleic acid sequence encoding a full length CH2 domain upstream of the endogenous 5′hsR1 can be nucleic acid encoding an IgG1 CH2 domain. The IgH allele can include an endogenous 3′RR. The first nucleic acid sequence encoding a full length CH2 domain upstream of the endogenous 3′RR can be nucleic acid encoding an IgG CH2 domain. The first nucleic acid sequence encoding a full length CH2 domain upstream of the endogenous 3′RR can be nucleic acid encoding an IgG1 CH2 domain. The IgH allele can include an endogenous 3 CBE. The first nucleic acid sequence encoding a full length CH2 domain upstream of the endogenous 3′CBE can be nucleic acid encoding an IgG CH2 domain. The first nucleic acid sequence encoding a full length CH2 domain upstream of the endogenous 3′CBE can be nucleic acid encoding an IgG1 CH2 domain. The at least one allele of the genome can lack at least a portion of the endogenous Ig heavy chain variable region. The at least one allele of the genome can lack all the exons of the endogenous Ig heavy chain variable region. In some cases, both alleles of the genome lack all the exons of the endogenous Ig heavy chain variable region. In some cases, neither allele of the genome comprises an endogenous exon of an Ig heavy chain variable region. The IgH allele can include, in addition to the one or more Ig VH gene segments selected from the group, exogenous nucleic acid encoding one or more human Ig VH gene segments. The IgH allele can include three or more human Ig VH gene segments. The IgH allele can include 26 or more human Ig VH gene segments. The IgH allele can include 65 or more human Ig VH gene segments. The IgH allele can include 129 human Ig VH gene segments. The IgH allele can include at least 44 functional human Ig VH gene segments. The IgH allele can include at least 58 functional human Ig VH gene segments. The IgH allele can include 13 or more human Ig DH gene segments. The IgH allele can include 27 human Ig DH gene segments. The IgH allele can include three or more human Ig VH gene segments. The IgH allele comprises 9 human Ig VH gene segments. The IgH allele can include 129 or more human Ig VH gene segments, 27 or more human Ig DH gene segments, and 9 or more human Ig VH gene segments. The IgH allele can include two or more of the the Ig VH gene segments selected from the group. The non IgH allele can include three or more of the Ig VH gene segments selected from the group. The IgH allele can include four or more of the Ig VH gene segments selected from the group. The IgH allele can include five or more of the Ig VH gene segments selected from the group. The IgH allele can include six or more of the Ig VH gene segments selected from the group. The IgH allele can include seven or more of the Ig VH gene segments selected from the group. The IgH allele can include eight or more of the Ig VH gene segments selected from the group. The non IgH allele can include nine or more of the Ig VH gene segments selected from the group. The IgH allele can include ten or more of the Ig VH gene segments selected from the group. The IgH allele can include 11 or more of the Ig VH gene segments selected from the group. The IgH allele can include 12 or more of the Ig VH gene segments selected from the group. The IgH allele can include 13 or more of the Ig VH gene segments selected from the group. The IgH allele can include an Ig VH gene segment that encodes the amino acid sequence set forth in SEQ ID NO:74, an Ig VH gene segment that encodes the amino acid sequence set forth in SEQ ID NO:75, an Ig VH gene segment that encodes the amino acid sequence set forth in SEQ ID NO:76, an Ig VH gene segment that encodes the amino acid sequence set forth in SEQ ID NO:77, an Ig VH gene segment that encodes the amino acid sequence set forth in SEQ ID NO:78, an Ig VH gene segment that encodes the amino acid sequence set forth in SEQ ID NO:79, an Ig VH gene segment that encodes the amino acid sequence set forth in SEQ ID NO:80, an Ig VH gene segment that encodes the amino acid sequence set forth in SEQ ID NO:81, an Ig VH gene segment that encodes the amino acid sequence set forth in SEQ ID NO:82, an Ig VH gene segment that encodes the amino acid sequence set forth in SEQ ID NO:83, an Ig VH gene segment that encodes the amino acid sequence set forth in SEQ ID NO:84, an Ig VH gene segment that encodes the amino acid sequence set forth in SEQ ID NO:85, an Ig VH gene segment that encodes the amino acid sequence set forth in SEQ ID NO:86, and an Ig VH gene segment that encodes the amino acid sequence set forth in SEQ ID NO:87. The IgH allele can include an endogenous regulatory element for each of the one or more Ig VH gene segments. The endogenous regulatory element can be an endogenous promoter sequence of the non-human animal. The IgH allele can include an endogenous exon encoding a leader sequence for each of the one or more Ig VH gene segments. The endogenous exon can be an endogenous L1 exon of the non-human animal. The endogenous exon can be an endogenous L2 exon of the non-human animal. The IgH allele can include endogenous L1 and L2 exons encoding a leader sequence for each of the one or more Ig VH gene segments. The IgH allele can include at least one exogenous recombinase site recognition nucleic acid sequence. The at least one exogenous recombinase site recognition nucleic acid sequence can be located upstream of the endogenous nucleic acid encoding the CH2 or CH3 domain of the IgG subclass. The IgH allele can include one, two, three, four, five, six, seven, eight, nine, or ten different exogenous recombinase site recognition nucleic acid sequences. The IgH allele include at least three different exogenous recombinase site recognition nucleic acid sequences. The IgH allele can include at least five different exogenous recombinase site recognition nucleic acid sequences. Each of the different exogenous recombinase site recognition nucleic acid sequences can be located less than 2.5 Mb upstream of an endogenous Eμ. Each of the different exogenous recombinase site recognition nucleic acid sequences can be located less than 2.0 Mb, less than 1.5 Mb, less than 1.0 Mb, less than 500 kb, or less than 250 kb upstream of an endogenous Eμ. Each of the different exogenous recombinase site recognition nucleic acid sequences can be located less than 200 kb, less than 100 kb, less than 50 kb, less than 25 kb, or less than 10 kb upstream of an endogenous Eμ. The non-human animal can be a mouse or rat. The non-human animal can be a mouse.

In another aspect, this document features methods for producing a non-human animal. The methods can include, or consist essentially of, (a) introducing exogenous nucleic acid comprising one or more Ig VH gene segments selected from the group consisting of an Ig VH gene segment that encodes the amino acid sequence set forth in SEQ ID NO:74, an Ig VH gene segment that encodes the amino acid sequence set forth in SEQ ID NO:75, an Ig VH gene segment that encodes the amino acid sequence set forth in SEQ ID NO:76, an Ig VH gene segment that encodes the amino acid sequence set forth in SEQ ID NO:77, an Ig VH gene segment that encodes the amino acid sequence set forth in SEQ ID NO:78, an Ig VH gene segment that encodes the amino acid sequence set forth in SEQ ID NO:79, an Ig VH gene segment that encodes the amino acid sequence set forth in SEQ ID NO:80, an Ig VH gene segment that encodes the amino acid sequence set forth in SEQ ID NO:81, an Ig VH gene segment that encodes the amino acid sequence set forth in SEQ ID NO:82, an Ig VH gene segment that encodes the amino acid sequence set forth in SEQ ID NO:83, an Ig VH gene segment that encodes the amino acid sequence set forth in SEQ ID NO:84, an Ig VH gene segment that encodes the amino acid sequence set forth in SEQ ID NO:85, an Ig VH gene segment that encodes the amino acid sequence set forth in SEQ ID NO:86, and an Ig VH gene segment that encodes the amino acid sequence set forth in SEQ ID NO:87 into an endogenous IgH locus in a stem cell of a non-human animal: (b) implanting the stem cell into a blastocyst: (c) implanting the blastocyst into a pseudo-pregnant non-human animal to obtain a chimeric non-human animal: (d) crossing the chimeric non-human animal to a wild-type non-human animal to produce offspring: (e) screening the offspring for heterozygosity; and (f) identifying a founder non-human animal carrying the one or more Ig VH gene segments. The stem cell can be an embryonic stem cell.

In another aspect, this document features methods for producing an antibody in a non-human animal described herein. The methods can include, or consist essentially of, administering an antigen to the non-human animal, wherein one or more B cells in the non-human animal produce an antibody comprising a variable domain encoded by one of the Ig VH gene segments. The antibody can be a single domain antibody (sdAb). The antibody can include an amino acid sequence selected from SEQ ID NOs: 74-87.

In another aspect, this document features methods for obtaining a B cell that produces an antibody capable of binding to an antigen. The methods can include, or consist essentially of, (a) administering the antigen to a non-human animal of any one of claims 1-82, wherein one or more B cells in the non-human animal produce the antibody, and wherein the antibody comprises a variable domain comprising the amino acid sequence encoded by one of the Ig VH gene segments, and (b) isolating the one or more B cells from the non-human animal. The antibody can be a sdAb. The antibody can include an amino acid sequence selected from SEQ ID NOs: 74-87.

In another aspect, this document features a non-human animal, wherein the genome of the non-human animal comprises an immunoglobulin heavy chain (IgH) allele, wherein the IgH allele comprises an endogenous nucleic acid encoding a CH2 or CH3 domain of an IgG subclass, wherein the IgH allele lacks nucleic acid encoding at least a portion of an endogenous CH1 domain of the IgG subclass or at least a portion of an endogenous regulatory element that drives expression of the endogenous CH1 domain, wherein the IgH allele comprises nucleic acid encoding one or more T cell receptor (TCR) variable domains, wherein the non-human animal is capable of producing an antibody-like molecule, wherein the antibody-like molecule comprises the CH2 or CH3 domain of the IgG subclass and one of the TCR variable domains and lacks the CH1 domain. The IgH allele can lack endogenous nucleic acid encoding at least a portion of an IgM constant domain, endogenous nucleic acid encoding at least a portion of an IgD constant domain, endogenous nucleic acid encoding at least a portion of an IgE constant domain, or endogenous nucleic acid encoding at least a portion of an IgA constant domain. The IgH allele of the non-human animal can include endogenous nucleic acid encoding the CH2 domain and the CH3 domain of the IgG subclass. The IgH allele of the non-human animal can include endogenous nucleic acid encoding a hinge domain of the IgG subclass. The IgG subclass can be an IgG2 subclass. The IgG subclass can be an IgG2a, IgG2b, IgG2c, IgG3, or IgG4 subclass. The IgG subclass can be an IgG1 subclass. The IgH allele can lack endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain. The IgH allele can lack endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain. The IgH allele can lack endogenous nucleic acid encoding each of the IgG2 constant domains, endogenous nucleic acid encoding each of the IgG3 constant domains, or endogenous nucleic acid encoding each of the IgG4 constant domains. The IgH allele can lack endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains. The IgH allele can lack endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain. The IgH allele can lack endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains. The IgH allele can lack endogenous nucleic acid encoding each of the IgM constant domains. The IgH allele can lack endogenous nucleic acid encoding each of the IgD constant domains. The IgH allele can lack endogenous nucleic acid encoding each of the IgE constant domains. The IgH allele can lack endogenous nucleic acid encoding IgA CH1 and CH2 constant domains. The IgH allele can lack nucleic acid encoding the endogenous CH1 domain. The IgH allele can lack the endogenous regulatory element. The IgH allele can include an endogenous Eμ. The first nucleic acid sequence encoding a full length CH2 domain downstream of the endogenous Eμ can be nucleic acid encoding an IgG CH2 domain. The first nucleic acid sequence encoding a full length CH2 domain downstream of the endogenous Eμ can be nucleic acid encoding an IgG1 CH2 domain. The IgH allele can include an endogenous Sμ, an endogenous Iμ promoter, an endogenous Iμ exon, or a combination thereof. The first nucleic acid sequence encoding a full length CH2 domain downstream of the endogenous Sμ, the endogenous Iμ promoter, or the endogenous Iμ exon can be nucleic acid encoding an IgG CH2 domain. The first nucleic acid sequence encoding a full length CH2 domain downstream of the endogenous Sμ, the endogenous Iμ promoter, or the endogenous Iμ exon can be nucleic acid encoding an IgG1 CH2 domain. The IgH allele can include an endogenous 3′γ1E. The IgH allele can lack endogenous nucleic acid encoding a full length CH2 domain downstream of the endogenous 3′γ1E. The IgH allele can include an endogenous 5′hsR1. The first nucleic acid sequence encoding a full length CH2 domain upstream of the endogenous 5′hsR1 can be nucleic acid encoding an IgG CH2 domain. The first nucleic acid sequence encoding a full length CH2 domain upstream of the endogenous 5 hsR1 can be nucleic acid encoding an IgG1 CH2 domain. The IgH allele can include an endogenous 3′RR. The first nucleic acid sequence encoding a full length CH2 domain upstream of the endogenous 3′RR can be nucleic acid encoding an IgG CH2 domain. The non-human animal of claim 31, wherein the first nucleic acid sequence encoding a full length CH2 domain upstream of the endogenous 3′RR is nucleic acid encoding an IgG1 CH2 domain. The IgH allele can include an endogenous 3′CBE. The first nucleic acid sequence encoding a full length CH2 domain upstream of the endogenous 3 CBE can be nucleic acid encoding an IgG CH2 domain. The first nucleic acid sequence encoding a full length CH2 domain upstream of the endogenous 3′CBE can be nucleic acid encoding an IgG1 CH2 domain. The at least one allele of the genome can lack at least a portion of the endogenous Ig heavy chain variable region. The at least one allele of the genome can lack all the exons of the endogenous Ig heavy chain variable region. In some cases, both alleles of the genome lack all the exons of the endogenous Ig heavy chain variable region. In some cases, neither allele of the genome comprises an endogenous exon of an Ig heavy chain variable region. The IgH allele can include, in addition to the nucleic acid encoding one or more TCR variable domains, exogenous nucleic acid encoding one or more human Ig VH gene segments. The IgH allele can include three or more human Ig VH gene segments. The IgH allele can include 26 or more human Ig VH gene segments. The IgH allele can include 65 or more human Ig VH gene segments. The IgH allele can include 129 human Ig VH gene segments. The IgH allele can include 13 or more human Ig DH gene segments. The IgH allele can include 27 human Ig DH gene segments. The IgH allele can include three or more human Ig VH gene segments. The IgH allele can include 9 human Ig VH gene segments. The IgH allele can include 126 or more human Ig VH gene segments, 27 or more human Ig DH gene segments, and 9 or more human Ig VH gene segments. The one or more TCR variable domains can be TCR Vα domains. The one or more TCR variable domains can include five or more TCR Vα domains. The IgH allele can include nucleic acid encoding one or more TCR Jα domains. The one or more TCR variable domains can be TCR Vβ domains. The one or more TCR variable domains can include five or more TCR Vβ domains. The IgH allele can include nucleic acid encoding one or more TCR Dβ domains. The IgH allele can include nucleic acid encoding one or more TCR Jβ domains. The one or more TCR variable domains can be TCR Vγ domains. The one or more TCR variable domains can include five or more TCR Vγ domains. The IgH allele can include nucleic acid encoding one or more TCR Jγ domains. The one or more TCR variable domains can be TCR Vδ domains. The one or more TCR variable domains can include five or more TCR Vδ domains. The IgH allele can include nucleic acid encoding one or more TCR Dò domains. The IgH allele can include nucleic acid encoding one or more TCR Jβ domains. The one or more TCR variable domains can be human TCR variable domains. The one or more TCR Dβ or Do domains can be human TCR Dβ or Do domains. The one or more TCR Jα, Jβ, Jγ, or Jβ domains can be human TCR Jα, Jβ, Jγ, or Jδ domains. The IgH allele can lack nucleic acid encoding TCR D or J domains. The IgH allele can lack nucleic acid encoding TCR D and J domains. The IgH allele can include at least one exogenous recombinase site recognition nucleic acid sequence. The at least one exogenous recombinase site recognition nucleic acid sequence can be located upstream of the endogenous nucleic acid encoding the CH2 or CH3 domain of the IgG subclass. The IgH allele can include one, two, three, four, five, six, seven, eight, nine, or ten different exogenous recombinase site recognition nucleic acid sequences. The IgH allele can include at least three different exogenous recombinase site recognition nucleic acid sequences. The IgH allele can include at least five different exogenous recombinase site recognition nucleic acid sequences. The different exogenous recombinase site recognition nucleic acid sequences can be located less than 2.5 Mb upstream of an endogenous Eμ. The different exogenous recombinase site recognition nucleic acid sequences can be located less than 2.0 Mb, less than 1.5 Mb, less than 1.0 Mb, less than 500 kb, or less than 250 kb upstream of an endogenous Eμ. The different exogenous recombinase site recognition nucleic acid sequences can be located less than 200 kb, less than 100 kb, less than 50 kb, less than 25 kb, or less than 10 kb upstream of an endogenous Eμ. The non-human animal can be a mouse or rat. The non-human animal can be a mouse.

In another aspect, this document features methods for producing a non-human animal described herein. The methods can include, or consist essentially of, (a) introducing exogenous nucleic acid encoding one or more TCR variable domains into an endogenous IgH locus in a stem cell of a non-human animal: (b) implanting the stem cell into a blastocyst: (c) implanting the blastocyst into a pseudo-pregnant non-human animal to obtain a chimeric non-human animal: (d) crossing the chimeric non-human animal to a wild-type non-human animal to produce offspring: (e) screening the offspring for heterozygosity; and (f) identifying a founder non-human animal carrying the exogenous nucleic acid. The stem cell can be an embryonic stem cell.

In another aspect, this document features methods for producing an antibody-like molecule in a non-human animal described herein. The methods can include, or consist essentially of, administering an antigen to the non-human animal, wherein one or more B cells in the non-human animal produce an antibody-like molecule comprising one of the one or more TCR variable domains. The antibody-like molecule can be a single chain antibody-like molecule.

In another aspect, this document features methods for obtaining a B cell that produces an antibody-like molecule capable of binding to an antigen. The methods can include, or consist essentially of, (a) administering the antigen to a non-human animal described herein, wherein one or more B cells in the non-human animal produce the antibody-like molecule, and wherein the antibody-like molecule comprises one of the one or more TCR variable domains, and (b) isolating the one or more B cells from the non-human animal. The antibody-like molecule can be a single chain antibody-like molecule.

In another aspect, this document features a non-human animal, wherein the genome of the non-human animal comprises an immunoglobulin heavy chain (IgH) allele, wherein the IgH allele comprises an endogenous nucleic acid encoding a CH2 or CH3 domain of an IgG subclass, wherein the IgH allele lacks nucleic acid encoding at least a portion of an endogenous CH1 domain of the IgG subclass or at least a portion of an endogenous regulatory element that drives expression of the endogenous CH1 domain, wherein the IgH allele comprises nucleic acid encoding a first amino acid sequence of a FN3 polypeptide, wherein the IgH allele comprises nucleic acid encoding one or more human Ig variable D domains, wherein the IgH allele comprises nucleic acid encoding a second amino acid sequence of a FN3 polypeptide, wherein the non-human animal is capable of producing an antibody-like molecule, wherein the antibody-like molecule comprises the CH2 or CH3 domain of the IgG subclass, the first amino acid sequence, one of the human Ig variable D domains, and the second amino acid sequence and lacks the CH1 domain. The IgH allele can lack endogenous nucleic acid encoding at least a portion of an IgM constant domain, endogenous nucleic acid encoding at least a portion of an IgD constant domain, endogenous nucleic acid encoding at least a portion of an IgE constant domain, or endogenous nucleic acid encoding at least a portion of an IgA constant domain. The IgH allele of the non-human animal can include endogenous nucleic acid encoding the CH2 domain and the CH3 domain of the IgG subclass. The IgH allele of the non-human animal can include endogenous nucleic acid encoding a hinge domain of the IgG subclass. The IgG subclass can be an IgG2 subclass. The IgG subclass can be an IgG2a, IgG2b, IgG2c, IgG3, or IgG4 subclass. The IgG subclass can be an IgG1 subclass. The IgH allele can lack endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain. The IgH allele can lack endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain. The IgH allele can lack endogenous nucleic acid encoding each of the IgG2 constant domains, endogenous nucleic acid encoding each of the IgG3 constant domains, or endogenous nucleic acid encoding each of the IgG4 constant domains. The IgH allele can lack endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains. The IgH allele can lack endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain. The IgH allele can lack endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains. The IgH allele can lack endogenous nucleic acid encoding each of the IgM constant domains. The IgH allele can lack endogenous nucleic acid encoding each of the IgD constant domains. The IgH allele can lack endogenous nucleic acid encoding each of the IgE constant domains. The IgH allele can lack endogenous nucleic acid encoding IgA CH1 and CH2 constant domains. The IgH allele can lack nucleic acid encoding the endogenous CH1 domain. The IgH allele can lack the endogenous regulatory element. The IgH allele can include an endogenous Eμ. The first nucleic acid sequence encoding a full length CH2 domain downstream of the endogenous Eμ can be nucleic acid encoding an IgG CH2 domain. The first nucleic acid sequence encoding a full length CH2 domain downstream of the endogenous Eμ can be nucleic acid encoding an IgG1 CH2 domain. The IgH allele can include an endogenous Sμ, an endogenous Iμ promoter, an endogenous Iμ exon, or a combination thereof. The first nucleic acid sequence encoding a full length CH2 domain downstream of the endogenous Sμ, the endogenous Iμ promoter, or the endogenous Iμ exon can be nucleic acid encoding an IgG CH2 domain. The the first nucleic acid sequence encoding a full length CH2 domain downstream of the endogenous Sμ, the endogenous Iμ promoter, or the endogenous Iμ exon can be nucleic acid encoding an IgG1 CH2 domain. The IgH allele can include an endogenous 3′γ1E. The IgH allele can lack endogenous nucleic acid encoding a full length CH2 domain downstream of the endogenous 3′γ1E. The IgH allele can include an endogenous 5 hsR1. The first nucleic acid sequence encoding a full length CH2 domain upstream of the endogenous 5′hsR1 can be nucleic acid encoding an IgG CH2 domain. The first nucleic acid sequence encoding a full length CH2 domain upstream of the endogenous 5′hsR1 can be nucleic acid encoding an IgG1 CH2 domain. The IgH allele can include an endogenous 3′RR. The first nucleic acid sequence encoding a full length CH2 domain upstream of the endogenous 3′RR can be nucleic acid encoding an IgG CH2 domain. The first nucleic acid sequence encoding a full length CH2 domain upstream of the endogenous 3′RR can be nucleic acid encoding an IgG1 CH2 domain. The IgH allele can include an endogenous 3 CBE. The first nucleic acid sequence encoding a full length CH2 domain upstream of the endogenous 3′CBE can be nucleic acid encoding an IgG CH2 domain. The first nucleic acid sequence encoding a full length CH2 domain upstream of the endogenous 3′CBE can be nucleic acid encoding an IgG1 CH2 domain. The at least one allele of the genome can lack at least a portion of the endogenous Ig heavy chain variable region. The at least one allele of the genome can lack all the exons of the endogenous Ig heavy chain variable region. In some cases, both alleles of the genome can lack all the exons of the endogenous Ig heavy chain variable region. In some cases, neither allele of the genome can include an endogenous exon of an Ig heavy chain variable region. The FN3 polypeptide can be a human ¹⁰FN3 polypeptide. The first amino acid sequence can include the amino acid sequence set forth in any one of SEQ ID NOs:C24 and C26. The second amino acid sequence can include the amino acid sequence set forth in any one of SEQ ID NOs:C25 and C27. The IgH allele can include nucleic acid encoding two or more human Ig variable D domains. The IgH allele can include nucleic acid encoding three or more human Ig variable D domains. The IgH allele can include nucleic acid encoding four or more human Ig variable D domains. The IgH allele can include nucleic acid encoding five or more human Ig variable D domains. The IgH allele can include nucleic acid encoding six or more human Ig variable D domains. The IgH allele can include nucleic acid encoding seven or more human Ig variable D domains. The IgH allele can include nucleic acid encoding eight or more human Ig variable D domains. The IgH allele can include at least one exogenous recombinase site recognition nucleic acid sequence. The at least one exogenous recombinase site recognition nucleic acid sequence can be located upstream of the endogenous nucleic acid encoding the CH2 or CH3 domain of the IgG subclass. The IgH allele can include one, two, three, four, five, six, seven, eight, nine, or ten different exogenous recombinase site recognition nucleic acid sequences. The IgH allele can include at least three different exogenous recombinase site recognition nucleic acid sequences. The IgH allele can include at least five different exogenous recombinase site recognition nucleic acid sequences. Each of the different exogenous recombinase site recognition nucleic acid sequences can be located less than 2.5 Mb upstream of an endogenous Eμ. Each of the different exogenous recombinase site recognition nucleic acid sequences can be located less than 2.0 Mb, less than 1.5 Mb, less than 1.0 Mb, less than 500 kb, or less than 250 kb upstream of an endogenous Eμ. Each of the different exogenous recombinase site recognition nucleic acid sequences can be located less than 200 kb, less than 100 kb, less than 50 kb, less than 25 kb, or less than 10 kb upstream of an endogenous Eμ. The non-human animal can be a mouse or rat. The non-human animal can be a mouse.

In another aspect, this document features methods for producing a non-human animal described herein. The methods can include, or consist essentially of, (a1) introducing exogenous nucleic acid encoding the first amino acid sequence of the FN3 polypeptide into an endogenous IgH locus in a stem cell of a non-human animal whose genome comprises an endogenous IgH locus comprising the nucleic acid encoding one or more human Ig variable D domains and nucleic acid encoding the second amino acid sequence of the FN3 polypeptide: (b1) implanting the stem cell into a blastocyst: (c1) implanting the blastocyst into a pseudo-pregnant non-human animal to obtain a chimeric non-human animal; (d1) crossing the chimeric non-human animal to a non-human animal to produce offspring; (e1) screening the offspring for heterozygosity; and (f1) identifying a founder non-human animal carrying the exogenous nucleic acid: or (a2) introducing exogenous nucleic acid encoding the second amino acid sequence of the FN3 polypeptide into an endogenous IgH locus in a stem cell of a non-human animal whose genome comprises an endogenous IgH locus comprising the nucleic acid encoding one or more human Ig variable D domains and nucleic acid encoding the first amino acid sequence of the FN3 polypeptide: (b1) implanting the stem cell into a blastocyst: (c1) implanting the blastocyst into a pseudo-pregnant non-human animal to obtain a chimeric non-human animal; (d1) crossing the chimeric non-human animal to a non-human animal to produce offspring; (e1) screening the offspring for heterozygosity; and (f1) identifying a founder non-human animal carrying the exogenous nucleic acid. The stem cell can be an embryonic stem cell.

In another aspect, this document features methods for producing an antibody-like molecule in a non-human animal described herein. The methods can include, or consist essentially of, administering an antigen to the non-human animal, wherein one or more B cells in the non-human animal produce an antibody-like molecule comprising the first amino acid sequence of the FN3 polypeptide, one of the human Ig variable D domains, and the second amino acid sequence of the FN3 polypeptide. The antibody-like molecule can be a single chain antibody-like molecule.

In another aspect, this document features methods for obtaining a B cell that produces an antibody-like molecule capable of binding to an antigen. The methods can include, or consist essentially of, (a) administering the antigen to a non-human animal described herein, wherein one or more B cells in the non-human animal produce the antibody-like molecule, and wherein the antibody-like molecule comprises the first amino acid sequence of the FN3 polypeptide, one of the human Ig variable D domains, and the second amino acid sequence of the FN3 polypeptide, and (b) isolating the one or more B cells from the non-human animal. The antibody-like molecule can be a single chain antibody-like molecule.

In another aspect, this document features a non-human animal, wherein the genome of said non-human animal comprises an immunoglobulin heavy chain (IgH) allele, wherein said IgH allele comprises an endogenous nucleic acid encoding a CH2 or CH3 domain of an IgG subclass, wherein said IgH allele lacks nucleic acid encoding at least a portion of an endogenous CH1 domain of said IgG subclass or at least a portion of an endogenous regulatory element that drives expression of said endogenous CH1 domain, wherein said IgH allele comprises one or more modified human Ig JH gene segments that do not encode for at least one naturally-occurring tryptophan residue, wherein said non-human animal is capable of producing an antibody, wherein said antibody (a) comprises said CH2 or CH3 domain of said IgG subclass. (b) comprises a variable region comprising a JH domain comprising the amino acid sequence encoded by one of said modified human Ig JH gene segments, and (c) lacks said CH1 domain. The IgH allele can lack endogenous nucleic acid encoding at least a portion of an IgM constant domain, endogenous nucleic acid encoding at least a portion of an IgD constant domain, endogenous nucleic acid encoding at least a portion of an IgE constant domain, or endogenous nucleic acid encoding at least a portion of an IgA constant domain. The IgH allele of said non-human animal can include endogenous nucleic acid encoding said CH2 domain and said CH3 domain of said IgG subclass. The IgH allele of said non-human animal can include endogenous nucleic acid encoding a hinge domain of said IgG subclass. The IgG subclass can be an IgG2 subclass. The IgG subclass can be an IgG2a, IgG2b, IgG2c, IgG3, or IgG4 subclass. The IgG subclass can be an IgG1 subclass. The IgH allele can lack endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain. The IgH allele can lack endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain. The IgH allele can lack endogenous nucleic acid encoding each of the IgG2 constant domains, endogenous nucleic acid encoding each of the IgG3 constant domains, or endogenous nucleic acid encoding each of the IgG4 constant domains. The IgH allele can lack endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains. The IgH allele can lack endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain. The IgH allele can lack endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains. The IgH allele can lack endogenous nucleic acid encoding each of the IgM constant domains. The IgH allele can lack endogenous nucleic acid encoding each of the IgD constant domains. The IgH allele can lack endogenous nucleic acid encoding each of the IgE constant domains. The IgH allele can lack endogenous nucleic acid encoding IgA CH1 and CH2 constant domains. The IgH allele can lack nucleic acid encoding said endogenous CH1 domain. The IgH allele can lack said endogenous regulatory element. The IgH allele can include an endogenous Eμ. The first nucleic acid sequence encoding a full length CH2 domain downstream of said endogenous Eμ can be nucleic acid encoding an IgG CH2 domain. The first nucleic acid sequence encoding a full length CH2 domain downstream of said endogenous Eμ can be nucleic acid encoding an IgG1 CH2 domain. The IgH allele can include an endogenous Sμ, an endogenous Iμ promoter, an endogenous Iμ exon, or a combination thereof. The first nucleic acid sequence encoding a full length CH2 domain downstream of said endogenous Sμ, said endogenous Iμ promoter, or said endogenous Iμ exon can be nucleic acid encoding an IgG CH2 domain. The first nucleic acid sequence encoding a full length CH2 domain downstream of said endogenous Sμ, said endogenous Iμ promoter, or said endogenous Iμ exon can be nucleic acid encoding an IgG1 CH2 domain. The IgH allele can include an endogenous 3′γ1E. The IgH allele can lack endogenous nucleic acid encoding a full length CH2 domain downstream of said endogenous 3′γ1E. The IgH allele can include an endogenous 5′hsR1. The first nucleic acid sequence encoding a full length CH2 domain upstream of said endogenous 5 hsR1 can be nucleic acid encoding an IgG CH2 domain. The first nucleic acid sequence encoding a full length CH2 domain upstream of said endogenous 5′hsR1 can be nucleic acid encoding an IgG1 CH2 domain. The IgH allele can include an endogenous 3′RR. The first nucleic acid sequence encoding a full length CH2 domain upstream of said endogenous 3′RR can be nucleic acid encoding an IgG CH2 domain. The first nucleic acid sequence encoding a full length CH2 domain upstream of said endogenous 3′RR can be nucleic acid encoding an IgG1 CH2 domain. The IgH allele can include an endogenous 3′CBE. The first nucleic acid sequence encoding a full length CH2 domain upstream of said endogenous 3′CBE can be nucleic acid encoding an IgG CH2 domain. The first nucleic acid sequence encoding a full length CH2 domain upstream of said endogenous 3 CBE can be nucleic acid encoding an IgG1 CH2 domain. The at least one allele of said genome can lack at least a portion of the endogenous Ig heavy chain variable region. The at least one allele of said genome can lack all the exons of the endogenous Ig heavy chain variable region. In some cases, both alleles of said genome can lack all the exons of the endogenous Ig heavy chain variable region. In some cases, neither allele of said genome comprises an endogenous exon of an Ig heavy chain variable region. The IgH allele can include exogenous nucleic acid encoding one or more human Ig VH gene segments. The IgH allele can include three or more human Ig VH gene segments. The IgH allele can include 26 or more human Ig VH gene segments. The IgH allele can include 65 or more human Ig VH gene segments. The IgH allele can include 129 human Ig VH gene segments. The IgH allele can include 13 or more human Ig DH gene segments. The IgH allele can include 27 human Ig DH gene segments. The IgH allele can include three or more of said modified human Ig JH gene segments. The IgH allele can include five or more of said modified human Ig JH gene segments. The IgH allele can include 129 or more human Ig VH gene segments. 27 or more human Ig DH gene segments, and 6 or more of said modified human Ig JH gene segments. The one or more modified human Ig JH gene segments can be selected from the group consisting of a modified human Ig JH gene segment that encodes an Ig JH domain having the amino acid sequence set forth in SEQ ID NO: 105 and a modified human Ig JH gene segment that encodes an Ig JH domain having the amino acid sequence set forth in SEQ ID NO: 106, wherein the “X” residue within each sequence identifier is an amino acid other than tryptophan. The “X” residue within each sequence identifier can be arginine (R), lysine (K), tyrosine (Y), serine(S), threonine (T), histidine (H), or glutamine (Q). The “X” residue within each sequence identifier can be arginine (R), lysine (K), tyrosine (Y), serine(S), threonine (T), or glutamine (Q). The “X” residue within each sequence identifier can be arginine (R), lysine (K), serine(S), threonine (T), histidine (H), or glutamine (Q). The “X” residue within each sequence identifier can be arginine (R), tyrosine (Y), serine(S), threonine (T), histidine (H), or glutamine (Q). The “X” residue within each sequence identifier can be tyrosine (Y), serine(S), or threonine (T). The “X” residue within each sequence identifier can be tyrosine (Y) or serine(S). The “X” residue within each sequence identifier can be arginine (R). The “X” residue within each sequence identifier can be lysine (K). The “X” residue within each sequence identifier can be tyrosine (Y). The “X” residue within each sequence identifier can be serine(S). The “X” residue within each sequence identifier can be threonine (T). The “X” residue within each sequence identifier can be histidine (H). The “X” residue within each sequence identifier can be glutamine (Q). The IgH allele can include at least one exogenous recombinase site recognition nucleic acid sequence. The at least one exogenous recombinase site recognition nucleic acid sequence can be located upstream of said endogenous nucleic acid encoding said CH2 or CH3 domain of said IgG subclass. The IgH allele can include one, two, three, four, five, six, seven, eight, nine, or ten different exogenous recombinase site recognition nucleic acid sequences. The IgH allele can include at least three different exogenous recombinase site recognition nucleic acid sequences. The IgH allele can include at least five different exogenous recombinase site recognition nucleic acid sequences. Each of said different exogenous recombinase site recognition nucleic acid sequences can be located less than 2.5 Mb upstream of an endogenous Eμ. Each of said different exogenous recombinase site recognition nucleic acid sequences can be located less than 2.0 Mb, less than 1.5 Mb, less than 1.0 Mb, less than 500 kb, or less than 250 kb upstream of an endogenous Eμ. Each of said different exogenous recombinase site recognition nucleic acid sequences can be located less than 200 kb, less than 100 kb, less than 50 kb, less than 25 kb, or less than 10 kb upstream of an endogenous Eμ. The non-human animal can be a mouse or rat. The non-human animal can be a mouse.

In another aspect, this document features methods for producing a non-human animal described herein. The methods can include, or consist essentially of, (a) introducing exogenous nucleic acid comprising one or more modified human Ig JH gene segments that do not encode for at least one naturally-occurring tryptophan residue, into an endogenous IgH locus in a stem cell of a non-human animal: (b) implanting said stem cell into a blastocyst: (c) implanting said blastocyst into a pseudo-pregnant non-human animal to obtain a chimeric non-human animal: (d) crossing said chimeric non-human animal to a wild-type non-human animal to produce offspring: (e) screening the offspring for heterozygosity; and (f) identifying a founder non-human animal carrying said one or more modified human Ig JH gene segments. The stem cell can be an embryonic stem cell.

In another aspect, this document features methods for producing an antibody in a non-human animal described herein. The methods can include, or consist essentially of, administering an antigen to said non-human animal, wherein one or more B cells in said non-human animal produce an antibody comprising a JH domain encoded by one of said modified human Ig JH gene segments. The antibody can be a single domain antibody (sdAb).

In another aspect, this document features methods for obtaining a B cell that produces an antibody capable of binding to an antigen. The methods can include, or consist essentially of, (a) administering said antigen to a non-human animal of any one of claims 1-75, wherein one or more B cells in said non-human animal produce said antibody, and wherein said antibody comprises a JH domain encoded by one of said modified human Ig JH gene segments, and (b) isolating said one or more B cells from said non-human animal. The antibody can be a sdAb. The one or more modified human Ig JH gene segments do not encode for any naturally-occurring tryptophan residues.

In another aspect, this document features a non-human animal, wherein the genome of the non-human animal comprises an immunoglobulin heavy chain (IgH) allele, wherein the IgH allele comprises an endogenous nucleic acid encoding a CH2 or CH3 domain of an IgG subclass, wherein the IgH allele lacks nucleic acid encoding at least a portion of an endogenous CH1 domain of the IgG subclass or at least a portion of an endogenous regulatory element that drives expression of the endogenous CH1 domain, wherein the IgH allele comprises one or more Ig VH gene segments selected from the group consisting of an Ig VH gene segment that encodes the amino acid sequence set forth in SEQ ID NO:119, an Ig VH gene segment that encodes the amino acid sequence set forth in SEQ ID NO: 120, an Ig VH gene segment that encodes the amino acid sequence set forth in SEQ ID NO: 121, an Ig VH gene segment that encodes the amino acid sequence set forth in SEQ ID NO: 122, and an Ig VH gene segment that encodes the amino acid sequence set forth in SEQ ID NO:123, wherein the non-human animal is capable of producing an antibody, wherein the antibody (a) comprises the CH2 or CH3 domain of the IgG subclass and a variable domain comprising the amino acid sequence encoded by one of the Ig VH gene segments and (b) lacks the CH1 domain. The IgH allele can lack endogenous nucleic acid encoding at least a portion of an IgM constant domain, endogenous nucleic acid encoding at least a portion of an IgD constant domain, endogenous nucleic acid encoding at least a portion of an IgE constant domain, or endogenous nucleic acid encoding at least a portion of an IgA constant domain. The IgH allele of the non-human animal can include endogenous nucleic acid encoding the CH2 domain and the CH3 domain of the IgG subclass. The IgH allele of the non-human animal can include endogenous nucleic acid encoding a hinge domain of the IgG subclass. The IgG subclass can be an IgG2 subclass. The IgG subclass can be an IgG2a, IgG2b, IgG2c. IgG3, or IgG4 subclass. The IgG subclass can be an IgG1 subclass. The IgH allele can lack endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain. The IgH allele can lack endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain. The IgH allele can lack endogenous nucleic acid encoding each of the IgG2 constant domains, endogenous nucleic acid encoding each of the IgG3 constant domains, or endogenous nucleic acid encoding each of the IgG4 constant domains. The IgH allele can lack endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains. The IgH allele can lack endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain. The IgH allele can lack endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains. The IgH allele can lack endogenous nucleic acid encoding each of the IgM constant domains. The IgH allele can lack endogenous nucleic acid encoding each of the IgD constant domains. The IgH allele can lack endogenous nucleic acid encoding each of the IgE constant domains. The IgH allele can lack endogenous nucleic acid encoding IgA CH1 and CH2 constant domains. The IgH allele can lack nucleic acid encoding the endogenous CH1 domain. The IgH allele can lack the endogenous regulatory element. The IgH allele can include an endogenous Eμ. The first nucleic acid sequence encoding a full length CH2 domain downstream of the endogenous Eμ can be nucleic acid encoding an IgG CH2 domain. The first nucleic acid sequence encoding a full length CH2 domain downstream of the endogenous Eμ can be nucleic acid encoding an IgG1 CH2 domain. The IgH allele can include an endogenous Sμ, an endogenous Iμ promoter, an endogenous Iμ exon, or a combination thereof. The first nucleic acid sequence encoding a full length CH2 domain downstream of the endogenous Sμ, the endogenous Iμ promoter, or the endogenous Iμ exon can be nucleic acid encoding an IgG CH2 domain. The first nucleic acid sequence encoding a full length CH2 domain downstream of the endogenous Sμ, the endogenous Iμ promoter, or the endogenous Iμ exon can be nucleic acid encoding an IgG1 CH2 domain. The IgH allele can include an endogenous 3′γ1E. The IgH allele can lack endogenous nucleic acid encoding a full length CH2 domain downstream of the endogenous 3′γ1E. The IgH allele can include an endogenous 5′hsR1. The first nucleic acid sequence encoding a full length CH2 domain upstream of the endogenous 5′hsR1 can be nucleic acid encoding an IgG CH2 domain. The first nucleic acid sequence encoding a full length CH2 domain upstream of the endogenous 5′hsR1 can be nucleic acid encoding an IgG1 CH2 domain. The IgH allele can include an endogenous 3′RR. The first nucleic acid sequence encoding a full length CH2 domain upstream of the endogenous 3′RR can be nucleic acid encoding an IgG CH2 domain. The first nucleic acid sequence encoding a full length CH2 domain upstream of the endogenous 3′RR can be nucleic acid encoding an IgG1 CH2 domain. The IgH allele can include an endogenous 3 CBE. The first nucleic acid sequence encoding a full length CH2 domain upstream of the endogenous 3 CBE can be nucleic acid encoding an IgG CH2 domain. The first nucleic acid sequence encoding a full length CH2 domain upstream of the endogenous 3 CBE can be nucleic acid encoding an IgG1 CH2 domain. The at least one allele of the genome can lack at least a portion of the endogenous Ig heavy chain variable region. The at least one allele of the genome can lack all the exons of the endogenous Ig heavy chain variable region. In some cases, both alleles of the genome lack all the exons of the endogenous Ig heavy chain variable region. In some cases, neither allele of the genome can include an endogenous exon of an Ig heavy chain variable region. The IgH allele can include, in addition to the one or more Ig VH gene segments selected from the group, exogenous nucleic acid encoding one or more human Ig VH gene segments. The IgH allele can include three or more human Ig VH gene segments. The IgH allele can include 26 or more human Ig VH gene segments. The IgH allele can include 65 or more human Ig VH gene segments. The IgH allele can include 129 human Ig VH gene segments. The IgH allele can include 13 or more human Ig DH gene segments. The IgH allele can include 27 human Ig DH gene segments. The IgH allele can include three or more human Ig JH gene segments. The IgH allele can include 9 human Ig JH gene segments. The IgH allele can include 129 or more human Ig VH gene segments. 27 or more human Ig DH gene segments, and 9 or more human Ig JH gene segments. The IgH allele can include two or more of the Ig VH gene segments selected from the group. The IgH allele can include three or more of the Ig VH gene segments selected from the group. The IgH allele can include four or more of the Ig VH gene segments selected from the group. The IgH allele can include the Ig VH gene segment that encodes the amino acid sequence set forth in SEQ ID NO:119, the Ig VH gene segment that encodes the amino acid sequence set forth in SEQ ID NO:120, the Ig VH gene segment that encodes the amino acid sequence set forth in SEQ ID NO:121, the Ig VH gene segment that encodes the amino acid sequence set forth in SEQ ID NO: 122, and the Ig VH gene segment that encodes the amino acid sequence set forth in SEQ ID NO:123. The IgH allele can include an endogenous regulatory element for each of the one or more Ig VH gene segments. The endogenous regulatory element can be an endogenous promoter sequence of the non-human animal. The IgH allele can include an endogenous exon encoding a leader sequence for each of the one or more Ig VH gene segments. The endogenous exon can be an endogenous L1 exon of the non-human animal. The endogenous exon can be an endogenous L2 exon of the non-human animal. The IgH allele can include endogenous L1 and L2 exons encoding a leader sequence for each of the one or more Ig VH gene segments. The IgH allele can include at least one exogenous recombinase site recognition nucleic acid sequence. The at least one exogenous recombinase site recognition nucleic acid sequence can be located upstream of the endogenous nucleic acid encoding the CH2 or CH3 domain of the IgG subclass. The IgH allele can include one, two, three, four, five, six, seven, eight, nine, or ten different exogenous recombinase site recognition nucleic acid sequences. The IgH allele can include at least three different exogenous recombinase site recognition nucleic acid sequences. The IgH allele can include at least five different exogenous recombinase site recognition nucleic acid sequences. Each of the different exogenous recombinase site recognition nucleic acid sequences can be located less than 2.5 Mb upstream of an endogenous Eμ. Each of the different exogenous recombinase site recognition nucleic acid sequences can be located less than 2.0 Mb, less than 1.5 Mb, less than 1.0 Mb, less than 500 kb, or less than 250 kb upstream of an endogenous Eμ. Each of the different exogenous recombinase site recognition nucleic acid sequences can be located less than 200 kb, less than 100 kb, less than 50 kb, less than 25 kb, or less than 10 kb upstream of an endogenous Eμ. The non-human animal can be a mouse or rat. The non-human animal can be a mouse.

In another aspect, this document features methods for producing a non-human animal described herein. The methods can include, or consist essentially of, (a) introducing exogenous nucleic acid comprising one or more Ig VH gene segments selected from the group consisting of an Ig VH gene segment that encodes the amino acid sequence set forth in SEQ ID NO:119, an Ig VH gene segment that encodes the amino acid sequence set forth in SEQ ID NO: 120, an Ig VH gene segment that encodes the amino acid sequence set forth in SEQ ID NO: 121, an Ig VH gene segment that encodes the amino acid sequence set forth in SEQ ID NO: 122, and an Ig VH gene segment that encodes the amino acid sequence set forth in SEQ ID NO: 123 into an endogenous IgH locus in a stem cell of a non-human animal; (b) implanting the stem cell into a blastocyst; (c) implanting the blastocyst into a pseudo-pregnant non-human animal to obtain a chimeric non-human animal; (d) crossing the chimeric non-human animal to a wild-type non-human animal to produce offspring; (e) screening the offspring for heterozygosity; and (f) identifying a founder non-human animal carrying the one or more Ig VH gene segments. The stem cell can be an embryonic stem cell.

In another aspect, this document features methods for producing an antibody in a non-human animal described herein. The methods can include, or consist essentially of, administering an antigen to the non-human animal, wherein one or more B cells in the non-human animal produce an antibody comprising a variable domain encoded by one of the Ig VH gene segments. The antibody can be a single domain antibody (sdAb). The antibody can include an amino acid sequence selected from SEQ ID NOs: 119-123.

In another aspect, this document features methods for obtaining a B cell that produces an antibody capable of binding to an antigen. The methods can include, or consist essentially of, (a) administering the antigen to a non-human animal described herein, wherein one or more B cells in the non-human animal produce the antibody, and wherein the antibody can include a variable domain comprising the amino acid sequence encoded by one of the Ig VH gene segments, and (b) isolating the one or more B cells from the non-human animal. The antibody can be a sdAb. The antibody can include an amino acid sequence selected from SEQ ID NOs: 119-123.

In another aspect, this document features a chimeric mouse generated from a mouse embryo comprising at least two different mouse cells having different genomes, wherein the genome of a first cell of the at least two different mouse cells comprises a genomic modification that prevents cells derived from the first cell from producing endogenous mouse immunoglobulins, wherein the genome of a second cell of the at least two different mouse cells comprises an IgH locus comprising a nucleic acid sequence encoding an input nanobody domain, wherein the chimeric mouse comprises cells originating from the first cell and cells originating from the second cell, wherein the chimeric mouse produces heavy chain only antibodies containing the input nanobody domain and optionally one or more variants of the heavy chain only antibody that underwent affinity maturation within the chimeric mouse, and wherein the chimeric mouse does not produce endogenous mouse immunoglobulins. The genomic modification of the first cell can comprise a homozygous disruption of a nucleic acid sequence encoding a recombination activating gene 2 (Rag2) polypeptide. The genomic modification of the first cell can comprise a homozygous disruption of a regulatory nucleic acid sequence that regulates expression of a Rag2 polypeptide. The genomic modification of the first cell can comprise a homozygous disruption of nucleic acid encoding immunoglobulin (Ig) domains needed for expression of endogenous mouse antibodies. The genomic modification of the first cell can comprise a homozygous disruption of a regulatory nucleic acid sequence that regulates expression of endogenous mouse antibodies. The IgH locus can comprise an endogenous nucleic acid encoding a CH2 or CH3 domain of an IgG subclass, wherein the IgH locus lacks nucleic acid encoding at least a portion of an endogenous CH1 domain of the IgG subclass or at least a portion of an endogenous regulatory element that drives expression of the endogenous CH1 domain, wherein the heavy chain only antibody (a) comprises the CH2 or CH3 domain of the IgG subclass and (b) lacks the CH1 domain. The IgH locus can comprise endogenous nucleic acid encoding a hinge, the CH2 domain, and the CH3 domain of the IgG subclass. The IgH locus can comprise endogenous nucleic acid encoding a hinge domain of the IgG subclass. The IgG subclass can be an IgG2 subclass. The IgG subclass can be an IgG2a, IgG2b, IgG2c, IgG3, or IgG4 subclass. The IgG subclass can be an IgG1 subclass. The IgH locus can lack endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain. The IgH locus can lack endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain. The IgH locus can lack endogenous nucleic acid encoding each of the IgG2 constant domains, endogenous nucleic acid encoding each of the IgG3 constant domains, or endogenous nucleic acid encoding each of the IgG4 constant domains. The IgH locus can lack endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains. The IgH locus can lack endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains. The IgH locus can lack endogenous nucleic acid encoding each of the IgM constant domains. The IgH locus can lack endogenous nucleic acid encoding each of the IgD constant domains. The IgH locus can lack endogenous nucleic acid encoding each of the IgE constant domains. The IgH locus can lack endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain. The IgH locus can lack endogenous nucleic acid encoding IgA CH1 and CH2 constant domains. The IgH locus can lack nucleic acid encoding the endogenous CH1 domain. The IgH locus can lack the endogenous regulatory element. The IgH locus can comprise an endogenous Eμ. The first nucleic acid sequence encoding a full length CH2 domain downstream of the endogenous Eμ can be nucleic acid encoding an IgG CH2 domain. The first nucleic acid sequence encoding a full length CH2 domain downstream of the endogenous Eμ can be nucleic acid encoding an IgG1 CH2 domain. The IgH locus can comprise an endogenous Sμ, an endogenous Iμ promoter, an endogenous Iμ exon, or a combination thereof. The first nucleic acid sequence encoding a full length CH2 domain downstream of the endogenous Sμ, the endogenous Iμ promoter, or the endogenous Iμ exon can be nucleic acid encoding an IgG CH2 domain. The first nucleic acid sequence encoding a full length CH2 domain downstream of the endogenous Su, the endogenous Iμ promoter, or the endogenous Iμ exon can be nucleic acid encoding an IgG1 CH2 domain. The IgH locus can comprise an endogenous 3′γ1E. The IgH locus can lack endogenous nucleic acid encoding a full length CH2 domain downstream of the endogenous 3′γ1E. The IgH locus can comprise an endogenous 5 hsR1. The first nucleic acid sequence encoding a full length CH2 domain upstream of the endogenous 5 hsR1 can be nucleic acid encoding an IgG CH2 domain. The first nucleic acid sequence encoding a full length CH2 domain upstream of the endogenous 5′hsR1 can be nucleic acid encoding an IgG1 CH2 domain. The IgH locus can comprise an endogenous 3′RR. The first nucleic acid sequence encoding a full length CH2 domain upstream of the endogenous 3′RR can be nucleic acid encoding an IgG CH2 domain. The first nucleic acid sequence encoding a full length CH2 domain upstream of the endogenous 3′RR can be nucleic acid encoding an IgG1 CH2 domain. The IgH locus can comprise an endogenous 3 CBE. The first nucleic acid sequence encoding a full length CH2 domain upstream of the endogenous 3 CBE can be nucleic acid encoding an IgG CH2 domain. The first nucleic acid sequence encoding a full length CH2 domain upstream of the endogenous 3′CBE can be nucleic acid encoding an IgG1 CH2 domain. At least one allele of the genome of the second cell can lack at least a portion of the endogenous Ig heavy chain variable region. At least one allele of the genome of the second cell can lack all the exons of the endogenous Ig heavy chain variable region. Both alleles of the genome of the second cell can lack all the exons of the endogenous Ig heavy chain variable region. Neither allele of the genome of the second cell can comprise an endogenous exon of an Ig heavy chain variable region. One allele of the genome of the second cell can comprise the IgH locus. One allele of the genome of the second cell can comprise the IgH locus, and the other allele of the genome of the second cell can comprise the IgH locus with the exception that it does not comprise the nucleic acid sequence encoding the input nanobody domain. The genomic modification of the first cell can comprise a homozygous disruption of a nucleic acid sequence encoding a Rag1 polypeptide. The genomic modification of the first cell can comprise a homozygous disruption of a regulatory nucleic acid sequence that regulates expression of a Rag1 polypeptide.

In another aspect, this document features a method for making a chimeric mouse. The method comprises, consists essentially of, or consists of developing the chimeric mouse within the uterus of a surrogate female mouse from an implanted mouse embryo comprising at least two different mouse cells having different genomes, wherein the genome of a first cell of the at least two different mouse cells comprises a genomic modification that prevents cells derived from the first cell from producing endogenous mouse immunoglobulins, wherein the genome of a second cell of the at least two different mouse cells comprises an IgH locus comprising a nucleic acid sequence encoding an input nanobody domain, wherein the chimeric mouse comprises cells originating from the first cell and cells originating from the second cell, wherein the chimeric mouse produces heavy chain only antibodies containing the input nanobody domain and optionally one or more variants of the heavy chain only antibodies that underwent affinity maturation within the chimeric mouse, and wherein the chimeric mouse does not produce endogenous mouse immunoglobulins. The genomic modification of the first cell can comprise a homozygous disruption of a nucleic acid sequence encoding a Rag2 polypeptide. The genomic modification of the first cell can comprise a homozygous disruption of a regulatory nucleic acid sequence that regulates expression of a Rag2 polypeptide. The genomic modification of the first cell can comprise a homozygous disruption of nucleic acid encoding immunoglobulin (Ig) domains needed for expression of endogenous mouse antibodies. The genomic modification of the first cell can comprise a homozygous disruption of a regulatory nucleic acid sequence that regulates expression of endogenous mouse antibodies. The IgH locus can comprise an endogenous nucleic acid encoding a CH2 or CH3 domain of an IgG subclass, wherein the IgH locus lacks nucleic acid encoding at least a portion of an endogenous CH1 domain of the IgG subclass or at least a portion of an endogenous regulatory element that drives expression of the endogenous CH1 domain, wherein the heavy chain only antibody (a) comprises the CH2 or CH3 domain of the IgG subclass and (b) lacks the CH1 domain. The IgH locus can comprise endogenous nucleic acid encoding a hinge, the CH2 domain, and the CH3 domain of the IgG subclass. The IgH locus can comprise endogenous nucleic acid encoding a hinge domain of the IgG subclass. The IgG subclass can be an IgG2 subclass. The IgG subclass can be an IgG2a. IgG2b. IgG2c. IgG3, or IgG4 subclass. The IgG subclass can be an IgG1 subclass. The IgH locus can lack endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain. The IgH locus can lack endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain. The IgH locus can lack endogenous nucleic acid encoding each of the IgG2 constant domains, endogenous nucleic acid encoding each of the IgG3 constant domains, or endogenous nucleic acid encoding each of the IgG4 constant domains. The IgH locus can lack endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains. The IgH locus can lack endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains. The IgH locus can lack endogenous nucleic acid encoding each of the IgM constant domains. The IgH locus can lack endogenous nucleic acid encoding each of the IgD constant domains. The IgH locus can lack endogenous nucleic acid encoding each of the IgE constant domains. The IgH locus can lack endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain. The IgH locus can lack endogenous nucleic acid encoding IgA CH1 and CH2 constant domains. The IgH locus can lack nucleic acid encoding the endogenous CH1 domain. The IgH locus can lack the endogenous regulatory element. The IgH locus can comprise an endogenous Eμ. The first nucleic acid sequence encoding a full length CH2 domain downstream of the endogenous Eμ can be nucleic acid encoding an IgG CH2 domain. The first nucleic acid sequence encoding a full length CH2 domain downstream of the endogenous Eμ can be nucleic acid encoding an IgG1 CH2 domain. The IgH locus can comprise an endogenous Sμ, an endogenous Iμ promoter, an endogenous Iμ exon, or a combination thereof. The first nucleic acid sequence encoding a full length CH2 domain downstream of the endogenous Sμ, the endogenous Iμ promoter, or the endogenous Iμ exon can be nucleic acid encoding an IgG CH2 domain. The first nucleic acid sequence encoding a full length CH2 domain downstream of the endogenous Sμ, the endogenous Iμ promoter, or the endogenous Iμ exon can be nucleic acid encoding an IgG1 CH2 domain. The IgH locus can comprise an endogenous 3′γ1E. The IgH locus can lack endogenous nucleic acid encoding a full length CH2 domain downstream of the endogenous 3′γ1E. The IgH locus can comprise an endogenous 5′hsR1. The first nucleic acid sequence encoding a full length CH2 domain upstream of the endogenous 5′hsR1 can be nucleic acid encoding an IgG CH2 domain. The first nucleic acid sequence encoding a full length CH2 domain upstream of the endogenous 5′hsR1 can be nucleic acid encoding an IgG1 CH2 domain. The IgH locus can comprise an endogenous 3′RR. The first nucleic acid sequence encoding a full length CH2 domain upstream of the endogenous 3′RR can be nucleic acid encoding an IgG CH2 domain. The first nucleic acid sequence encoding a full length CH2 domain upstream of the endogenous 3′RR can be nucleic acid encoding an IgG1 CH2 domain. The IgH locus can comprise an endogenous 3 CBE. The first nucleic acid sequence encoding a full length CH2 domain upstream of the endogenous 3′CBE can be nucleic acid encoding an IgG CH2 domain. The first nucleic acid sequence encoding a full length CH2 domain upstream of the endogenous 3 CBE can be nucleic acid encoding an IgG1 CH2 domain. At least one allele of the genome of the second cell can lack at least a portion of the endogenous Ig heavy chain variable region. At least one allele of the genome of the second cell can lack all the exons of the endogenous Ig heavy chain variable region. Both alleles of the genome of the second cell can lack all the exons of the endogenous Ig heavy chain variable region. Neither allele of the genome of the second cell can comprise an endogenous exon of an Ig heavy chain variable region. One allele of the genome of the second cell can comprise the IgH locus. One allele of the genome of the second cell can comprise the IgH locus, and the other allele of the genome of the second cell can comprise the IgH locus with the exception that it does not comprise the nucleic acid sequence encoding the input nanobody domain. The genomic modification of the first cell can comprise a homozygous disruption of a nucleic acid sequence encoding a Rag1 polypeptide. The genomic modification of the first cell can comprise a homozygous disruption of a regulatory nucleic acid sequence that regulates expression of a Rag1 polypeptide.

In another aspect, this document features a method for obtaining a variant of a heavy chain only antibody containing an input nanobody domain that underwent in vivo affinity maturation. The method comprises (a) administering an antigen recognized by a nanobody containing the input nanobody domain or an antibody containing the input nanobody domain to a chimeric mouse generated from a mouse embryo comprising at least two different mouse cells having different genomes, wherein the genome of a first cell of the at least two different mouse cells comprises a genomic modification that prevents cells derived from the first cell from producing endogenous mouse immunoglobulins, wherein the genome of a second cell of the at least two different mouse cells comprises an IgH locus comprising a nucleic acid sequence encoding the input nanobody domain, wherein the chimeric mouse comprises cells originating from the first cell and cells originating from the second cell, wherein the chimeric mouse produces heavy chain only antibodies containing the input nanobody domain and one or more variants of the heavy chain only antibody containing the input nanobody domain that underwent affinity maturation within the chimeric mouse, wherein the chimeric mouse does not produce endogenous mouse immunoglobulins, and (b) obtaining at least one of the one or more variants or a B cell producing at least one of the one or more variants from the chimeric mouse, thereby obtaining the variant of the heavy chain only antibody containing the input nanobody domain that underwent in vivo affinity maturation. The genomic modification of the first cell can comprise a homozygous disruption of a nucleic acid sequence encoding a Rag2 polypeptide. The genomic modification of the first cell can comprise a homozygous disruption of a regulatory nucleic acid sequence that regulates expression of a Rag2 polypeptide The genomic modification of the first cell can comprise a homozygous disruption of nucleic acid encoding immunoglobulin (Ig) domains needed for expression of endogenous mouse antibodies. The genomic modification of the first cell can comprise a homozygous disruption of a regulatory nucleic acid sequence that regulates expression of endogenous mouse antibodies. The IgH locus can comprise an endogenous nucleic acid encoding a CH2 or CH3 domain of an IgG subclass, wherein the IgH locus lacks nucleic acid encoding at least a portion of an endogenous CH1 domain of the IgG subclass or at least a portion of an endogenous regulatory element that drives expression of the endogenous CH1 domain, wherein the heavy chain only antibody (a) comprises the CH2 or CH3 domain of the IgG subclass and (b) lacks the CH1 domain. The IgH locus can comprise endogenous nucleic acid encoding a hinge, the CH2 domain, and the CH3 domain of the IgG subclass. The IgH locus can comprise endogenous nucleic acid encoding a hinge domain of the IgG subclass. The IgG subclass can be an IgG2 subclass. The IgG subclass can be an IgG2a, IgG2b, IgG2c, IgG3, or IgG4 subclass. The IgG subclass can be an IgG1 subclass. The IgH locus can lack endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain. The IgH locus can lack endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain. The IgH locus can lack endogenous nucleic acid encoding each of the IgG2 constant domains, endogenous nucleic acid encoding each of the IgG3 constant domains, or endogenous nucleic acid encoding each of the IgG4 constant domains. The IgH locus can lack endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains. The IgH locus can lack endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains. The IgH locus can lack endogenous nucleic acid encoding each of the IgM constant domains. The IgH locus can lack endogenous nucleic acid encoding each of the IgD constant domains. The IgH locus can lack endogenous nucleic acid encoding each of the IgE constant domains. The IgH locus can lack endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain. The IgH locus can lack endogenous nucleic acid encoding IgA CH1 and CH2 constant domains. The IgH locus can lack nucleic acid encoding the endogenous CH1 domain. The IgH locus can lack the endogenous regulatory element. The IgH locus can comprise an endogenous Eμ. The first nucleic acid sequence encoding a full length CH2 domain downstream of the endogenous Eμ can be nucleic acid encoding an IgG CH2 domain. The first nucleic acid sequence encoding a full length CH2 domain downstream of the endogenous Eμ can be nucleic acid encoding an IgG1 CH2 domain. The IgH locus can comprise an endogenous Sμ, an endogenous Iμ promoter, an endogenous Iμ exon, or a combination thereof. The first nucleic acid sequence encoding a full length CH2 domain downstream of the endogenous Sμ, the endogenous Iμ promoter, or the endogenous Iμ exon can be nucleic acid encoding an IgG CH2 domain. The first nucleic acid sequence encoding a full length CH2 domain downstream of the endogenous Sμ, the endogenous Iμ promoter, or the endogenous Iμ exon can be nucleic acid encoding an IgG1 CH2 domain. The IgH locus can comprise an endogenous 3′γ1E. The IgH locus can lack endogenous nucleic acid encoding a full length CH2 domain downstream of the endogenous 3′γ1E. The IgH locus can comprise an endogenous 5′hsR1. The first nucleic acid sequence encoding a full length CH2 domain upstream of the endogenous 5 hsR1 can be nucleic acid encoding an IgG CH2 domain. The first nucleic acid sequence encoding a full length CH2 domain upstream of the endogenous 5′hsR1 can be nucleic acid encoding an IgG1 CH2 domain. The IgH locus can comprise an endogenous 3′RR. The first nucleic acid sequence encoding a full length CH2 domain upstream of the endogenous 3′RR can be nucleic acid encoding an IgG CH2 domain. The first nucleic acid sequence encoding a full length CH2 domain upstream of the endogenous 3′RR can be nucleic acid encoding an IgG1 CH2 domain. The IgH locus can comprise an endogenous 3 CBE. The first nucleic acid sequence encoding a full length CH2 domain upstream of the endogenous 3′CBE can be nucleic acid encoding an IgG CH2 domain. The first nucleic acid sequence encoding a full length CH2 domain upstream of the endogenous 3′CBE can be nucleic acid encoding an IgG1 CH2 domain. At least one allele of the genome of the second cell can lack at least a portion of the endogenous Ig heavy chain variable region. At least one allele of the genome of the second cell can lack all the exons of the endogenous Ig heavy chain variable region. Both alleles of the genome of the second cell can lack all the exons of the endogenous Ig heavy chain variable region. Neither allele of the genome of the second cell can comprise an endogenous exon of an Ig heavy chain variable region. One allele of the genome of the second cell can comprise the IgH locus. One allele of the genome of the second cell can comprise the IgH locus, and the other allele of the genome of the second cell can comprise the IgH locus with the exception that it does not comprise the nucleic acid sequence encoding the input nanobody domain. The genomic modification of the first cell can comprise a homozygous disruption of a nucleic acid sequence encoding a Rag1 polypeptide. The genomic modification of the first cell can comprise a homozygous disruption of a regulatory nucleic acid sequence that regulates expression of a Rag1 polypeptide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1D illustrate production of heavy chain only antibodies from Singularity Musculus mice. FIG. 1A) The genomic structure of the Igh locus of a wild-type (WT) mouse. Mouse VH, DH, and JH, and CH genes are indicated with dark or light boxes, along with the intronic enhancer Eμ and super-enhancer 3′RR in ovals. FIG. 1B) The engineered Singularity Musculus (SM) allele where all other CH genes as well as the CH1 exon of IgG1 are deleted. FIG. 1C) Tetrameric mouse IgG1 produced from the WT allele. (FIG. 1D) CH1-truncated heavy chain only IgG1 are produced from the Singularity Musculus allele, from which nanobodies can be derived.

FIG. 2 illustrates an exemplary genomic locus of mouse Igh. Shown is a schematic map of the ˜220 kb CH region containing the indicated regulatory elements.

FIGS. 3A-3E illustrate a procedure for the generation of the Singularity Musculus allele. FIG. 3A shows the mouse wild type Igh locus. FIG. 3B shows a constant region of mouse Igh locus. The region to be deleted in the first round of engineering is indicated in dashed box. FIG. 3C shows deletion of Ighm-Ighg1 CH1 exon via CRISPR mediated NHEJ. The region to be deleted in the second round of engineering is indicated in dashed box. FIG. 3D shows the allele with Ighg2b-Igha exons 1-3 deleted and the drug selection cassette inserted via CRISPR mediate HDR. FIG. 3E shows removal of selection cassette by expression of Flp recombinase.

FIG. 4 illustrates exemplary genomic structures of wild-type mouse Igh allele and engineered Singularity Musculus and Singularity HyperDock alleles.

FIGS. 5A-5D illustrate the generation of the Singularity HyperDock allele from a Singularity Musculus allele. FIG. 5A shows a Singularity Musculus allele. FIG. 5B) The Singularity HyperDock allele is generated by deleting all mouse V_{H}, D_{H}, J_{H} genes (2.58 Mb) and inserting a docking cassette for sequential RMCE via CRISPR mediated HDR. FIG. 5C shows Synteny validation of the Singularity HyperDock allele via expression of Flp recombinase. FIG. 5D shows removal of the selection marker via expression of ΦC31 recombinase.

FIGS. 6A-6B illustrate production of human-mouse chimeric heavy chain only antibodies from exemplary Singularity Sapiens mice. FIG. 6A is a schematic of an exemplary human VH-mouse IgG1-ΔCH1 chimeric antibody that can be used to generate a human VH nanobody. FIG. 6B shows exemplary versions of Singularity Sapiens mice produced by sequential introduction of human V, D, J genes onto the Singularity HyperDock allele. Human VH-CH1-truncated heavy chain only IgG1 will be produced from the Singularity Sapiens mice, from which human VH nanobodies can be derived.

FIGS. 7A-7D illustrate the generation of the Singularity Sapiens allelic series (SSV1-3). The Singularity Sapiens allelic series is generated by inserting engineered human IGH BAC1-3 via sequential RMCE using a list of heterospecific lox sites to swap alternating selection cassettes (neo and hyg) upon expression of Cre recombinase.

FIGS. 8A-8D are schematics of a Singularity Sapiens allelic series (SSV4 and 5) showing sequential integration of human IGH-BAC4 and IGH-BAC5 via RMCE into an allele containing human IGH-BAC1, human IGH-BAC2, and human IGH-BAC3, followed by removal of the selection marker cassette with via expression of ΦC31 recombinase.

FIG. 9 illustrates human IGH BACs based on human genome GRCh38/hg38 assembly GENCODE Genes Track (Version 36, October 2020), showing the positions of human gene segments for variable heavy (IGHV), diversity heavy (IGHD), joining heavy (IGHJ), and constant heavy IGHM and IGHD in the IGH gene locus. Five BAC constructs (hIGH BAC1-5, boundaries marked in dashed boxes) carrying human IGHV. IGHD, and IGHJ gene segments were engineered with the corresponding source BACs (solid boxes) via recombineering. The engineered BACs were then used to reconstruct the entire human V-D-J genomic region into the Singularity HyperDock allele via RMCE as described herein. The numbers of V, D, and J gene segments included in each engineered BAC construct are indicated.

FIG. 10 illustrates an example of BAC recombineering. Source BACs are modified by bacterial homologous recombination (recombineering) to incorporate the appropriate selectable markers and recombination sites at the desired positions. Shown is an engineering process of hIgH-BAC1.

FIG. 11 illustrates VH exon validation, showing a PCR-based validation of Singularity Sapiens (SSV4) containing 37 functional human VH exons integrated at the Igh locus. PCR results were run on a Qiagen Qiaxel DNA High resolution cartridge. Top and bottom bands denoted Qiagen QX alignment marker 15 bp/3 kb (Cat #929522) that was run alongside Qiagen QX size marker 100 bp-2.5 kb (Cat #929559). The PCR products were verified by sanger sequencing to match the corresponding VH genes.

FIG. 12 illustrates an exemplary method of complex BAC recombineering. Source BACs are sequentially modified by bacterial homologous recombination (recombineering) to incorporate the appropriate selectable markers and recombination sites at the desired positions. An example is shown for the engineering process for hIGH-BAC5 from three sourced BACs.

FIGS. 13A-13B illustrate engineering of a mutant mouse lacking the Kappa light chain. FIG. 13A is a schematic showing deletion of the mouse IG Kappa and insertion of a docking site via CRISPR mediated HDR. FIG. 13B is genotyping PCR results confirming IGK HyperDock/KO allele in F1 mice.

FIGS. 14A-14B illustrate engineering of a mutant mouse lacking the Lambda light chain. FIG. 14A is a schematic showing deletion of the entire mouse IG Lambda locus via CRISPR mediated NHEJ. FIG. 14B is a PCR result confirming the generation of the IGL KO allele in ES cells.

FIGS. 15A-15D illustrate that Singularity Musculus mice produce HcAbs of CH1 truncated IgG1 only. Schematics of the Igh locus in WT (FIG. 15A) and SM (FIG. 15B) mice. Validation of Singularity Musculus mice is shown by RT-PCR (spleens) (FIG. 15C) and Western blots (plasma) (FIG. 15D).

FIGS. 16A-16D illustrate that Singularity Sapiens mice produce human-mouse chimeric heavy chain IgG1s. FIG. 16A shows a Singularity Musculus allele. FIG. 16B shows a Singularity Sapiens V1 allele, which contains all human J_{H}, all human D_{H}, and 3 human V_{H} genes. FIG. 16C is an RT-PCR analysis showing the specific expression of human-mouse chimeric IgG1ΔCH1 transcripts in Singularity Sapiens V1 mice. FIG. 16D shows sequencing validated the production of human-mouse chimeric transcripts (SEQ ID NO: 1).

FIGS. 17A-17B. FIG. 17A is a schematic of an exemplary human VH-mouse IgG1-ΔCH1 chimeric antibody that can be used to generate a human VH nanobody. FIG. 17B shows Western blot of IgM and IgG1 in immunized WT and Singularity Sapiens mice (SSV1).

FIGS. 18A-18B illustrate spleen morphology and IgM and IgG expression in B cells of in Singularity Musculus mice. FIG. 18A shows spleens of wild type and Singularity Musculus mice. FIG. 18B shows a flow cytometry analysis of splenocytes demonstrating the absence of IgM but normal IgG expression in CD19 positive B cells.

FIGS. 19A-19B illustrate a flow cytometry analysis of splenocytes using B cell markers in Singularity Sapiens mice. FIG. 19A shows a flow cytometry analysis demonstrating the presence of IgM⁺IgD⁺ B cells in wildtype mice but their absence in Singularity Sapiens mice (SSV2). FIG. 19B shows a flow cytometry analysis demonstrating the differential abundance of IgG1⁺ B cells in wildtype mice and in Singularity Sapiens mice (SSV2).

FIGS. 20A-20B illustrate that Singularity Musculus mice mount robust humoral immune responses upon antigen challenge. FIG. 20A shows ELISA results of plasma samples from pre-bleed wild-type and Singularity Musculus animals. FIG. 20B shows ELISA results of plasma samples from Day 28 terminal bleed of the same animals immunized with SARS-COV2 Spike Active Trimer protein (SAT) as compared to a commercial control antibody against SARS-COV2 Spike protein S1 subunit (S1 mAb Control).

FIGS. 21A-21B illustrate that Singularity Musculus mice and Singularity Sapiens mice mount robust humoral immune responses upon a variety of antigen challenges. FIG. 21A shows ELISA results of plasma samples from Day 51 terminal bleed wildtype (WT), Singularity Musculus (SM), and Singularity Sapiens (SSV1) animals upon antigen challenge to SAT. FIG. 21B shows ELISA results of plasma samples from Day 51 terminal bleed animals immunized with human PD-L1 as compared to a commercial human PD-L1 antibody

FIG. 22 is a schematic illustration of IgG1 transcripts from WT and Singularity Musculus mice and primer locations for 5′RACE amplification for next generation sequencing analysis.

FIGS. 23A-23C illustrate that Singularity Musculus mice exhibit comparable antibody diversity as in the wild-type mice. V_{H} diversity (FIG. 23A); J_{H} diversity (FIG. 23B); and CDR3 length diversity (FIG. 23C) of all clonotypes identified from two wild type and two Singularity Musculus mice immunized with SAT are shown.

FIGS. 24A-24C illustrate IGHV diversity of clonotypes identified in WT and SM mice. FIGS. 24A and 24B show IGHV usage in SM mice immunized with the indicated antigens. (FIG. 24C) More IGHV segments are accessible in SM mice than WT mice.

FIGS. 25A-25B illustrate IGHJ utilization in WT and SM mice. FIG. 25A shows IGHJ usage in SM mice immunized with the indicated antigens. (FIG. 25B) Different usage of certain IGHJ segments in SM mice compared to WT mice was observed.

FIGS. 26A-26B illustrate CDR3 length distribution in WT and SM mice. FIG. 26A shows the distribution of CDR3 length among clonotypes in SM and WT mice in response to the indicated antigen. FIG. 26B shows the average CDR3 length observed in SM and WT mice.

FIG. 27 illustrates somatic hypermutation in the Singularity Musculus mice. The histograms show the number of amino acid changes at each position in the heavy-chain variable region, as compared to the corresponding germline sequence, for the top 100 most abundant nanobody clonotypes identified from one naïve and three Singularity Musculus mice immunized with SAT. V_{H} residue position numbering is based on the IMGT scheme. Most significant changes occurred in the CDR regions.

FIG. 28 illustrates a flow chart for an exemplary NGS-guided, single cell-independent nanobody discovery process.

FIG. 29 illustrates a phylogenetic tree of selected clonotypes identified by next generation sequencing of HcAb repertoire of Singularity Musculus mice immunized with SAT. Top ranked (based on abundance) clonotypes were selected for each immunized animal for high throughput synthesis, cloning, expression, and ELISA screening for antigen affinity followed by competitive ELISA for inhibitor (neutralizing) activity for spike-ACE2 receptor binding. Antigen-specific clones are indicated in grey shading, and neutralizing clones are indicated with an asterisk.

FIGS. 30A-30B illustrate a schematic of the vector used for expressing nanobodies. FIG. 30A shows the plasmid map of the pFUSE-hIgG1-Fc2 expression vector and the restriction sites (EcoRI and NcoI) for inserting VH sequence. FIG. 30B shows an exemplary Nb-human Fc fusion that can be generated from the pFUSE-hIgG1-Fc2 expression vector.

FIG. 31 illustrates ELISA screens for binders in immunized WT and SM mice. The numbers of clonotypes screened and binders identified from WT and SM mice after immunization with indicated antigens are provided. Binding results (ELISA results OD₄₅₀) for each clonotype are also provided.

FIG. 32 contains pie charts from data in FIG. 31 showing the proportion of binders having the indicated binding affinity obtained from WT and SM mice. Each chart shows the proportion of binders as determined by ELISA of nanobody binding to the indicated antigen.

FIGS. 33A-33B illustrate exemplary antibody structures under unreduced and reduced conditions of WT IgG1s and Nb-human Fc fusions (FIG. 33A), and confirmation of size reduction with SDS-PAGE gels of a S1 mAb control (WT tetrameric IgG1) and purified SAT nanobody-Fc fusions (heavy chain only IgG1) (FIG. 33B). The expressed Nb-Fc human fusions are homodimers.

FIGS. 34A-34B illustrate SDS-PAGE gels of purified SAT human nanobody-human Fc fusions (heavy chain only IgG1). FIG. 34A shows gel run under non-reduced conditions. FIG. 34B shows gel run under reduced conditions. The expressed human Nb-human Fc fusions are observed as homodimers.

FIG. 35 illustrates size exclusion chromatography of two human nanobody-human Fc fusion proteins. Purified human Nb-human Fc fusion proteins against SAT antigen were run through a size exclusion column to assess protein aggregation.

FIGS. 36A-36B illustrate characterization of purified SAT Nb-human Fc fusions for antigen binding affinity and SARS-COV2 neutralization potency against a RBD Nb-Fc control (HAb8-S). FIG. 36A shows EC₅₀ values for binding affinity. FIG. 36B shows IC₅₀ values for neutralization potency.

FIGS. 37A-37B illustrate phylogenetic relations (FIG. 37A) and somatic hypermutation analysis (FIG. 37B) of closely related VH sequences identified using two SARS-COV2 neutralizing nanobodies (indicated with asterisk). Closely related, low abundance clonotypes were identified for secondary screening for high affinity and high potency nanobodies. The sequences of the nanobody clones in FIG. 37B from top to bottom are set forth in SEQ ID NOs: 2-12, respectively (Table 6).

FIG. 38 is a table presenting binding kinetics of mouse and human SAT nanobody-human Fc fusion molecules. Binding of mouse and human Nb-human Fc fusion proteins to the recombinant SAT protein was assayed by Bio-Layer Interferometry (BLI) using the Octet. These results demonstrate that the engineered mice provided herein can be used to obtain high affinity mouse nanobodies and high affinity human nanobodies.

FIG. 39 illustrates binding kinetics of exemplary human nanobody-human Fc fusion molecules. Sensograms of purified human Nb-human Fc fusion proteins in the presence of recombinant SAT obtained by BLI indicates high affinity.

FIG. 40 illustrates melting peaks of human nanobody-human Fc fusion molecules. Melting curves of purified human SAT Nb-human Fc fusion proteins were generated via pFUSE-hIgG1-Fc2 expression vector in Expi293F cells. These results demonstrate that the human Nb-human Fc molecules can exhibit thermos-stability similar to that of known natural nanobodies.

FIGS. 41A-41B illustrate cell binding assay results of mouse nanobody-human Fc fusion molecules and human nanobody-human Fc fusion molecules. FIG. 41A is an exemplary result showing positive control and negative control of HEK293 expressing SARS-Cov2 Spike proteins (top panel) or HEK293 (bottom panel) incubated in the presence of purified mouse or human Nb-human Fc fusion proteins. Cell binding was assessed using fluorescent secondary antibody against the Fc region of the Nb-Fc molecule. FIG. 41B shows summary results of the cell binding data for mouse and human Nb-human Fc fusion proteins.

FIG. 42 contains graphs showing the cell binding results for all Nb-human Fc fusions of FIG. 41A-41B. Top panel, mouse Nb-human Fcs; bottom panel, human Nb-human Fcs.

FIGS. 43A-43C illustrate hIGH-BAC5* and PCR confirmation of functional VH gene segments, and targeted sequencing validation of the Singularity Sapiens V5 allele containing hIGH-BAC1 through hIGH-BAC5*. FIG. 43A shows the VH elements in hIGH-BAC5* (boundaries marked in dashed boxes) based on human genome GRCh38/hg38 assembly GENCODE Genes Track (Version 36, October 2020). FIG. 43B contains a PCR analysis showing functional VH exon validation in engineered hIGH-BAC5*. FIG. 43C shows results from targeted sequencing of all functional human IgH V, D, J genes incorporated at the SSV5 allele. Biotinylated probe sets against each human IgH V, D, J genes were used for target enrichment to construct NGS libraries following standard protocols.

FIG. 44 contains an exemplary design of a singularity sapiens hVH+ array for a non-human animal. Shown is a schematic of a genetic construct (hVH+ array) containing 14 VH domains (e.g., human VH domains) designed to use mouse VH elements (e.g., mouse VH regulatory elements) as genetic scaffolds. An individual VH domain (e.g., an individual human VH domain) can be grafted onto a framework of a non-human VH segment (e.g., a mouse VH segment) that can include an upstream 250 bp promoter containing regulatory elements (e.g., a TATA box such as a mouse TATA box, an octamer such as a mouse octamer, and a heptamer such as a mouse heptamer), a leader exon 1 (e.g., a mouse leader exon 1), an intron (e.g., a mouse intron), a leader exon 2 (e.g., a mouse leader exon 2), and recombination signal sequences (e.g., 23RSS such as a mouse 23RSS).

FIGS. 45A-45B illustrate the genetic engineering of Singularity Sapiens V6 (SSV6) allele. FIG. 45A is a schematic illustrating an exemplary integration of a synthetic construct such as a human VH+ array that contains flanking disparate lox elements and a selection marker into the IgH locus of a Singularity Sapiens SSV5 (generated with either hIGH-BAC5 or hIGH-BAC5*) allele containing 44 functional human VH domains and all human heavy diversity (D_{H}) and (J_{H}) elements via recombination mediated cassette exchange (RMCE). FIG. 45B shows the validation of the complete integration of hVH+ array at the SSV6 allele by overlapping PCR. The PCR products (1-7) were verified by anger sequencing to match the corresponding VH genes.

FIGS. 46A-46D illustrate the genetic engineering of a Singularity Sapacos allele. FIG. 46A is a schematic of a genetic construct (Sapacos VHH array) containing 5 V_{H}H_{S} of the alpaca (Vicugna pacos) designed to use human VH elements as genetic scaffolds. Individual V_{H}H elements were grafted onto a framework of a selective human VH that includes an upstream promoter (e.g., a 250 bp upstream promoter) containing regulatory elements (e.g., a TATA box, an octamer, and a heptamer), a leader exon 1, an intron, a leader exon 2, and recombination signal sequences (e.g., 23RSS). FIG. 46B is a schematic showing that this synthetic construct (Sapacos VHH array) containing flanking disparate lox elements and a selection marker can be integrated into the Igh locus of a Singularity Sapiens allele containing all human VD and VJ elements via RMCE. FIG. 46C shows long range PCR validating the Singularity Sapiens DJ dock allele in F1 heterozygous pups, which is further confirmed by amplicon sequencing. FIG. 46D shows the validation of Singularity Sapacos allele by overlapping PCR in embryonic stem cells (PCR1-3) and in F1 heterozygous pups (PCR4).

FIGS. 47A-47C illustrate the genetic engineering of a Singularity Savnars allele. FIG. 47A is a schematic of a genetic construct (Savnars VNAR array) containing two germline VNARs from the nurse shark designed to use human VH elements as genetic scaffolds. Individual VNAR elements was grafted onto a framework of a selective human VH that includes an upstream promotor (e.g., a 250 bp upstream promoter) containing regulatory elements (e.g., a TATA box, an octamer, and a heptamer), a leader exon 1, an intron, a leader exon 2, and recombination signal sequences (e.g., 23RSS). FIG. 47B is a schematic showing that this synthetic construct (Savnars VNAR array) containing flanking disparate lox elements and a selection marker can be integrated into the Igh locus of the Singularity Sapiens allele containing all human VD and VJ elements via RMCE. FIG. 47C shows the correctly engineered embryonic stem cells have been verified by PCR1 as indicated followed by Sanger sequencing.

FIGS. 48A-48B illustrate RAG1/RAG-2 mediated VDJ recombination. FIG. 48A contains RAG1/RAG2-mediated recombination signal sequences for 12RSS (12 nt spacer: SEQ ID NO:98) and 23RSS (23 nt spacer: SEQ ID NO:99) associated with the variable segments at the human loci for IGH and TCR. FIG. 48B contains schematics demonstrating the 12/23 rule for VDJ recombination.

FIGS. 49A-49D illustrate the generation of an exemplary singularity sapiens TCR using TCRBV, TCRBD, and TCRBJ gene segments. FIG. 49A contains a map of the TCRB locus showing the boundary of BAC1 containing 7 TCRBV. 1 TCRBD1, and six TCRBJ segments in an exemplary hTCRB-VDJ-BAC1. FIG. 49B contains a schematic showing the insertion of hTCRB-VDJ-BAC1 via recombinase-mediated cassette exchange (RMCE) using heterospecific lox sites to swap alternating selection cassettes (neo and hyg) upon expression of Cre recombinase. FIG. 49C shows PCR confirmation of the correct and complete integration of hTCRB-VDJ-BAC1 at the corresponding lox sites and the presence of all 7 TCRBV gene segments. The PCR fragments were further verified by sanger sequencing. FIG. 49D shows genotyping PCR of F1 heterozygous pups.

FIGS. 50A-50B illustrate the generation of an exemplary singularity sapiens TCR using TCRBV, IGHD, and IGHJ gene segments. FIG. 50A contains a map of the TCRB locus showing the boundary of BAC1 containing 7 TCRBV gene segments to be used in engineering hTCRB-V-BAC1. FIG. 50B contains a schematic showing the insertion of hTCRB-V-BAC1 via RMCE using heterospecific lox sites to swap alternating selection cassettes (neo and hyg) upon expression of Cre recombinase.

FIGS. 51A-51B illustrate similarities between a ¹⁰FN3 amino acid sequence and an amino acid sequence of a heavy chain variable region and biophysical properties of ¹⁰FN3 constructs. FIG. 51A shows the common hypervariable loops and complementarity-determining region (CDR) 1, CDR2, and CDR3 for HcAb HEL4 (HEL4: SEQ ID NO:95) and comparable hypervariable loops BC, DE, and FG in ¹⁰FN3 (SEQ ID NO:96). FIG. 51B illustrates the amino acid sequences of ¹⁰FN3M1 (SEQ ID NO: 96) and ¹⁰FN3M2 (SEQ ID NO:97) and cartoons of the ¹⁰FN3M1 and the ¹⁰FN3M2 using the human Ig hinge, CH2, and CH3 framework for transient expression in CHO cells.

FIGS. 52A-52B contain an exemplary design of a ¹⁰FN3VM1 synthetic construct. FIG. 52A shows RAG1/RAG2-mediated recombination signal sequences for 12RSS (12 nt spacer: SEQ ID NO:98) and 23RSS (23 nt spacer: SEQ ID NO:99) and their attachment to exemplary ¹⁰FN3 VM and JM segments (shown as SEQ ID NO:100). FIG. 52B illustrates how the VDJ recombination generates a ¹⁰FN3 “VM” segment (containing residues 1-80 of SEQ ID NO:96) and a ¹⁰FN3 “JM” (containing residues 81-95 of SEQ ID NO:96). Shown atop ¹⁰FN3VM1 is a mouse VH element acting as a genetic scaffold that includes an upstream 250 bp promoter, a leader exon 1, an intron, a leader exon 2, and recombination signal sequences (e.g., 23RSS).

FIG. 53 Generation of the Singularity Sapiens Monobody allele, which leads toa mouse having the ability to generate antibody-like molecules described herein. Shown is a two-step process for deleting the human J_{H}S cluster and replacing it with a ¹⁰FN3JM element using a CRISPR-Cas9 system. This is followed by recombinase mediated cassette exchange (RMCE)-mediated insertion of the ¹⁰FN3VM1 construct using heterospecific lox sites to swap alternating selection cassettes (neo and hyg) upon expression of Cre recombinase. The correctly engineered embryonic stem cells have been verified by PCR1-3 as indicated followed by Sanger sequencing.

FIGS. 54A-54B. Generation of the Singularity Sapiens W2X allele. FIG. 54A contains a schematic of an exemplary synthetic JH-W2X construct where the human IGHJ3 and IGHJ4 gene segments contained a nucleic acid substitution (indicated with asterisk) such that the tryptophan residue at the Kabat position 103 was changed to arginine and histidine, respectively. Alternatively, other non-aggregating amino acids including threonine, lysine, tyrosine, serine, or glutamine can be used. These modified IGHJ gene segments are referred to by a W2X substitution. FIG. 54B contains a schematic illustrating replacement of the endogenous IGHJ3 and IGHJ4 with the JH-W2X construct of a singularity sapiens allele (top) using a CRISPR-Cas9 system to generate the singularity sapiens W2X (bottom). The modified IGHJ3 and IGHJ4 are indicated with asterisks.

FIG. 55 contains a schematic of an exemplary synthetic camelized human V_{H} gene segment array. The synthetic human V_{H} gene segment array shown includes five human V_{H} gene segments modified to encode V37Y, G44E. L45R, and W47G amino acid substitutions in FR2 (camelization). Each modified human V_{H} gene segment has an upstream 250 bp promoter containing regulatory elements (e.g., a TATA box, an octamer, and a heptamer), a leader exon 1, an intron, a leader exon 2, and 100 bp downstream sequence containing the recombination signal sequences (e.g., 23RSS).

FIG. 56. Generation of the Singularity Sapiens Camelized allele. Shown is a schematic illustrating the integration of a construct encoding an exemplary synthetic camelized human VH gene segment array (CHVHs) and containing flanking disparate lox elements into the IgH locus of a Singularity Sapiens DJ dock allele via recombination-mediated cassette exchange (RMCE). The correctly engineered embryonic stem cells have been verified by PCR1 as indicated followed by Sanger sequencing.

FIG. 57 contains a schematic of an exemplary input nanobody domain for in vivo affinity maturation (e.g., a Syn Nbx construct). The Syn Nbx construct contains (i) a coding sequence for a selected nanobody (Nb CDS (V-D-J)), (ii) the promoter, 5′ UTR, and lead exons of an exemplary mouse IgH VH gene segment (e.g., a mouse IGHV1-26), and (iii) a splice donor sequence of an exemplary mouse IgH JH gene segment (e.g., a mouse IGHJ1 splice donor sequence).

FIG. 58 is a schematic showing integration of a Syn Nbx construct containing disparate lox elements and a selection marker (top) into the IgH locus of a singularity sapiens hyperdock allele via recombinase-mediated cassette exchange (RMCE) to generate a singularity Nbx allele (bottom).

FIG. 59 is a schematic showing the “Trojan mouse” approach to generate chimeric mice using B-cell deficient embryos and engineered embryonic stem cells. Since no antibodies are produced from B-cell deficient host mouse, the humoral immune responses of the resulting chimeric mouse are mediated solely by antibodies generated from the B-cells derived from the engineered ES cells, eliminating the need for lengthy multigenerational breeding otherwise required to produce homozygous mice. This “Trojan mouse” approach is broadly applicable for generating chimeric cohorts directly from engineered ES cells carrying IgH mutations for immunization.

### DETAILED DESCRIPTION

This document provides information on how to generate and utilize genetically modified or engineered non-human animals (e.g., genetically modified or engineered mice) as well as methods and materials for making antibodies using such animals.

### Engineered Non-Human Animals Having Expanded Variable Heavy Chain Segments

In one aspect, this document relates to genetically modified or engineered non-human animals (e.g., genetically modified or engineered mice) that produce antibodies (e.g., sdAbs and/or heavy chain only antibodies) having a chimeric heavy chain that includes (1) a non-human CH2 domain and/or a non-human CH3 domain (and optionally a non-human Ig hinge) and (2) a VH domain such as a human VH domain encoded by a VH gene segment set forth in Example 6 (e.g., a VH domain set forth in any one of SEQ ID NOs: 74-87), and methods of making and using the same. For example, this document describes genetically engineered non-human animals of a particular species (e.g., a mouse species) that produce antibodies (e.g., sdAbs and/or heavy chain only antibodies) having one or two chimeric heavy chain that include (1) a CH2 domain of that same species (e.g., a mouse CH2 domain) and/or a CH3 domain (and optionally a non-human Ig hinge) of that same species (e.g., a mouse CH2 domain) and (2) a VH domain such as a human VH domain encoded by a VH gene segment set forth in Example 6 (e.g., a VH domain set forth in any one of SEQ ID NOs: 74-87).

Antibodies produced by a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can be any appropriate type of antibody. In some cases, an antibody can include at least one VH domain. Examples of antibodies that can be produced by a non-human animal provided herein include, without limitation, heavy chain only antibodies. Examples of antibodies that can be designed or generated from an antibody produced by a non-human animal provided herein include, without limitation, sdAbs, Fabs, Fab′, F(ab′)₂, Fd, Fvs, single-chain variable fragments (scFvs), heavy chain antibodies (also referred to as heavy chain only antibodies), and disulfide-linked Fvs (sdFv). In some cases, non-human animals (e.g., mice) provided herein can, when exposed to one or more antigens, produce IgG1 heavy chain only antibodies. IgG2 heavy chain only antibodies. IgG3 heavy chain only antibodies. IgG4 heavy chain only antibodies, or combinations thereof.

A chimeric heavy chain of an antibody (e.g., a heavy chain only antibody) produced by a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 and/or a non-human CH3 domain (and optionally a non-human Ig hinge) and (2) a VH domain such as a VH domain set forth in any one of SEQ ID NOs: 74-87) can include any appropriate VH domain (e.g., any appropriate human VH domain). Examples of human VH domains that can be included in a chimeric heavy chain produced by a non-human animal provided herein include, without limitation, an IGHV1-8*01 encoded amino acid sequence (SEQ ID NO:74), an IGHV3-9*01 encoded amino acid sequence (SEQ ID NO:75), an IGHV4-31*03 encoded amino acid sequence (SEQ ID NO:76), an IGHV4-30-4*01 encoded amino acid sequence (SEQ ID NO:77), an IGHV4-38-2*02 encoded amino acid sequence (SEQ ID NO:78), an IGHV3-43D*04 encoded amino acid sequence (SEQ ID NO:79), an IGHV3-35*02 encoded amino acid sequence (SEQ ID NO:80), an IGHV3-62*04 encoded amino acid sequence (SEQ ID NO:81), an IGHV3-16*02 encoded amino acid sequence (SEQ ID NO: 82), an IGHV3-38*02 encoded amino acid sequence (SEQ ID NO:83), an IGHV3-38-3*01 encoded amino acid sequence (SEQ ID NO:84), an IGHV1-38-4*01 encoded amino acid sequence (SEQ ID NO:85), an IGHV8-51-1*02 encoded amino acid sequence (SEQ ID NO:86), and an IGHV7-81*01 encoded amino acid sequence (SEQ ID NO:87).

A non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can be any type of non-human animal. Examples of non-human animals that can be designed to produce antibodies (e.g., sdAbs and/or heavy chain only antibodies) having one or two chimeric heavy chains that include (1) a non-human CH2 domain and/or a non-human CH3 domain (and optionally a non-human Ig hinge) and (2) a VH domain (e.g., a VH domain set forth in any one of SEQ ID NOs: 74-87) as described herein include, without limitation, mice, rats, rabbits, guinea pigs, zebrafish, flies (e.g., Drosophila melanogaster), pigs, sheep, non-human primates (e.g., monkeys), and bovine species. In some cases, a non-human animal designed to produce antibodies (e.g., sdAbs and/or heavy chain only antibodies) having one or two chimeric heavy chains that include (1) a mouse CH2 domain and/or a mouse CH3 domain (and optionally a non-human Ig hinge) and (2) a VH domain (e.g., a VH domain set forth in any one of SEQ ID NOs: 74-87) can be a mouse.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can be designed to produce antibodies that lack CH1 domains and that lack light chains. For example, a non-human animal (e.g., a mouse) provided herein can be a non-human animal whose genome has (e.g., is genetically engineered to have) one or more disruptions in an endogenous nucleic acid sequence encoding an CH1 domain of an IgG1 C-region gene (e.g., C_{γ1}), such that an IgG antibody encoded by the non-human animal lacks a CH1 domain and lacks light chains. In some cases, the genome of a non-human animal provided herein can lack nucleic acid encoding at least a portion of an endogenous CH1 domain. In some cases, the genome of a non-human animal provided herein can lack at least a portion of an endogenous regulatory element that drives expression of an endogenous CH1 domain.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can be designed to have a deletion of endogenous nucleic acid encoding a CH1 domain of a constant region (e.g., of an IgG C-region such as a CH1 domain of an IgG1 C-region, a CH1 domain of an IgG2a C-region, a CH1 domain of an IgG2b C-region, and/or a CH1 domain of an IgG3 C-region). The CH1 domain can contain multiple exons. In some cases, endogenous exon 1 of a CH1 domain of a constant region such as an IgG C-region can be deleted such that the engineered non-human animal (e.g., mouse) produces IgMΔCH1, IgGΔCH1, IgDΔCH1, IgAΔCH1, and/or IgEΔCH1 heavy chain only antibodies.

When making one or more genetic modifications to delete all or part of the nucleic acid encoding a CH1 domain (e.g., a CH1 domain of an IgG1 C-region, a CH1 domain of an IgG2a C-region, a CH1 domain of an IgG2b C-region, and/or a CH1 domain of an IgG3 C-region) such that the engineered non-human animal produces IgΔCH1 heavy chain only antibodies (e.g., IgGΔCH1 heavy chain only antibodies), the endogenous nucleic acid encoding a hinge domain, a heavy chain CH2 domain, and a heavy chain CH3 domain (and optionally a non-human Ig hinge) can remain intact. For example, to make a mouse that produces IgG1ΔCH1 heavy chain only antibodies, the genome of that mouse can lack exon 1 (and/or additional portions) of the CH1 domain of IgG1 while retaining the endogenous mouse nucleic acid needed to express the hinge domain, a heavy chain CH2 domain, and a heavy chain CH3 domain of IgG1, thereby resulting in a mouse that is capable of producing IgG1ΔCH1 heavy chain only antibodies.

Additional endogenous nucleic acid components that can be deleted from the genome of a non-human animal (e.g., a genetically engineered non-human animal such as a mouse as described herein that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) to create a non-human animal as described herein include, without limitation, the introns and/or exons of the IgM constant domains (e.g., the μ constant domain locus), the introns and/or exons of the IgD constant domains (e.g., the δ constant domain locus), the introns and/or exons of the IgE constant domains (e.g., the ε constant domain locus), and/or the introns and/or exons of the IgA constant domains (e.g., the α constant domain locus). For example, a non-human animal described herein can be designed to lack the introns and exons of the IgM constant domains (e.g., the μ constant domain locus), the introns and exons of the IgD constant domains (e.g., the δ constant domain locus), the introns and exons of the IgE constant domains (e.g., the ε constant domain locus), and the introns and exons of the IgA constant domains (e.g., the α constant domain locus).

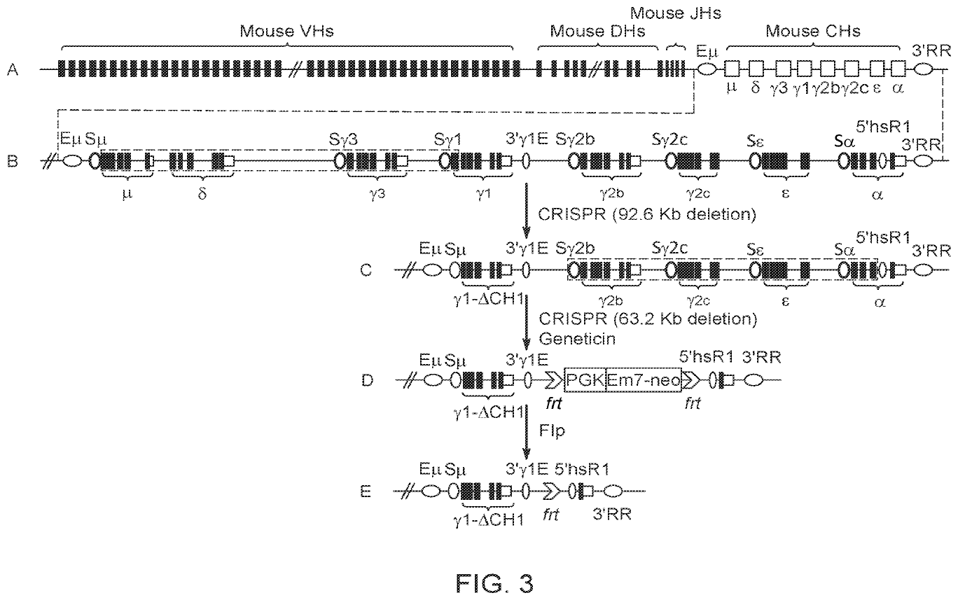

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) to produce only IgG1ΔCH1 heavy chain only antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus). An example of a genetic engineering approach to create a mouse that produces only IgG1ΔCH1 heavy chain only antibodies is set forth in FIGS. 3A-3E.

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) to produce only IgG2aΔCH1 heavy chain only antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ1 constant domains (e.g., the γ1 constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) to produce only IgG2bΔCH1 heavy chain antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ1 constant domains (e.g., the γ1 constant domain locus), and the endogenous (if endogenously present) introns and/or exons of the Igγ2c constant domains (e.g., the γ2c constant domain locus).

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) to produce only IgG2cΔCH1 heavy chain antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ1 constant domains (e.g., the γ1 constant domain locus).

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) to produce only IgG3ΔCH1 heavy chain antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ1 constant domains (e.g., the γ1 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

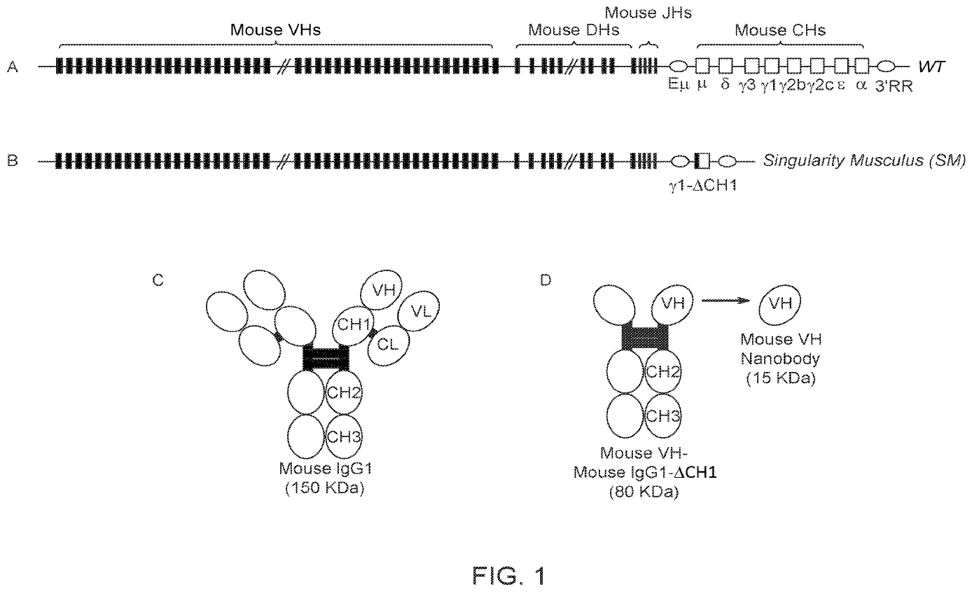

As described herein, retaining and/or creating new positioning for certain endogenous enhancer or regulatory elements of a non-human animal can result in a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) that produces effectively large collections of and/or amounts of diverse heavy chain only antibodies (e.g., heavy chain only antibodies such as chimeric human/mouse heavy chain only antibodies). For example, a non-human animal provided herein can be designed to retain the μ enhancer (Eμ), the μ switch region (Sμ), and/or the μ promoter containing I-exon (Iμ) that are endogenously found upstream of the nucleic acid encoding the IgM constant domains. In some cases, non-human animals (e.g., mice) provided herein can be designed such that the retained endogenous Eμ, Sμ, and/or Iμ elements are in a genomic position such that the first nucleic acid sequence downstream of the retained Eμ. Sμ, and/or Iμ elements that encodes a full-length endogenous Ig constant domain is one that encodes a CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 1B and FIG. 3C where the nucleic acids of the endogenous mouse Eμ, Sμ, and Iμ elements are repositioned to be upstream of the nucleic acid encoding the endogenous IgG1 CH2 domain.

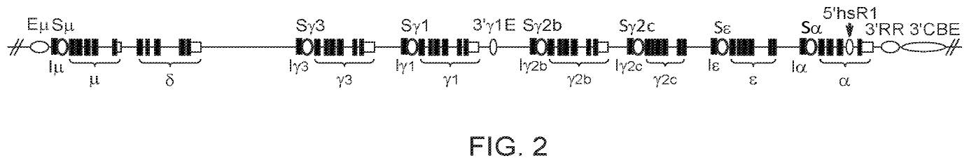

In another example, a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can be designed to retain the 3′RR and/or 3 CBE elements that are endogenously found downstream of the nucleic acid encoding the IgA constant domains. In some cases, non-human animals (e.g., mice) as described herein can be designed such that the retained endogenous 3′RR and/or 3 CBE elements are in a genomic position such that the first nucleic acid sequence upstream of the retained 3′RR and/or 3 CBE elements that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 2B and FIG. 3E where the nucleic acid of the endogenous mouse 3′RR element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 3′RR element.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can be designed to retain the 3′γ1E element that is endogenously found between, for example, the IgG1 and IgG2b loci. In some cases, non-human animals (e.g., mice) provided herein can be designed such that the retained endogenous 3′γ1E element is in a genomic position such that nucleic acid encoding two, one, or no full-length endogenous Ig CH2 domains is located between the retained endogenous 3′γ1E element and a retained endogenous 3′RR element and/or a retained endogenous 3′CBE element. An example of this genomic configuration is set forth in FIG. 3E where the nucleic acid of the endogenous mouse 3′γ1E element is repositioned to be upstream of a retained endogenous 3′RR element such that no other nucleic acid encoding a full-length IgG CH2 domain is located between the endogenous mouse 3′γ1E element and the endogenous 3′RR element.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can be designed to retain the 5′hsR1 element that is endogenously found within the IgA constant domain locus. In some cases, non-human animals (e.g., mice) provided herein can be designed such that the retained endogenous 5′hsR1 element is in a genomic position such that the first nucleic acid sequence upstream of the retained 5′hsR1 element that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 3E where the nucleic acid of the endogenous mouse 5′hsR1 element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 5 hsR1 element.

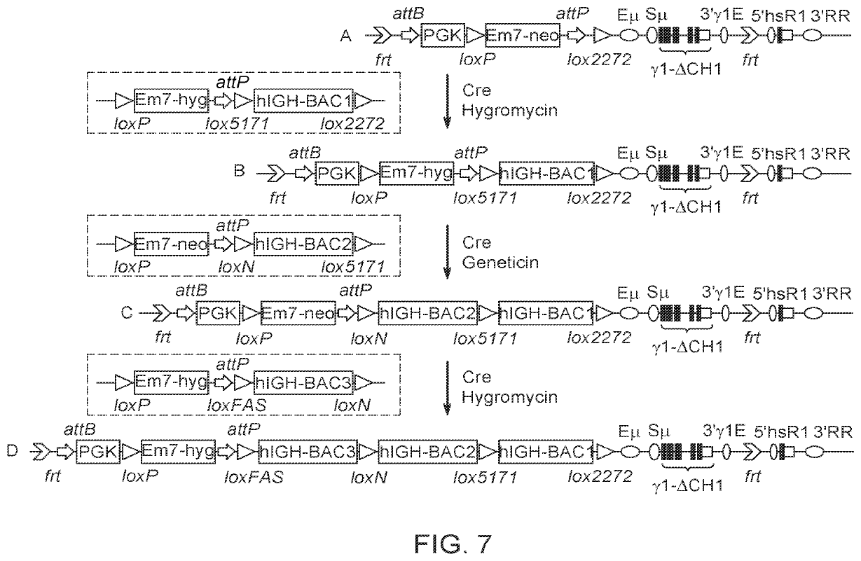

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can be designed to have (a) a VH locus (e.g., a VH locus including one or more human VH gene segments) followed by (b) an endogenous Eμ element and/or an endogenous element Iμ and/or an endogenous Su element followed by (c) nucleic acid that encodes endogenous IgG hinge. CH2 domain, and CH3 domain in the absence of the endogenous CH1 domain for that IgG followed by (e) an endogenous 3′γ1E element, an endogenous 3′RR element, and an endogenous 3′CBE element, while lacking the endogenous nucleic acid that encodes at least one full-length CH2 domain or CH3 domain of each of IgM, IgD, IgE, and IgA. An example of this genomic configuration is set forth in FIG. 3E. See, also, FIGS. 7, 8, 43B, 44, and 46.

In some cases, instead of retaining an endogenous enhancer or regulatory element as described herein, one or more exogenous enhancer or regulatory elements can be engineered into a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87). For example, in some cases, a mouse can be designed as described herein where the endogenous mouse Eμ element is removed and replaced with a human Eμ element.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can be designed to produce antibodies that lack endogenous VH domains. For example, non-human animals provided herein can be non-human animals whose genomes have (e.g., are genetically engineered to have) one or more disruptions in an endogenous nucleic acid sequence encoding a VH locus, such that antibodies encoded by the non-human animal can lack endogenous VH domains. In some cases, at least one allele of the genome of a non-human animal provided herein can lack at least a portion of the endogenous nucleic acid encoding one or more VH domains. In some cases, at least one allele of the genome of a non-human animal provided herein can lack all the exons of the endogenous nucleic acid encoding VH domains. In some cases, both alleles of the genome of a non-human animal provided herein can lack all the exons of the endogenous Ig heavy chain variable region. In some cases, neither allele of the genome of a non-human animal provided herein contains an endogenous exon of a nucleic acid encoding a VH domain.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can be designed to have a variable region locus that is not endogenous to that non-human animal. For example, a mouse as described herein can be designed to have a non-mouse VH locus (e.g., a VH locus including one or more human VH gene segments).

A non-mouse VH locus (e.g., a VH locus including one or more human VH gene segments and/or one or more VH gene segments encoding a VH domain set forth in any of SEQ ID NOs: 74-87) present in the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can have any appropriate number of exogenous VH gene segments. In some cases, a non-mouse VH locus can include two, three, four, five, six, seven, eight, nine, ten, 11, 12, 13, 14, or more VH gene segments that encode VH domains that are not mouse VH domains.

A non-mouse VH locus (e.g., a VH locus including one or more human VH gene segments and/or one or more VH gene segments encoding a VH domain set forth in any of SEQ ID NOs: 74-87) present in the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can have any appropriate VH gene segments. In some cases, a non-mouse VH locus (e.g., a VH locus including one or more human VH gene segments and/or one or more VH gene segments encoding a VH domain set forth in any of SEQ ID NOs: 74-87) present in the genome of a non-human animal provided herein can include exogenous nucleic acid encoding one or more (e.g., one, two, three, four, five, six, seven, eight, nine, ten, 11, 12, 14, 15, 25, 35, 50, 65, 80, 100, 125, or more) human VH gene segments and/or one or more (e.g., one, two, three, four, five, six, seven, eight, nine, ten, 11, 12, or 14) VH gene segments encoding a VH domain set forth in any one of SEQ ID NOs: 74-87. For example, an IgH allele of the genome of a non-human animal provided herein can include exogenous nucleic acid encoding from 1 to 129 human VH gene segments. In some cases, a non-mouse VH locus (e.g., a VH locus including one or more human VH gene segments and/or one or more VH gene segments encoding a VH domain set forth in any of SEQ ID NOs: 74-87) present in the genome of a non-human animal provided herein can include exogenous nucleic acid encoding one or more (e.g., one, two, three, four, five, six, seven, eight, nine, ten, 11, 12, 13, 14, 15, 25, 27, or more) human DH gene segments. For example, an IgH allele of the genome of a non-human animal provided herein can include exogenous nucleic acid encoding from 1 to 27 human DH gene segments. In some cases, a non-mouse VH locus (e.g., a VH locus including one or more human VH gene segments and/or one or more VH gene segments encoding a VH domain set forth in any of SEQ ID NOs: 74-87) present in the genome of a non-human animal provided herein can include exogenous nucleic acid encoding one or more (e.g., one, two, three, four, five, six, seven, eight, nine, ten, 11, 12, 13, 14, or more) human VH gene segments. For example, an IgH allele of the genome of a non-human animal provided herein can include exogenous nucleic acid encoding from 1 to 9 human VH gene segments. In some cases, at least one IgH allele of the genome of a non-human animal provided herein can include exogenous nucleic acid encoding 129 or more human VH gene segments, 27 or more human Ig DH gene segments, and 9 or more human Ig VH gene segments.

A non-mouse VH locus (e.g., a VH locus including one or more human VH gene segments and/or one or more VH gene segments encoding a VH domain set forth in any of SEQ ID NOs: 74-87) present in the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can have any appropriate one or more VH gene segments. Examples of VH gene segments that can be included in a genome of a non-human animal provided herein include, without limitation, the VH gene segments set forth at IGHV1-8*01 (SEQ ID NO:18), IGHV3-9*01 (SEQ ID NO:20). IGHV4-31*03 (SEQ ID NO:22). IGHV4-30-4*01 (SEQ ID NO: 24), IGHV4-38-2*02 (SEQ ID NO:26), IGHV3-43D*04 (SEQ ID NO:28), of IGHV3-35*02 (SEQ ID NO:30), IGHV3-62*04 (SEQ ID NO:32), IGHV3-16*02 (SEQ ID NO: 34), IGHV3-38*02 (SEQ ID NO:36), IGHV3-38-3*01 (SEQ ID NO:38), IGHV1-38-4*01 (SEQ ID NO:40). IGHV8-51-1*02 (SEQ ID NO:42), and IGHV7-81*01 (SEQ ID NO: 44). Examples of VH gene segments that can be included in a genome of a non-human animal provided herein also include, without limitation, VH gene segments that encode the amino acid sequence encoded by IGHV1-8*01, which amino acid sequence is set forth in SEQ ID NO:74; VH gene segments that encode the amino acid sequence encoded by IGHV3-9*01, which amino acid sequence is set forth in SEQ ID NO:75; VH gene segments that encode the amino acid sequence encoded by IGHV4-31*03, which amino acid sequence is set forth in SEQ ID NO:76; VH gene segments that encode the amino acid sequence encoded by IGHV4-30-4*01, which amino acid sequence is set forth in SEQ ID NO:77; VH gene segments that encode the amino acid sequence encoded by IGHV4-38-2*02, which amino acid sequence is set forth in SEQ ID NO:78; VH gene segments that encode the amino acid sequence encoded by IGHV3-43D*04, which amino acid sequence is set forth in SEQ ID NO:79; VH gene segments that encode the amino acid sequence encoded by IGHV3-35*02, which amino acid sequence is set forth in SEQ ID NO: 80; VH gene segments that encode the amino acid sequence encoded by IGHV3-62*04, which amino acid sequence is set forth in SEQ ID NO:81; VH gene segments that encode the amino acid sequence encoded by IGHV3-16*02, which amino acid sequence is set forth in SEQ ID NO:82; VH gene segments that encode the amino acid sequence encoded by IGHV3-38*02, which amino acid sequence is set forth in SEQ ID NO:83; VH gene segments that encode the amino acid sequence encoded by IGHV3-38-3*01, which amino acid sequence is set forth in SEQ ID NO:84; VH gene segments that encode the amino acid sequence encoded by IGHV1-38-4*01, which amino acid sequence is set forth in SEQ ID NO:85; VH gene segments that encode the amino acid sequence encoded by IGHV8-51-1*02, which amino acid sequence is set forth in SEQ ID NO:86; and VH gene segments that encode the amino acid sequence encoded by IGHV7-81*01, which amino acid sequence is set forth in SEQ ID NO:87. In some cases, nucleic acid sequences of VH gene segments that can be included in a genome of a non-human animal provided herein can be as described in Example 6.

In some cases, a non-mouse VH locus (e.g., a VH locus including one or more human VH gene segments and/or one or more VH gene segments encoding a VH domain set forth in any of SEQ ID NOs: 74-87) present in the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can include one or more regulatory elements for each VH gene segment in the non-mouse VH locus (e.g., to control expression and/or recombination for each VH gene segment). The regulatory element(s) can be endogenous or exogenous. Examples of regulatory elements that can be used to control expression and/or recombination for each VH gene segment include, without limitation, promoters, enhancers, transcription factor binding sites, splice sites, recombination signal sequences, leader exon 1, leader exon 2, signal peptide sequences, and introns.

In some cases, a non-mouse VH locus (e.g., a VH locus including one or more human VH gene segments and/or one or more VH gene segments encoding a VH domain set forth in any of SEQ ID NOs: 74-87) present in the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can include one or more additional components. The additional component(s) can be endogenous or exogenous. For example, a non-mouse VH locus (e.g., a VH locus including one or more human VH gene segments and/or one or more VH gene segments encoding a VH domain set forth in any of SEQ ID NOs: 74-87) present in the genome of a non-human animal provided herein can include nucleic acid (e.g., an endogenous exon) encoding a leader sequence (e.g., such that antibodies such as sdAbs and/or heavy chain only antibodies produced by the non-human animal include the encoded leader sequence). In some cases, a non-mouse VH locus (e.g., a VH locus including one or more human VH gene segments and/or one or more VH gene segments encoding a VH domain set forth in any of SEQ ID NOs: 74-87) present in the genome of a non-human animal provided herein can include nucleic acid (e.g., an endogenous exon) encoding a leader sequence for each VH gene segment in the non-mouse VH locus. Examples of leader sequences that can be included in a non-mouse VH locus (e.g., a VH locus including one or more human VH gene segments and/or one or more VH gene segments encoding a VH domain set forth in any of SEQ ID NOs: 74-87) present in the genome of a non-human animal provided herein include, without limitation, an L1 exon of the non-human animal (e.g., a mouse L1 exon), and L2 exon of the non-human animal (e.g., a mouse L2 exon), and other peptide signal sequences.

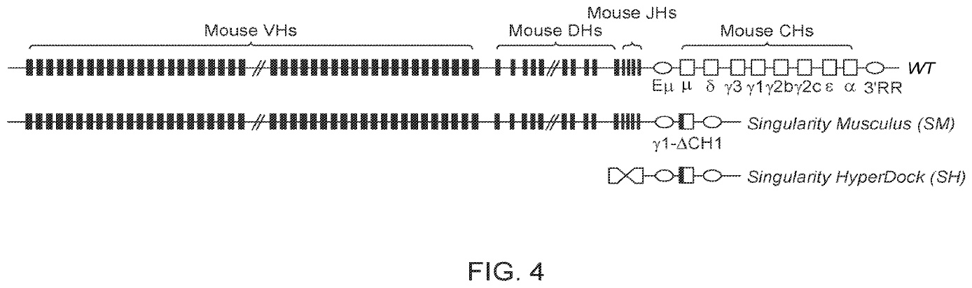

Any appropriate method can be used to generate a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87). In some cases, nucleic acid comprising one or more human VH gene segments and/or one or more VH gene segments encoding a VH domain set forth in any of SEQ ID NOs: 74-87 (e.g., synthetic array of VH gene segments encoding one or more VH domains set forth in any of SEQ ID NOs: 74-87) can be inserted into the genome of a non-human animal (e.g., a mouse) that lacks nucleic acid encoding at least a portion of an endogenous CH1 (or lacks at least a portion of an endogenous regulatory element that drives expression of an endogenous CH1 domain) and optionally lacks at least a portion of the endogenous nucleic acid encoding one or more (e.g., all) endogenous VH domains. For example, a VH locus (e.g., nucleic acid comprising one or more human VH gene segments and/or one or more VH gene segments encoding a VH domain set forth in any of SEQ ID NOs: 74-87) can be inserted into the genome of an engineered mouse as set forth in FIGS. 4, 5D, and 6B (also referred to as a Singularity HyperDock non-human animal or a Singularity HyperDock mouse). In some cases, nucleic acid comprising one or more human VH segments and/or one or more VH gene segments encoding a VH domain set forth in any of SEQ ID NOs: 74-87 can include at least one recombination site (e.g., at least one nucleic acid sequence that can be recognized by a recombinase) on each end (e.g., at the 5′ end and at the 3′ end) of the one or more human VH segments and/or the one or more VH gene segments encoding a VH domain set forth in any of SEQ ID NOs: 74-87. In some cases, the non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of the endogenous nucleic acid encoding one or more (e.g., all) VH domains (e.g., a Singularity HyperDock mouse) can include at least one recombination site, such that a recombinase can facilitate the insertion of nucleic acid comprising one or more human VH segments and/or the one or more VH gene segments encoding a VH domain set forth in any of SEQ ID NOs: 74-87 into the genome of that non-human animal.

In some cases, nucleic acid comprising one or more human VH gene segments and/or the one or more VH gene segments encoding a VH domain set forth in any of SEQ ID NOs: 74-87 can be present on a vector. Examples of vectors that can include nucleic acid comprising one or more human VH gene segments and/or the one or more VH gene segments encoding a VH domain set forth in any of SEQ ID NOs: 74-87 include, without limitation, yeast artificial chromosomes (YACs), bacterial artificial chromosomes (BACs), human artificial chromosomes (HACs), transchromosomes (e.g., whole or fragmented transchromosomes), P1-derived artificial chromosome (PACs), plasmids, and phagemids.

A non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of endogenous nucleic acid encoding one or more VH domains can include any appropriate recombination site(s). In some cases, a recombinase site can be an exogenous recombinase site. Examples of recombination sites that can be present in the genome of a non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of endogenous nucleic acid encoding one or more VH domains include, without limitation, frt, loxP, M2, M3, lox2271, lox2372, loxFAS, loxN, lox5171, lox2272, attB, and attP. Additional examples of recombination sites that can be present in the genome of a non-human animal (e.g., a mouse) provided herein can be as shown in Table 1.

TABLE 1
Exemplary recombination site recognition sequences.

| | | SEQ | | SEQ | | SEQ |
|---|---|---|---|---|---|---|
| | | ID | | ID | | ID |
| Lox Site | 13 bp Recognition Region | NO | 8 bp Spacer Region | NO | 13 bp Recognition Region | NO |
| loxP(wildtype) | ATAACTTCGTATA | 124 | GCATACAT | 142 | TATACGAAGTTAT | 160 |
| lox2272 | ATAACTTCGTATA | 125 | GgATACtT | 143 | TATACGAAGTTAT | 161 |
| lox5171 | ATAACTTCGTATA | 126 | GtAcACAT | 144 | TATACGAAGTTAT | 162 |
| loxN | ATAACTTCGTATA | 127 | GtATACCT | 145 | TATACGAAGTTAT | 163 |
| loxFAS | ATAACTTCGTATA | 128 | GaAaggta | 146 | TATACGAAGTTAT | 164 |
| lox2372 | ATAACTTCGTATA | 129 | GgATACcT | 147 | TATACGAAGTTAT | 165 |
| M2 | ATAACTTCGTATA | 130 | tggTttcT | 148 | TATACGAAGTTAT | 166 |
| M3 | ATAACTTCGTATA | 131 | taATACca | 149 | TATACGAAGTTAT | 167 |
| M7 | ATAACTTCGTATA | 132 | ttcTAtcT | 150 | TATACGAAGTTAT | 168 |
| M11 | ATAACTTCGTATA | 133 | agATAgaa | 151 | TATACGAAGTTAT | 169 |
| lox66 | ATAACTTCGTATA | 134 | GCATACAT | 152 | TATACGAAcggta | 170 |
| lox71 | taccgTTCGTATA | 135 | GCATACAT | 153 | TATACGAAGTTAT | 171 |
| lox72 | taccgTTCGTATA | 136 | GCATACAT | 154 | TATACGAAcggta | 172 |
| lox511 | ATAACTTCGTATA | 137 | GtATACAT | 155 | TATACGAAGTTAT | 173 |
| loxBri | ATAACTTCGTATA | 138 | aacTAtAc | 156 | TATACGAAGTTAT | 174 |
| lox2271 | ATAACTTCGTATA | 139 | GtATACET | 157 | TATACGAAGTTAT | 175 |
| lox5372 | ATAACTTCGTATA | 140 | GgAgACAT | 158 | TATACGAAGTTAT | 176 |
| lox4171 | ATAACTTCGTATA | 141 | GtATgCAT | 159 | TATACGAAGTTAT | 177 |

A genome of a non-human animal (e.g., a mouse) described herein can include any appropriate number of recombination sites. For example, a non-human animal (e.g., a mouse) described herein can include one, two, three, four, five, six, seven, eight, nine, ten, or more recombination sites. When a non-human animal (e.g., a mouse) as described herein includes two or more recombination sites in its genome, the recombination sites can each be a different recombination site. For example, a non-human animal (e.g., a mouse) described herein can include at least 3 different recombination sites within its genome. For example, a non-human animal (e.g., a mouse) described herein can include at least 5 different recombination sites within its genome.

A recombination site in a non-human animal (e.g., a mouse) described herein can be at any appropriate location within the genome of the non-human animal. In some cases, one or more recombination sites within a genome of a non-human animal as described herein can be upstream of endogenous nucleic acid encoding a CH2 domain or a CH3 domain. In some cases, one or more recombination sites within a genome of a non-human animal described herein can be upstream (e.g., less than 2.5 Mb upstream) of an endogenous Eμ element. When a genome of a non-human animal (e.g., a mouse) described herein includes different recombination sites (e.g., different exogenous recombination sites), each of the recombination sites can be located less than 2.0 Mb, less than 1.5 Mb, less than 1.0 Mb, less than 500 kb, less than 250 kb, less than 200 kb, less than 100 kb, less than 50 kb, less than 25 kb, or less than 10 kb upstream of an endogenous Eμ element.

Any appropriate method can be used to make a non-human animal as described herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87). For example, gene editing techniques (e.g., CRISPR/Cas gene editing, TALEN gene editing, and/or zinc finger-based gene editing), recombination techniques (e.g., sequential Recombinase Mediated Cassette Exchange (RMCE)), and combinations thereof can be used to make a non-human animal as described herein. In some cases, a nucleic acid molecule (e.g., an exogenous nucleic acid molecule) comprising one or more Ig VH gene segments (e.g., 1, 2, 3, 4, 5, 6, 7, 8, 9, 10, 11, 12, 13, or 14 VH gene segments that encode VH domains as set forth in SEQ ID NOs: 74-87) can be introduced into a stem cell of a non-human animal (e.g., a mouse), the stem cell can be implanted into a blastocyst of the same species of non-human animal, and the blastocyst can be implanted into a pseudo-pregnant female of the same species of non-human animal to obtain a chimeric non-human animal, crossing the chimeric non-human animal to a wild-type non-human animal to produce offspring, screening the offspring for heterozygosity, and identifying a founder non-human animal carrying the one or more VH gene segments within its genome. In some cases, a non-human animal provided herein can be made as described in the Examples.

This document also provides methods for producing populations of antibodies (e.g., sdAbs and/or heavy chain only antibodies) having expanded VH diversity. For example, a non-human animal described herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can be administered one or more antigens (e.g., a composition including one or more antigens), such that one or more B cells in the non-human animal produce an antibody (e.g., a heavy chain only antibody) that includes a chimeric heavy chain that includes (1) a non-human CH2 domain encoded by a nucleic acid endogenous to the non-human animal and/or a non-human CH3 domain encoded by a nucleic acid endogenous to the non-human animal and (2) a VH domain encoded by a nucleic acid exogenous to the non-human animal. In some cases, one or more B cells can be isolated from the non-human animal.

Antibodies against any appropriate antigen can be produced by a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87). In some cases, an antigen can be an endogenous antigen or self-antigen. In some cases, an antigen can be an exogenous antigen. An antigen can be any appropriate type of molecule (e.g., a peptide, a lipid, or a nucleic acid). Examples of antigens that can be used to immunize a non-human animal provided herein can be as listed in Table 2.

TABLE 2
Examples of antigens that can be used to immunize a non-human animal provided herein.

| | | | | | | # of | | Average |
|---|---|---|---|---|---|---|---|---|
| | | | | | | Clonotypes | # of | CDR3 |
| | | Sample | Total Raw | Successfully | Overlapped | with >=5 | IGHVs | length (# |
| Antigen | Genotype | ID | Reads | Aligned | and Aligned | reads | mapped | a.a.) |
| SARS-CoV2 | WT | 342 | 2,291,736 | 2,017,892 | 1,643,418 | 1,316 | 79 | 13.8 |
| Spike | | | | | | | | |
| SARS-CoV2 | WT | 345 | 2,148,017 | 1,828,914 | 1,516,225 | 2,611 | 99 | 13.3 |
| Spike | | | | | | | | |
| SARS-CoV2 | SM | 263 | 2,330,741 | 2,051,048 | 1,794,151 | 8,097 | 103 | 13.3 |
| Spike | | | | | | | | |
| SARS-CoV2 | SM | 320 | 2,503,240 | 2,040,036 | 1,794,759 | 11,565 | 108 | 13.6 |
| Spike | | | | | | | | |
| SARS-CoV2 | SM | 521 | 2,500,000 | 1,045,815 | 903,252 | 30,242 | 109 | 13.7 |
| Spike | | | | | | | | |
| PD-L1 | WT | 718-1 | 1,672,139 | 1,474,961 | 1,328,428 | 2,063 | 90 | 14.3 |
| PD-L1 | WT | 718-2 | 1,252,533 | 1,147,322 | 1,035,530 | 1,151 | 87 | 14.4 |
| PD-L1 | SM | 719-1 | 1,911,053 | 1,506,500 | 1,403,299 | 17,864 | 113 | 13.9 |
| PD-L1 | SM | 719-2 | 2,593,888 | 1,944,486 | 1,880,851 | 12,516 | 111 | 13.9 |
| Rabbit IgG | WT | 988 | 1,260,250 | 699,909 | 641,341 | 2,011 | 94 | 14.0 |
| Rabbit IgG | SM | 989 | 4,045,040 | 2,095,584 | 2,019,297 | 47,959 | 121 | 14.0 |
| Rabbit IgG | SM | 990 | 3,964,810 | 2,121,189 | 2,049,181 | 59,718 | 121 | 13.9 |
| Rabbit IgG | SM | 991 | 4,367,973 | 2,217,479 | 2,145,265 | 45,563 | 123 | 13.9 |
| Rat IgG | SM | 992 | 1,789,898 | 845,743 | 809,492 | 30,691 | 118 | 13.8 |
| Rat IgG | SM | 993 | 2,338,614 | 1,076,919 | 1,038,454 | 40,024 | 122 | 14.0 |
| Rat IgG | SM | 1024 | 2,027,019 | 1,031,155 | 983,144 | 37,096 | 120 | 14.0 |
| Goat IgG | SM | 1043 | 1,338,913 | 1,017,750 | 971,713 | 8,999 | 105 | 14.1 |
| Goat IgG | SM | 1044 | 1,448,252 | 1,031,304 | 987,794 | 6,276 | 111 | 14.1 |

In some cases, immunizing a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) with one or more antigen described herein can activate at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 non-human B cell clones in the non-human animal.

In some cases, immunizing a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) with one or more antigens described herein can lead to production of polyclonal antiserum including antibodies (e.g., heavy chain only antibodies) having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87 that can bind (e.g., specifically bind) at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 antigens of the administered immunogenic composition.

This document also provides antibodies (e.g., sdAbs and/or heavy chain only antibodies) produced by immunization of a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87). Antibodies (e.g., sdAbs and/or heavy chain only antibodies) produced by a non-human animal provided herein can be obtained using any appropriate method. For example, heavy chain only antibodies can be obtained from the blood of a non-human animal provided herein. In some cases, a non-human animal provided herein can be immunized with a particular antigen (e.g., a SARS-COV-2 antigen) such that the non-human animal produces antibodies (e.g., sdAbs and/or heavy chain only antibodies) against that antigen, and the antibodies produced can be assessed for the desired properties (e.g., binding properties, neutralization properties, and/or solubility properties). In some cases, blood can be collected from a non-human animal provided herein that has been immunized as described herein with a particular antigen multiple times (e.g., after each of multiple immunizations, multiple times after a single immunization, multiple times in between immunizations, or any combination thereof). Blood can be collected from a non-human animal provided herein that has been immunized as described herein any suitable amount of time following an immunization. For example, blood can be collected from a non-human animal provided herein that has been immunized at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more, after an immunization. In some cases, blood can be collected at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most at most 35, at most 42, at most 49, or at most 56 days after an immunization. In some cases, blood can be collected about 1, 2, 3, 4, 5, 6, 7, 8, 9, 10, 11, 12, 14, 16, 17, 18, 20, 21, 22, 23, 24, 25, 26, 27, 28, 29, 30, 31, 32, 33, 34, 35, 36, 37, 38, 39, 40, 41, or 42 days, or more after an immunization.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can retain its endogenous non-human immune cells. For example, a non-human animal provided herein can retain its non-human T cells, B cells, and/or antigen-presenting cells.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (1) a non-human CH2 domain and/or a non-human CH3 domain (and optionally a non-human Ig hinge) and (2) a VH domain such as a VH domain set forth in any one of SEQ ID NOs: 74-87) can include any feature or any combination of features (or any methods of making can be performed) as disclosed in U.S. Patent Application Publication No. 2017/0233459, which is hereby incorporated by reference in its entirety. For example, a non-human animal provided herein can include any feature or any combination of features (or any methods of making can be performed) as disclosed in Kuroiwa et al., Nat. Biotechnol., 27 (2): 173-81 (2009); Matsushita et al., PLos ONE, 9 (3):e90383 (2014); Hooper et al., Sci. Transl. Med., 6 (264): 264ra162 (2014); Matsushit et al., PLOS ONE, 10 (6):e 0130699 (2015); Luke et al., Sci. Transl. Med., 8 (326): 326ra21 (2016); Dye et al., Sci. Rep., 6:24897 (2016); Gardner et al., J. Virol., 91 (14) (2017); Stein et al., Antiviral Res., 146:164-173 (2017); Silver, Clin. Infect. Dis., 66 (7): 1116-1119 (2018); Beigel et al., Lancet Infect. Dis., 18 (4): 410-418 (2018); Luke et al., J. Inf. Dis., 218 (suppl_5):S636-S648 (2018), each of which is hereby incorporated by reference in its entirety.

In some cases, an amino acid sequence described herein can include one or more amino acid modifications (e.g., the articulated number of amino acid modifications). Such amino acid modifications can include, without limitation, amino acid substitutions, amino acid deletions, amino acid additions, and combinations thereof. In some cases, an amino acid modification can be made to improve the binding and/or contact with an antigen and/or to improve a functional activity of an antibody (e.g., a sdAb and/or a heavy chain only antibody) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding an antibody or a fragment thereof. See, for example, Zoller, Curr. Opin. Biotechnol. 3:348-354 (1992). Both naturally occurring and non-naturally occurring amino acids (e.g., artificially-derivatized amino acids) can be used to generate an amino acid sequence variant from an amino acid sequence described herein.

### Engineered Non-Human Animals for Producing Antibody-Like Molecules

In another aspect, this document relates to genetically modified or engineered non-human animals (e.g., genetically modified or engineered mice) that produce antibody-like molecules that include (1) non-human CH2 domains and/or non-human CH3 domains (e.g., endogenous CH2 and/or CH3 domains), and optionally a non-human Ig hinge, and (2) TCR variable domains (e.g., human TCR variable domains), and methods of making and using the same. For example, this document provides genetically engineered non-human animals of a particular species (e.g., a mouse species) that produce antibody-like molecules that include (1) CH2 domains of that same species (e.g., mouse CH2 domains) and/or CH3 domains of that same species (e.g., mouse CH2 domains) and (2) TCR variable domains (e.g., human TCR variable domains).

An antibody-like molecule produced by a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can include any appropriate TCR variable domain (e.g., any appropriate human TCR variable domain). In some cases, a TCR variable domain can be a human TCR variable domain. Examples of TCR variable domains that can be included in an antibody-like molecule produced by a non-human animal provided herein include, without limitation, TCRAV domains (e.g., human TCR Va domains), TCRBV domains (e.g., human TCR Vβ domains), TCRGV domains (e.g., human TCR Vγ domains), and TCRDV domains (e.g., human TCR Vδ domains).

In some cases, an antibody-like molecule produced by a non-human animal provided herein can include, in addition to a TCR variable domain (e.g., a human TCR Vβ domain), a TCR D domain and/or a TCR J domain. Examples of TCR D domains that can be included as part of an antibody-like molecule produced by a non-human animal provided herein include, without limitation, TCRBD domains (e.g., human TCR Dβ domains) and TCRDD domains (e.g., human TCR Dδ domains). Examples of TCR J domains that can be included as part of an antibody-like molecule produced by a non-human animal provided herein include, without limitation, TCRAJ domains (e.g., human TCR Jα domains), TCRBJ domains (e.g., human TCR Jβ domains), TCRGJ domains (e.g., human TCR Jγ domains), and TCRDJ domains (e.g., human TCR Jβ domains).

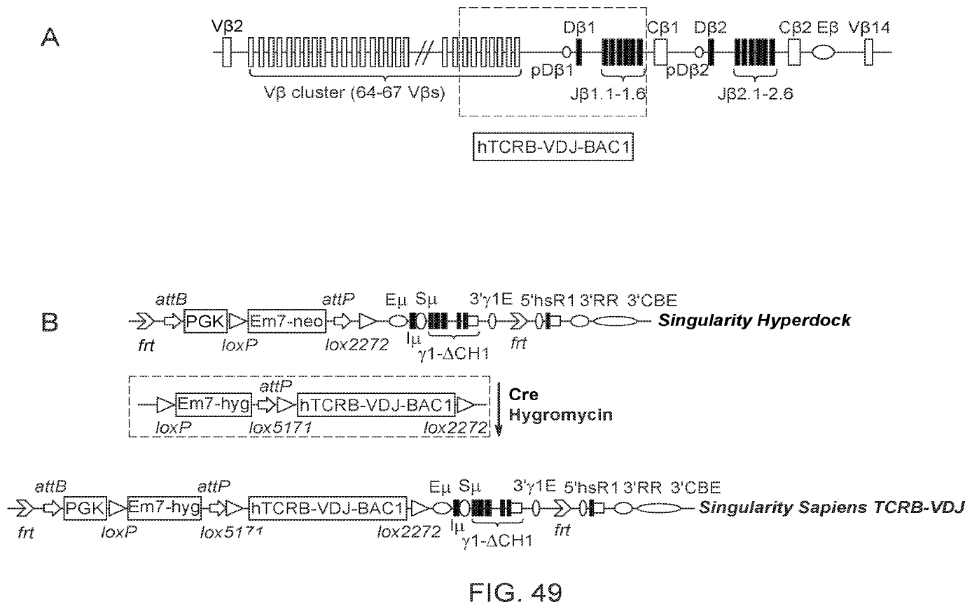

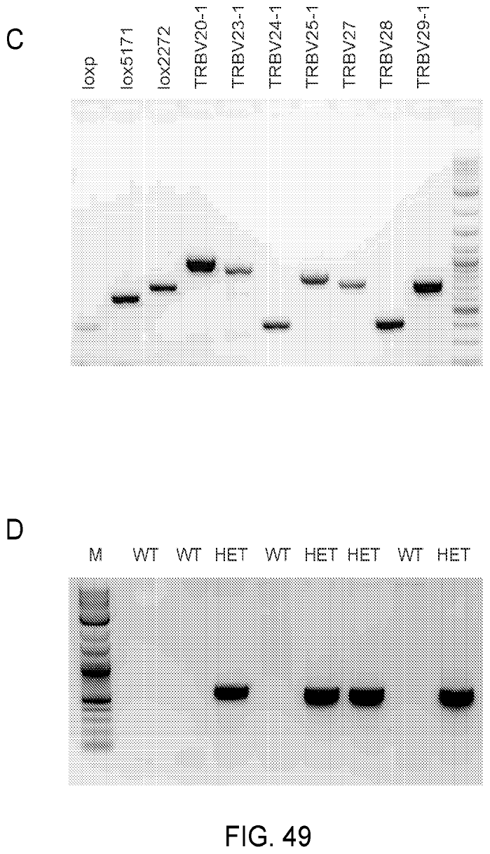

In some cases, an antibody-like molecule produced by a non-human animal provided herein can include, in addition to a TCR variable domain (e.g., a human TCR Vβ domain), an Ig D domain (e.g., a human Ig D domain) and/or an Ig J domain (e.g., a human Ig J domain). See, e.g., FIG. 49B.

In some cases, an antibody-like molecule produced by a non-human animal provided herein can include a chimeric antibody-like molecule. For example, an antibody-like molecule produced by a non-human animal provided herein can include one or more Ig domains encoded by a nucleic acid endogenous to the non-human animal (e.g., endogenous CH2 and CH3 domains), and optionally a non-human Ig hinge, and one or more TCR domains encoded by a nucleic acid exogenous to the non-human animal (e.g., any appropriate human TCR variable domain). For example, an antibody-like molecule produced by a non-human animal provided herein can include (1) an endogenous Ig hinge, CH2 domain, and/or CH3 domain, and optionally a non-human Ig hinge, (2) an exogenous TCR variable domain such as a TCR V domain encoded by a nucleic acid exogenous to the non-human animal, (3) an exogenous TCR D domain encoded by a nucleic acid endogenous to the non-human animal, and (4) an exogenous TCR J domain encoded by a nucleic acid endogenous to the non-human animal.

An antibody-like molecule produced by a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can include any appropriate number of TCR domains. In some cases, an antibody-like molecule produced by a non-human animal provided herein can include (1) a TCR variable domain and (2) a TCR D domain and/or a TCR J domain.

A non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain can be any type of non-human animal. Examples of non-human animals that can be designed to produce antibody-like molecules that include (1) a non-human CH2 domain and/or a non-human CH3 domain, and optionally a non-human Ig hinge, and (2) a TCR variable domain such as a human TCR variable domain as described herein include, without limitation, mice, rats, rabbits, guinea pigs, zebrafish, flies (e.g., Drosophila melanogaster), pigs, sheep, non-human primates (e.g., monkeys), and bovine species. In some cases, a non-human animal designed to produce antibody-like molecules that include (1) a mouse CH2 domain and/or a mouse CH3 domain, and optionally a non-human Ig hinge, and (2) a TCR variable domain such as a human TCR variable domain can be a mouse.

In some cases, a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can be designed to produce antibody-like molecules that lack Ig CH1 domains and that lack light chains. For example, a non-human animal (e.g., a mouse) provided herein can be a non-human animal whose genome has (e.g., is genetically engineered to have) one or more disruptions in an endogenous nucleic acid sequence encoding an CH1 domain of an IgG1 C-region gene (e.g., C_{γ1}), such that an IgG antibody-like molecule encoded by the non-human animal lacks a CH1 domain and lacks light chains. In some cases, the genome of a non-human animal provided herein can lack nucleic acid encoding at least a portion of an endogenous Ig CH1 domain. In some cases, the genome of a non-human animal provided herein can lack at least a portion of an endogenous regulatory element that drives expression of an endogenous Ig CH1 domain.

In some cases, a non-human animal provided herein (e.g., a genetically engineered non-human animal such as a mouse that can, when exposed to one or more antigens, produce an antibody-like molecule that include (1) a non-human CH2 domain and/or a non-human CH3 domain, and optionally a non-human Ig hinge, and (2) a TCR variable domain such as a human TCR variable domain) can be designed to have a deletion of endogenous nucleic acid encoding a CH1 domain of a constant region (e.g., of an IgG C-region such as a CH1 domain of an IgG1 C-region, a CH1 domain of an IgG2a C-region, a CH1 domain of an IgG2b C-region, and/or a CH1 domain of an IgG3 C-region). The CH1 domain can contain multiple exons. In some cases, endogenous exon 1 of a CH1 domain of a constant region such as an IgG C-region can be deleted such that the engineered non-human animal (e.g., mouse) produces antibody-like molecules such as TCR-IgMΔCH1 molecules, TCR-IgGΔCH1 molecules, TCR-IgDΔCH1 molecules, TCR-IgAΔCH1 molecules, and/or TCR-IgEΔCH1 molecules.

When making one or more genetic modifications to delete all or part of the nucleic acid encoding a CH1 domain (e.g., a CH1 domain of an IgG1 C-region, a CH1 domain of an IgG2a C-region, a CH1 domain of an IgG2b C-region, and/or a CH1 domain of an IgG3 C-region) such that the engineered non-human animal produces antibody-like molecules such as TCR-IgΔCH1 molecules, the endogenous nucleic acid encoding an Ig hinge domain, a heavy chain CH2 domain, and a heavy chain CH3 domain can remain intact. For example, to make a mouse that produces TCR-IgG1ΔCH1 antibody-like molecules, the genome of that mouse can lack exon 1 (and/or additional portions) of the CH1 domain of IgG1 while retaining the endogenous mouse nucleic acid needed to express the hinge domain, a heavy chain CH2 domain, and a heavy chain CH3 domain of IgG1, thereby resulting in a mouse that is capable of producing TCR-IgG1ΔCH1 antibody-like molecules.

Additional endogenous nucleic acid components that can be deleted from the genome of a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) to create a non-human animal provided herein include, without limitation, the introns and/or exons of the IgM constant domains (e.g., the μ constant domain locus), the introns and/or exons of the IgD constant domains (e.g., the δ constant domain locus), the introns and/or exons of the IgF constant domains (e.g., the ε constant domain locus), and/or the introns and/or exons of the IgA constant domains (e.g., the α constant domain locus). For example, a non-human animal provided herein can be designed to lack the introns and exons of the IgM constant domains (e.g., the μ constant domain locus), the introns and exons of the IgD constant domains (e.g., the δ constant domain locus), the introns and exons of the IgE constant domains (e.g., the ε constant domain locus), and the introns and exons of the IgA constant domains (e.g., the α constant domain locus).

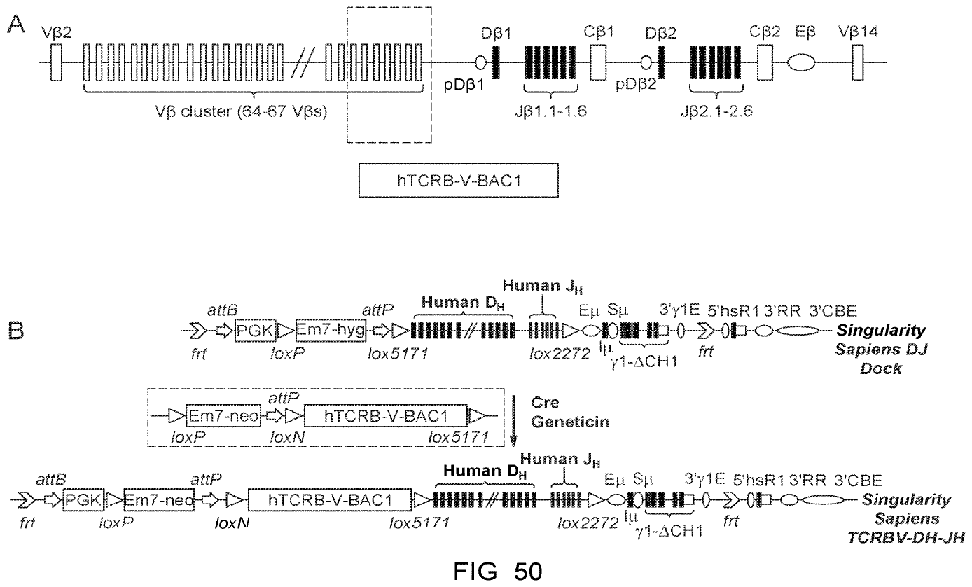

In some cases, when designing a non-human animal provided herein (e.g., a genetically engineered non-human animal such as a mouse that can, when exposed to one or more antigens, produce an antibody-like molecule that include (1) a non-human CH2 domain and/or a non-human CH3 domain, and optionally a non-human Ig hinge, and (2) a TCR variable domain such as a human TCR variable domain) to produce only TCR-IgG1ΔCH1 antibody-like molecules, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus). An example of a genetic engineering approach to create a mouse that produces TCR-IgG1ΔCH1 antibody-like molecules is set forth in FIGS. 49B and 50B.

In some cases, when designing a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) to produce TCR-IgG2aΔCH1 antibody-like molecules, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ1 constant domains (e.g., the γ1 constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

In some cases, when designing a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) to produce only TCR-IgG2bΔCH1 antibody-like molecules, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ1 constant domains (e.g., the γ1 constant domain locus), and the endogenous (if endogenously present) introns and/or exons of the Igγ2c constant domains (e.g., the γ2c constant domain locus).

In some cases, when designing a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) to produce only TCR-IgG2cΔCH1 antibody-like molecules, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ1 constant domains (e.g., the γ1 constant domain locus).

In some cases, when designing a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) to produce only TCR-IgG3ΔCH1 antibody-like molecules, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ1 constant domains (e.g., the γ1 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

As described herein, retaining and/or creating new positioning for certain endogenous enhancer or regulatory elements of a non-human animal can result in a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) that produces effectively large collections of and/or amounts of diverse antibody-like molecules. For example, a non-human animal provided herein can be designed to retain the μ enhancer (Eμ), the μ switch region (Sμ), and/or the μ promoter containing I-exon (Ip) that are endogenously found upstream of the nucleic acid encoding the IgM constant domains. In some cases, non-human animals (e.g., mice) provided herein can be designed such that the retained endogenous Eμ, Sμ, and/or Iμ elements are in a genomic position such that the first nucleic acid sequence downstream of the retained Eμ, Sμ, and/or Iμ elements that encodes a full-length endogenous Ig constant domain is one that encodes a CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 1B and FIG. 3C where the nucleic acids of the endogenous mouse Eμ, Sμ, and Iμ elements are repositioned to be upstream of the nucleic acid encoding the endogenous IgG1 CH2 domain.

In another example, a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can be designed to retain the 3′RR and/or 3′CBE elements that are endogenously found downstream of the nucleic acid encoding the IgA constant domains. In some cases, non-human animals (e.g., mice) provided herein can be designed such that the retained endogenous 3′RR and/or 3′CBF elements are in a genomic position such that the first nucleic acid sequence upstream of the retained 3′RR and/or 3′CBE elements that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 2B and FIG. 3E where the nucleic acid of the endogenous mouse 3′RR element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 3′RR element.

In some cases, a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can be designed to retain the 3′γ1E element that is endogenously found between, for example, the IgG1 and IgG2b loci. In some cases, non-human animals (e.g., mice) provided herein can be designed such that the retained endogenous 3′γ1E element is in a genomic position such that nucleic acid encoding two, one, or no full-length endogenous Ig CH2 domains is located between the retained endogenous 3′γ1E element and a retained endogenous 3′RR element and/or a retained endogenous 3′CBE element. An example of this genomic configuration is set forth in FIG. 3E where the nucleic acid of the endogenous mouse 3′γ1E element is repositioned to be upstream of a retained endogenous 3′RR element such that no other nucleic acid encoding a full length IgG CH2 domain is located between the endogenous mouse 3′γ1E element and the endogenous 3′RR element.

In some cases, a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can be designed to retain the 5′hsR1 element that is endogenously found within the IgA constant domain locus. In some cases, non-human animals (e.g., mice) provided herein can be designed such that the retained endogenous 5′hsR1 element is in a genomic position such that the first nucleic acid sequence upstream of the retained 5′hsR1 element that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 3E where the nucleic acid of the endogenous mouse 5′hsR1 element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 5′hsR1 element.

In some cases, a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can be designed to have (a) at least a portion of the endogenous VH locus replaced with nucleic acid encoding one or more TCR variable domains followed by (b) an endogenous Eμ element and/or an endogenous element Iμ and/or an endogenous Sμ element followed by (c) nucleic acid that encodes an endogenous IgG hinge, CH2 domain, and CH3 domain in the absence of the endogenous CH1 domain for that IgG followed by (d) an endogenous 3′γ1E element, an endogenous 3′RR element, and an endogenous 3′CBE element, while lacking the endogenous nucleic acid that encodes at least one full-length CH2 domain or CH3 domain of each of IgM, IgD, IgE, and IgA. An example of this genomic configuration is set forth in FIGS. 49B and 50B.

In some cases, instead of retaining an endogenous enhancer or regulatory element as described herein, one or more exogenous enhancer or regulatory elements can be engineered into a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain). For example, in some cases, a mouse can be designed as described herein where the endogenous mouse Eμ element is removed and replaced with a human Eμ element.

In some cases, a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can be designed to produce antibody-like molecules that lack endogenous Ig VH domains. For example, non-human animals provided herein can be non-human animals whose genomes have (e.g., are genetically engineered to have) one or more disruptions in an endogenous nucleic acid sequence encoding an Ig VH locus, such that antibody-like molecules encoded by the non-human animal lack endogenous Ig VH domains. In some cases, at least one allele of the genome of a non-human animal provided herein can lack at least a portion of the endogenous nucleic acid encoding one or more Ig VH domains. In some cases, at least one allele of the genome of a non-human animal provided herein can lack all the exons of the endogenous nucleic acid encoding Ig VH domains. In some cases, both alleles of the genome of a non-human animal provided herein can lack all the exons of the endogenous Ig heavy chain variable region. In some cases, neither allele of the genome of a non-human animal provided herein contains an endogenous exon of a nucleic acid encoding an Ig VH domain.

In some cases, a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can be designed to have a variable region Ig locus that was modified to include TCR variable domain gene segments that are not endogenous to that non-human animal. For example, a mouse provided herein can be designed to have an Ig VH locus that was modified to include one or more human TCR variable domain gene segments.

In some cases, an Ig locus present in the genome of a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can have any appropriate number of exogenous TCR variable domain gene segments. In some cases, an Ig locus present in the genome of a mouse provided herein can include two, three, four, five, six, seven, eight, nine, ten, 11, 12, 13, 14, or more TCR variable domain gene segments that encode TCR variable domains that are not mouse TCR variable domains.

In some cases, an Ig locus present in the genome of a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can have any appropriate TCR variable domain gene segments. In some cases, an Ig locus present in the genome of a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can include exogenous nucleic acid encoding one or more (e.g., one, two, three, four, five, six, seven, eight, nine, ten, or more) human TCR variable domain (e.g., TCRB variable domain) gene segments. In some cases, an Ig locus present in the genome of a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can include exogenous nucleic acid encoding one or more (e.g., one, two, three, four, five, six, seven, eight, nine, ten, 11, 12, 13, 14, 15, 25, 27, or more) human Ig DH gene segments. For example, an IgH allele of the genome of a non-human animal provided herein can include exogenous nucleic acid encoding from 1 to 27 human Ig DH gene segments. In some cases, an Ig locus present in the genome of a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can include exogenous nucleic acid encoding one or more (e.g., one, two, three, four, five, six, seven, eight, nine, ten, 11, 12, 13, 14, or more) human Ig VH gene segments. For example, an IgH allele of the genome of a non-human animal provided herein can include exogenous nucleic acid encoding from 1 to 9 human Ig VH gene segments. In some cases, at least one IgH allele of the genome of a non-human animal provided herein can include exogenous nucleic acid encoding 7 or more human TCR variable domain gene segments, one or more human Ig DH gene segments, and one or more human Ig VH gene segments.

In some cases, an Ig locus present in the genome of a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can have any appropriate one or more TCR variable domain gene segments. In some cases, a TCR variable domain gene segment can be a TCRA variable domain gene segment (e.g., a human TCRA variable domain gene segment). In some cases, a TCR variable domain gene segment can be a TCRB variable domain gene segment (e.g., a human TCRB variable domain gene segment). In some cases, a TCR variable domain gene segment can be a TCRD variable domain gene segment (e.g., a human TCRD variable domain gene segment). In some cases, a TCR variable domain gene segment can be a TCRG variable domain gene segment (e.g., a human TCRG variable domain gene segment). Examples of TCR variable domain gene segments that can be included in a genome of a non-human animal provided herein include, without limitation, the human TCR variable domain gene segment referred to as TRBV20-1, the human TCR variable domain gene segment referred to as TRBV23-1, the human TCR variable domain gene segment referred to as TRBV24-1, the human TCR variable domain gene segment referred to as TRBV25-1, the human TCR variable domain gene segment referred to as TRBV27, the human TCR variable domain gene segment referred to as TRBV28, and the human TCR variable domain gene segment referred to as TRBV29-1. Examples of TCR variable domain gene segments that can be included in a genome of a non-human animal provided herein also include, without limitation, TCR variable domain gene segments that encode the amino acid sequence encoded by TRBV20-1, TCR variable domain gene segments that encode the amino acid sequence encoded by TRBV23-1, TCR variable domain gene segments that encode the amino acid sequence encoded by TRBV24-1, TCR variable domain gene segments that encode the amino acid sequence encoded by TRBV25-1, TCR variable domain gene segments that encode the amino acid sequence encoded by TRBV27, TCR variable domain gene segments that encode the amino acid sequence encoded by TRBV28, and TCR variable domain gene segments that encode the amino acid sequence encoded by TRBV29-1. In some cases, nucleic acid sequences of TCR variable domain gene segments that can be included in a genome of a non-human animal can be as described in Lefranc et al., Nucleic Acids Res., 43:D413-D422 (2015)).

In some cases, an Ig locus present in the genome of a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can include one or more regulatory elements for each TCR variable domain gene segment inserted into the non-mouse Ig locus (e.g., to control expression and/or recombination for each TCR variable domain gene segment). The regulatory element(s) can be endogenous or exogenous. For example, the regulatory element(s) can be human regulatory element(s). Examples of regulatory elements that can be used to control expression and/or recombination for each TCR variable domain gene segment include, without limitation, promoters, enhancers, transcription factor binding sites, splice sites, recombination signal sequences, leader exon 1, leader exon 2, signal peptide sequences, and introns.

Any appropriate method can be used to generate a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain). In some cases, nucleic acid comprising one or more human TCR variable domain gene segments (e.g., synthetic array of TCR variable domain gene segments) can be inserted into the genome of a non-human animal (e.g., a mouse) that lacks nucleic acid encoding at least a portion of an endogenous CH1 (or lacks at least a portion of an endogenous regulatory element that drives expression of an endogenous CH1 domain) and optionally lacks at least a portion of the endogenous nucleic acid encoding one or more (e.g., all) endogenous VH domains. For example, a TCR variable domain locus (e.g., nucleic acid comprising one or more human TCR variable domain gene segments) can be inserted into the genome of an engineered mouse as set forth in FIGS. 4, 5D, and 6B (also referred to as a Singularity HyperDock non-human animal or a Singularity HyperDock mouse). In some cases, nucleic acid comprising one or more human TCR variable domain gene segments can include at least one recombination site (e.g., at least one nucleic acid sequence that can be recognized by a recombinase) on each end (e.g., at the 5′ end and at the 3′ end) of the one or more human TCR variable domain gene segments. In some cases, the non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of the endogenous nucleic acid encoding one or more (e.g., all) VH domains (e.g., a Singularity HyperDock mouse) can include at least one recombination site, such that a recombinase can facilitate the insertion of nucleic acid comprising one or more human TCR variable domain gene segments into the genome of that non-human animal.

In some cases, nucleic acid comprising one or more human TCR variable domain gene segments can be present on a vector. Examples of vectors that can include nucleic acid comprising one or more human TCR variable domain gene segments include, without limitation, yeast artificial chromosomes (YACs), bacterial artificial chromosomes (BACs), human artificial chromosomes (HACs), transchromosomes (e.g., whole transchromosomes and fragmented transchromosomes), P1-derived artificial chromosome (PACs), plasmids, and phagemids.

A non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of endogenous nucleic acid encoding one or more VH domains can include any appropriate recombination site(s). In some cases, a recombinase site can be an exogenous recombinase site. Examples of recombination sites that can be present in the genome of a non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of endogenous nucleic acid encoding one or more VH domains include, without limitation, frt, loxP, M2, M3, lox2271, lox2372, loxFAS, loxN, lox5171, lox2272, attB, and attP. Additional examples of recombination sites that can be present in the genome of a non-human animal (e.g., a mouse) provided herein can be as shown in Table 1.

A genome of a non-human animal (e.g., a mouse) described herein can include any appropriate number of recombination sites. For example, a non-human animal (e.g., a mouse) described herein can include one, two, three, four, five, six, seven, eight, nine, ten, or more recombination sites. When a non-human animal (e.g., a mouse) described herein includes two or more recombination sites in its genome, the recombination sites can each be a different recombination site. For example, a non-human animal (e.g., a mouse) described herein can include at least 3 different recombination sites within its genome. For example, a non-human animal (e.g., a mouse) described herein can include at least 5 different recombination sites within its genome.

A recombination site in a non-human animal (e.g., a mouse) described herein can be at any appropriate location within the genome of the non-human animal. In some cases, one or more recombination sites within a genome of a non-human animal described herein can be upstream of endogenous nucleic acid encoding an Ig CH2 domain or an Ig CH3 domain. In some cases, one or more recombination sites within a genome of a non-human animal described herein can be upstream (e.g., less than 2.5 Mb upstream) of an endogenous Eu element. When a genome of a non-human animal (e.g., a mouse) described herein includes different recombination sites (e.g., different exogenous recombination sites), each of the recombination sites can be located less than 2.0 Mb, less than 1.5 Mb, less than 1.0 Mb, less than 500 kb, less than 250 kb, less than 200 kb, less than 100 kb, less than 50 kb, less than 25 kb, or less than 10 kb upstream of an endogenous Eμ element.

Any appropriate method can be used to make a non-human animal described herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain). For example, gene editing techniques (e.g., CRISPR/Cas gene editing, TALEN gene editing, and/or zinc finger-based gene editing), recombination techniques (e.g., sequential Recombinase Mediated Cassette Exchange (RMCE)), and combinations thereof can be used to make a non-human animal described herein. In some cases, a nucleic acid molecule (e.g., an exogenous nucleic acid molecule) comprising one or more TCR variable domain gene segments (e.g., 1, 2, 3, 4, 5, 6, 7, 8, 9, 10, or more human TCR variable domain gene segments) can be introduced into a stem cell of a non-human animal (e.g., a mouse), the stem cell can be implanted into a blastocyst of the same species of non-human animal, and the blastocyst can be implanted into a pseudo-pregnant female of the same species of non-human animal to obtain a chimeric non-human animal, crossing the chimeric non-human animal to a wild-type non-human animal to produce offspring, screening the offspring for heterozygosity, and identifying a founder non-human animal carrying the one or more TCR variable domain gene segments within its genome. In some cases, a non-human animal provided herein can be made as described in the Examples.

This document also provides methods for producing populations of antibody-like molecules that include (1) a non-human CH2 domain and/or a non-human CH3 domain, and optionally a non-human Ig hinge, and (2) a TCR variable domain such as a human TCR variable domain. For example, a non-human animal described herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can be administered one or more antigens (e.g., a composition including one or more antigens), such that one or more B cells in the non-human animal produce an antibody-like molecule that includes (1) a non-human CH2 domain and/or a non-human CH3 domain, and optionally a non-human Ig hinge, and (2) a TCR variable domain such as a human TCR variable domain. In some cases, one or more B cells can be isolated from the non-human animal.

Antibody-like molecules against any appropriate antigen can be produced by a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain). In some cases, an antigen can be an endogenous antigen or self-antigen. In some cases, an antigen can be an exogenous antigen. An antigen can be any appropriate type of molecule (e.g., a peptide, a lipid, or a nucleic acid). Examples of antigens that can be used to immunize a non-human animal provided herein can be as listed in Table 2.

In some cases, immunizing a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) with one or more antigen described herein can activate at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 non-human B cell clones in the non-human animal.

In some cases, immunizing a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) with one or more antigens described herein can lead to production of polyclonal antiserum including antibody-like molecules that include (1) a non-human CH2 domain and/or a non-human CH3 domain, and optionally a non-human Ig hinge, and (2) a TCR variable domain such as a human TCR variable domain and that can bind (e.g., specifically bind) at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 antigens of the administered immunogenic composition.

This document also provides antibody-like molecules produced by immunization of a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain). Antibody-like molecules produced by a non-human animal provided herein can be obtained using any appropriate method. For example, antibody-like molecules can be obtained from the blood of a non-human animal provided herein. In some cases, a non-human animal provided herein can be immunized with a particular antigen (e.g., a SARS-CoV-2 antigen) such that the non-human animal produces antibody-like molecules against that antigen, and the antibody-like molecules produced can be assessed for the desired properties (e.g., binding properties, neutralization properties, and/or solubility properties). In some cases, blood can be collected from a non-human animal provided herein that has been immunized as described herein with a particular antigen multiple times (e.g., after each of multiple immunizations, multiple times after a single immunization, multiple times in between immunizations, or any combination thereof). Blood can be collected from a non-human animal provided herein that has been immunized as described herein any suitable amount of time following an immunization. For example, blood can be collected from a non-human animal provided herein that has been immunized at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more, after an immunization. In some cases, blood can be collected at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most at most 35, at most 42, at most 49, or at most 56 days after an immunization. In some cases, blood can be collected about 1, 2, 3, 4, 5, 6, 7, 8, 9, 10, 11, 12, 14, 16, 17, 18, 20, 21, 22, 23, 24, 25, 26, 27, 28, 29, 30, 31, 32, 33, 34, 35, 36, 37, 38, 39, 40, 41, or 42 days, or more after an immunization.

In some cases, a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can retain its endogenous non-human immune cells. For example, a non-human animal provided herein can retain its non-human T cells, B cells, and/or antigen-presenting cells.

In some cases, a non-human animal provided herein (e.g., a genetically engineered non-human animal such as a mouse that can, when exposed to one or more antigens, produce an antibody-like molecule that includes (1) a non-human CH2 domain and/or a non-human CH3 domain, and optionally a non-human Ig hinge, and (2) a TCR variable domain such as a human TCR variable domain) can include any feature or any combination of features (or any methods of making can be performed) as disclosed in U.S. Patent Application Publication No. 2017/0233459, which is hereby incorporated by reference in its entirety. For example, a non-human animal provided herein can include any feature or any combination of features (or any methods of making can be performed) as disclosed in Kuroiwa et al., Nat. Biotechnol., 27 (2): 173-81 (2009); Matsushita et al., PLos ONE, 9 (3):e90383 (2014); Hooper et al., Sci. Transl. Med., 6 (264): 264ra162 (2014); Matsushit et al., PLOS ONE, 10 (6): c0130699 (2015); Luke et al., Sci. Transl. Med., 8 (326): 326ra21 (2016); Dye et al., Sci. Rep., 6:24897 (2016); Gardner et al., J. Virol., 91 (14) (2017); Stein et al., Antiviral Res., 146:164-173 (2017); Silver, Clin. Infect. Dis., 66 (7): 1116-1119 (2018); Beigel et al., Lancet Infect. Dis., 18 (4): 410-418 (2018); Luke et al., J. Inf. Dis., 218 (suppl_5):S636-S648 (2018), each of which is hereby incorporated by reference in its entirety.

In some cases, an amino acid sequence of a human TCR variable domain described herein can include one or more amino acid modifications. Such amino acid modifications can include, without limitation, amino acid substitutions, amino acid deletions, amino acid additions, and combinations thereof. In some cases, an amino acid modification can be made to improve the binding and/or contact with an antigen and/or to improve a functional activity of an antibody-like molecule provided herein. In some cases, an amino acid substitution within a human TCR variable domain described herein can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within a human TCR variable domain described herein can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to a human TCR variable domain described herein) can include site-specific mutagenesis or random mutagenesis (e.g., by PCR) of a nucleic acid encoding an antibody or a fragment thereof. See, for example, Zoller, Curr. Opin. Biotechnol. 3:348-354 (1992). Both naturally occurring and non-naturally occurring amino acids (e.g., artificially-derivatized amino acids) can be used to generate an amino acid sequence variant from an amino acid sequence of a human TCR variable domain described herein.

### Engineered Non-Human Animals for Producing Antibody-Like Molecules Containing Fibronectin Sequences

In another aspect, this document relates to genetically modified or engineered non-human animals (e.g., genetically modified or engineered mice) that produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge, and methods of making and using the same. For example, this document provides genetically engineered non-human animals of a particular species (e.g., a mouse species) that produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge.

Antibody-like molecules produced by a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can be any appropriate type of antibody-like molecule. Examples of antibody-like molecules that can be produced by a non-human animal provided herein include, without limitation, antibody-like molecules containing non-human IgG1 CH2 domain and/or a non-human IgG1 CH3 domain (e.g., endogenous IgG CH2 and/or CH3 domains), and optionally a non-human Ig hinge.

An antibody-like molecule produced by a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can be any appropriate size. In some cases, an antibody-like molecule produced by a non-human animal provided herein can be less than 150 kDa. For example, an antibody-like molecule produced by a non-human animal provided herein can be from about 10 kDa to about 150 kDa (e.g., from about 10 kDa to about 120 kDa, from about 10 kDa to about 100 kDa, from about 10 kDa to about 80 kDa, from about 10 kDa to about 50 kDa, from about 10 kDa to about 25 kDa, from about 10 kDa to about 20 kDa, from about 10 kDa to about 15 kDa, from about 10 kDa to about 12 kDa, from about 15 kDa to about 150 kDa, from about 25 kDa to about 150 kDa, from about 50 kDa to about 150 kDa, from about 75 kDa to about 150 kDa, from about 100 kDa to about 150 kDa, from about 12 kDa to about 125 kDa, from about 15 kDa to about 100 kDa, from about 20 kDa to about 75 kDa, from about 25 kDa to about 50 kDa, from about 12 kDa to about 25 kDa, from about 15 kDa to about 50 kDa, from about 25 kDa to about 75 kDa, from about 50 kDa to about 100 kDa, or from about 75 kDa to about 125 kDa).

An antibody-like molecule produced by a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can include any appropriate first amino acid sequence of a FN3 polypeptide (e.g., a ¹⁰FN3 polypeptide) and any appropriate second amino acid sequence of a FN3 polypeptide (e.g., a ¹⁰FN3 polypeptide). When using a human ¹⁰FN3 polypeptide, the first and second amino acid sequences of a human ¹⁰FN3 polypeptide included within an antibody-like molecule described herein can be as set forth in Table 3.

TABLE 3
Segments based on a human ¹⁰FN3 polypeptide.

Human ¹⁰FN3 polypeptide:
QQSTVSDVPRDLEVVAATPTSLLISWDAPAVTVRYYRITYGETGGNSPVQEFTVPGSKSTATISGLKPGVDYTITVYAVTGRGDSP
ASSKPISINYRTEIDKPSQ (SEQ ID NO: 96)

| | | SEQ | Second amino acid | SEQ |
|---|---|---|---|---|
| | First amino acid sequence for antibody-like | ID | sequence for antibody-like | ID |
| Construct | molecule provided herein | NO: | molecule provided herein | NO: |
| A | QQSTVSDVPRDLEVVAATPTSLLISWDAPAVTVRYYRITYGETGGN | 101 | PISINYRTEIDKP | 102 |
| | SPVQEFTVPGSKSTATISGLKPGVDYTITVYAVT | | | |
| B | QQSTVSDVPRDLEVVAATPTSLLISWDAPAVTVRYYRITYGETGGN | 103 | PISINYRTEIDKPSQ | 104 |
| | SPVQEFTVPGSKSTATISGLKPGVGGSGGSGGSGGDYTITVYAVT | | | |

In some cases, a variant of a human FN3 polypeptide (e.g., a human ¹⁰FN3 polypeptide) can be used as the first and/or second amino acid sequence of a human ¹⁰FN3 polypeptide included within an antibody-like molecule described herein. A variant of a human FN3 polypeptide (e.g., a variant of a human ¹⁰FN3 polypeptide) can have the amino acid sequence of SEQ ID NO:C19 with one or more (e.g., e.g., one, two, three, four, five, six, seven, eight, nine, ten, or more) amino acid deletions, one or more (e.g., e.g., one, two, three, four, five, six, seven, eight, nine, ten, or more) amino acid additions, one or more (e.g., e.g., one, two, three, four, five, six, seven, eight, nine, ten, or more) amino acid substitutions, or combinations thereof, provided that the variant retains the ability to act as a scaffold for a heavy chain variable domain (e.g., a heavy chain variable domain which can undergo affinity maturation in response to antigen challenge).

A non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can be any type of non-human animal. Examples of non-human animals that can be designed to produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge as described herein include, without limitation, mice, rats, rabbits, guinea pigs, zebrafish, flies (e.g., Drosophila melanogaster), pigs, sheep, non-human primates (e.g., monkeys), and bovine species. In some cases, a non-human animal designed to produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge, can be a mouse.

In some cases, a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can be designed to produce antibody-like molecules that lack CH1 domains and/or that lack Ig light chains. For example, a non-human animal (e.g., a mouse) provided herein can be a non-human animal whose genome has (e.g., is genetically engineered to have) one or more disruptions in an endogenous nucleic acid sequence encoding an CH1 domain of an IgG1 C-region gene (e.g., C_{γ1}), such that an IgG antibody-like molecule encoded by the non-human animal lacks a CH1 domain and lacks light chains. In some cases, the genome of a non-human animal provided herein can lack nucleic acid encoding at least a portion of an endogenous Ig CH1 domain. In some cases, the genome of a non-human animal provided herein can lack at least a portion of an endogenous regulatory element that drives expression of an endogenous Ig CH1 domain.

In some cases, a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can be designed to have a deletion of endogenous nucleic acid encoding a CH1 domain of a constant region (e.g., of an IgG C-region such as a CH1 domain of an IgG1 C-region, a CH1 domain of an IgG2a C-region, a CH1 domain of an IgG2b C-region, and/or a CH1 domain of an IgG3 C-region). The CH1 domain can contain multiple exons. In some cases, endogenous exon 1 of a CH1 domain of a constant region such as an IgG C-region can be deleted such that the engineered non-human animal (e.g., mouse) produces antibody-like molecules such as FN3-IgMΔCH1 molecules, FN3-IgGΔCH1 molecules, FN3-IgDΔCH1 molecules, FN3-IgAΔCH1 molecules, and/or FN3-IgEΔCH1 molecules.

When making one or more genetic modifications to delete all or part of the nucleic acid encoding a CH1 domain (e.g., a CH1 domain of an IgG1 C-region, a CH1 domain of an IgG2a C-region, a CH1 domain of an IgG2b C-region, and/or a CH1 domain of an IgG3 C-region) such that the engineered non-human animal produces antibody-like molecules such as FN3-IgΔCH1 molecules, the endogenous nucleic acid encoding a hinge domain, a heavy chain CH2 domain, and a heavy chain CH3 domain can remain intact. For example, to make a mouse that produces FN3-IgG1ΔCH1 antibody-like molecules, the genome of that mouse can lack exon 1 (and/or additional portions) of the CH1 domain of IgG1 while retaining the endogenous mouse nucleic acid needed to express the hinge domain, a heavy chain CH2 domain, and a heavy chain CH3 domain of IgG1, thereby resulting in a mouse that is capable of producing FN3-IgG1ΔCH1 antibody-like molecules.

Additional endogenous nucleic acid components that can be deleted from the genome of a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) to create a non-human animal provided herein include, without limitation, the introns and/or exons of the IgM constant domains (e.g., the μ constant domain locus), the introns and/or exons of the IgD constant domains (e.g., the δ constant domain locus), the introns and/or exons of the IgE constant domains (e.g., the ε constant domain locus), and/or the introns and/or exons of the IgA constant domains (e.g., the α constant domain locus). For example, a non-human animal provided herein can be designed to lack the introns and exons of the IgM constant domains (e.g., the μ constant domain locus), the introns and exons of the IgD constant domains (e.g., the δ constant domain locus), the introns and exons of the IgE constant domains (e.g., the ε constant domain locus), and the introns and exons of the IgA constant domains (e.g., the α constant domain locus).

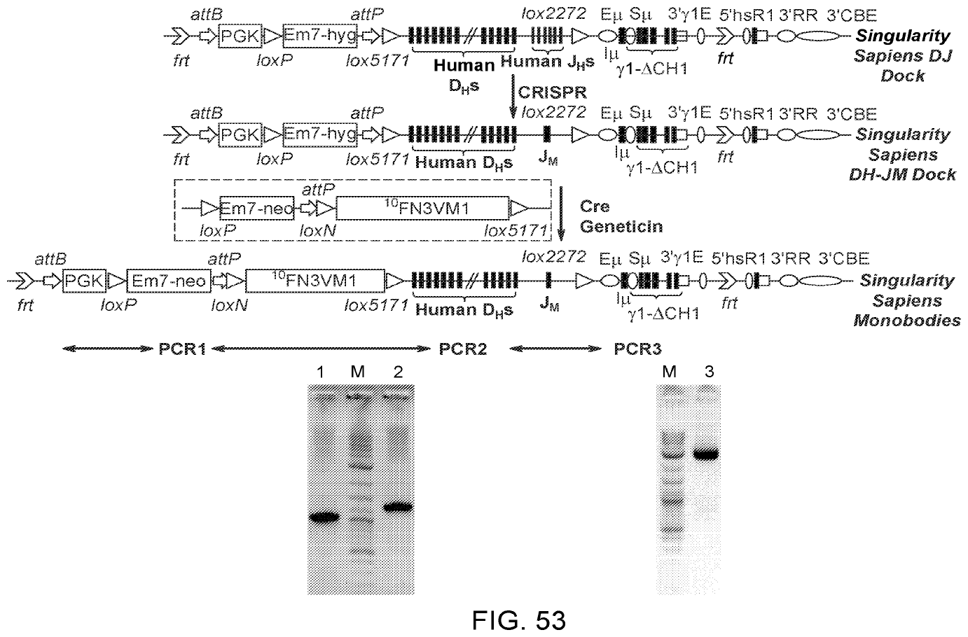

In some cases, when designing a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) to produce a FN3-IgG1ΔCH1 antibody-like molecule, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus). An example of a genetic engineering approach to create a mouse that produces FN3-IgG1ΔCH1 antibody-like molecules is set forth in FIG. 53.

In some cases, when designing a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) to produce FN3-IgG2aΔCH1 antibody-like molecules, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ1 constant domains (e.g., the γ1 constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

In some cases, when designing a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) to produce FN3-IgG2bΔCH1 antibody-like molecules, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ1 constant domains (e.g., the γ1 constant domain locus), and the endogenous (if endogenously present) introns and/or exons of the Igγ2c constant domains (e.g., the γ2c constant domain locus).

In some cases, when designing a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) to produce FN3-IgG2cΔCH1 antibody-like molecules, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ1 constant domains (e.g., the γ1 constant domain locus).

In some cases, when designing a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) to produce FN3-IgG3ΔCH1 antibody-like molecules, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ1 constant domains (e.g., the γ1 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

As described herein, retaining and/or creating new positioning for certain endogenous enhancer or regulatory elements of a non-human animal can result in a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) that produces effectively large collections of and/or amounts of diverse antibody-like molecules (e.g., antibody-like molecules containing FN3 amino acid sequences). For example, a non-human animal provided herein can be designed to retain the μ enhancer (Eμ), the μ switch region (Sμ), and/or the μ promoter containing I-exon (Iμ) that are endogenously found upstream of the nucleic acid encoding the IgM constant domains. In some cases, non-human animals (e.g., mice) provided herein can be designed such that the retained endogenous Eμ, Su, and/or Iμ elements are in a genomic position such that the first nucleic acid sequence downstream of the retained Eμ, Sμ, and/or Iμ elements that encodes a full-length endogenous Ig constant domain is one that encodes a CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 1B and FIG. 3C where the nucleic acids of the endogenous mouse Eμ, Sμ, and Iμ elements are repositioned to be upstream of the nucleic acid encoding the endogenous IgG1 CH2 domain.

In another example, a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can be designed to retain the 3′RR and/or 3′CBE elements that are endogenously found downstream of the nucleic acid encoding the IgA constant domains. In some cases, non-human animals (e.g., mice) provided herein can be designed such that the retained endogenous 3′RR and/or 3′CBE elements are in a genomic position such that the first nucleic acid sequence upstream of the retained 3′RR and/or 3′CBE elements that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 2B and FIG. 3E where the nucleic acid of the endogenous mouse 3′RR element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 3′RR element.

In some cases, a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can be designed to retain the 3′γ1E element that is endogenously found between, for example, the IgG1 and IgG2b loci. In some cases, non-human animals (e.g., mice) provided herein can be designed such that the retained endogenous 3′γ1E element is in a genomic position such that nucleic acid encoding two, one, or no full-length endogenous Ig CH2 domains is located between the retained endogenous 3′γ1E element and a retained endogenous 3′RR element and/or a retained endogenous 3′CBE element. An example of this genomic configuration is set forth in FIG. 3E where the nucleic acid of the endogenous mouse 3′γ1E element is repositioned to be upstream of a retained endogenous 3′RR element such that no other nucleic acid encoding a full length IgG CH2 domain is located between the endogenous mouse 3′γ1E element and the endogenous 3′RR element.

In some cases, a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can be designed to retain the 5′hsR1 element that is endogenously found within the IgA constant domain locus. In some cases, non-human animals (e.g., mice) provided herein can be designed such that the retained endogenous 5′hsR1 element is in a genomic position such that the first nucleic acid sequence upstream of the retained 5′hsR1 element that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 3E where the nucleic acid of the endogenous mouse 5′hsR1 element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 5′hsR1 element.

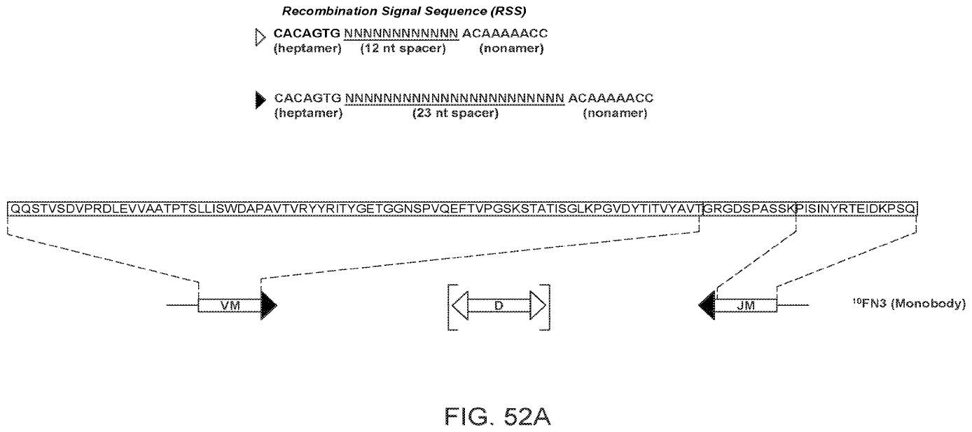

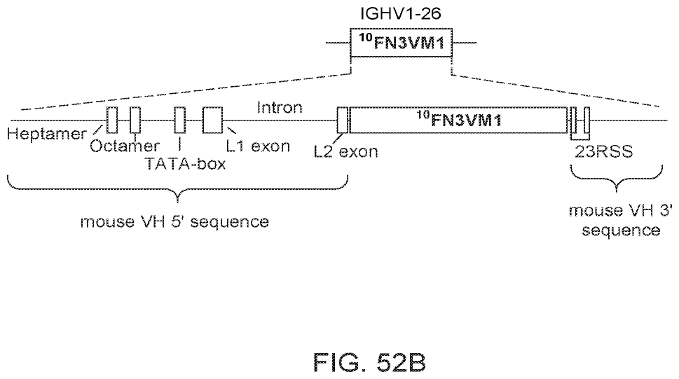

In some cases, a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can be designed to have (a) at least a portion of the endogenous VH locus replaced with nucleic acid encoding a first amino acid sequence of a FN3 polypeptide (e.g., a ¹⁰FN3 polypeptide), followed by nucleic acid encoding one or more human Ig variable D domains, followed by nucleic acid encoding a second amino acid sequence of a FN3 polypeptide (e.g., a ¹⁰FN3 polypeptide) followed by an endogenous Eu element and/or an endogenous element Iμ and/or an endogenous Sμ element followed by nucleic acid that encodes an endogenous IgG hinge, CH2 domain, and CH3 domain in the absence of the endogenous CH1 domain for that IgG followed by an endogenous 3′γ1E element, an endogenous 3′RR element, and an endogenous 3′CBE element, while lacking the endogenous nucleic acid that encodes at least one full-length CH2 domain or CH3 domain of each of IgM, IgD, IgE, and IgA. An example of this genomic configuration is set forth in FIG. 52.

In some cases, instead of retaining an endogenous enhancer or regulatory element as described herein, one or more exogenous enhancer or regulatory elements can be engineered into a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge). For example, in some cases, a mouse can be designed as described herein where the endogenous mouse Eμ element is removed and replaced with a human Eu element.

In some cases, a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can be designed to produce antibody-like molecules that lack endogenous Ig VH domains. For example, non-human animals provided herein can be non-human animals whose genomes have (e.g., are genetically engineered to have) one or more disruptions in an endogenous nucleic acid sequence encoding an Ig VH locus, such that antibody-like molecules encoded by the non-human animal lack endogenous Ig VH domains. In some cases, at least one allele of the genome of a non-human animal provided herein can lack at least a portion of the endogenous nucleic acid encoding one or more Ig VH domains. In some cases, at least one allele of the genome of a non-human animal provided herein can lack all the exons of the endogenous nucleic acid encoding Ig VH domains. In some cases, both alleles of the genome of a non-human animal provided herein can lack all the exons of the endogenous Ig heavy chain variable region. In some cases, neither allele of the genome of a non-human animal provided herein contains an endogenous exon of a nucleic acid encoding an Ig VH domain.

In some cases, a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can be designed to have a variable region Ig locus that was modified to include nucleic acid encoding one or more segments of an FN3 polypeptide that is not endogenous to that non-human animal. For example, a mouse provided herein can be designed to have an Ig VH locus that was modified to include one or more nucleic acid sequences encoding at least a portion of a human FN3 polypeptide.

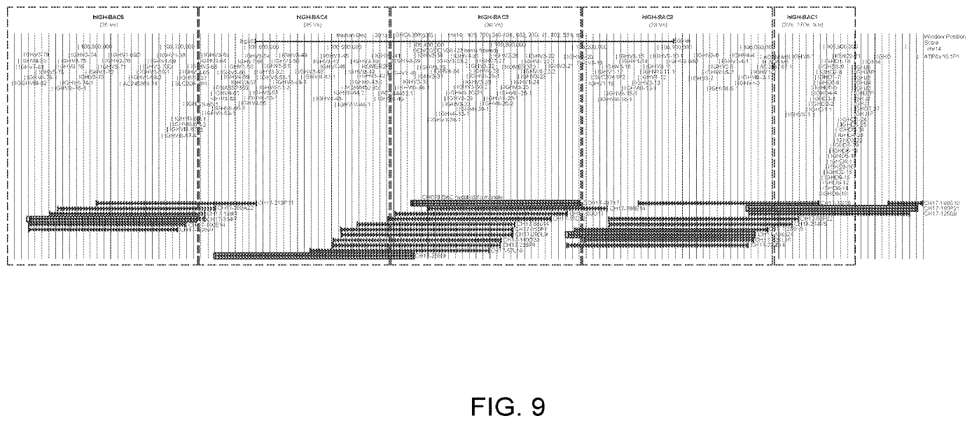

In some cases, an Ig locus present in the genome of a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can have any appropriate number of exogenous Ig variable D gene segments (e.g., 1, 2, 3, 4, 5, 6, 7, 8, 9, 10, 11, 12, 13, 14, 15, 1, 17, 18, 19, 20, 21, 22, 23, 24, 25, 26, or 27 human Ig variable D gene segments such as those human gene segments shown in FIG. 9) located between a first amino acid sequence of a FN3 polypeptide (e.g., a ¹⁰FN3 polypeptide) and a second amino acid sequence of a FN3 polypeptide (e.g., a ¹⁰FN3 polypeptide). In some cases, an Ig locus present in the genome of a mouse provided herein can include one, two, three, four, five, six, seven, eight, or nine human Ig variable D gene segments that are not mouse Ig variable D gene segments.

In some cases, an Ig locus present in the genome of a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can have any appropriate one or more human Ig variable D domain gene segments. Examples of human Ig variable D domain gene segments that can be included in a genome of a non-human animal provided herein include, without limitation, the human Ig variable D domain gene segment referred to as IGHD7-27, the human Ig variable D domain gene segment referred to as IGHD1-26, the human Ig variable D domain gene segment referred to as IGHD6-25, the human Ig variable D domain gene segment referred to as IGHD5-24, the human Ig variable D domain gene segment referred to as IGHD4-23, the human Ig variable D domain gene segment referred to as IGHD3-22, the human Ig variable D domain gene segment referred to as IGHD2-21, the human Ig variable D domain gene segment referred to as IGHD1-20, the human Ig variable D domain gene segment referred to as IGHD6-19, the human Ig variable D domain gene segment referred to as IGHD5-18, the human Ig variable D domain gene segment referred to as IGHD4-17, the human Ig variable D domain gene segment referred to as IGHD3-16, the human Ig variable D domain gene segment referred to as IGHD2-15, the human Ig variable D domain gene segment referred to as IGHD1-14, the human Ig variable D domain gene segment referred to as IGHD6-13, the human Ig variable D domain gene segment referred to as IGHD5-12, the human Ig variable D domain gene segment referred to as IGHD4-11, the human Ig variable D domain gene segment referred to as IGHD3-10, the human Ig variable D domain gene segment referred to as IGHD3-9, the human Ig variable D domain gene segment referred to as IGHD2-8, the human Ig variable D domain gene segment referred to as IGHD1-7, the human Ig variable D domain gene segment referred to as IGHD6-6, the human Ig variable D domain gene segment referred to as IGHD5-5, the human Ig variable D domain gene segment referred to as IGHD4-4, the human Ig variable D domain gene segment referred to as IGHD3-3, the human Ig variable D domain gene segment referred to as IGHD2-2, and human Ig variable D domain gene segment referred to as IGHD1-1. Examples of human Ig variable D domain gene segments that can be included in a genome of a non-human animal provided herein also include, without limitation, human Ig variable D domain gene segments that encode the amino acid sequence encoded by IGHD7-27, human Ig variable D domain gene segments that encode the amino acid sequence encoded by IGHD1-26, human Ig variable D domain gene segments that encode the amino acid sequence encoded by IGHD6-25, human Ig variable D domain gene segments that encode the amino acid sequence encoded by IGHD5-24, human Ig variable D domain gene segments that encode the amino acid sequence encoded by IGHD4-23, human Ig variable D domain gene segments that encode the amino acid sequence encoded by IGHD3-22, human Ig variable D domain gene segments that encode the amino acid sequence encoded by IGHD2-21, human Ig variable D domain gene segments that encode the amino acid sequence encoded by IGHD1-20, human Ig variable D domain gene segments that encode the amino acid sequence encoded by IGHD6-19, human Ig variable D domain gene segments that encode the amino acid sequence encoded by IGHD5-18, human Ig variable D domain gene segments that encode the amino acid sequence encoded by IGHD4-17, human Ig variable D domain gene segments that encode the amino acid sequence encoded by IGHD3-16, human Ig variable D domain gene segments that encode the amino acid sequence encoded by IGHD2-15, human Ig variable D domain gene segments that encode the amino acid sequence encoded by IGHD1-14, human Ig variable D domain gene segments that encode the amino acid sequence encoded by IGHD6-13, human Ig variable D domain gene segments that encode the amino acid sequence encoded by IGHD5-12, human Ig variable D domain gene segments that encode the amino acid sequence encoded by IGHD4-11, human Ig variable D domain gene segments that encode the amino acid sequence encoded by IGHD3-10, human Ig variable D domain gene segments that encode the amino acid sequence encoded by IGHD3-9, human Ig variable D domain gene segments that encode the amino acid sequence encoded by IGHD2-8, human Ig variable D domain gene segments that encode the amino acid sequence encoded by IGHD1-7, human Ig variable D domain gene segments that encode the amino acid sequence encoded by IGHD6-6, human Ig variable D domain gene segments that encode the amino acid sequence encoded by IGHD5-5, human Ig variable D domain gene segments that encode the amino acid sequence encoded by IGHD4-4, human Ig variable D domain gene segments that encode the amino acid sequence encoded by IGHD3-3, human Ig variable D domain gene segments that encode the amino acid sequence encoded by IGHD2-2, and human Ig variable D domain gene segments that encode the amino acid sequence encoded by IGHD1-1.

In some cases, an Ig locus present in the genome of a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can include one or more regulatory elements for each nucleic acid sequence encoding a portion of a FN3 polypeptide inserted into the non-mouse Ig locus (e.g., to control expression and/or recombination for the first and second amino acid sequences of a FN3 polypeptide (e.g., a ¹⁰FN3 polypeptide)). The regulatory element(s) can be endogenous or exogenous. For example, the regulatory element(s) can be human regulatory element(s). Examples of regulatory elements that can be used to control expression and/or recombination for each nucleic acid sequence encoding a portion of a FN3 polypeptide include, without limitation, promoters, enhancers, transcription factor binding sites, splice sites, recombination signal sequences, leader exon 1, leader exon 2, signal peptide sequences, and introns.

Any appropriate method can be used to generate a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge). In some cases, nucleic acid encoding a first amino acid sequence of a FN3 polypeptide (e.g., a ¹⁰FN3 polypeptide), nucleic acid encoding one or more human Ig variable D domains, and nucleic acid encoding a second amino acid sequence of a FN3 polypeptide (e.g., a ¹⁰FN3 polypeptide) can be inserted into the genome of a non-human animal (e.g., a mouse) that lacks nucleic acid encoding at least a portion of an endogenous CH1 (or lacks at least a portion of an endogenous regulatory element that drives expression of an endogenous CH1 domain) and optionally lacks at least a portion of the endogenous nucleic acid encoding one or more (e.g., all) endogenous VH domains. For example, nucleic acid encoding a first amino acid sequence of a human FN3 polypeptide (e.g., a human ¹⁰FN3 polypeptide), nucleic acid encoding one or more human Ig variable D domains, and nucleic acid encoding a second amino acid sequence of a human FN3 polypeptide (e.g., a human ¹⁰FN3 polypeptide) can be inserted into the genome of an engineered mouse as set forth in FIGS. 4, 5D, and 6B (also referred to as a Singularity HyperDock non-human animal or a Singularity HyperDock mouse). In some cases, nucleic acid encoding a first amino acid sequence of a FN3 polypeptide (e.g., a ¹⁰FN3 polypeptide), nucleic acid encoding one or more human Ig variable D domains, and nucleic acid encoding a second amino acid sequence of a FN3 polypeptide (e.g., a ¹⁰FN3 polypeptide) can include at least one recombination site (e.g., at least one nucleic acid sequence that can be recognized by a recombinase) on each end (e.g., at the 5′ end and at the 3′ end) of those nucleic acids. In some cases, the non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of the endogenous nucleic acid encoding one or more (e.g., all) VH domains (e.g., a Singularity HyperDock mouse) can include at least one recombination site, such that a recombinase can facilitate the insertion of nucleic acid encoding a first and/or second amino acid sequence of a FN3 polypeptide (e.g., a ¹⁰FN3 polypeptide) into the genome of that non-human animal.

In some cases, nucleic acid encoding a first and/or second amino acid sequence of a FN3 polypeptide (e.g., a ¹⁰FN3 polypeptide) can be present on a vector. Examples of vectors that can include nucleic acid encoding a first and/or second amino acid sequence of a FN3 polypeptide (e.g., a ¹⁰FN3 polypeptide) include, without limitation, yeast artificial chromosomes (YACs), bacterial artificial chromosomes (BACs), human artificial chromosomes (HACs), transchromosomes (e.g., whole transchromosomes and fragmented transchromosomes), P1-derived artificial chromosome (PACs), plasmids, and phagemids.

A non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of endogenous nucleic acid encoding one or more VH domains can include any appropriate recombination site(s). In some cases, a recombinase site can be an exogenous recombinase site. Examples of recombination sites that can be present in the genome of a non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of endogenous nucleic acid encoding one or more VH domains include, without limitation, frt, loxP, M2, M3, ox2271, lox2372, loxFAS, loxN, lox5171, lox2272, attB, and attP. Additional examples of recombination sites that can be present in the genome of a non-human animal (e.g., a mouse) provided herein can be as shown in Table 1.

A genome of a non-human animal (e.g., a mouse) described herein can include any appropriate number of recombination sites. For example, a non-human animal (e.g., a mouse) described herein can include one, two, three, four, five, six, seven, eight, nine, ten, or more recombination sites. When a non-human animal (e.g., a mouse) described herein includes two or more recombination sites in its genome, the recombination sites can each be a different recombination site. For example, a non-human animal (e.g., a mouse) described herein can include at least three different recombination sites within its genome. For example, a non-human animal (e.g., a mouse) described herein can include at least five different recombination sites within its genome.

A recombination site in a non-human animal (e.g., a mouse) described herein can be at any appropriate location within the genome of the non-human animal. In some cases, one or more recombination sites within a genome of a non-human animal described herein can be upstream of endogenous nucleic acid encoding an Ig CH2 domain or an Ig CH3 domain. In some cases, one or more recombination sites within a genome of a non-human animal described herein can be upstream (e.g., less than 2.5 Mb upstream) of an endogenous Eu element. When a genome of a non-human animal (e.g., a mouse) described herein includes different recombination sites (e.g., different exogenous recombination sites), each of the recombination sites can be located less than 2.0 Mb, less than 1.5 Mb, less than 1.0 Mb, less than 500 kb, less than 250 kb, less than 200 kb, less than 100 kb, less than 50 kb, less than 25 kb, or less than 10 kb upstream of an endogenous Eμ element.

Any appropriate method can be used to make a non-human animal described herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge). For example, gene editing techniques (e.g., CRISPR/Cas gene editing, TALEN gene editing, and/or zinc finger-based gene editing), recombination techniques (e.g., sequential Recombinase Mediated Cassette Exchange (RMCE)), and combinations thereof can be used to make a non-human animal described herein. In some cases, a nucleic acid molecule (e.g., an exogenous nucleic acid molecule) encoding a first and/or second amino acid sequence of a FN3 polypeptide (e.g., a ¹⁰FN3 polypeptide) can be introduced into a stem cell of a non-human animal (e.g., a mouse), the stem cell can be implanted into a blastocyst of the same species of non-human animal, and the blastocyst can be implanted into a pseudo-pregnant female of the same species of non-human animal to obtain a chimeric non-human animal, crossing the chimeric non-human animal to a wild-type non-human animal to produce offspring, screening the offspring for heterozygosity, and identifying a founder non-human animal carrying the nucleic acid encoding a first and/or second amino acid sequence of a FN3 polypeptide (e.g., a ¹⁰FN3 polypeptide) within its genome. In some cases, a non-human animal provided herein can be made as described in the Examples.

This document also provides methods for producing populations of antibody-like molecules that include (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge. For example, a non-human animal described herein (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge, can be administered one or more antigens (e.g., a composition including one or more antigens), such that one or more B cells in the non-human animal produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge. In some cases, one or more B cells can be isolated from the non-human animal.

Antibody-like molecules against any appropriate antigen can be produced by a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge). In some cases, an antigen can be an endogenous antigen or self-antigen. In some cases, an antigen can be an exogenous antigen. An antigen can be any appropriate type of molecule (e.g., a peptide, a lipid, or a nucleic acid). Examples of antigens that can be used to immunize a non-human animal provided herein can be as listed in Table 2.

In some cases, immunizing a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) with one or more antigen described herein can activate at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 non-human B cell clones in the non-human animal.

In some cases, immunizing a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) with one or more antigens described herein can lead to production of polyclonal antiserum including antibody-like molecules that include (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge, and that can bind (e.g., specifically bind) at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 antigens of the administered immunogenic composition.

This document also provides antibody-like molecules produced by immunization of a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge). Antibody-like molecules produced by a non-human animal provided herein can be obtained using any appropriate method. For example, antibody-like molecules can be obtained from the blood of a non-human animal provided herein. In some cases, a non-human animal provided herein can be immunized with a particular antigen (e.g., a SARS-COV-2 antigen) such that the non-human animal produces antibody-like molecules against that antigen, and the antibody-like molecules produced can be assessed for the desired properties (e.g., binding properties, neutralization properties, and/or solubility properties). In some cases, blood can be collected from a non-human animal provided herein that has been immunized as described herein with a particular antigen multiple times (e.g., after each of multiple immunizations, multiple times after a single immunization, multiple times in between immunizations, or any combination thereof). Blood can be collected from a non-human animal provided herein that has been immunized as described herein any suitable amount of time following an immunization. For example, blood can be collected from a non-human animal provided herein that has been immunized at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more, after an immunization. In some cases, blood can be collected at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most at most 35, at most 42, at most 49, or at most 56 days after an immunization. In some cases, blood can be collected about 1, 2, 3, 4, 5, 6, 7, 8, 9, 10, 11, 12, 14, 16, 17, 18, 20, 21, 22, 23, 24, 25, 26, 27, 28, 29, 30, 31, 32, 33, 34, 35, 36, 37, 38, 39, 40, 41, or 42 days, or more after an immunization.

In some cases, a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can retain its endogenous non-human immune cells. For example, a non-human animal provided herein can retain its non-human T cells, B cells, and/or antigen-presenting cells.

In some cases, a non-human animal provided herein (e.g., a genetically engineered non-human animal such as a mouse that can, when exposed to one or more antigens, produce an antibody-like molecule that includes (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a non-human Ig CH2 domain and/or a non-human Ig CH3 domain (e.g., endogenous IgG CH2 and/or IgG CH3 domains), and optionally a non-human Ig hinge) can include any feature or any combination of features (or any methods of making can be performed) as disclosed in U.S. Patent Application Publication No. 2017/0233459, which is hereby incorporated by reference in its entirety. For example, a non-human animal provided herein can include any feature or any combination of features (or any methods of making can be performed) as disclosed in Kuroiwa et al., Nat. Biotechnol., 27 (2): 173-81 (2009); Matsushita et al., PLos ONE, 9 (3):e90383 (2014); Hooper et al., Sci. Transl. Med., 6 (264): 264ra162 (2014); Matsushit et al., PLOS ONE, 10 (6):e0130699 (2015); Luke et al., Sci. Transl. Med., 8 (326): 326ra21 (2016); Dye et al., Sci. Rep., 6:24897 (2016); Gardner et al., J. Virol., 91 (14) (2017); Stein et al., Antiviral Res., 146:164-173 (2017); Silver, Clin. Infect. Dis., 66 (7): 1116-1119 (2018); Beigel et al., Lancet Infect. Dis., 18 (4): 410-418 (2018); Luke et al., J. Inf. Dis., 218 (suppl 5):S636-S648 (2018), each of which is hereby incorporated by reference in its entirety.

In some cases, a first and/or second amino acid sequence of a FN3 polypeptide (e.g., a human FN3 polypeptide such as a human ¹⁰FN3 polypeptide) described herein can include one or more amino acid modifications. Such amino acid modifications can include, without limitation, amino acid substitutions, amino acid deletions, amino acid additions, and combinations thereof. In some cases, an amino acid modification can be made to improve the binding and/or contact with an antigen and/or to improve a functional activity of an antibody-like molecule provided herein. In some cases, an amino acid substitution within a first and/or second amino acid sequence of a FN3 polypeptide (e.g., a human FN3 polypeptide such as a human ¹⁰FN3 polypeptide) described herein can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within a first and/or second amino acid sequence of a FN3 polypeptide (e.g., a human FN3 polypeptide such as a human ¹⁰FN3 polypeptide) described herein can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to a first and/or second amino acid sequence of a FN3 polypeptide (e.g., a human FN3 polypeptide such as a human 10FN3 polypeptide) described herein) can include site-specific mutagenesis or random mutagenesis (e.g., by PCR) of a nucleic acid encoding an antibody or a fragment thereof. See, for example, Zoller, Curr. Opin. Biotechnol. 3:348-354 (1992). Both naturally occurring and non-naturally occurring amino acids (e.g., artificially-derivatized amino acids) can be used to generate an amino acid sequence variant from an amino acid sequence of a first and/or second amino acid sequence of a FN3 polypeptide (e.g., a human FN3 polypeptide such as a human ¹⁰FN3 polypeptide) described herein.

Engineered Non-Human Animals for Producing Antibodies with Improved Properties

In another aspect, this document relates to genetically modified or engineered non-human animals (e.g., genetically modified or engineered mice) that produce antibodies (e.g., sdAbs and/or heavy chain only antibodies) having a chimeric heavy chain that includes (a) a non-human CH2 domain and/or a non-human CH3 domain (and optionally a non-human Ig hinge) and (b) a variable region that includes a human JH domain (e.g., a human JH3 domain or a human JH4 domain) lacking all or at least one tryptophan amino acid residue(s) such as a human JH domain encoded by a JH gene segment set forth in Example 17 (e.g., a JH domain set forth in any one of SEQ ID NOs:D18-D19), and methods of making and using the same. For example, this document provides genetically engineered non-human animals of a particular species (e.g., a mouse species) that produce antibodies (e.g., sdAbs and/or heavy chain only antibodies) having one or two chimeric heavy chain that include (a) a CH2 domain of that same species (e.g., a mouse CH2 domain) and/or a CH3 domain of that same species (e.g., a mouse CH2 domain) and (b) a variable region that includes a human JH domain (e.g., a human JH3 domain or a human JH4 domain) lacking all or at least one tryptophan amino acid residue(s).

Antibodies produced by a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can be any appropriate type of antibody. Examples of antibodies that can be produced by a non-human animal provided herein include, without limitation, heavy chain only antibodies. Examples of antibodies that can be designed or generated from an antibody produced by a non-human animal provided herein include, without limitation, sdAbs, Fabs, Fab′, F(ab′)₂, Fd, Fvs, single-chain variable fragments (scFvs), heavy chain only antibodies, and disulfide-linked Fvs (sdFv). In some cases, non-human animals (e.g., mice) provided herein can, when exposed to one or more antigens, produce IgG1 heavy chain only antibodies, IgG2 heavy chain only antibodies, IgG3 heavy chain only antibodies, IgG4 heavy chain only antibodies, or combinations thereof.

An antibody produced by a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 (and optionally a non-human Ig hinge) domain and (b) a variable region that includes a human JH domain lacking all or at least one tryptophan amino acid residue(s)) can have any appropriate stability. In some cases, an antibody produced by a non-human animal provided herein have increased stability (e.g., compared to an antibody such as a sdAb or heavy chain only antibody having one or two chimeric heavy chains that include (a) a non-human CH2 domain and/or a non-human CH3 (and optionally a non-human Ig hinge) domain and (b) a variable region that includes a human JH domain that does not lack all or at least one tryptophan amino acid residue(s) as described herein).

A chimeric heavy chain of an antibody (e.g., a heavy chain only antibody) produced by a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can include any appropriate human JH domain. Examples of human JH domains that can be included in a chimeric heavy chain produced by a non-human animal provided herein include, without limitation, a a human JH3 domain modified to have the amino acid sequence set forth in SEQ ID NO: 105, and a human JH4 domain modified to have the amino acid sequence set forth in SEQ ID NO: 106. In some cases, a JH domain that can be included in a variable region of a chimeric heavy chain produced by a non-human animal provided herein can be as described in Example 17.

A non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can be any type of non-human animal. Examples of non-human animals that can be designed to produce antibodies (e.g., sdAbs and/or heavy chain only antibodies) having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s) as described herein include, without limitation, mice, rats, rabbits, guinea pigs, zebrafish, flies (e.g., Drosophila melanogaster), pigs, sheep, non-human primates (e.g., monkeys), and bovine species. In some cases, a non-human animal designed to produce antibodies (e.g., sdAbs and/or heavy chain only antibodies) having one or two chimeric heavy chains that include (a) a mouse CH2 domain and/or a mouse CH3 domain (and optionally a non-human Ig hinge) and (b) a variable region that includes a human JH domain lacking all or at least one tryptophan amino acid residue(s) can be a mouse.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can be designed to produce antibodies that lack CH1 domains and that lack light chains. For example, a non-human animal (e.g., a mouse) provided herein can be a non-human animal whose genome has (e.g., is genetically engineered to have) one or more disruptions in an endogenous nucleic acid sequence encoding an CH1 domain of an IgG1 C-region gene (e.g., C_{γ1}), such that an IgG antibody encoded by the non-human animal lacks a CH1 domain and lacks light chains. In some cases, the genome of a non-human animal provided herein can lack nucleic acid encoding at least a portion of an endogenous CH1 domain. In some cases, the genome of a non-human animal provided herein can lack at least a portion of an endogenous regulatory element that drives expression of an endogenous CH1 domain.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can be designed to have a deletion of endogenous nucleic acid encoding a CH1 domain of a constant region (e.g., of an IgG C-region such as a CH1 domain of an IgG1 C-region, a CH1 domain of an IgG2a C-region, a CH1 domain of an IgG2b C-region, and/or a CH1 domain of an IgG3 C-region). The CH1 domain can contain multiple exons. In some cases, endogenous exon 1 of a CH1 domain of a constant region such as an IgG C-region can be deleted such that the engineered non-human animal (e.g., mouse) produces IgMΔCH1, IgGΔCH1, IgDΔCH1, IgAΔCH1, and/or IgEΔCH1 heavy chain only antibodies.

When making one or more genetic modifications to delete all or part of the nucleic acid encoding a CH1 domain (e.g., a CH1 domain of an IgG1 C-region, a CH1 domain of an IgG2a C-region, a CH1 domain of an IgG2b C-region, and/or a CH1 domain of an IgG3 C-region) such that the engineered non-human animal produces IgΔCH1 heavy chain only antibodies (e.g., IgGΔCH1 heavy chain only antibodies), the endogenous nucleic acid encoding a hinge domain, a heavy chain CH2 domain, and a heavy chain CH3 domain can remain intact. For example, to make a mouse that produces IgG1ΔCH1 heavy chain only antibodies, the genome of that mouse can lack exon 1 (and/or additional portions) of the CH1 domain of IgG1 while retaining the endogenous mouse nucleic acid needed to express the hinge domain, a heavy chain CH2 domain, and a heavy chain CH3 domain of IgG1, thereby resulting in a mouse that is capable of producing IgG1ΔCH1 heavy chain only antibodies.

Additional endogenous nucleic acid components that can be deleted from the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) to create a non-human animal provided herein include, without limitation, the introns and/or exons of the IgM constant domains (e.g., the μ constant domain locus), the introns and/or exons of the IgD constant domains (e.g., the δ constant domain locus), the introns and/or exons of the IgE constant domains (e.g., the ε constant domain locus), and/or the introns and/or exons of the IgA constant domains (e.g., the α constant domain locus). For example, a non-human animal provided herein can be designed to lack the introns and exons of the IgM constant domains (e.g., the μ constant domain locus), the introns and exons of the IgD constant domains (e.g., the δ constant domain locus), the introns and exons of the IgE constant domains (e.g., the ε constant domain locus), and the introns and exons of the IgA constant domains (e.g., the α constant domain locus).

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) to produce only IgG1ΔCH1 heavy chain only antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus). An example of a genetic engineering approach to create a mouse that produces only IgG1ΔCH1 heavy chain only antibodies is set forth in FIGS. 3A-3E.

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) to produce only IgG2aΔCH1 heavy chain only antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ1 constant domains (e.g., the γ1 constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) to produce only IgG2bΔCH1 heavy chain antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igy 1 constant domains (e.g., the γ1 constant domain locus), and the endogenous (if endogenously present) introns and/or exons of the Igγ2c constant domains (e.g., the γ2c constant domain locus).

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) to produce only IgG2cΔCH1 heavy chain antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ1 constant domains (e.g., the γ1 constant domain locus).

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) to produce only IgG3ΔCH1 heavy chain antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ1 constant domains (e.g., the γ1 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

As described herein, retaining and/or creating new positioning for certain endogenous enhancer or regulatory elements of a non-human animal can result in a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) that produces effectively large collections of and/or amounts of diverse heavy chain only antibodies (e.g., heavy chain only antibodies such as chimeric human/mouse heavy chain only antibodies). For example, a non-human animal provided herein can be designed to retain the μ enhancer (Eμ), the μ switch region (Sμ), and/or the μ promoter containing I-exon (Iμ) that are endogenously found upstream of the nucleic acid encoding the IgM constant domains. In some cases, non-human animals (e.g., mice) provided herein can be designed such that the retained endogenous Eμ, Sμ, and/or Iμ elements are in a genomic position such that the first nucleic acid sequence downstream of the retained Eμ, Sμ, and/or Iμ elements that encodes a full-length endogenous Ig constant domain is one that encodes a CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 1B and FIG. 3C where the nucleic acids of the endogenous mouse Eμ, Sμ, and Iμ elements are repositioned to be upstream of the nucleic acid encoding the endogenous IgG1 CH2 domain.

In another example, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can be designed to retain the 3′RR and/or 3′CBE elements that are endogenously found downstream of the nucleic acid encoding the IgA constant domains. In some cases, non-human animals (e.g., mice) provided herein can be designed such that the retained endogenous 3′RR and/or 3′CBE elements are in a genomic position such that the first nucleic acid sequence upstream of the retained 3′RR and/or 3′CBE elements that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 2B and FIG. 3E where the nucleic acid of the endogenous mouse 3′RR element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 3′RR element.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can be designed to retain the 3′γ1E element that is endogenously found between, for example, the IgG1 and IgG2b loci. In some cases, non-human animals (e.g., mice) provided herein can be designed such that the retained endogenous 3′γ1E element is in a genomic position such that nucleic acid encoding two, one, or no full-length endogenous Ig CH2 domains is located between the retained endogenous 3′γ1E element and a retained endogenous 3′RR element and/or a retained endogenous 3′CBE element. An example of this genomic configuration is set forth in FIG. 3E where the nucleic acid of the endogenous mouse 3′γ1E element is repositioned to be upstream of a retained endogenous 3′RR element such that no other nucleic acid encoding a full length IgG CH2 domain is located between the endogenous mouse 3′γ1E element and the endogenous 3′RR element.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can be designed to retain the 5′hsR1 element that is endogenously found within the IgA constant domain locus. In some cases, non-human animals (e.g., mice) provided herein can be designed such that the retained endogenous 5′hsR1 element is in a genomic position such that the first nucleic acid sequence upstream of the retained 5′hsR1 element that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 3E where the nucleic acid of the endogenous mouse 5′hsR1 element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 5′hsR1 element.

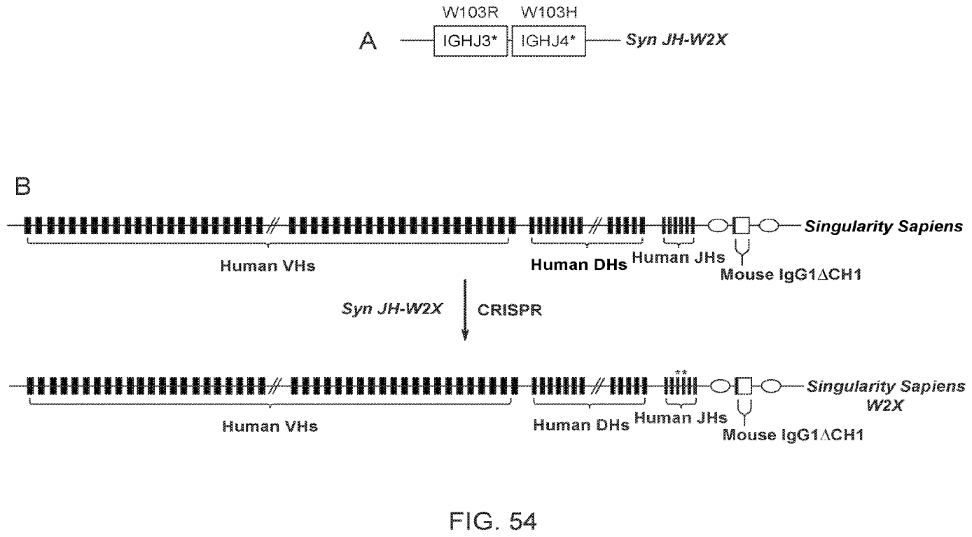

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can be designed to have (a) a heavy chain variable region locus (e.g., a heavy chain variable region locus including (i) one or more human VH gene segments, (ii) one or more human DH gene segments, and (iii) one or more modified human JH gene segments that do not encode tryptophan amino acid residues) followed by (b) an endogenous Eμ element and/or an endogenous element Iμ and/or an endogenous Sμ element followed by (c) nucleic acid that encodes endogenous IgG hinge, CH2 domain, and CH3 domain in the absence of the endogenous CH1 domain for that IgG followed by (e) an endogenous 3′γ1E element, an endogenous 3′RR element, and an endogenous 3′CBE element, while lacking the endogenous nucleic acid that encodes at least one full-length CH2 domain or CH3 domain of each of IgM, IgD, IgE, and IgA. An example of this genomic configuration is set forth in FIG. 3F. See, also, FIG. 54B.

In some cases, instead of retaining an endogenous enhancer or regulatory element as described herein, one or more exogenous enhancer or regulatory elements can be engineered into a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)). For example, in some cases, a mouse can be designed as described herein where the endogenous mouse Eμ element is removed and replaced with a human Eμ element.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can be designed to produce antibodies that lack the endogenous VH. For example, non-human animals provided herein can be non-human animals whose genomes have (e.g., are genetically engineered to have) one or more disruptions in an endogenous nucleic acid sequence encoding a VH locus, such that an antibody encoded by the non-human animal lacks the endogenous VH. In some cases, at least one allele of the genome of a non-human animal provided herein can lack at least a portion of the endogenous nucleic acid encoding a VH. In some cases, at least one allele of the genome of a non-human animal provided herein can lack all the exons of the endogenous nucleic acid encoding VH domains. In some cases, both alleles of the genome of a non-human animal provided herein can lack all the exons of the endogenous Ig heavy chain variable region. In some cases, neither allele of the genome of a non-human animal provided herein contains an endogenous exon of a nucleic acid encoding a VH domain.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can be designed to have a variable region locus that is not endogenous to that non-human animal. For example, a mouse provided herein can be designed to have a non-mouse heavy chain variable region locus (e.g., a heavy chain variable region locus including (i) one or more human VH gene segments, (ii) one or more human DH gene segments, and (iii) one or more modified human J_{H} gene segments that do not encode tryptophan amino acid residues).

A non-mouse JH locus (e.g., a JH locus including one or more modified human JH gene segments that do not encode tryptophan amino acid residues) present in the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can have any appropriate number of exogenous JH gene segments. In some cases, a non-mouse JH locus can include two, three, four, five, six, or more JH gene segments that each encode a human JH domain modified to lack all or at least one tryptophan amino acid residue(s).

Any one or more of the human JH gene segments incorporated into the genome of a non-human animal provided herein can be modified such that the tryptophan residues typically encoded are replaced with another amino acid such as alanine, arginine, asparagine, aspartic acid, cysteine, glutamine, glutamic acid, glycine, histidine, isoleucine, leucine, lysine, methionine, phenylalanine, proline, serine, threonine, tyrosine, or valine. In some cases, one or more of the human JH gene segments incorporated into the genome of a non-human animal provided herein can be modified such that the tryptophan residues typically encoded are replaced with threonine, arginine, tyrosine, serine, or glutamine. In some cases, one or more of the human JH gene segments incorporated into the genome of a non-human animal provided herein can be modified such that the tryptophan residues typically encoded are replaced with histidine. In some cases, one or more of the human JH gene segments incorporated into the genome of a non-human animal provided herein can be modified such that the tryptophan residues typically encoded are replaced with another amino acid as described in Example 17.

In some cases, a non-mouse JH locus (e.g., a JH locus including one or more modified human JH gene segments that do not encode tryptophan amino acid residues) present in the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can include one or more regulatory elements for each JH gene segment in the non-mouse JH locus (e.g., to control expression and/or recombination for each JH gene segment). The regulatory element(s) can be endogenous or exogenous. Examples of regulatory elements that can be used to control expression and/or recombination for each JH gene segment include, without limitation, promoters, enhancers, transcription factor binding sites, splice sites, recombination signal sequences, leader exon 1, leader exon 2, signal peptide sequences, and introns.

Any appropriate method can be used to generate a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)). In some cases, nucleic acid comprising one or more modified human JH gene segments can be inserted into the genome of a non-human animal (e.g., a mouse) that lacks nucleic acid encoding at least a portion of an endogenous CH1 (or lacks at least a portion of an endogenous regulatory element that drives expression of an endogenous CH1 domain) and optionally lacks at least a portion of the endogenous nucleic acid encoding one or more (e.g., all) endogenous VH domains. For example, a JH locus (e.g., nucleic acid comprising one or more modified human JH gene segments) can be inserted into the genome of an engineered mouse is set forth in FIGS. 4, 5D, and 6B (also referred to as a Singularity HyperDock non-human animal or a Singularity HyperDock mouse). In some cases, nucleic acid comprising one or more modified human JH gene segments can include at least one recombination site (e.g., at least one nucleic acid sequence that can be recognized by a recombinase) on each end (e.g., at the 5′ end and at the 3′ end) of the one or more modified human JH gene segments. In some cases, the non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of the endogenous nucleic acid encoding one or more (e.g., all) VH domains (e.g., a Singularity HyperDock mouse) can include at least one recombination site, such that a recombinase can facilitate the insertion of nucleic acid comprising one or more modified human JH gene segments into the genome of that non-human animal.

In some cases, nucleic acid comprising one or more modified human JH gene segments can be present on a vector. Examples of vectors that can include nucleic acid comprising one or more modified human JH gene segments include, without limitation, yeast artificial chromosomes (YACs), bacterial artificial chromosomes (BACs), human artificial chromosomes (HACs), transchromosomes (e.g., whole transchromosomes and fragmented transchromosomes), P1-derived artificial chromosome (PACs), plasmids, and phagemids.

A non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of endogenous nucleic acid encoding one or more VH domains can include any appropriate recombination site(s). In some cases, a recombinase site can be an exogenous recombinase site. Examples of recombination sites that can be present in the genome of a non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of endogenous nucleic acid encoding one or more VH domains include, without limitation, frt, loxP, M2, M3, lox2271, lox2372, loxFAS, loxN, lox5171, lox2272, attB, and attP. Additional examples of recombination sites that can be present in the genome of a non-human animal (e.g., a mouse) provided herein can be as shown in Table 1.

A genome of a non-human animal (e.g., a mouse) described herein can include any appropriate number of recombination sites. For example, a non-human animal (e.g., a mouse) described herein can include one, two, three, four, five, six, seven, eight, nine, ten, or more recombination sites. When a non-human animal (e.g., a mouse) described herein includes two or more recombination sites in its genome, the recombination sites can each be a different recombination site. For example, a non-human animal (e.g., a mouse) described herein can include at least three different recombination sites within its genome. For example, a non-human animal (e.g., a mouse) described herein can include at least five different recombination sites within its genome.

A recombination site in a non-human animal (e.g., a mouse) described herein can be at any appropriate location within the genome of the non-human animal. In some cases, one or more recombination sites within a genome of a non-human animal described herein can be upstream of endogenous nucleic acid encoding a CH2 domain or a CH3 domain. In some cases, one or more recombination sites within a genome of a non-human animal described herein can be upstream (e.g., less than 2.5 Mb upstream) of an endogenous Eμ. When a genome of a non-human animal (e.g., a mouse) described herein includes different recombination sites (e.g., different exogenous recombination sites), each of the recombination sites can be located less than 2.0 Mb, less than 1.5 Mb, less than 1.0 Mb, less than 500 kb, less than 250 kb, less than 200 kb, less than 100 kb, less than 50 kb, less than 25 kb, or less than 10 kb upstream of an endogenous Eμ.

Any appropriate method can be used to make a non-human animal described herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)). For example, gene editing techniques (e.g., CRISPR/Cas gene editing, TALEN gene editing, and/or zinc finger-based gene editing), recombination techniques (e.g., sequential Recombinase Mediated Cassette Exchange (RMCE)), and combinations thereof can be used to make a non-human animal described herein. In some cases, a nucleic acid molecule (e.g., an exogenous nucleic acid molecule) comprising one or more modified human JH gene segments can be introduced into a stem cell of a non-human animal (e.g., a mouse), the stem cell can be implanted into a blastocyst of the same species of non-human animal, and the blastocyst can be implanted into a pseudo-pregnant female of the same species of non-human animal to obtain a chimeric non-human animal, crossing the chimeric non-human animal to a wild-type non-human animal to produce offspring, screening the offspring for heterozygosity, and identifying a founder non-human animal carrying the one or more modified human JH gene segments within its genome. In some cases, a non-human animal provided herein can be made as described in the Examples.

This document also provides methods for producing populations of antibodies (e.g., sdAbs and/or heavy chain only antibodies) having improved stability (e.g., having a melting temperature of less than about 70° C.). For example, a non-human animal described herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can be administered one or more antigens (e.g., a composition including one or more antigens), such that one or more B cells in the non-human animal produce an antibody (e.g., a heavy chain only antibody) that includes a chimeric heavy chain that includes (a) a non-human CH2 domain and/or a non-human CH3 domain (and optionally a non-human Ig hinge) and (b) a variable region that includes a human JH domain lacking all or at least one tryptophan amino acid residue(s). In some cases, one or more B cells can be isolated from the non-human animal.

Antibodies against any appropriate antigen can be produced by a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)). In some cases, an antigen can be an endogenous antigen or self-antigen. In some cases, an antigen can be an exogenous antigen. An antigen can be any appropriate type of molecule (e.g., a peptide, a lipid, or a nucleic acid). Examples of antigens that can be used to immunize a non-human animal provided herein can be as listed in Table 2.

In some cases, immunizing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) with one or more antigen described herein can activate at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 non-human B cell clones in the non-human animal.

In some cases, immunizing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) with one or more antigens described herein can lead to production of polyclonal antiserum including antibodies (e.g., heavy chain only antibodies) (1) that have one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s) and (2) that can bind (e.g., specifically bind) at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 antigens of the administered immunogenic composition.

This document also provides antibodies (e.g., sdAbs and/or heavy chain only antibodies) produced by immunization of a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)). Antibodies (e.g., sdAbs and/or heavy chain only antibodies) produced by a non-human animal provided herein can be obtained using any appropriate method. For example, heavy chain only antibodies can be obtained from the blood of a non-human animal provided herein.

In some cases, a non-human animal provided herein can be immunized with a particular antigen (e.g., a SARS-COV-2 antigen) such that the non-human animal produces antibodies (e.g., sdAbs and/or heavy chain only antibodies) against that antigen, and the antibodies produced can be assessed for the desired properties (e.g., binding properties, neutralization properties, and/or solubility properties). In some cases, blood can be collected from a non-human animal provided herein that has been immunized as described herein with a particular antigen multiple times (e.g., after each of multiple immunizations, multiple times after a single immunization, multiple times in between immunizations, or any combination thereof). Blood can be collected from a non-human animal provided herein that has been immunized as described herein any suitable amount of time following an immunization. For example, blood can be collected from a non-human animal provided herein that has been immunized at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more, after an immunization. In some cases, blood can be collected at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most at most 35, at most 42, at most 49, or at most 56 days after an immunization. In some cases, blood can be collected about 1, 2, 3, 4, 5, 6, 7, 8, 9, 10, 11, 12, 14, 16, 17, 18, 20, 21, 22, 23, 24, 25, 26, 27, 28, 29, 30, 31, 32, 33, 34, 35, 36, 37, 38, 39, 40, 41, or 42 days, or more after an immunization.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can retain its endogenous non-human immune cells. For example, a non-human animal provided herein can retain its non-human T cells, B cells, and/or antigen-presenting cells.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human JH domain lacking all or at least one tryptophan amino acid residue(s)) can include any feature or any combination of features (or any methods of making can be performed) as disclosed in U.S. Patent Application Publication No. 2017/0233459, which is hereby incorporated by reference in its entirety. For example, a non-human animal provided herein can include any feature or any combination of features (or any methods of making can be performed) as disclosed in Kuroiwa et al., Nat. Biotechnol., 27 (2): 173-81 (2009); Matsushita et al., PLos ONE, 9 (3):e90383 (2014); Hooper et al., Sci. Transl. Med., 6 (264): 264ra162 (2014); Matsushit et al., PLOS ONE, 10 (6):e0130699 (2015); Luke et al., Sci. Transl. Med., 8 (326): 326ra21 (2016); Dye et al., Sci. Rep., 6:24897 (2016); Gardner et al., J. Virol., 91 (14) (2017); Stein et al., Antiviral Res., 146:164-173 (2017); Silver, Clin. Infect. Dis., 66 (7): 1116-1119 (2018); Beigel et al., Lancet Infect. Dis., 18 (4): 410-418 (2018); Luke et al., J. Inf. Dis., 218 (suppl_5):S636-S648 (2018), each of which is hereby incorporated by reference in its entirety.

In some cases, an amino acid sequence described herein can include one or more additional amino acid modifications that are in addition to the changes from tryptophan. Such amino acid modifications can include, without limitation, amino acid substitutions, amino acid deletions, amino acid additions, and combinations thereof. In some cases, an amino acid modification, including those that change tryptophan to another amino acid, can be made to improve the binding and/or contact with an antigen and/or to improve a functional activity of an antibody (e.g., a sdAb and/or a heavy chain only antibody) provided herein. In some cases, an amino acid substitution, including those that change tryptophan to another amino acid, can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution, including those that change tryptophan to another amino acid, can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding an antibody or a fragment thereof. See, for example, Zoller, Curr. Opin. Biotechnol. 3:348-354 (1992). Both naturally occurring and non-naturally occurring amino acids (e.g., artificially-derivatized amino acids) can be used to generate an amino acid sequence variant from an amino acid sequence described herein.

Additional Engineered Non-Human Animals for Producing Antibodies with Improved Properties

In another aspect, this document relates to genetically modified or engineered non-human animals (e.g., genetically modified or engineered mice) that produce antibodies (e.g., sdAbs and/or heavy chain only antibodies) having a chimeric heavy chain that includes (a) a non-human CH2 domain and/or a non-human CH3 domain (and optionally a non-human Ig hinge) and (b) a variable region that includes a human VH domain having a FR2 containing one, two, three, four, or more amino acid modifications (e.g., one, two, three, four, or more amino acid substitutions) such as a human VH domain encoded by a VH gene segment set forth in Example 20 (e.g., a VH domain set forth in any one of SEQ ID NOs: 119-123), and methods of making and using the same. For example, this document provides genetically engineered non-human animals of a particular species (e.g., a mouse species) that produce antibodies (e.g., sdAbs and/or heavy chain only antibodies) having one or two chimeric heavy chain that include (a) a CH2 domain of that same species (e.g., a mouse CH2 domain) and/or a CH3 domain of that same species (e.g., a mouse CH2 domain) and (b) a variable region that includes a human VH domain having a FR2 containing one, two, three, four, or more amino acid modifications (e.g., one, two, three, four, or more amino acid substitutions) such as a human VH domain encoded by a VH gene segment set forth in Example 20 (e.g., a VH domain set forth in any one of SEQ ID NOs: 119-123).

Antibodies produced by a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can be any appropriate type of antibody. In some cases, an antibody can include a VH domain having a modified FR2 as described herein. Examples of antibodies that can be produced by a non-human animal provided herein include, without limitation, heavy chain only antibodies. Examples of antibodies that can be design or generated from an antibody produced by a non-human animal provided herein include, without limitation, sdAbs, Fabs, Fab′, F(ab′)₂, Fd, Fvs, single-chain variable fragments (scFvs), heavy chain only antibodies, and disulfide-linked Fvs (sdFv). In some cases, non-human animals (e.g., mice) provided herein can, when exposed to one or more antigens, produce IgG1 heavy chain only antibodies, IgG2 heavy chain only antibodies, IgG3 heavy chain only antibodies, IgG4 heavy chain only antibodies, or combinations thereof.

An antibody produced by a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can have any appropriate solubility. In some cases, an antibody produced by a non-human animal provided herein have an increased solubility.

A chimeric heavy chain of an antibody (e.g., a heavy chain only antibody) produced by a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can include any appropriate VH domain (e.g., any appropriate human VH domain). Examples of human VH domains that can be included in a chimeric heavy chain produced by a non-human animal provided herein include, without limitation, an IGHV3-11 VH domain modified to have the amino acid sequence set forth in SEQ ID NO:119, an IGHV3-21 VH domain modified to have the amino acid sequence set forth in SEQ ID NO:120, an IGHV3-23 VH domain modified to have the amino acid sequence set forth in SEQ ID NO: 121, an IGHV4-39 VH domain modified to have the amino acid sequence set forth in SEQ ID NO:122, and an IGHV3-74 VH domain modified to have the amino acid sequence set forth in SEQ ID NO: 123. In some cases, a VH domain that can be included in a chimeric heavy chain produced by a non-human animal provided herein can be as described in Example 20.

A non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can be any type of non-human animal. Examples of non-human animals that can be designed to produce antibodies (e.g., sdAbs and/or heavy chain only antibodies) having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions as described herein include, without limitation, mice, rats, rabbits, guinea pigs, zebrafish, flies (e.g., Drosophila melanogaster), pigs, sheep, non-human primates (e.g., monkeys), and bovine species. In some cases, a non-human animal designed to produce antibodies (e.g., sdAbs and/or heavy chain only antibodies) having one or two chimeric heavy chains that include (a) a mouse CH2 domain and/or a mouse CH3 domain (and optionally a non-human Ig hinge) and (b) a variable region that includes a human VH domain having a FR2 containing one, two, three, four, or more amino acid modifications such as one, two, three, four, or more amino acid substitutions can be a mouse.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can be designed to produce antibodies that lack CH1 domains and that lack light chains. For example, a non-human animal (e.g., a mouse) provided herein can be a non-human animal whose genome has (e.g., is genetically engineered to have) one or more disruptions in an endogenous nucleic acid sequence encoding an CH1 domain of an IgG1 C-region gene (e.g., C_{γ1}), such that an IgG antibody encoded by the non-human animal lacks a CH1 domain and lacks light chains. In some cases, the genome of a non-human animal provided herein can lack nucleic acid encoding at least a portion of an endogenous CH1 domain. In some cases, the genome of a non-human animal provided herein can lack at least a portion of an endogenous regulatory element that drives expression of an endogenous CH1 domain.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can be designed to have a deletion of endogenous nucleic acid encoding a CH1 domain of a constant region (e.g., of an IgG C-region such as a CH1 domain of an IgG1 C-region, a CH1 domain of an IgG2a C-region, a CH1 domain of an IgG2b C-region, and/or a CH1 domain of an IgG3 C-region). The CH1 domain can contain multiple exons. In some cases, endogenous exon 1 of a CH1 domain of a constant region such as an IgG C-region can be deleted such that the engineered non-human animal (e.g., mouse) produces IgMΔCH1, IgGΔCH1, IgDΔCH1, IgAΔCH1, and/or IgEΔCH1 heavy chain only antibodies.

When making one or more genetic modifications to delete all or part of the nucleic acid encoding a CH1 domain (e.g., a CH1 domain of an IgG1 C-region, a CH1 domain of an IgG2a C-region, a CH1 domain of an IgG2b C-region, and/or a CH1 domain of an IgG3 C-region) such that the engineered non-human animal produces IgΔCH1 heavy chain only antibodies (e.g., IgGΔCH1 heavy chain only antibodies), the endogenous nucleic acid encoding a hinge domain, a heavy chain CH2 domain, and a heavy chain CH3 domain can remain intact. For example, to make a mouse that produces IgG1ΔCH1 heavy chain only antibodies, the genome of that mouse can lack exon 1 (and/or additional portions) of the CH1 domain of IgG1 while retaining the endogenous mouse nucleic acid needed to express the hinge domain, a heavy chain CH2 domain, and a heavy chain CH3 domain of IgG1, thereby resulting in a mouse that is capable of producing IgG1ΔCH1 heavy chain only antibodies.

Additional endogenous nucleic acid components that can be deleted from the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) to create a non-human animal provided herein include, without limitation, the introns and/or exons of the IgM constant domains (e.g., the μ constant domain locus), the introns and/or exons of the IgD constant domains (e.g., the δ constant domain locus), the introns and/or exons of the IgE constant domains (e.g., the ε constant domain locus), and/or the introns and/or exons of the IgA constant domains (e.g., the α constant domain locus). For example, a non-human animal provided herein can be designed to lack the introns and exons of the IgM constant domains (e.g., the μ constant domain locus), the introns and exons of the IgD constant domains (e.g., the δ constant domain locus), the introns and exons of the IgE constant domains (e.g., the ε constant domain locus), and the introns and exons of the IgA constant domains (e.g., the α constant domain locus).

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) to produce only IgG1ΔCH1 heavy chain only antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus). An example of a genetic engineering approach to create a mouse that produces only IgG1ΔCH1 heavy chain only antibodies is set forth in FIGS. 3A-3E.

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) to produce only IgG2aΔCH1 heavy chain only antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ1 constant domains (e.g., the yl constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) to produce only IgG2bΔCH1 heavy chain antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ1 constant domains (e.g., the γ1 constant domain locus), and the endogenous (if endogenously present) introns and/or exons of the Igγ2c constant domains (e.g., the γ2c constant domain locus).

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) to produce only IgG2cΔCH1 heavy chain antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ1 constant domains (e.g., the γ1 constant domain locus).

In some cases, when designing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) to produce only IgG3ΔCH1 heavy chain antibodies, the genome of that non-human animal can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ1 constant domains (e.g., the γ1 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

As described herein, retaining and/or creating new positioning for certain endogenous enhancer or regulatory elements of a non-human animal can result in a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) that produces effectively large collections of and/or amounts of diverse heavy chain only antibodies (e.g., heavy chain only antibodies such as chimeric human/mouse heavy chain only antibodies). For example, a non-human animal provided herein can be designed to retain the μ enhancer (Eμ), the μ switch region (Sμ), and/or the μ promoter containing I-exon (Ip) that are endogenously found upstream of the nucleic acid encoding the IgM constant domains. In some cases, non-human animals (e.g., mice) provided herein can be designed such that the retained endogenous Eμ, Sμ, and/or Iμ elements are in a genomic position such that the first nucleic acid sequence downstream of the retained Eμ, Sμ, and/or Iμ elements that encodes a full-length endogenous Ig constant domain is one that encodes a CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 1B and FIG. 3C′ where the nucleic acids of the endogenous mouse Eμ, Sμ, and Iμ elements are repositioned to be upstream of the nucleic acid encoding the endogenous IgG1 CH2 domain.

In another example, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can be designed to retain the 3′RR and/or 3′CBE elements that are endogenously found downstream of the nucleic acid encoding the IgA constant domains. In some cases, non-human animals (e.g., mice) provided herein can be designed such that the retained endogenous 3′RR and/or 3′CBE elements are in a genomic position such that the first nucleic acid sequence upstream of the retained 3′RR and/or 3′CBE elements that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 2B and FIG. 3E where the nucleic acid of the endogenous mouse 3′RR element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 3′RR element.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can be designed to retain the 3′γ1E element that is endogenously found between, for example, the IgG1 and IgG2b loci. In some cases, non-human animals (e.g., mice) provided herein can be designed such that the retained endogenous 3′γ1E element is in a genomic position such that nucleic acid encoding two, one, or no full-length endogenous Ig CH2 domains is located between the retained endogenous 3′γ1E element and a retained endogenous 3′RR element and/or a retained endogenous 3′CBE element. An example of this genomic configuration is set forth in FIG. 3E where the nucleic acid of the endogenous mouse 3′γ1E element is repositioned to be upstream of a retained endogenous 3′RR element such that no other nucleic acid encoding a full length IgG CH2 domain is located between the endogenous mouse 3′γ1E element and the endogenous 3′RR element.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can be designed to retain the 5′hsR1 element that is endogenously found within the IgA constant domain locus. In some cases, non-human animals (e.g., mice) provided herein can be designed such that the retained endogenous 5′hsR1 element is in a genomic position such that the first nucleic acid sequence upstream of the retained 5′hsR1 element that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 3E where the nucleic acid of the endogenous mouse 5′hsR1 element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 5′hsR1 element.

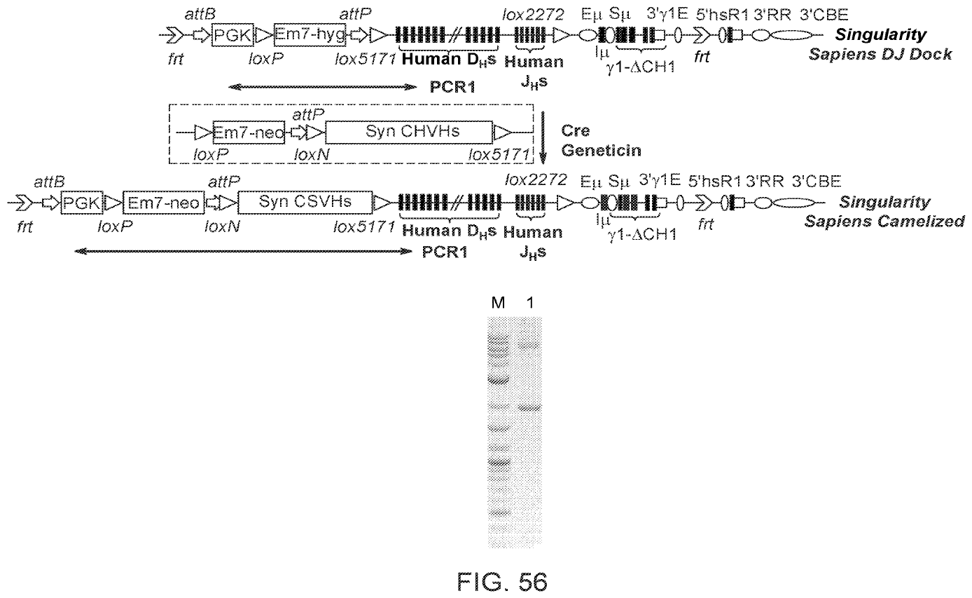

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can be designed to have (a) a VH locus (e.g., a VH locus including one or more human VH gene segments encoding a FR2 containing one, two, three, four, or more amino acid substitutions) followed by (b) an endogenous Eμ element and/or an endogenous element Iμ and/or an endogenous Sμ element followed by (c) nucleic acid that encodes endogenous IgG hinge, CH2 domain, and CH3 domain in the absence of the endogenous CH1 domain for that IgG followed by (e) an endogenous 3′γ1E element, an endogenous 3′RR element, and an endogenous 3′CBE element, while lacking the endogenous nucleic acid that encodes at least one full-length CH2 domain or CH3 domain of each of IgM, IgD, IgE, and IgA. An example of this genomic configuration is set forth in FIG. 3E. See, also, FIG. 56.

In some cases, instead of retaining an endogenous enhancer or regulatory element as described herein, one or more exogenous enhancer or regulatory elements can be engineered into a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions). For example, in some cases, a mouse can be designed as described herein where the endogenous mouse Eμ element is removed and replaced with a human Eμ element.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can be designed to produce antibodies that lack the endogenous VH. For example, non-human animals provided herein can be non-human animals whose genomes have (e.g., are genetically engineered to have) one or more disruptions in an endogenous nucleic acid sequence encoding a VH locus, such that an antibody encoded by the non-human animal lacks the endogenous VH. In some cases, at least one allele of the genome of a non-human animal provided herein can lack at least a portion of the endogenous nucleic acid encoding a VH. In some cases, at least one allele of the genome of a non-human animal provided herein can lack all the exons of the endogenous nucleic acid encoding VH domains. In some cases, both alleles of the genome of a non-human animal provided herein can lack all the exons of the endogenous Ig heavy chain variable region. In some cases, neither allele of the genome of a non-human animal provided herein contains an endogenous exon of a nucleic acid encoding a VH domain.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can be designed to have a variable region locus that is not endogenous to that non-human animal. For example, a mouse provided herein can be designed to have a non-mouse VH locus (e.g., a VH locus including one or more human VH gene segments).

A non-mouse VH locus (e.g., a VH locus including one or more human VH gene segments and/or one or more VH gene segments encoding a VH domain set forth in any of SEQ ID NOs: 119-123) present in the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions can have any appropriate number of exogenous VH gene segments. In some cases, a non-mouse VH locus can include two, three, four, five, six, seven, eight, nine, ten, 11, 12, 13, 14, 20, 50, 75, 100, 125, 129, or more VH gene segments that encode VH domains that are not mouse VH domains. In some cases, a non-mouse VH locus can include two, three, four, five, six, seven, eight, nine, ten, 11, 12, 13, 14, 20, 50, 75, 100, 125, 129, or more VH gene segments that encode modified human VH domains having a FR2 containing one, two, three, four, or more amino acid modifications (e.g., one, two, three, four, or more amino acid substitutions) as described herein.

A non-mouse VH locus (e.g., a VH locus including one or more human VH gene segments and/or one or more VH gene segments encoding a VH domain set forth in any of SEQ ID NOs: 119-123) present in the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can have any appropriate VH gene segments. In some cases, a non-mouse VH locus present in the genome of a non-human animal provided herein can include exogenous nucleic acid encoding one or more (e.g., one, two, three, four, five, six, seven, eight, nine, ten, 11, 12, 14, 15, 25, 35, 50, 65, 80, 100, 125, 129, or more) human VH gene segments and/or one or more (e.g., one, two, three, four, five, six, seven, eight, nine, ten. 11, 12, 14, 15, 25, 35, 50, 65, 80, 100, 125, 129, or more) modified VH gene segments encoding modified human VH domains having a FR2 containing one, two, three, four, or more amino acid modifications (e.g., one, two, three, four, or more amino acid substitutions) as described herein.

A non-mouse VH locus present in the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can have any appropriate one or more VH gene segments. Examples of VH gene segments that can be included in a genome of a non-human animal provided herein include, without limitation, the modified IGHV3-11 VH gene segment set forth in SEQ ID NO: 114, the modified IGHV3-21 VH gene segment set forth in SEQ ID NO:115, the IGHV3-23 VH gene segment set forth in SEQ ID NO:116, the modified IGHV4-39 VH gene segment set forth in SEQ ID NO:117, and the modified IGHV3-74 VH gene segment set forth in SEQ ID NO:118. Examples of VH gene segments that can be included in a genome of a non-human animal provided herein also include, without limitation, VH gene segments that encode the amino acid sequence encoded by a modified IGHV3-11, which amino acid sequence is set forth in SEQ ID NO: 119; VH gene segments that encode the amino acid sequence encoded by a modified IGHV3-21, which amino acid sequence is set forth in SEQ ID NO: 120; VH gene segments that encode the amino acid sequence encoded by a modified IGHV3-23, which amino acid sequence is set forth in SEQ ID NO: 121; VH gene segments that encode the amino acid sequence encoded by a modified IGHV4-39, which amino acid sequence is set forth in SEQ ID NO: 122; and VH gene segments that encode the amino acid sequence encoded by a modified IGHV3-74, which amino acid sequence is set forth in SEQ ID NO: 123. In some cases, nucleic acid sequences of VH gene segments that can be included in a genome of a non-human animal provided herein can be as described in Example 20. In some cases, nucleic acid sequences of VH gene segments that can be included in a genome of a non-human animal provided herein can encode a modified VH domain as described in Example 20.

In some cases, a non-mouse VH locus present in the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can include one or more regulatory elements for each VH gene segment in the non-mouse VH locus (e.g., to control expression and/or recombination for each VH gene segment). The regulatory element(s) can be endogenous or exogenous. Examples of regulatory elements that can be used to control expression and/or recombination for each VH gene segment include, without limitation, promoters, enhancers, transcription factor binding sites, splice sites, recombination signal sequences, leader exon 1, leader exon 2, signal peptide sequences, and introns.

In some cases, a non-mouse VH locus present in the genome of a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can include one or more additional components. The additional component(s) can be endogenous or exogenous. For example, a non-mouse VH locus present in the genome of a non-human animal provided herein can include nucleic acid (e.g., an endogenous exon) encoding a leader sequence (e.g., such that antibodies such as sdAbs and/or heavy chain only antibodies produced by the non-human animal include the encoded leader sequence). In some cases, a non-mouse VH locus present in the genome of a non-human animal provided herein can include nucleic acid (e.g., an endogenous exon) encoding a leader sequence for each VH gene segment in the non-mouse VH locus. Examples of leader sequences that can be included in a non-mouse VH locus present in the genome of a non-human animal provided herein include, without limitation, an L1 exon of the non-human animal (e.g., a mouse L1 exon), and L2 exon of the non-human animal (e.g., a mouse L2 exon), and peptide signal sequences.

Any appropriate method can be used to generate a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions). In some cases, nucleic acid comprising one or more human VH gene segments and/or VH gene segments encoding modified human VH domains having a FR2 containing one, two, three, four, or more amino acid substitutions as described herein can be inserted into the genome of a non-human animal (e.g., a mouse) that lacks nucleic acid encoding at least a portion of an endogenous CH1 (or lacks at least a portion of an endogenous regulatory element that drives expression of an endogenous CH1 domain) and optionally lacks at least a portion of the endogenous nucleic acid encoding one or more (e.g., all) endogenous VH domains. For example, a VH locus (e.g., nucleic acid comprising one or more human VH gene segments and/or VH gene segments encoding modified human VH domains having a FR2 containing one, two, three, four, or more amino acid substitutions as described herein) can be inserted into the genome of an engineered mouse is set forth in FIGS. 4, 5D, and 6B (also referred to as a Singularity HyperDock non-human animal or a Singularity HyperDock mouse). In some cases, nucleic acid comprising one or more human VH gene segments and/or VH gene segments encoding modified human VH domains having a FR2 containing one, two, three, four, or more amino acid substitutions as described herein can include at least one recombination site (e.g., at least one nucleic acid sequence that can be recognized by a recombinase) on each end (e.g., at the 5′ end and at the 3′ end) of the one or more one or more human VH gene segments and/or VH gene segments encoding modified human VH domains having a FR2 containing one, two, three, four, or more amino acid substitutions as described herein. In some cases, the non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of the endogenous nucleic acid encoding one or more (e.g., all) VH domains (e.g., a Singularity HyperDock mouse) can include at least one recombination site, such that a recombinase can facilitate the insertion of nucleic acid comprising one or more human VH gene segments and/or VH gene segments encoding modified human VH domains having a FR2 containing one, two, three, four, or more amino acid substitutions as described herein into the genome of that non-human animal.

In some cases, nucleic acid comprising one or more human VH gene segments and/or VH gene segments encoding modified human VH domains having a FR2 containing one, two, three, four, or more amino acid substitutions as described herein can be present on a vector. Examples of vectors that can include nucleic acid comprising one or more human VH gene segments and/or VH gene segments encoding modified human VH domains having a FR2 containing one, two, three, four, or more amino acid substitutions as described herein include, without limitation, yeast artificial chromosomes (YACs), bacterial artificial chromosomes (BACs), human artificial chromosomes (HACs), transchromosomes (e.g., whole transchromosomes and fragmented transchromosomes), P1-derived artificial chromosome (PACs), plasmids, and phagemids.

A non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of endogenous nucleic acid encoding one or more VH domains can include any appropriate recombination site(s). In some cases, a recombinase site can be an exogenous recombinase site. Examples of recombination sites that can be present in the genome of a non-human animal (e.g., a mouse) that lacks nucleic acid encoding at least a portion of an endogenous CH1 (or lacks at least a portion of an endogenous regulatory element that drives expression of an endogenous CH1 domain) and lacks at least a portion of endogenous nucleic acid encoding one or more VH domains include, without limitation, frt, loxP, M2, M3, lox2271, lox2372, loxFAS, loxN, lox5171, lox2272, attB, and attP. Additional examples of recombination sites that can be present in the genome of a non-human animal (e.g., a mouse) provided herein can be as shown in Table 1.

A genome of a non-human animal (e.g., a mouse) described herein can include any appropriate number of recombination sites. For example, a non-human animal (e.g., a mouse) described herein can include one, two, three, four, five, six, seven, eight, nine, ten, or more recombination sites. When a non-human animal (e.g., a mouse) described herein includes two or more recombination sites in its genome, the recombination sites can each be a different recombination site. For example, a non-human animal (e.g., a mouse) described herein can include at least three different recombination sites within its genome. For example, a non-human animal (e.g., a mouse) described herein can include at least five different recombination sites within its genome.

A recombination site in a non-human animal (e.g., a mouse) described herein can be at any appropriate location within the genome of the non-human animal. In some cases, one or more recombination sites within a genome of a non-human animal described herein can be upstream of endogenous nucleic acid encoding a CH2 domain or a CH3 domain. In some cases, one or more recombination sites within a genome of a non-human animal described herein can be upstream (e.g., less than 2.5 Mb upstream) of an endogenous Eμ element. When a genome of a non-human animal (e.g., a mouse) described herein includes different recombination sites (e.g., different exogenous recombination sites), each of the recombination sites can be located less than 2.0 Mb, less than 1.5 Mb, less than 1.0 Mb, less than 500 kb, less than 250 kb, less than 200 kb, less than 100 kb, less than 50 kb, less than 25 kb, or less than 10 kb upstream of an endogenous Eμ element.

Any appropriate method can be used to make a non-human animal described herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions). For example, gene editing techniques (e.g., CRISPR/Cas gene editing, TALEN gene editing, and/or zinc finger-based gene editing), recombination techniques (e.g., sequential Recombinase Mediated Cassette Exchange (RMCE)), and combinations thereof can be used to make a non-human animal described herein. In some cases, a nucleic acid molecule (e.g., an exogenous nucleic acid molecule) comprising one or more human VH gene segments and/or VH gene segments encoding modified human VH domains having a FR2 containing one, two, three, four, or more amino acid substitutions as described herein can be introduced into a stem cell of a non-human animal (e.g., a mouse), the stem cell can be implanted into a blastocyst of the same species of non-human animal, and the blastocyst can be implanted into a pseudo-pregnant female of the same species of non-human animal to obtain a chimeric non-human animal, crossing the chimeric non-human animal to a wild-type non-human animal to produce offspring, screening the offspring for heterozygosity, and identifying a founder non-human animal carrying the one or more VH gene segments within its genome. In some cases, a non-human animal provided herein can be made as described in the Examples.

This document also provides methods for producing populations of antibodies (e.g., sdAbs and/or heavy chain only antibodies) having improved solubility (e.g., as compared to an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 that lacks the amino acid modifications described herein). For example, a non-human animal described herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can be administered one or more antigens (e.g., a composition including one or more antigens), such that one or more B cells in the non-human animal produce an antibody (e.g., a heavy chain only antibody) that includes a chimeric heavy chain that includes (a) a non-human CH2 domain encoded by a nucleic acid endogenous to the non-human animal and/or a non-human CH3 domain encoded by a nucleic acid endogenous to the non-human animal and (b) a variable region that includes a human VH domain having a FR2 containing one, two, three, four, or more amino acid substitutions that is encoded by a nucleic acid exogenous to the non-human animal. In some cases, one or more B cells can be isolated from the non-human animal.

Antibodies against any appropriate antigen can be produced by a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions). In some cases, an antigen can be an endogenous antigen or self-antigen. In some cases, an antigen can be an exogenous antigen. An antigen can be any appropriate type of molecule (e.g., a peptide, a lipid, or a nucleic acid). Examples of antigens that can be used to immunize a non-human animal provided herein can include those listed in Table 2.

In some cases, immunizing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) with one or more antigen described herein can activate at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 non-human B cell clones in the non-human animal.

In some cases, immunizing a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) with one or more antigens described herein can lead to production of polyclonal antiserum including antibodies (e.g., heavy chain only antibodies) having one or two chimeric heavy chains (a) that include (i) a non-human CH2 domain and/or a non-human CH3 domain and (ii) a variable region that includes a human VH domain having a FR2 containing one, two, three, four, or more amino acid modifications (e.g., amino acid substitutions) and (b) that can bind (e.g., specifically bind) at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 antigens of the administered immunogenic composition.

This document also provides antibodies (e.g., sdAbs and/or heavy chain only antibodies) produced by immunization of a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions). Antibodies (e.g., sdAbs and/or heavy chain only antibodies) produced by a non-human animal provided herein can be obtained using any appropriate method. For example, heavy chain only antibodies can be obtained from the blood of a non-human animal provided herein. In some cases, a non-human animal provided herein can be immunized with a particular antigen (e.g., a SARS-COV-2 antigen) such that the non-human animal produces antibodies (e.g., sdAbs and/or heavy chain only antibodies) against that antigen, and the antibodies produced can be assessed for the desired properties (e.g., binding properties, neutralization properties, and/or solubility properties). In some cases, blood can be collected from a non-human animal provided herein that has been immunized as described herein with a particular antigen multiple times (e.g., after each of multiple immunizations, multiple times after a single immunization, multiple times in between immunizations, or any combination thereof). Blood can be collected from a non-human animal provided herein that has been immunized as described herein any suitable amount of time following an immunization. For example, blood can be collected from a non-human animal provided herein that has been immunized at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more, after an immunization. In some cases, blood can be collected at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most at most 35, at most 42, at most 49, or at most 56 days after an immunization. In some cases, blood can be collected about 1, 2, 3, 4, 5, 6, 7, 8, 9, 10, 11, 12, 14, 16, 17, 18, 20, 21, 22, 23, 24, 25, 26, 27, 28, 29, 30, 31, 32, 33, 34, 35, 36, 37, 38, 39, 40, 41, or 42 days, or more after an immunization.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can retain its endogenous non-human immune cells. For example, a non-human animal provided herein can retain its non-human T cells, B cells, and/or antigen-presenting cells.

In some cases, a non-human animal provided herein (e.g., a genetically engineered non-human animal such as a mouse that can, when exposed to one or more antigens, produce an antibody such as a sdAb or heavy chain only antibody having one or two chimeric heavy chains that include (a) a non-human CH2 domain and/or a non-human CH3 domain (and optionally a non-human Ig hinge) and (b) a variable region that includes a human VH domain having a FR2 containing one, two, three, four, or more amino acid modifications such as one, two, three, four, or more amino acid substitutions) can include any feature or any combination of features (or any methods of making can be performed) as disclosed in U.S. Patent Application Publication No. 2017/0233459, which is hereby incorporated by reference in its entirety. For example, a non-human animal provided herein can include any feature or any combination of features (or any methods of making can be performed) as disclosed in Kuroiwa et al., Nat. Biotechnol., 27 (2): 173-81 (2009); Matsushita et al., PLos ONE, 9 (3):e90383 (2014); Hooper et al., Sci. Transl. Med., 6 (264): 264ra162 (2014); Matsushit et al., PLOS ONE, 10 (6):e0130699 (2015); Luke et al., Sci. Transl. Med., 8 (326): 326ra21 (2016); Dye et al., Sci. Rep., 6:24897 (2016); Gardner et al., J. Virol., 91 (14) (2017); Stein et al., Antiviral Res., 146:164-173 (2017); Silver, Clin. Infect. Dis., 66 (7): 1116-1119 (2018); Beigel et al., Lancet Infect. Dis., 18 (4): 410-418 (2018); Luke et al., J. Inf. Dis., 218 (suppl_5):S636-S648 (2018), each of which is hereby incorporated by reference in its entirety.

In some cases, an amino acid sequence described herein can include one or more additional amino acid modifications that are in addition to the modification(s) in a FR2 as described herein. Such amino acid modifications can include, without limitation, amino acid substitutions, amino acid deletions, amino acid additions, and combinations thereof. In some cases, an amino acid modification, including those that change an amino acid in a FR2 as described herein, can be made to improve the binding and/or contact with an antigen and/or to improve a functional activity of an antibody (e.g., a sdAb and/or a heavy chain only antibody) provided herein. In some cases, an amino acid substitution, including those that change an amino acid in a FR2 as described herein, can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution, including those that change an amino acid in a FR2 as described herein, can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding an antibody or a fragment thereof. See, for example, Zoller, Curr. Opin. Biotechnol. 3:348-354 (1992). Both naturally occurring and non-naturally occurring amino acids (e.g., artificially-derivatized amino acids) can be used to generate an amino acid sequence variant from an amino acid sequence described herein.

Methods and Materials for Obtaining Variants of Antibodies that Underwent Affinity Maturation In Vivo

In another aspect, this document provides methods and materials involved in in vivo affinity maturation of antibodies of interest. For example, this document provides chimeric non-human animals (e.g., mice) generated from an embryo having (a) a first cell having one or more genomic modifications that prevent the first cell (and cells derived from the first cell) from producing immunoglobulins and (b) a second cell having an IgH locus that includes an exogenous nucleic acid sequence encoding a heavy chain variable region of an antibody of interest such that the chimeric non-human animal produces heavy chain antibodies containing the heavy chain variable region of the antibody of interest in addition to one or more variants of those heavy chain antibodies that underwent in vivo affinity maturation.

A chimeric non-human animal described herein can be any type of non-human animal. For example, a chimeric non-human animal described herein can be a mouse, rat, rabbit, guinea pig, pig, sheep, non-human primate (e.g., a monkey), or a bovine species.

As described herein, a chimeric non-human animal (e.g., mouse) can be generated from an embryo having at least two genetically different cells. The first cell can be that of a recipient embryo or blastocyst and can have one or more genomic modifications that prevent the first cell (and cells derived from the first cell) from producing immunoglobulins. Examples of genomic modifications that can prevent cells from producing immunoglobulins include, without limitation, Prkdc^{scid}, Rag1 inactivation, Rag1 knock outs, Rag1^{ti1Mom}, Rag2 inactivation, Rag2 knock outs, Rag2^{tm1}, IgH inactivation, IgH knock outs, and IgH VH/DH/JH knock outs.

In some cases, a recipient embryo or blastocyst can be obtained from non-human animal (e.g., a mouse) engineered to not produce immunoglobulins. For example, a recipient embryo or blastocyst can be obtained from a SCID mouse, a Rag1 knock out mouse, a Rag2 knock out mouse, or an IgH knock out mouse and used to create a chimeric mouse described herein. In such cases, each cell of the obtained recipient embryo or blastocyst can be a cell having the features of the first cell described herein.

The second cell can be a stem cell (e.g., an embryonic stem (ES) cell) having an IgH locus that includes an exogenous nucleic acid sequence encoding an input nanobody domain. Any appropriate antibody of interest can be a source of the input nanobody domain that is encoded by the exogenous nucleic acid sequence located at an IgH locus of the second cell (e.g., an engineered ES cell).

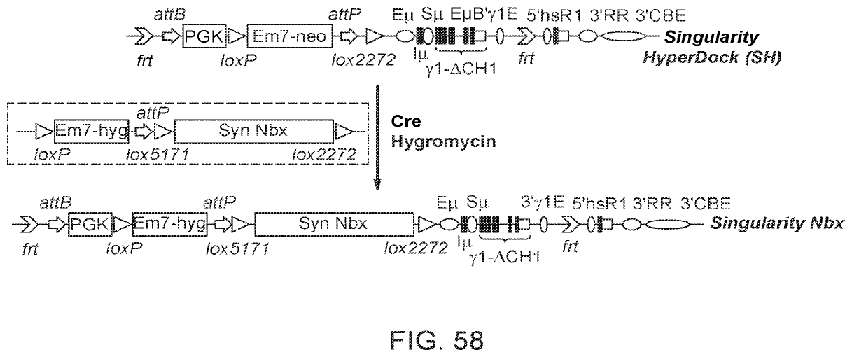

In some cases, the IgH locus of the second cell can be designed to (i) include endogenous hinge, constant CH2, and/or CH3 gene segments of an Ig class (e.g., IgG) and (ii) exclude an endogenous constant CH1 gene segment of that Ig class and/or a regulatory element controlling expression of that endogenous constant CH1 gene segment of that Ig class. For example, mouse stem cells (e.g., mouse ES cells) obtained from a Singularity HyperDock mouse (see, e.g., FIG. 4) can be genetically modified by inserting exogenous nucleic acid encoding an input nanobody domain within the IgH locus upstream of the endogenous γ1-ΔCH1-encoding sequences. Such genetically modified stem cells (e.g., ES cells) can then be used as the second cell to produce a chimeric mouse described herein. When the second cell is designed to (i) include endogenous hinge, constant CH2, and/or CH3 gene segments of an Ig class (e.g., IgG) and (ii) exclude an endogenous constant CH1 gene segment of that Ig class and/or a regulatory element controlling expression of that endogenous constant CH1 gene segment of that Ig class, then the input nanobody domain can be expressed by B cells derived from the second cell as the variable region of a heavy chain only antibody (e.g., a Nbx-IgΔCH1 such as a Nbx-IgG1-ΔCH1). See, e.g., FIG. 58.

In some cases, the IgH locus of the second cell can be designed to exclude endogenous VH, DH, and/or JH gene segments. For example, mouse stem cells (e.g., mouse ES cells) obtained from a Singularity HyperDock mouse (see, e.g., FIG. 4), which lacks endogenous mouse VH, DH, and JH gene segments, can be genetically modified by inserting exogenous nucleic acid encoding an input nanobody domain within the IgH locus upstream of the endogenous γ1-ΔCH1-encoding sequences. Such genetically modified stem cells (e.g., ES cells) can then be used as the second cell to produce a chimeric mouse described herein. When the second cell is designed to exclude endogenous VH, DH, and/or JH gene segments, then the resulting chimeric non-human animal (e.g., mouse) can express the input nanobody domain while expressing little or no endogenous immunoglobulins (e.g., endogenous mouse immunoglobulins). See, e.g., FIG. 58.

In some cases, the second cell used to generate a chimeric non-human animal provided herein (e.g., a mouse ES cell) can be a cell whose genome has (e.g., is genetically engineered to have) one or more disruptions and/or deletions in the endogenous nucleic acid sequence of the Ig VH locus. In some cases, both alleles of the genome of the second cell used to generate a chimeric non-human animal provided herein (e.g., a mouse ES cell) can lack all the exons of the endogenous Ig heavy chain variable region. In some cases, neither allele of the genome of the second cell used to generate a chimeric non-human animal provided herein (e.g., a mouse ES cell) contains an endogenous exon encoding a VH domain.

In some cases, a chimeric non-human animal (e.g., mouse) described herein can have the ability to express heavy chain only antibodies that include the input nanobody domain, while lacking the ability to express immunoglobulins endogenous to that non-human animal. For example, a chimeric mouse described herein can have the ability to express heavy chain only antibodies that include the input nanobody domain (e.g., Nbx-IgG1ΔCH1 heavy chain only antibodies), while lacking the ability to express endogenous mouse immunoglobulins.

In some cases, the second cell used to generate a chimeric non-human animal provided herein (e.g., a mouse ES cell) can include one or more regulatory elements for the exogenous nucleic acid sequence encoding the input nanobody domain. The regulatory element(s) can be endogenous or exogenous. Examples of regulatory elements that can be used to control expression and/or recombination for the exogenous nucleic acid sequence encoding the input nanobody domain include, without limitation, promoters, enhancers, transcription factor binding sites, splice sites, recombination signal sequences, leader exon 1, leader exon 2, signal peptide sequences, and introns.

In some cases, the second cell used to generate a chimeric non-human animal provided herein (e.g., a mouse ES cell) can include one or more additional components. The additional component(s) can be endogenous or exogenous. For example, the exogenous nucleic acid sequence encoding the input nanobody domain of the second cell can include nucleic acid (e.g., an endogenous exon or an exogenous exon) encoding a leader sequence. Examples of leader sequences that can be included for the exogenous nucleic acid sequence encoding the input nanobody domain of the second cell include, without limitation, an L1 exon of the non-human animal (e.g., a mouse L1 exon), an L2 exon of the non-human animal (e.g., a mouse L2 exon), and peptide signal sequences.

In some cases, the second cell used to generate a chimeric non-human animal provided herein (e.g., a mouse ES cell) can be designed to produce antibodies that include the input nanobody domain and that lack CH1 domains and that lack Ig light chains. For example, the second cell used to generate a chimeric non-human animal provided herein (e.g., a mouse ES cell) can be a cell whose genome has (e.g., is genetically engineered to have) one or more disruptions in an endogenous nucleic acid sequence encoding an CH1 domain of an IgG1 C-region gene (e.g., C_{γ1}), such that an IgG antibody encoded by the chimeric non-human animal contains the input nanobody domain and lacks a CH1 domain and lacks Ig light chains. In some cases, the genome of the second cell used to generate a chimeric non-human animal provided herein (e.g., a mouse ES cell) can lack nucleic acid encoding at least a portion of an endogenous CH1 domain. In some cases, the genome of the second cell used to generate a chimeric non-human animal provided herein (e.g., a mouse ES cell) can lack at least a portion of an endogenous regulatory element that drives expression of an endogenous CH1 domain.

In some cases, the genome of the second cell used to generate a chimeric non-human animal provided herein (e.g., a mouse ES cell) can be designed to have a deletion of endogenous nucleic acid encoding a CH1 domain of a constant region (e.g., of an IgG C-region such as a CH1 domain of an IgG1 C-region, a CH1 domain of an IgG2a C-region, a CH1 domain of an IgG2b C-region, and/or a CH1 domain of an IgG3 C-region). The CH1 domain can contain multiple exons. In some cases, endogenous exon 1 of a CH1 domain of a constant region such as an IgG C-region can be deleted such that a chimeric non-human animal described herein produces IgMΔCH1, IgGΔCH1, IgDΔCH1, IgAΔCH1, and/or IgEΔCH1 heavy chain only antibodies containing the input nanobody domain.

When making one or more genetic modifications to delete all or part of the nucleic acid encoding a CH1 domain (e.g., a CH1 domain of an IgG1 C-region, a CH1 domain of an IgG2a C-region, a CH1 domain of an IgG2b C-region, and/or a CH1 domain of an IgG3 C-region) in a cell to obtain the second cell used to generate a chimeric non-human animal provided herein (e.g., a mouse ES cell), the endogenous nucleic acid encoding a hinge domain, a heavy chain CH2 domain, and a heavy chain CH3 domain can remain intact. For example, to make a mouse that produces IgG1ΔCH1 heavy chain only antibodies, the genome of that mouse can lack exon 1 (and/or additional portions) of the CH1 domain of IgG1 while retaining the endogenous mouse nucleic acid needed to express the hinge domain, a heavy chain CH2 domain, and a heavy chain CH3 domain of IgG1, thereby resulting in a mouse that is capable of producing IgG1ΔCH1 heavy chain only antibodies.

Additional endogenous nucleic acid components that can be absent from the genome of the second cell used to generate a chimeric non-human animal provided herein (e.g., a mouse ES cell) include, without limitation, the introns and/or exons of the IgM constant domains (e.g., the μ constant domain locus), the introns and/or exons of the IgD constant domains (e.g., the δ constant domain locus), the introns and/or exons of the IgE constant domains (e.g., the ε constant domain locus), and/or the introns and/or exons of the IgA constant domains (e.g., the α constant domain locus). For example, the genome of the second cell used to generate a chimeric non-human animal provided herein (e.g., a mouse ES cell) can be designed to lack the introns and exons of the IgM constant domains (e.g., the μ constant domain locus), the introns and exons of the IgD constant domains (e.g., the δ constant domain locus), the introns and exons of the IgE constant domains (e.g., the ε constant domain locus), and the introns and exons of the IgA constant domains (e.g., the α constant domain locus).

In some cases, when designing a chimeric non-human animal provided herein to produce only IgG1ΔCH1 heavy chain only antibodies that include the input nanobody domain, the genome of the second cell used to generate a chimeric non-human animal provided herein (e.g., a mouse ES cell) can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus). An example of a genetic engineering approach to create a mouse that produces only IgG1ΔCH1 heavy chain only antibodies and that can be used as a starting point for obtaining the second cell used to generate a chimeric non-human animal provided herein (e.g., a mouse ES cell) is set forth in FIGS. 3A-3E.

In some cases, when designing a chimeric non-human animal provided herein to produce only IgG2aΔCH1 heavy chain only antibodies that include the input nanobody domain, the genome of the second cell used to generate a chimeric non-human animal provided herein (e.g., a mouse ES cell) can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ1 constant domains (e.g., the γ1 constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

In some cases, when designing a chimeric non-human animal provided herein to produce only IgG2bΔCH1 heavy chain antibodies that include the input nanobody domain, the genome of the second cell used to generate a chimeric non-human animal provided herein (e.g., a mouse ES cell) can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ1 constant domains (e.g., the γ1 constant domain locus), and the endogenous (if endogenously present) introns and/or exons of the Igγ2c constant domains (e.g., the γ2c constant domain locus).

In some cases, when designing a chimeric non-human animal provided herein to produce only IgG2cΔCH1 heavy chain antibodies that include the input nanobody domain, the genome of the second cell used to generate a chimeric non-human animal provided herein (e.g., a mouse ES cell) can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ1 constant domains (e.g., the γ1 constant domain locus).

In some cases, when designing a chimeric non-human animal provided herein to produce only IgG3ΔCH1 heavy chain antibodies that include the input nanobody domain, the genome of the second cell used to generate a chimeric non-human animal provided herein (e.g., a mouse ES cell) can be designed to lack (in addition to lacking endogenous introns and/or exons of the μ constant domain locus, endogenous introns and/or exons of the δ constant domain locus, endogenous introns and/or exons of the ε constant domain locus, and endogenous introns and/or exons the of α constant domain locus) the endogenous (if endogenously present) introns and/or exons of the Igγ1 constant domains (e.g., the γ1 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

As described herein, retaining and/or creating new positioning for certain endogenous enhancer or regulatory elements within the genome of the second cell used to generate a chimeric non-human animal provided herein (e.g., a mouse ES cell) can result in a chimeric non-human animal that produces effectively large collections of and/or amounts of heavy chain only antibodies having the input nanobody domain and variants thereof that underwent in vivo affinity maturation. For example, the genome of the second cell used to generate a chimeric non-human animal provided herein (e.g., a mouse ES cell) can be designed to retain the μ enhancer (Eμ), the μ switch region (Sμ), and/or the μ promoter containing I-exon (Iμ) that are endogenously found upstream of the nucleic acid encoding the IgM constant domains. In some cases, the genome of the second cell used to generate a chimeric non-human animal provided herein (e.g., a mouse ES cell) can be designed such that the retained endogenous Eμ, Sμ, and/or Iμ elements are in a genomic position such that the first nucleic acid sequence downstream of the retained Eμ, Sμ, and/or Iμ elements that encodes a full-length endogenous Ig constant domain is one that encodes a CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 1B and FIG. 3C where the nucleic acids of the endogenous mouse Eμ, Sμ, and Iμ elements are repositioned to be upstream of the nucleic acid encoding the endogenous IgG1 CH2 domain.

In another example, the genome of the second cell used to generate a chimeric non-human animal provided herein (e.g., a mouse ES cell) can be designed to retain the 3′RR and/or 3′CBE elements that are endogenously found downstream of the nucleic acid encoding the IgA constant domains. In some cases, the genome of the second cell used to generate a chimeric non-human animal provided herein (e.g., a mouse ES cell) can be designed such that the retained endogenous 3′RR and/or 3′CBE elements are in a genomic position such that the first nucleic acid sequence upstream of the retained 3′RR and/or 3′CBE elements that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 2B and FIG. 3E where the nucleic acid of the endogenous mouse 3′RR element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 3′RR element.

In some cases, the genome of the second cell used to generate a chimeric non-human animal provided herein (e.g., a mouse ES cell) can be designed to retain the 3′γ1E element that is endogenously found between, for example, the IgG1 and IgG2b loci. In some cases, the genome of the second cell used to generate a chimeric non-human animal provided herein (e.g., a mouse ES cell) can be designed such that the retained endogenous 3′γ1E element is in a genomic position such that nucleic acid encoding two, one, or no full-length endogenous Ig CH2 domains is located between the retained endogenous 3′γ1F element and a retained endogenous 3′RR element and/or a retained endogenous 3′CBE element. An example of this genomic configuration is set forth in FIG. 3E where the nucleic acid of the endogenous mouse 3′γ1E element is repositioned to be upstream of a retained endogenous 3′RR element such that no other nucleic acid encoding a full length IgG CH2 domain is located between the endogenous mouse 3′γ1E element and the endogenous 3′RR element.

In some cases, the genome of the second cell used to generate a chimeric non-human animal provided herein (e.g., a mouse ES cell) can be designed to retain the 5′hsR1 element that is endogenously found within the IgA constant domain locus. In some cases, the genome of the second cell used to generate a chimeric non-human animal provided herein (e.g., a mouse ES cell) can be designed such that the retained endogenous 5′hsR1 element is in a genomic position such that the first nucleic acid sequence upstream of the retained 5′hsR1 element that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 3E where the nucleic acid of the endogenous mouse 5′hsR1 element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 5′hsR1 element.

In some cases, instead of retaining an endogenous enhancer or regulatory element as described herein, one or more exogenous enhancer or regulatory elements can be engineered to be in the genome of the second cell used to generate a chimeric non-human animal provided herein (e.g., a mouse ES cell). For example, in some cases, the genome of the second cell used to generate a chimeric non-human animal provided herein (e.g., a mouse ES cell) can be designed as described herein where the endogenous mouse Eμ element is removed and replaced with a human Eμ element.

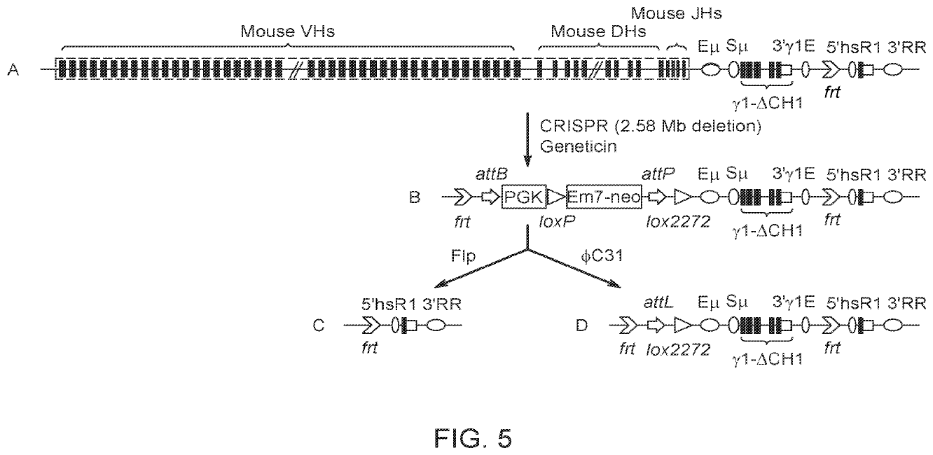

Any appropriate method can be used to obtain a cell that can be used as the second cell for generating a chimeric non-human animal provided herein (e.g., a mouse ES cell). For example, an exogenous nucleic acid sequence encoding an input nanobody domain can be inserted into the genome of a stem cell (e.g., an ES cell) obtained from a non-human animal (e.g., a mouse) having the IgH locus as set forth in FIG. 4 or FIG. 5D (also referred to as a Singularity HyperDock mouse). In some cases, an exogenous nucleic acid sequence encoding an input nanobody domain can include at least one recombination site (e.g., at least one nucleic acid sequence that can be recognized by a recombinase) on each end (e.g., at the 5′ end and at the 3′ end) of the an exogenous nucleic acid sequence encoding an input nanobody domain. In some cases, the genome of a cell (e.g., an ES cell) of a Singularity HyperDock mouse can include at least one recombination site within the IgH locus, such that a recombinase can facilitate the insertion of an exogenous nucleic acid sequence encoding an input nanobody domain into the genome of that cell.

In some cases, an exogenous nucleic acid sequence encoding an input nanobody domain can be present on a vector. Examples of vectors that can include an exogenous nucleic acid sequence encoding an input nanobody domain include, without limitation, yeast artificial chromosomes (YACs), bacterial artificial chromosomes (BACs), human artificial chromosomes (HACs), transchromosomes (e.g., whole transchromosomes and fragmented transchromosomes), P1-derived artificial chromosome (PACs), plasmids, and phagemids.

The genome of the second cell used to generate a chimeric non-human animal provided herein (e.g., a mouse ES cell) can include any appropriate recombination site(s). In some cases, a recombinase site can be an exogenous recombinase site. Examples of recombination sites that can be present in the genome of the second cell used to generate a chimeric non-human animal provided herein (e.g., a mouse ES cell) include, without limitation, frt, loxP, M2, M3, lox2372, loxFAS, loxN, lox5171, lox2272, attB, and attP. Additional examples of recombination sites that can be present in the genome of the second cell used to generate a chimeric non-human animal provided herein (e.g., a mouse ES cell) can be as shown in Table 1.

The genome of the second cell used to generate a chimeric non-human animal provided herein (e.g., a mouse ES cell) can include any appropriate number of recombination sites. For example, the genome of the second cell used to generate a chimeric non-human animal provided herein (e.g., a mouse ES cell) can include one, two, three, four, five, six, seven, eight, nine, ten, or more recombination sites. When the genome of the second cell used to generate a chimeric non-human animal provided herein (e.g., a mouse ES cell) includes two or more recombination sites in its genome, the recombination sites can each be a different recombination site. For example, the genome of the second cell used to generate a chimeric non-human animal provided herein (e.g., a mouse ES cell) can include at least three different recombination sites within its genome. For example, the genome of the second cell used to generate a chimeric non-human animal provided herein (e.g., a mouse ES cell) can include at least five different recombination sites within its genome.

A recombination site in the genome of the second cell used to generate a chimeric non-human animal provided herein (e.g., a mouse ES cell) can be at any appropriate location within the genome of the cell. In some cases, one or more recombination sites within the genome of the second cell used to generate a chimeric non-human animal provided herein (e.g., a mouse ES cell) can be upstream of endogenous nucleic acid encoding a hinge, a CH2 domain, or a CH3 domain. In some cases, one or more recombination sites within the genome of the second cell used to generate a chimeric non-human animal provided herein (e.g., a mouse ES cell) can be upstream (e.g., less than 2.5 Mb upstream) of an endogenous Eu. When the genome of the second cell used to generate a chimeric non-human animal provided herein (e.g., a mouse ES cell) includes different recombination sites (e.g., different exogenous recombination sites), each of the recombination sites can be located less than 2.0 Mb, less than 1.5 Mb, less than 1.0 Mb, less than 500 kb, less than 250 kb, less than 200 kb, less than 100 kb, less than 50 kb, less than 25 kb, or less than 10 kb upstream of an endogenous Eμ.

When making a chimeric non-human animal (e.g., a chimeric mouse) described herein, the first cell and the second cell can be from the same non-human animal species. For example, when using a recipient embryo or blastocyst obtained from a Rag2 knock out mouse as the source of the first cell, then the second cell can be a mouse ES cell. Typically, to make a chimeric non-human animal (e.g., a chimeric mouse) described herein, a population of second cells (e.g., a population of mouse ES cells) is injected into a recipient embryo or blastocyst that includes the first cells, and the resulting embryo or blastocyst is implanted into the uterus of a surrogate female (e.g., the uterus of a pseudopregnant female mouse). Typically, when making chimeric mice, the recipient embryos or blastocysts are chosen from a strain of mouse that has a different coat color than that of the ES cell strain. This can allow for easy identification of chimeric mice based on coat color.

This document also provides methods and materials for obtaining variants of heavy chain only antibodies that underwent in vivo affinity maturation within a chimeric non-human animal (e.g., mouse) as described herein. For example, a composition containing an antigen recognized by an antibody of interest can be administered to a chimeric non-human animal (e.g., mouse) as described herein that is designed to express heavy chain only antibodies that include an input nanobody domain that targets that antigen. Administration of that antigen can cause antigenic stimulation of B cells within the chimeric non-human animal, thereby promoting in vivo affinity maturation of the encoded heavy chain only antibodies that include an input nanobody domain. Such in vivo affinity maturation can lead to the generation of variants of the input nanobody domain. The in vivo generated variants and/or the B cells producing such variants can be obtained from the chimeric non-human animal, thereby providing enhanced versions of the antibody of interest. In some cases, an antibody (e.g., a nanobody, a heavy chain only antibody, or full Ig antibody) including the variable region of an in vivo affinity matured variant can exhibit enhanced binding and/or enhance performance characteristics as compared to the original input nanobody domain.

In some cases, administering a composition containing an antigen recognized by the input nanobody can activate at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 non-human B cell clones within the chimeric non-human animal.

Any appropriate method can be used to obtain an in vivo generated variant and/or the B cell producing such a variant from a chimeric non-human animal described herein. For example, heavy chain only antibodies can be obtained from the blood of a chimeric non-human animal provided herein. In some cases, blood can be collected from a chimeric non-human animal provided herein after each of multiple administrations of a composition containing an antigen recognized by the input nanobody, multiple times after a single such administration, multiple times in between such administrations, or any combination thereof. Blood can be collected from a chimeric non-human animal provided herein at any suitable amount of time following administration of a composition containing an antigen recognized by the input nanobody. For example, blood can be collected from a chimeric non-human animal provided herein at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more after administration of a composition containing an antigen recognized by the input nanobody. In some cases, blood can be collected at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most 35, at most 42, at most 49, or at most 56 days after administration of a composition containing an antigen recognized by the input nanobody. In some cases, blood can be collected about 1, 2, 3, 4, 5, 6, 7, 8, 9, 10, 11, 12, 14, 16, 17, 18, 20, 21, 22, 23, 24, 25, 26, 27, 28, 29, 30, 31, 32, 33, 34, 35, 36, 37, 38, 39, 40, 41, or 42 days, or more after administration of a composition containing an antigen recognized by the input nanobody.

The invention will be further described in the following examples, which do not limit the scope of the invention described in the claims.

### EXAMPLES

### Example 1: Genetic Engineering of Singularity Mice

### Genetic Engineering Procedures

The LVGN-YF ES cell line (40, XY) was established using blastocysts isolated from crosses of 129S6 and C57BL/6N mice and was used for all genetic engineering. This F1 hybrid ES cell line exhibited robust germline competence after multiple rounds of genetic modifications, and the use of a F1 hybrid ES cell line also allowed for use of strain-specific SNPs to identify sequential genetic modifications that occurred on the same chromosome. ES cells were cultured in Knockout DMEM supplemented with 20% ES cell-qualified fetal bovine serum (FBS), 0.1 mM MEM non-essential amino acids, 0.1 mM 2-mercaptoethanol, 1 mM sodium pyruvate, 2 mM GlutaMAX-I supplement, 100 units/mL penicillin-streptomycin, 25 nM MEK inhibitor PD98059 (Sigma), 3 nM GSK-3 inhibitor CHIR99021 (Sigma), and 1,000 units/mL mouse leukemia inhibitory factor (LIF, Sigma) on feeder cells. The hygroR/neoR/puroR triple-resistant feeder cell line LVGN-SHNPL was engineered from the SNL76/7 feeder cell line and was maintained in Knockout DMEM supplemented with 10% ES cell-qualified FBS, 0.1 mM MEM non-essential amino acids, 0.1 mM 2-mercaptoethanol, 1 mM sodium pyruvate, 2 mM GlutaMAX-I supplement, and 100 units/mL penicillin-streptomycin. Feeder cells were prepared by treating the proliferating LVGN-SHNPL cells for 3 hours with 10 mg/mL Mitomycin C (Sigma).

All transfections were done either with lipofection using Lipofectamine LTX (ThermoFisher), or with electroporation using a Bio-Rad Gene Pulser II apparatus. For lipofection, 0.1-1×10⁶ dissociated ES cells were mixed with 0.5-2.5 μg plasmid DNA-lipofectamine complex in Opti-MEM following instructions provided by the manufacturer and cultured overnight in growth medium, and antibiotic selection was applied 24 hours later as needed. For electroporation, 0.5-1.5×10⁷ ES cells were mixed with 10-30 μg plasmid and/or BAC DNA in PBS and electroporated at 250V/500 μF in a 4-mm gap cuvette and cultured overnight in growth medium, and antibiotic selection was applied 24 hours later as needed. The antibiotic concentrations used for selection were 250 μg/mL for Geneticin (G418), 200 μg/mL for Hygromycin B, and 5 μg/mL for Puromycin. For CRISPR-mediated gene editing, Cas9 and sgRNAs were delivered as separate plasmids, which were co-transfected with PGK-puro or PGK-hygro genes and selected for 2 days with the corresponding antibiotics to enrich for transfectants, or were co-transfected with the HDR donor template and selected for 10-14 days with the corresponding antibiotics to derive stably transfected clones. For removing selection marker cassettes flanked with recombination sites (lox-lox, frt-frt, or attB-attP), ES cells were transiently transfected with plasmids expressing the corresponding recombinase or integrase (Cre, Flp, or φC31, respectively) and plated at low density to isolate individual clones. For recombinase-mediated cassette exchange (RMCE), ES cells were co-transfected with the RMCE construct and a Cre expressing plasmid and selected with the corresponding antibiotics for 10-14 days. ES cell colonies were picked into 96-well plates for expansion and genotyping by PCR to screen for the desired mutations followed by Sanger sequencing. Sequence verified positive clones were expanded from 96-well to 24-well then to 6-well plates and cryopreserved.

Positive ES cell lines carrying the engineered mutations were used to produce chimeras following standard procedures. Briefly, blastocysts were isolated from superovulated C57BL/6N females at 3.5 dpc, microinjected with ES cells, and then transferred into the uterus of 2.5 dpc pseudopregnant Swiss Webster females for implantation. High-percentage chimeric males were mated with C57BL/6N females for germline transmission of the engineered mutations. Heterozygous F1 mice were identified with junctional PCR from genomic DNA isolated from biopsies. F1 mice were then intercrossed to generate F2 mice homozygous for the same mutation or crossed to other lines as needed. All engineered mice were maintained in a mixed 129S6 and C57BL/6N background.

### Generation of the Singularity Musculus Allele

Igh is one of the largest loci in the mouse genome, spanning several megabases (Mb) near the right end of chromosome 12 q arm. It encodes many disparate elements involved in the generation of virtually endless diversity of antibodies. The locus contains a variable region of more than 2.5 Mb that encodes hundreds of gene segments responsible for much of the antibody diversity and a much smaller 220 kb constant CH region encoding expression for several antibody classes and subtypes (FIG. 1A). The CH region has eight CH genes that encode different Ig isotypes: Cμ (Ighm), Cδ (Ighd), Cγ3 (Ighg3), Cγ1 (Ighg1), Cγ2b (Ighg2b), Cγ2a/2c (Ighg2a/2c), Cε (Ighe), and Cα (Igha). Regulatory elements flanking as well as situated throughout this region are involved in class switch recombination (CSR) and timely expression of the isotypes (FIG. 2).

In addition to the above elements, located upstream of each Ig isotype (except for IgD) are the I promoter/exons and S switch regions. The latter is involved in CSR, and the former is involved in germline transcription of its corresponding Ig isotype. Transcription of Ig isotype is highly regulated. In resting B cells, germline transcription (GLT) is restricted to that of Cμ, driven by the Eμ enhancer and constitutive Iμ promoter. In activated B cells in response to antigen encounter and cytokine, transcription of the downstream Ig isotype is activated at its I promoter containing the respective response elements. Simultaneous transcription at the Iμ promoter and the downstream I promoter of the activated Ig isotype results in AID-mediated CSR.

A step-by-step process was used to generate a Singularity musculus allele (FIG. 1B). Unlike the normal tetrameric antibodies produced by wildtype (WT) mice (FIG. 1C), mice homozygous for this allele produce only HCAbs (FIG. 1D), the genetic composition and diversity of which derive entirely from the natural immune repertoire of mice.

Generation of the Singularity musculus allele was accomplished via 3 rounds of genetic engineering in LVGN-YF ES cells (FIG. 3). The Igh locus of these ES cells encoded Cγ2c, which is similar to that found in C57BL/6N mice, rather than Cγ2a in the BALB/c strain (FIGS. 3A and 3B). The first round of modification was performed via CRISPR-mediated non-homologous end joining (NHEJ), which deleted a 92.6 Kb genome DNA fragment spanning Cμ, Cδ, Cγ3, and the first exon of Cγ1 (which encodes the CH1 domain of IgG1) (FIGS. 3B and 3C). The sgRNAs were designed to cut immediately downstream of the Cu switch region (Sμ) and upstream of the second exon of Cγ1 (which encodes the hinge domain of IgG1), thereby placing the truncated Cγ1 gene under the direct control of Iμ promoter and Sμ, rendering the otherwise cytokine-inducible transcription of IgG1 to become constitutive, as for the case of Ig M in WT allele. The second round of modification was performed via CRISPR-mediated homology directed repair (HDR), which removed a 63.2 Kb genomic DNA fragment spanning Cγ2b, Cγ2c, Cε, and the first three exons of Cα, while introducing a selection marker cassette (PGK/Em7-neo) flanked with frt sites (FIGS. 3C and 3D). CRISPR cut sites were selected to avoid removal of the 3′□1E element downstream of Cγ1 and the 5′hsR1 element within intron 3 of Ca. The third round of engineering utilized transient expression of Flp recombinase, which removed the selection marker cassette, leaving a single frt site to be used for synteny verification of later modifications (FIGS. 3D and 3E). Regulatory elements (including Eμ, Iμ, Sμ, 3′γ1E, 5′hsR1, 3′RR, and 3′CBE) were kept intact to allow high-level constitutive transcription of CH1-truncated IgG1 (IgG1ΔCH1) from the endogenous Igh allele (FIGS. 2 and 3E). The resulting Singularity Musculus mice thus only produce HCAbs of the IgG1 subtype; all other antibody classes (IgM, IgD, IgE, and IgA) and IgG subtypes (IgG2b, IgG2c, and IgG3) were eliminated to avoid any potential mechanisms compromising HCAb production and to facilitate nanobody discovery, expression, and purification.

### Generation of the Singularity HyperDock Allele

To expand the versatility of the Singularity platform and generate HCAbs from other species, the Singularity HyperDock allele was generated by deleting a 2.58 Mb genomic DNA fragment containing all mouse VH, DH, and JH genes (from upstream of Ighv86-1 to downstream of Ighj4) and inserting a docking cassette for sequential RMCE upstream of Eu via CRISPR-mediated HDR (FIGS. 4 and 5). The HDR template contained the left homology arm, an frt site, an attB site, a PGK promoter, a loxP site, an Em7-neo cassette, an attP site, and a lox2272 site followed by the right homology arm. The frt site was incorporated to verify the introduced RMCE docking cassette was on the same chromosome (C57BL/6N) as the Singularity Musculus allele upon expression of Flp recombinase. The wild type loxP site, instead of other heterospecific lox sites, was selected to place between the PGK promoter and the Em7-neo cassette to enable highly efficient RMCE events via selection marker swapping. These modifications resulted in a mouse VDJ-null Singularity HyperDock allele that contained RMCE docking sites for sequential introduction of BACs, cloned constructs, or synthetic fragments containing any combinatorial segments of V, D, or/and J genes of the heavy or light chain alleles from humans or other species (FIG. 5B).

### Generation of the Singularity Sapiens Allelic Series

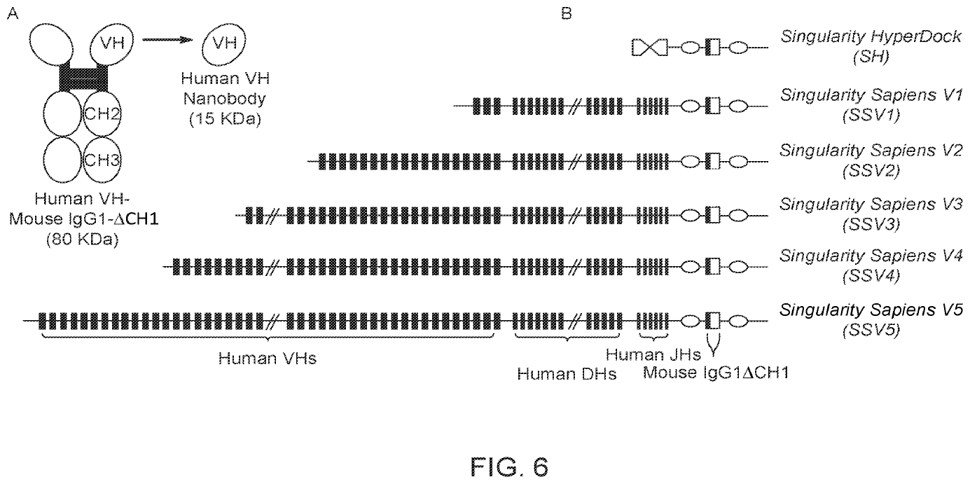

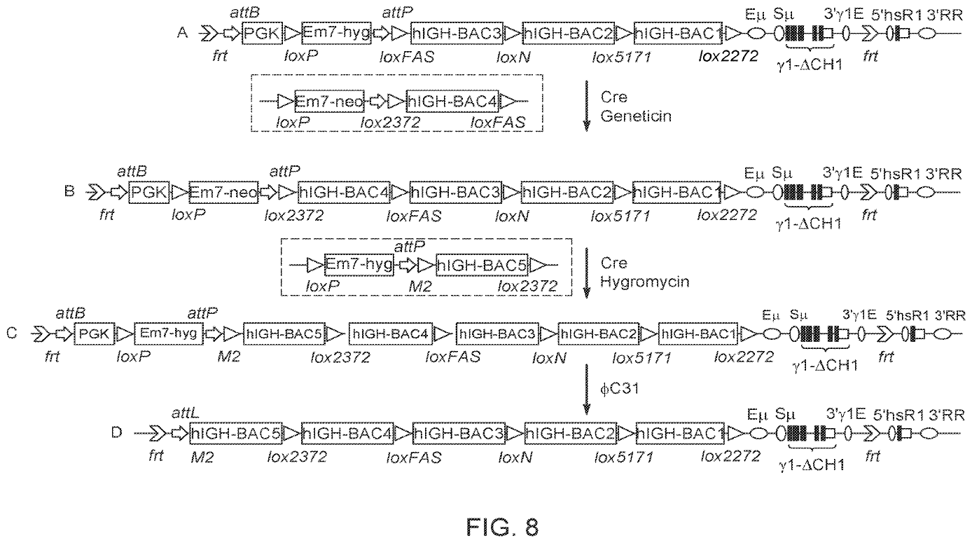

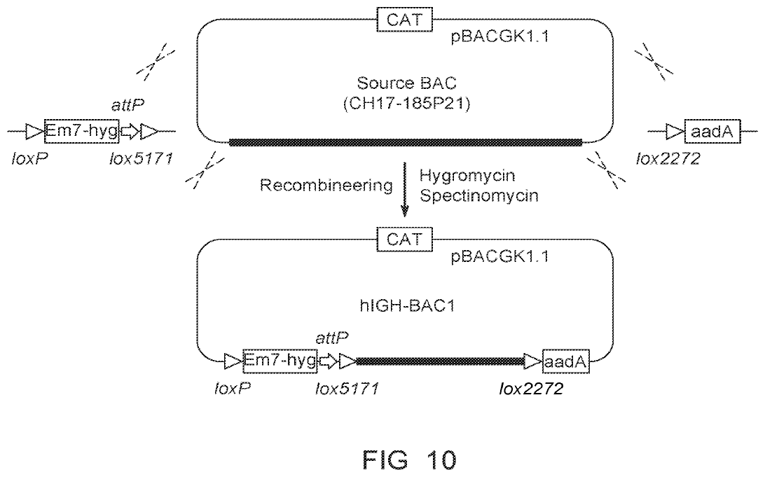

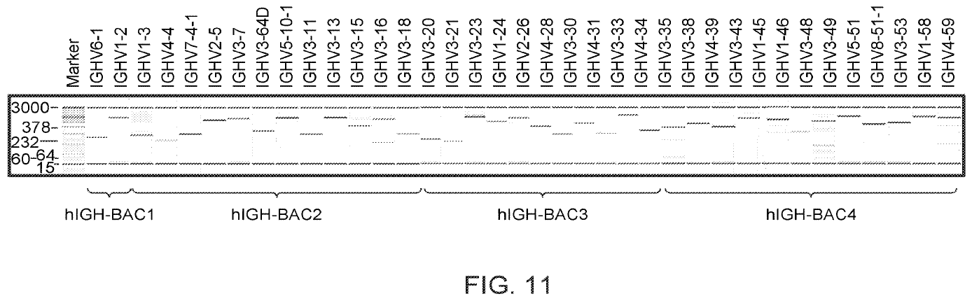

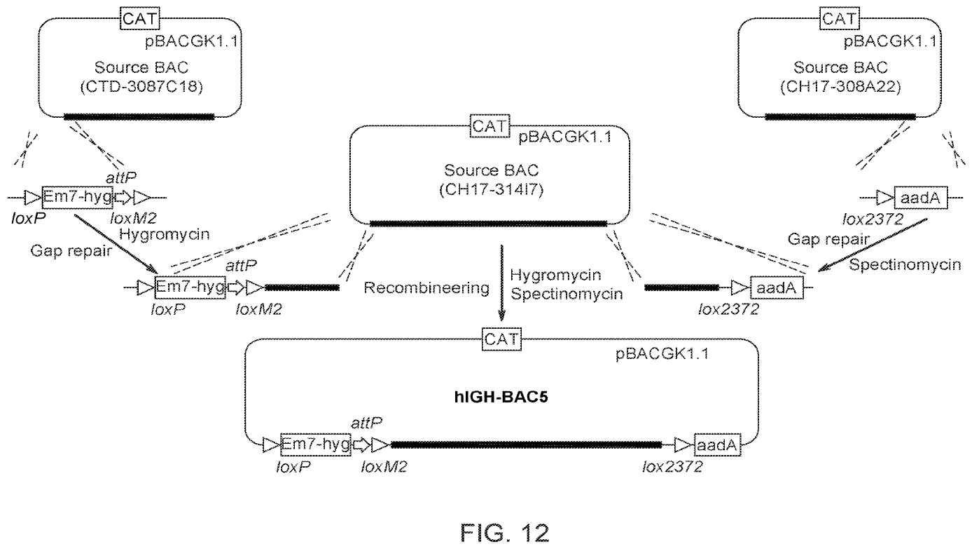

Engineered BACs containing human VH, DH, and JH genes were introduced into the Singularity HyperDock allele to generate the Singularity Sapiens allelic series (FIGS. 6-8). Overlapping IGH BAC clones from the CH17 BAC library and RPCI-11 library (BACPAC resources) (FIG. 9 and Table 4) were modified at both ends by bacterial homologous recombination (recombineering) to incorporate either the Em7-hyg or the Em7-neo cassettes to allow selection marker swapping, while introducing the corresponding heterospecific lox sites (Table 1) flanking the genomic fragment for sequential RMCE. Briefly, a synthetic gBlock (IDT or Twist) containing two 75-150 base pairs (bp) homology arms flanking proper lox site(s) and antibiotic resistance cassette was electroporated into an E. coli strain containing a heat inducible Red recombinase in an electroporation cuvette with 1-mm gap using a Bio-Rad GenePulser II apparatus at 1.75 kV, 25 μF, and 200 ohms. Next, 1.0 mL SOC medium was added to each cuvette and then transferred into a microfuge tube before incubating at 32° C. for 1 hour with shaking (200 rpm). Cells were subsequently plated onto LB agar plate with the corresponding antibiotics. The resulting colonies were screened by PCR with junctional primers followed by Sanger sequencing for verification. The recombineering process is illustrated in FIG. 10 for the first introduced BAC (hIGH-BAC1), which contained 3 human V_{H} genes (two functional; IGHV1-2 and IGHV6-1), 27 human DH genes, and 9 human J_{H} genes. A loxP-Em7-hyg-attP-lox5171 cassette was introduced via recombineering immediately upstream of the human IGHV1-2 gene at the 5′ end of the original BAC clone, followed by the introduction of a lox2272-aadA cassette immediately downstream of the human IGHJ6 gene at the 3′ end. The engineered BAC was then used for the first round of RMCE (between loxP and lox2272) to introduce the three human VH genes, all human DH genes, and all human JH genes immediately upstream of the mouse Igh intronic enhancer Eμ, while introducing a different heterospecific lox site (lox5171) for the next round of RMCE (FIGS. 7A and 7B). Subsequent overlapping BACs were modified in a similar fashion albeit using alternating selection markers (Em7-neo and Em7-hyg) and different heterospecific lox sites, with overlapping fragments trimmed off to assemble the human VDJ genomic region in a stepwise fashion (FIGS. 7C, 7D and 8). The original BAC clones and heterospecific lox sites used to reconstruct the complete human VDJ region can be found in Table 4 and Table 1, respectively. The wildtype loxP site, which exhibits high recombination efficiency, was used for each round of RMCE to pair with different heterospecific lox sites. All engineered BACs were confirmed by PacBio SMRT sequencing prior to transfection into ES cells. No mutation of significance was found except for several SNPs and small indels in the intergenic regions. The sequential RMCE processes resulted in the generation of a series of humanized singularity alleles with increasing VH diversity (SSV1-5). PCR analysis followed by Sanger sequencing of SSV4 mice with VH-specific primers confirmed the integration of all 37 functional VH elements (FIG. 11). The HIGH-BAC5 was engineered to contain sequences from three source BACs (FIG. 12; Table 4) and is introduced into SSV4 ES cells via RMCE to complete the construction of SSV5, which was designed to contain the complete human VH repertoire (126 VHs, 27 DHs, and 9 JHs genes).

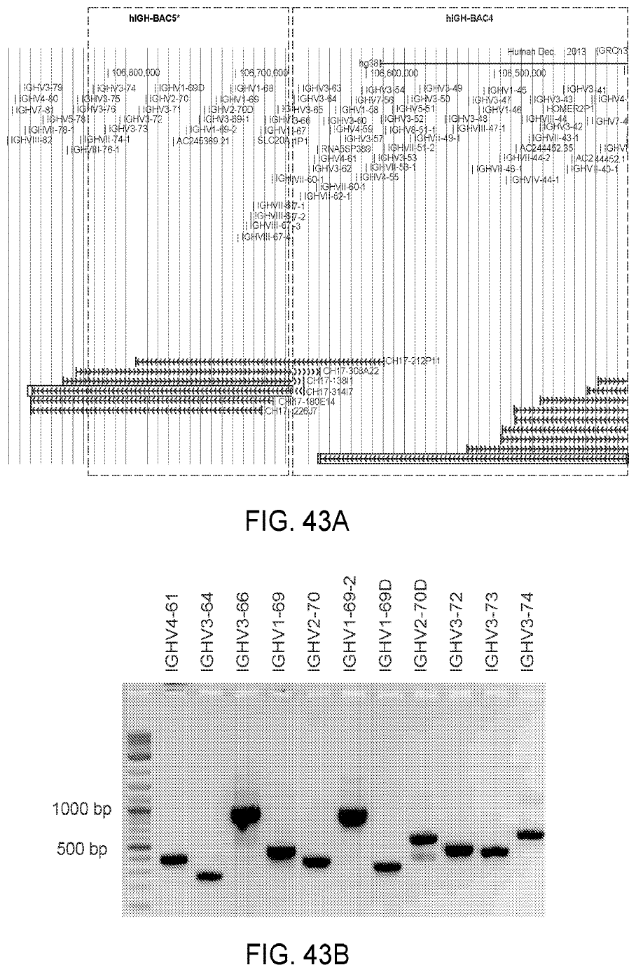

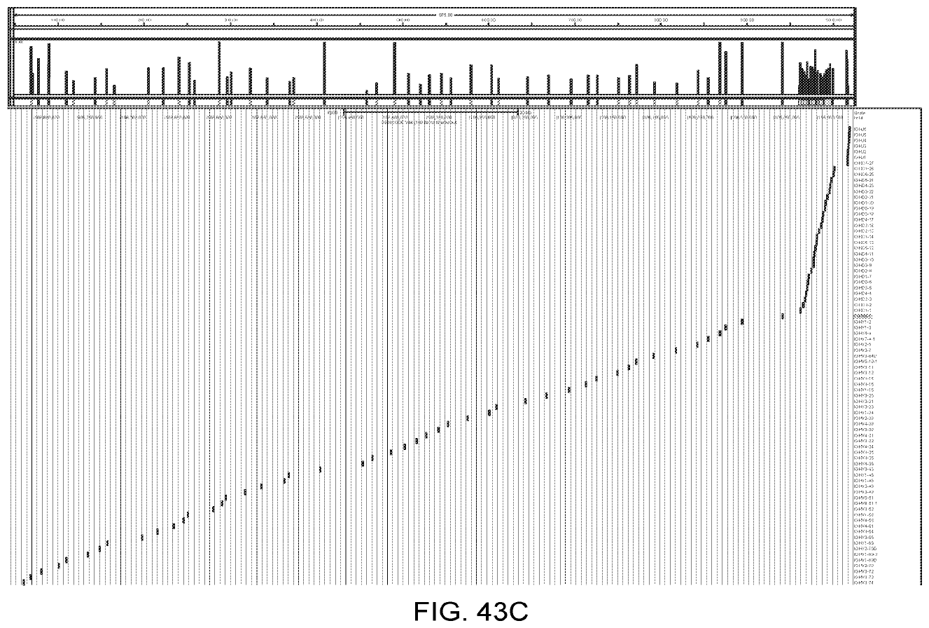

To create a Singularity Sapiens mouse containing BACs from only a CH17 BAC series, an alternative approach was used to create hIGH-BAC5*. Only BAC CH17-308A22 was used as a template to create hIGH-BAC5*, resulting in the exclusion of 10 non-functional VH gene segments (pseudogenes or ORFs only) present in the human IgH locus (FIG. 43A). PCR analysis confirmed that hIGH-BAC5* contained 11 functional VH gene segments (FIG. 43B).

TABLE 4
The human genome coordinates (GRCh38/hg38) of the engineered and source BACs
used in the engineering of the Singularity Sapiens allelic series SSV1-SSV5.

| Engineered BAC | Engineered BAC Coordinates | Source BAC | Source BAC Coordinates |
|---|---|---|---|
| hIGH-BAC1 | chr14: 105862806-105989907 | CH17-185P21 | chr14: 105786489-105990780 |
| hIGH-BAC2 | chr14: 105989908-106196572 | CH17-108J24 | chr14: 105982809-106209070 |
| hIGH-BAC3 | chr14: 106196573-106395796 | CH17-447I7 | chr14: 106194147-106395690 |
| hIGH-BAC4 | chr14: 106395797-106633503 | CH17-268I9 | chr14: 106395814-106634186 |
| hIGH-BAC5 | chr14: 106634307-106875815 | CH17-308A22 | chr14: 106634435-106824828 |
| | | CH17-314I7 | chr14: 106648191-106860132 |
| | | CTD-3087C18 | chr14: 106849834-106875815 |
| hIGH-BAC5* | chr14: 106635260-106815284 | CH17-308A22 | chr14: 106634435-106824828 |

### Generation of Igk and Igl Knockouts for Producing Light-Chain Free Singularity Mice

To prevent unwarranted interference of light chains with the generation of HCAbs in the Singularity mice, the murine light chains were removed by deleting the V and J gene segments from the IgK and IgL loci.

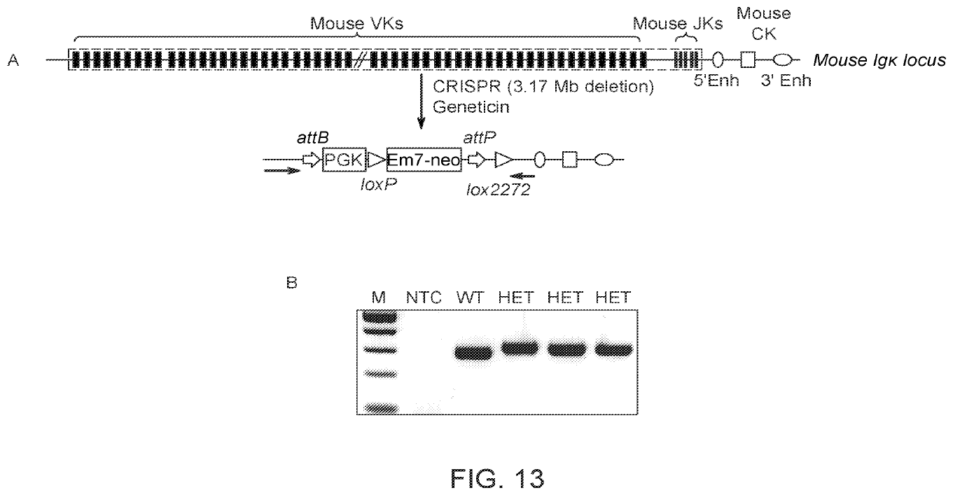

To remove the kappa light chains, a 3.17 Mb genomic DNA fragment that contains the entire mouse VK and JK gene segments was deleted and replaced with a docking cassette by CRISPR/Cas9 mediated HDR (FIG. 13A). Similar to the Singularity HyperDock at the Igh allele, the engineered Igk HyperDock/KO allele contains an attB site, a PGK promoter, a loxP site, an Em7-neo cassette, an attP site, and a lox2272 site upstream of the 5′ Enhancer element located at the 5′ end of the mouse CK gene, thus allowing sequential RMCE at the Igk locus. The engineered Igk HyperDock/KO mice were generated and confirmed by PCR and sequencing (FIG. 13B).

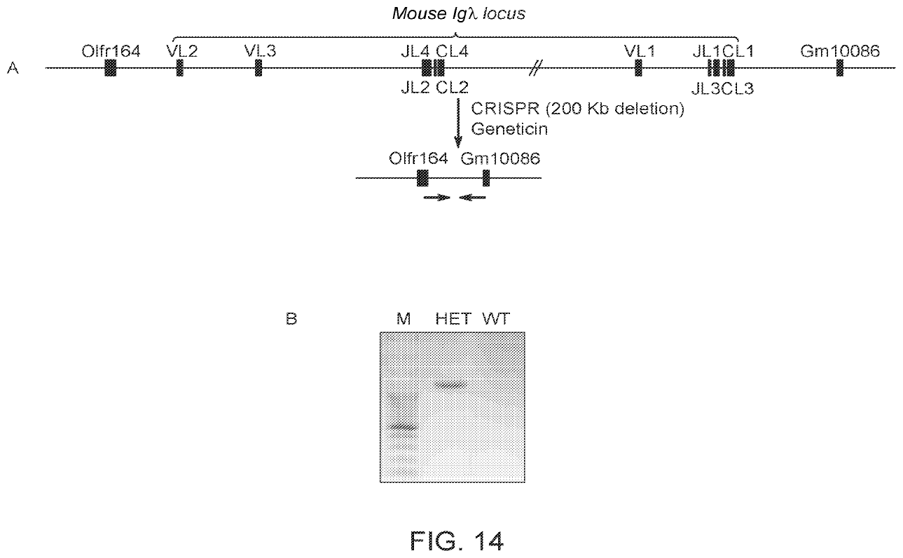

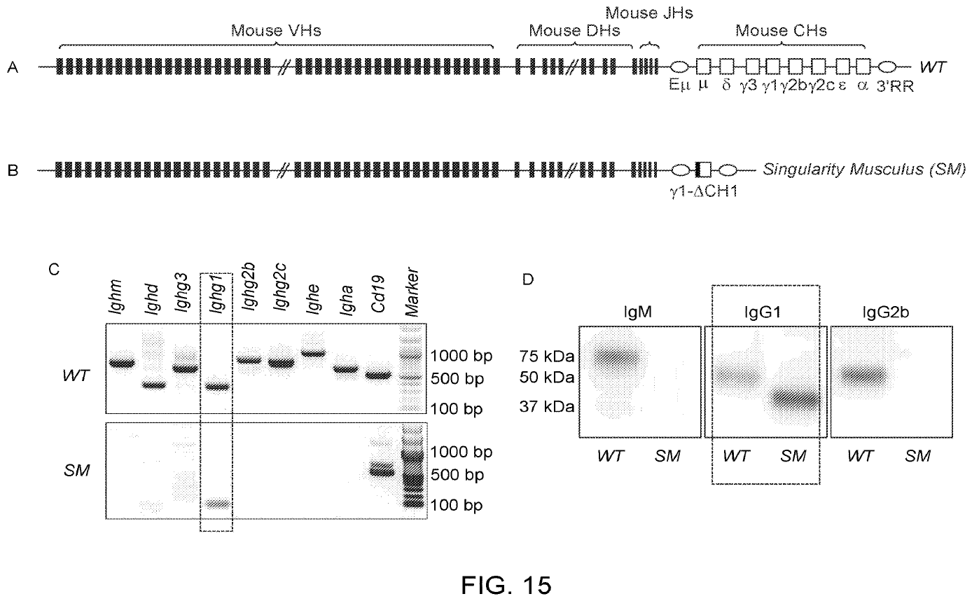

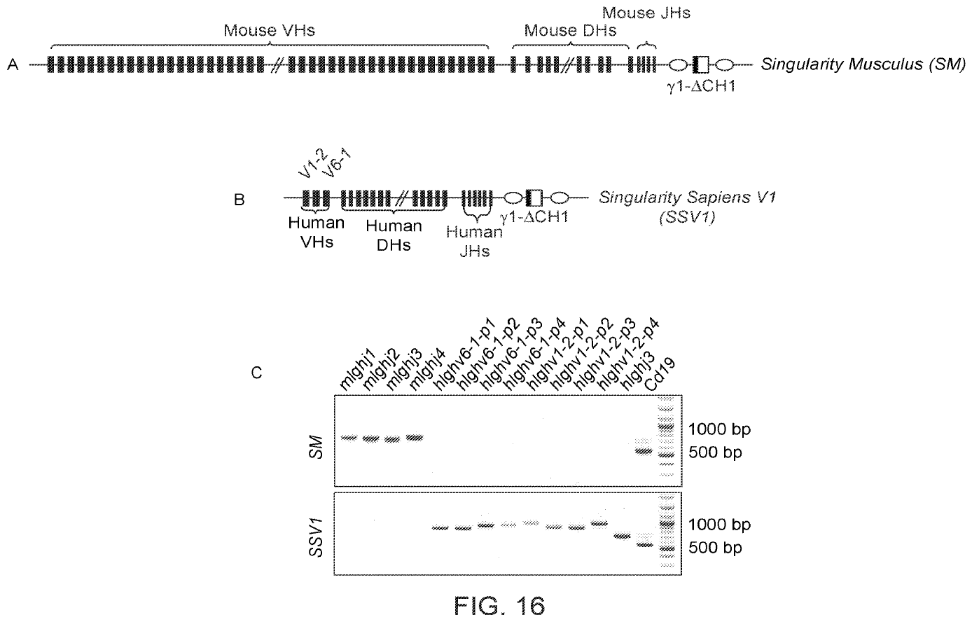

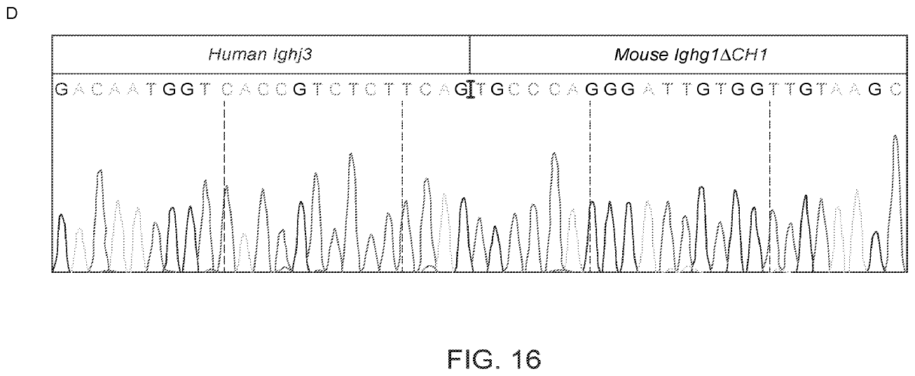

To remove the lambda light chains, a ˜200 kb genomic DNA fragment between Olfr164 and Gm10086, which contains the entire λ locus including VL1, VL2, VL3, and all of JL and CL gene segments, was deleted by CRISPR/Cas9 mediated NHEJ (FIG. 14A). The Igl KO ES cells were generated and confirmed by PCR and sequencing (FIG. 14B) and were used to generate Igl KO mice. Also, Igl HyperDock/KO ES cells and mice were generated and confirmed by PCR and sequencing in a similar fashion as Igk HyperDock/KO.

### Example 2: Characterization of Singularity Mice

### Singularity Mice Constitutively Express Only Heavy Chain Antibodies of CH1-Truncated IgG1

To confirm that, unlike WT mice that can express the full panel of Ig isotypes (FIG. 15A), the Singularity Musculus (SM) mice express only IgG1-ΔCH1 (FIG. 15B), transcription of different Ig classes and subtypes was analyzed. Total RNA was isolated from spleens of WT and SM mice using the Trizol reagent, and RNA concentration and quality was determined with Bioanalyzer. Reverse transcription was performed using Superscript IV Reverse Transcriptase (ThermoFisher) and oligo (dT) 20 primers according to the manufacturer's instructions. Expression of all Ig classes and subtypes was analyzed by RT-PCR according to standard procedures, with mouse B-cell marker Cd19 as an internal control. While Ighm, Ighd, Ighg3, Ighg1, Ighg2b, Ighg2c, Ighe, and Igha were all expressed in the WT mice, the Singularity musculus mice only expressed Ighg1 transcript of reduced size, lacking the CH1 sequence as verified with Sanger sequencing (FIG. 15C).

To examine the transcription of Ighg1 in Singularity Sapiens mice, RT-PCR was performed on cDNA that was reverse-transcribed from total spleen RNA of Singularity Sapiens (SSV1) mice. SSV1 derived from the Singularity Musculus platform through Singularity Igh HyperDock/KO (FIGS. 5 and 16A) was designed to contain the full panel of human D_{H} and J_{H} and two functional human V_{H} (IGHV6-1, IGHV1-2) (FIG. 16B). PCR primers were designed having a set of forward primers specific to human IGHV6-1, IGHV1-2, and IGHJ3 and a reverse primer specific to mouse Ighg1 CH2. Chimeric transcripts with human VDJ-mouse Ighg1-ΔCH1 were detected in the Singularity Sapiens (SSV1), but not in the Singularity Musculus mice (FIG. 16C), which were further verified by sequencing to be correctly spliced (FIG. 16D).

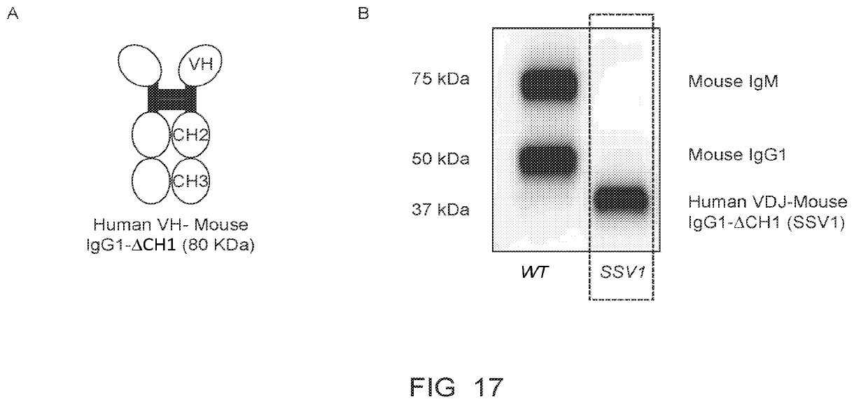

To examine the protein expression of different Ig classes and subtypes, immunoglobulins in plasma samples of immunized wild type and SM mice were purified with protein A/G magnetic beads, separated by reducing SDS-PAGE, and electrophoretically transferred onto Immobilon®-P membranes (Millipore Sigma) according to standard procedures. Immunodetection was conducted with HRP-conjugated secondary antibodies and enhanced chemiluminescence and autoradiography were performed using ECL Western blotting reagents. A truncated IgG1 of about 40 kDa was detected in SM mice (as compared to the full length IgG1 of about 50 kDa in the wild type mice), whereas IgM and IgG2b were not detected, as expected (FIG. 15D). Similarly, a truncated IgG1 of about 40 kDa, corresponding to the human VDJ-mouse IgG1-ΔCH1, was detected in immunized Singularity Sapiens mice (SSV1), which did not express IgM or full length IgG1 as seen in the wild type mice (FIG. 17).

### Upregulated IgG Expression in B Cells of Singularity Mice

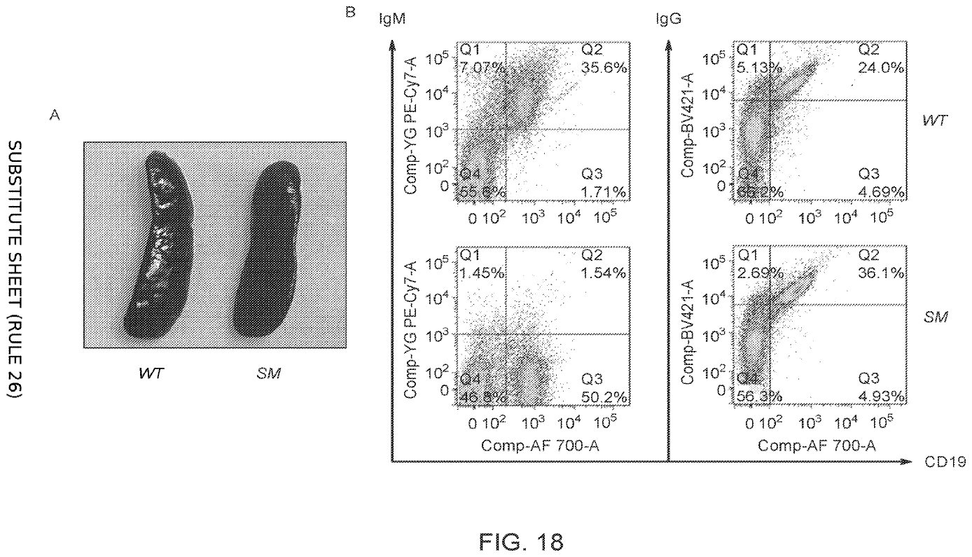

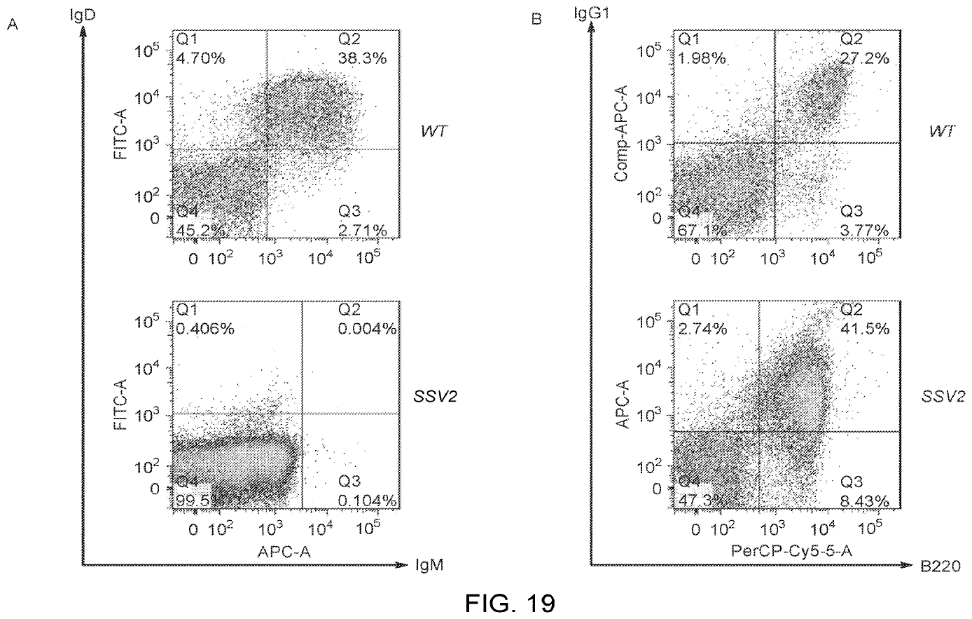

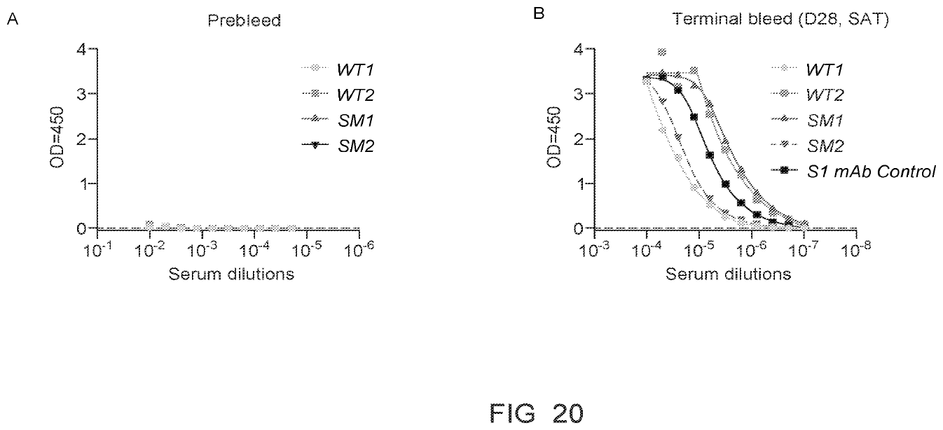

The spleens of Singularity Musculus mice were of similar shape and size as those of the wild type mice (FIG. 18A). To examine the expression of IgM and IgG on B cell membranes, single cell suspensions were prepared from spleens, treated with ACK lysis buffer to remove red blood cells, blocked with Fc blocker, and stained in FACS buffer (PBS with 1% FBS) with rat-anti-mouse IgM (PE-Cy7), rat-anti-mouse IgG (BV421), and rat-anti-mouse CD19 (AF700). Following staining, cells were analyzed by flow cytometry (BD LSR II). While no IgM⁺ B cells were detected in the Singularity Musculus mice, IgG⁺ B cells were detected at significantly higher proportion as compared to those in the wild type mice (FIG. 18B), consistent with the increased expression levels due to the constitutive high-level germline transcription of IgG1 (FIG. 15D). Similarly, FACS analysis of splenocytes from Singularity Sapiens (SSV2) and wild type mice was performed using the above mentioned procedure with rat-anti-mouse IgM (APC), rat-anti-mouse IgG1 (APC), rat-anti-mouse IgD (FITC), and rat-anti-mouse B220 (PerCP-Cy5.5). The analysis confirmed the absence of IgM⁺ or IgD⁺ B cells and significantly higher proportion of IgG1⁺ cells in SSV2 mice, as expected but they were found to be abundant in the wild type mice (FIG. 19A).

### Robust Humoral Immune Response in Singularity Mice

Protein antigens (Table 2) were prepared in phosphate-buffered saline (PBS) and freshly mixed 1:1 (v/v) with either Complete Freund's Adjuvant (Sigma Cat #5881, for priming injection) or Incomplete Freund's Adjuvant (Sigma Cat #5506, for boosting injections) by repeated passage through two connected syringes until a smooth emulsion was formed. Injections were performed using a 1-mL syringe and a 27-gauge needle into 4-12-week-old male or female mice. Priming and boosting injections were done at 2-week intervals at 10-25 μg antigen protein per mouse, subcutaneously into the left and right groin each (50 μL) and/or 100 μL intraperitoneally. Tail vein bleed was collected prior to each injection. A final boost was done in Week 4 or Week 6 intraperitoneally with antigen proteins without adjuvants. Animals were sacrificed 3-4 days later to collect terminal blood samples and havest tissues. Blood samples were processed into plasma according to standard procedures.

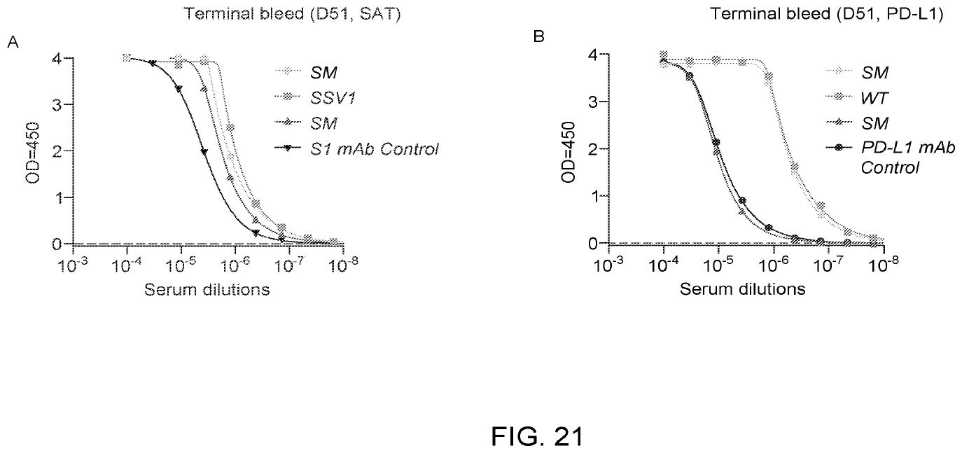

To assay for antibody titers in plasma, ELISA plates were coated with 1 μg/mL antigen protein diluted in PBS overnight at 4° C. Following repeated washing with PBST (PBS+0.05% Tween-20) and blocking with SuperBlock (Thermo Fisher), plasma samples were serially diluted in dilution buffer and applied to the plates. After unbound protein was removed through multiple washes, bound proteins were detected using a corresponding HRP-conjugated secondary antibody, developed with 3,3′,5,5′ tetramethylbenzidine (TMB) substrate (BM blue, Sigma) and stopped with 50 μL of 1 M H₂SO₄. Absorbance was read at 450 nm. Robust humoral immune responses comparable to those in wild type mice were observed in Singularity Musculus mice after 4 weeks (D28) with SAT immunization (FIGS. 20A and 20B), with significantly higher titers obtained after 6 weeks (D51) for both Singularity Musculus and Singularity Sapiens mice (SSV1) (FIG. 21A). Similar results were obtained with other immunogens such as PD-L1 (FIG. 21B).

### NGS Analysis of VH Repertoire in Singularity Mice

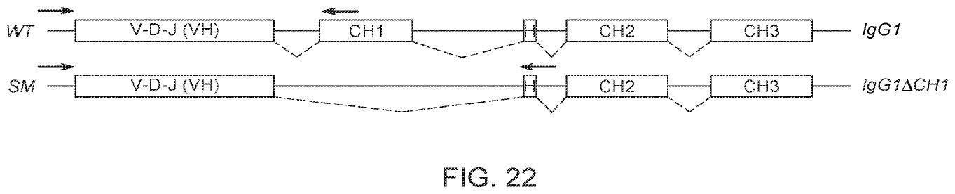

Total RNA was isolated by the Trizol reagent from spleens of wild type or Singularity Musculus mice immunized with different antigens, and RNA quality and concentration was determined with Bioanalyzer. The recombined variable region sequences (VH) of the wild type or Singularity mice were amplified by 5′ Rapid Amplification of cDNA Ends (5′RACE) for next generation sequencing (NGS). Briefly, reverse transcription was performed using Superscript IV Reverse Transcriptase, oligo (dT) 20 primers and a template switch primer that contains the 5′RACE adapter and unique molecular identifier (UMI) sequences (5′-CTACA-CTCTTTCCCTACACGACGCTCTTCCGATCTNNNNNNNNNNNNrGrGrGrGrG-3′; SEQ ID NO: 13). The products of template switch reverse transcription were then amplified in the first round of PCR reaction using the 5′RACE adapter forward primer (5′-CTACACTC-TTTCCCTACACGACGCTCTTCCGATCT-3′; SEQ ID NO: 14) and a reverse primer specific to the IgG1 CH2 domain (5′-GGTGGTTGTGCAGGCCCTCATG-3′; SEQ ID NO: 15). Purified products were further amplified in the second round of PCR using the 5′RACE adapter forward primer and a reverse primer specific either to the IgG1 CH1 domain (5′-CCATGGAGTTAGTTTGGGCAGCA-3′ for wild type IgG1 transcripts; SEQ ID NO: 16) or the IgG1 Hinge domain (5′-CAAGGCTTACAACCACAATCCCT-3′ for Singularity IgG1 transcripts; SEQ ID NO:17) to ensure that both mouse lines generated about 600 bp amplicons (FIG. 22). The resulting nested PCR products were further amplified in the third round of PCR reactions to incorporate the Illumina P5 and P7 adaptor sequences for NGS and barcodes to enable sample multiplexing. The final 5′RACE libraries were purified and sequenced using 2X 300 bp paired-end run on an Illumina MiSeq.

Paired-end sequence reads in fastq format were processed and aligned using KAligner, a specialized version of K-mer chaining algorithm (Liao et al., Nucleic Acids Res., 41 (10):e108 (2013)) to reference germline genes of VH, DH, and JH based on annotations from the international ImMunoGeneTics information system (IMGT, World Wide Web at imgt.org), and the CDRs regions were identified. The full-length in-frame sequences were further assembled into clonotypes when the CDR3 were identical and no more than 2 mismatched nucleotide residues were present among the sequences. The sequences with low quality were excluded from assembling. The clonotypes from each animal were ranked according to their abundance, and the clonotypes with less than 5 counts were not included for further analysis.

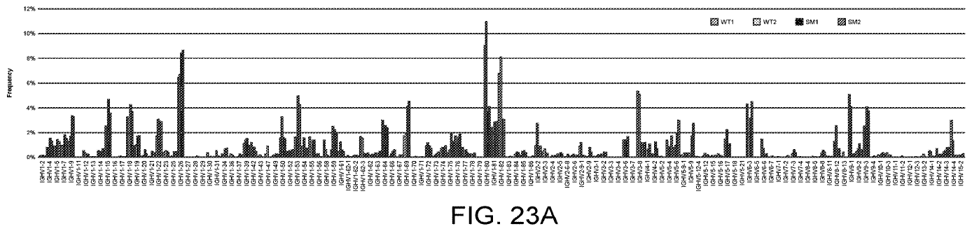

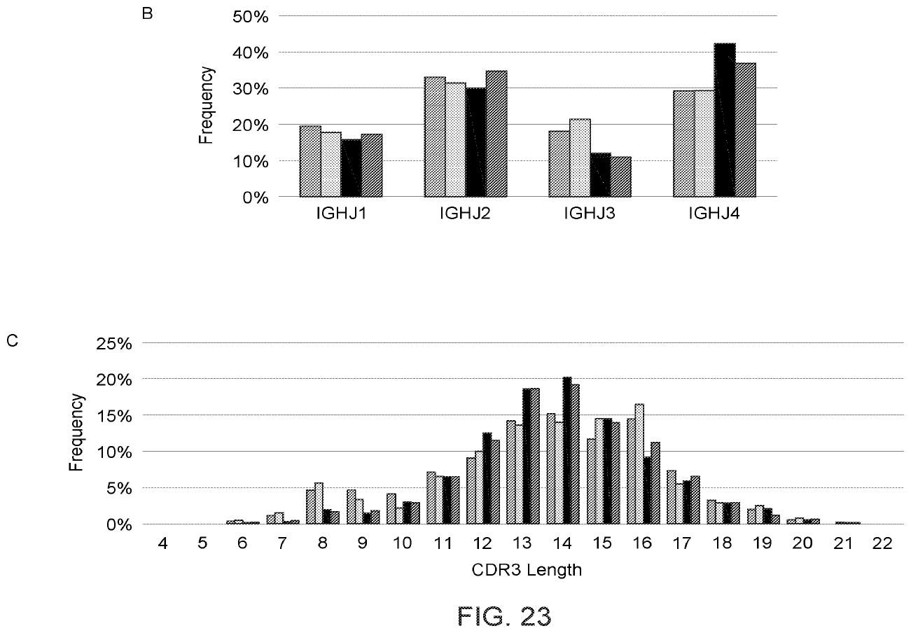

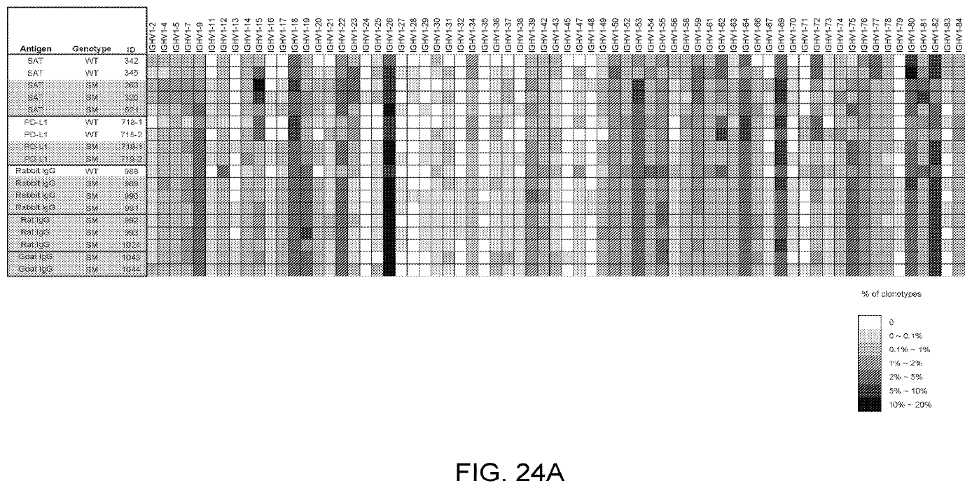

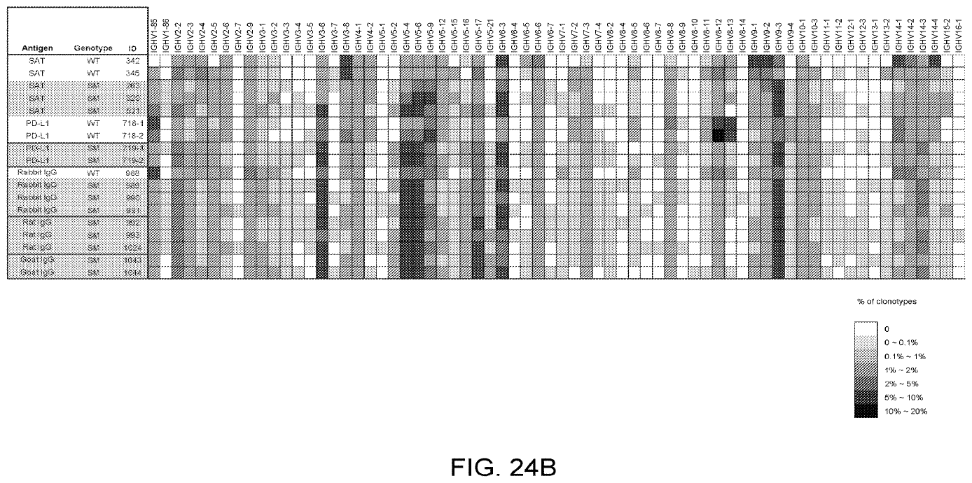

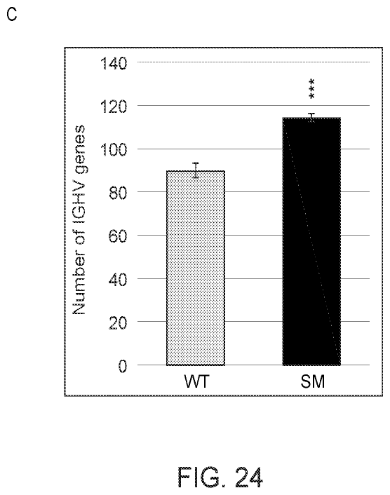

A total of 18 samples from wild type and Singularity Musculus mice immunized with SARS-Cov2 Spike Active Trimer SAT (R&D Systems; Catalog No. 10549-CV), PD-L1 (R&D Systems; Catalog No. 156-B7), Rabbit IgG (ThermoFisher; Catalog No. 02-6102), Rat IgG (ThermoFisher; Catalog No. 31933), or Goat IgG (ThermoFisher, Catalog No. 31245) were processed for NGS to determine their corresponding VH repertoire. More than one million reads per sample were recovered in all samples, roughly half of which successfully aligned to the Igh locus (Table 2). Notably, the number of clonotypes against all tested antigens in the Singularity Musculus mice were significantly higher than those seen in the WT mice, ranging from several fold to over 20-fold. Moreover, whereas 79-99 IGHV gene segments were utilized in WT mice against these antigens, a significantly higher number of IGHVs (103-122) were utilized in the Singularity Musculus mice, almost close to the theoretical limit (125 functional IGHVs based on mouse genome GRCm38/mm10 annotation) (FIGS. 23A and 24; Table 2). The ability of the Singularity Musculus mice to access a greater number of IGHV segments as compared to WT was highly significant across several tested antigens (FIG. 24), which may have resulted from the higher level of IgG1 GLTs in the Singularity Musculus mice as compared to the inducible, cytokine-dependent expression of IgG1 GLTs in the WT mice, as well as the removal of all other Ig classes and subtypes in SM mice, thereby enabling sole expression of IgG1 regardless of immunogens.

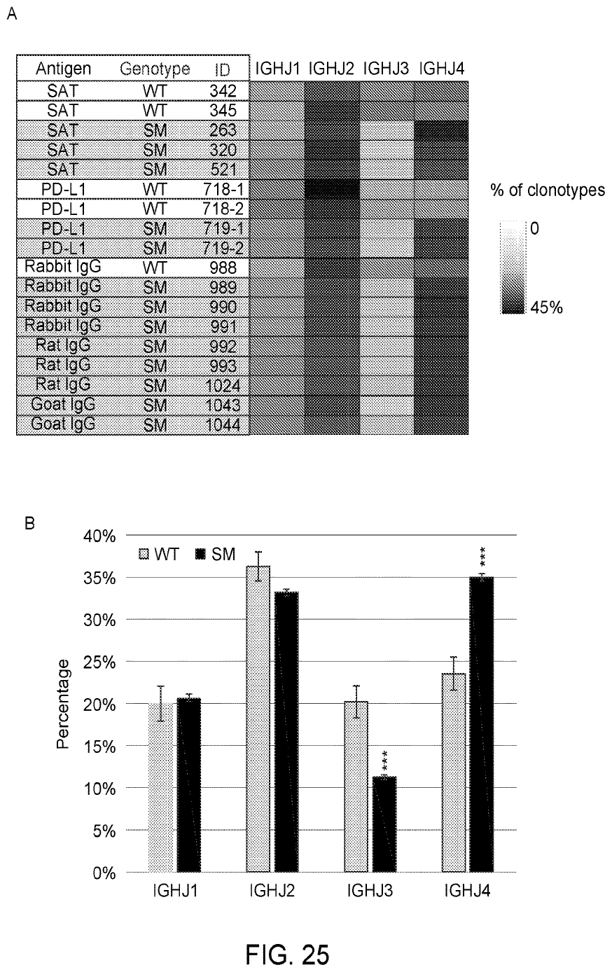

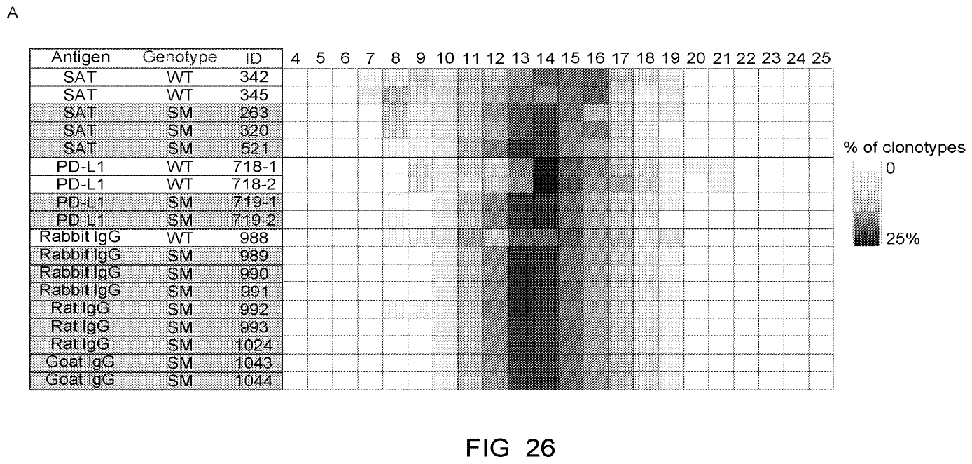

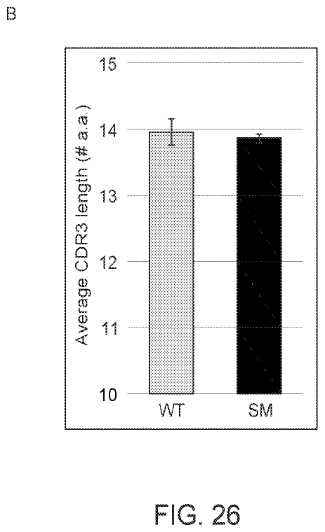

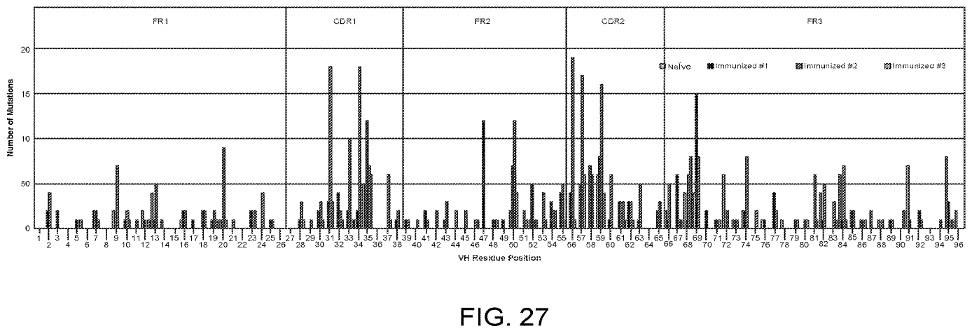

Analysis of VH sequences showed that compared to the WT mice, the Singularity mice exhibited similar diverse usage of the IGHV gene segments (FIGS. 23A and 24). The IGHV gene segments that yielded more abundant or less abundant clonotypes in the wild types also did so in the Singularity Musculus mice (FIGS. 24A-24C). While all 4 IghJ segments were used, IGHJ3 was discriminated against whereas IGHJ4 was favored in Singularity Musculus mice, which likely resulted from a structural preference for HCAb formation (FIGS. 23B and 25). No significant difference was observed in CDR3 size distribution among clonotypes, with average size being about 14 for both wild type and Singularity Musculus mice (including the invariable C and W residues at the CDR3 boundaries) (FIGS. 23C and 26).

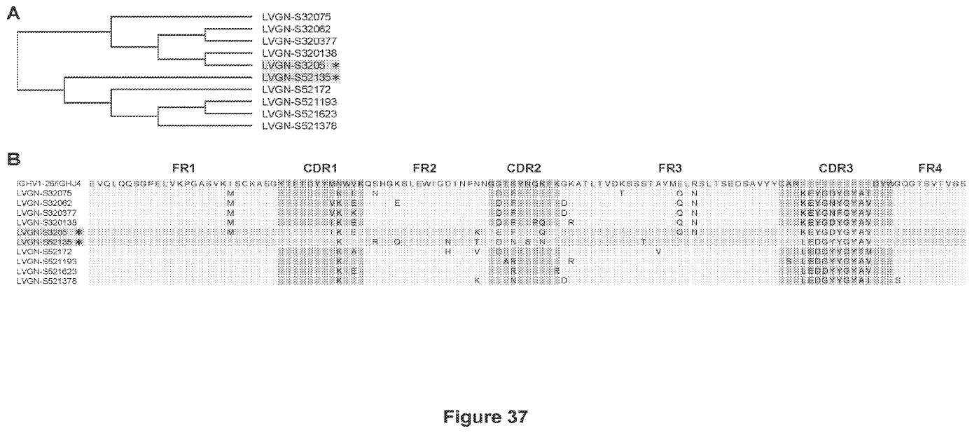

To identify somatic hypermutations, the top 100 ranked clonotype sequences from each Singularity Musculus mouse that were either naive or immunized with SAT were aligned to the corresponding germline IGHV sequences using IgBlast (World Wide Web at ncbi.nlm.nih.gov/igblast/). The mutation rate at each residue position according to the IMGT numbering was calculated and plotted (FIG. 27). While a low level of mutation rate was observed in the naive mice, immunized mice exhibited a significantly higher level of somatic hypermutations, which were highly enriched in the CDR regions (FIG. 27). This was further confirmed in later analysis of the complete VH sequences of validated nanobody binders (FIG. 37).

### Example 3: Nanobody Discovery with Singularity Mice

### Sequence-Driven, High-Throughput Screen for Nanobody Binders

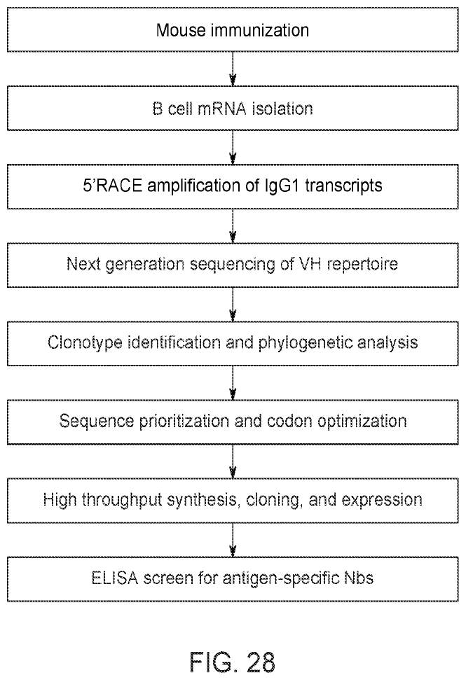

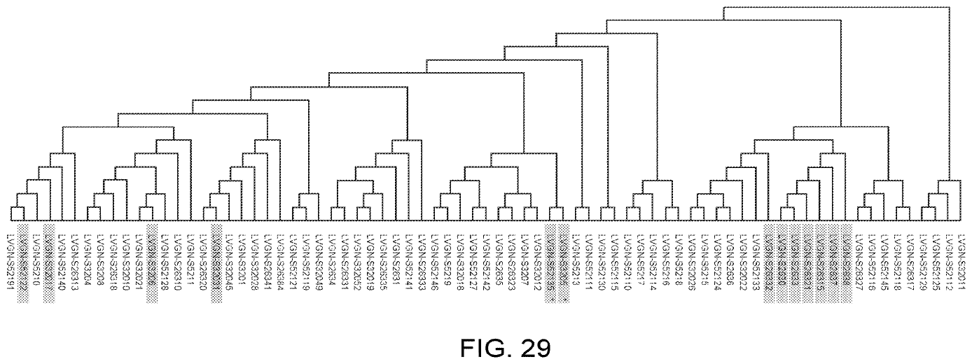

Next generation sequencing (NGS) and bioinformatic analysis was performed to profile and select VH sequences (clonotypes) from immunized Singularity mice, followed by gene synthesis, cloning, expression, and ELISA screening to identify nanobody binders (FIG. 28). Alternatively, nanobody binders can be identified with other methods including, without limitation, hybridoma, single B cell cloning, single B cell sequencing, and various display approaches such as bacterial display, yeast display, mammalian cell display, and phage display.

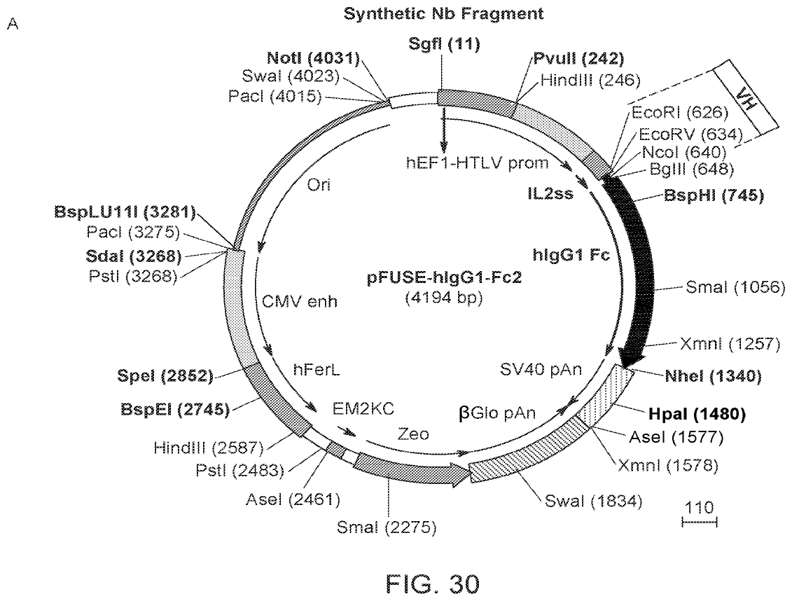

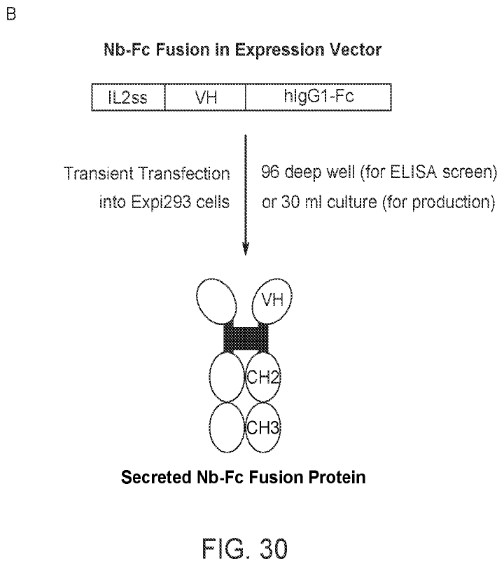

To select for candidate clonotypes for nanobody expression and binder screens, clonotypes from each animal were ranked according to their abundance and somatic hypermutation rate. Phylogenetic analysis of clonotype sequences was carried out with Clustal Omega (World Wide Web at ebi.ac.uk/Tools/msa/clustalo) to select representative sequences from different branches (FIG. 29). Candidate clonotype sequences (VHs) from the Singularity mice immunized with SAT were first human-codon optimized, flanked with cloning adapters, and synthesized as eBlock gene fragments (IDT). The synthesized eBlocks were then cloned into pFuse-hIgG1-Fc2 vector (Invivogen) by NEBuilder HiFi DNA assembly to generate in-frame fusions of an IL-2 signal peptide, VH, and the Fc domain (Hinge-CH2-CH3) of human IgG1 (FIGS. 30A-30B). Sequence-verified expression constructs were then transfected into Expi293F cells (ThermoFisher) in 96-well format to produce secreted nanobody-Fc fusions according to the manufacturer's instructions, and the culture supernatants were collected 6 days post-transfection and were used for ELISA screen to identify antigen-specific binders.

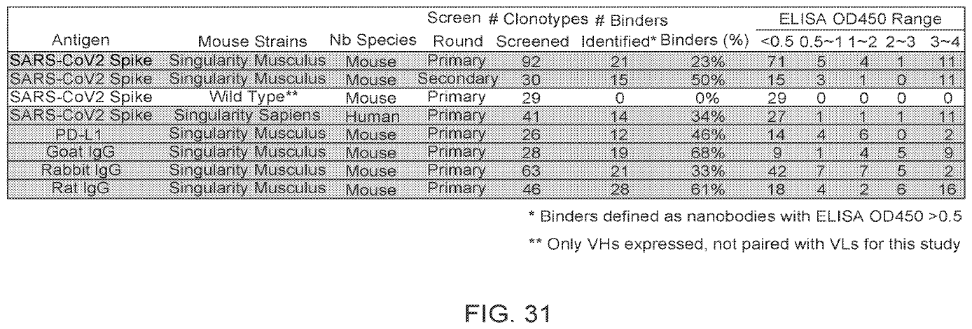

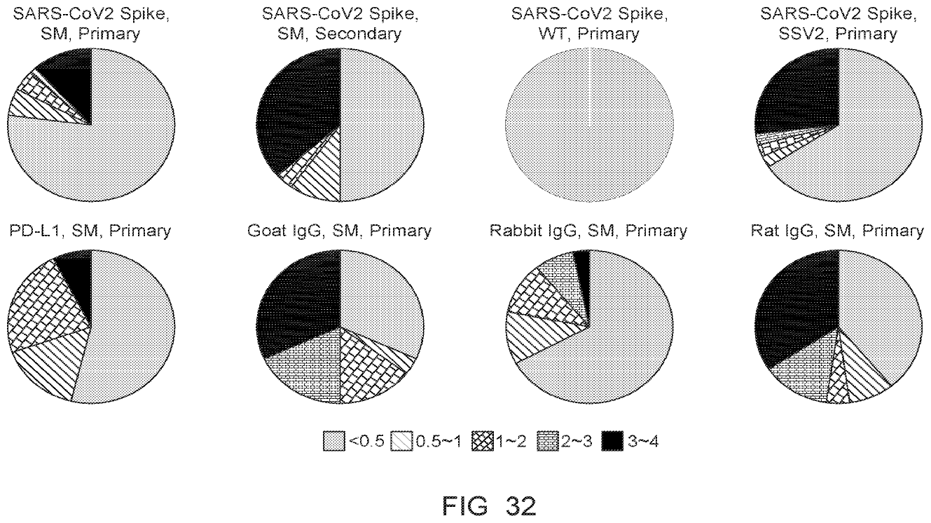

A screen of 92 clonotypes selected from Singularity Musculus mice immunized with SAT identified 21 (23%) binders (ELISA OD>0.5), among which 11 (52%) exhibited high level of binding (ELISA OD>3.0) (FIGS. 31 and 32). These VH sequences were then used to query the original clonotype sequence libraries by phylogenetic analysis to identify homologous VH sequences, which were then used for secondary screen for hit expansion. Fifteen (50%) out of 30 clonotypes screened (mostly of low abundance thus not included in the primary screen) were binders, among which 11 (73%) exhibited high affinity (FIGS. 31 and 32). By contrast, a control screen using clonotypes from wild type mice selected after the same criteria (high abundance and hypermutation rate) failed to identify any binder (0 out of 29), suggesting that functional nanobodies can only be derived from HCAbs produced in Singularity mice, but not possible from the conventional H2L2 antibodies produced in the wild type mice (FIGS. 31 and 32). SAT nanobody binder screen with Singularity Sapiens mice (SSV2) identified 14 out of 41 (34%) Nb-Fc binders of human VH sequences, suggesting that functional HCAbs were produced only in humanized mice following the same mechanisms, not in the WT mice (FIGS. 31 and 32).

ELISA screens for nanobodies against PD-L1, Goat IgG, Rabbit IgG, and Rat IgG identified 33%˜61% binders to the corresponding antigen, further demonstrating the high efficiency of the NGS-driven screening methods, which were uniquely suitable for nanobody discovery (FIGS. 31 and 32). Due to the single-chain nature of HCAbs, each clonotype identified represents a unique antibody, which was identified with bulk RNA-seq without using any single cell approach.

### Biophysical and Biochemical Properties of Purified Nb-Fcs

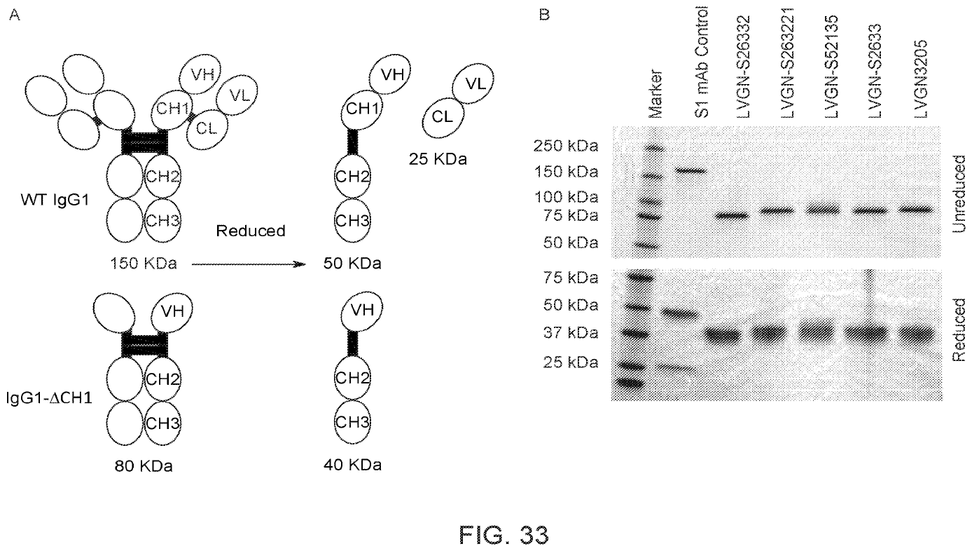

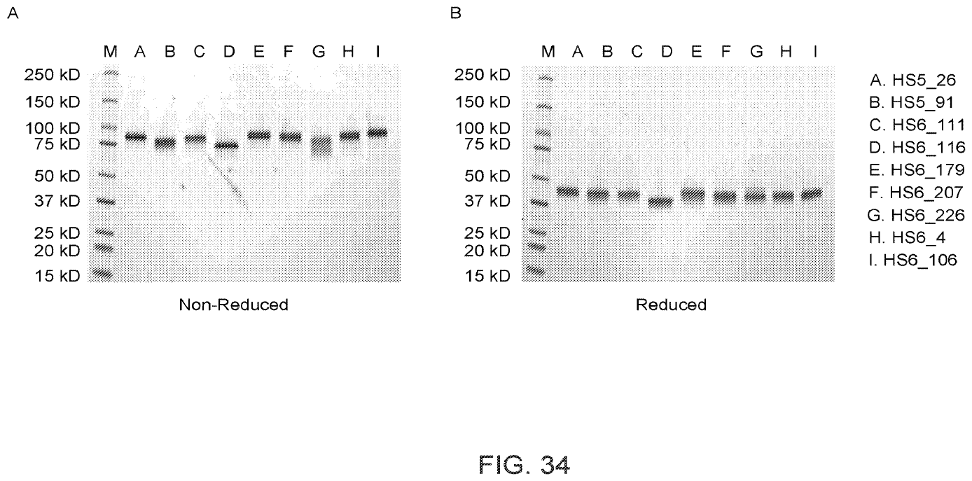

A selected set of ELISA positive SAT Nb-Fc constructs (from both mouse and human VH sequences; Table 5) were used to transfect a 30-mL Expi293F cell culture to produce nanobody-Fc fusions, which were then purified using protein A affinity chromatography following standard procedures. Briefly, six days post-transfection, cell culture supernatants were collected, filtered with 0.22 μm filters and loaded onto 0.5 ml, protein A columns (MabSelect SuRe, Cytiva) pre-equilibrated with PBS. After washing with 2 mL of PBS, bound proteins were eluted from the columns with 4 mL citric buffer (25 mM citrated acid, 150 mM sodium chloride, pH 3.5) and neutralized with additional of 1 M Tris-HCl (pH 8.8). The final buffer was exchanged into PBS with Vivospin Turbo (30,000 MWCO PES). SDS-PAGE analysis showed that, in contrast to conventional antibodies (about 150 KDa) with two heavy chains (about 50 KDa) and two light chains (about 25 KDa), the purified Nb-Fc fusions migrated at about 80 kDa under unreduced condition and at about 40 kDa under reduced condition, consistent with the expected sizes of VH-based Nb-Fcs as homodimers (FIGS. 33 and 34).

TABLE 5
Biochemical and biophysical properties of mouse and human SAT Nbs.

| SARS-Cov2 | | | | | | | | | K_{d} (M) | | |
|---|---|---|---|---|---|---|---|---|---|---|---|
| Spike | | | | | Yield | Tm1 | ELISA | ELISA | SPR | SPR | BL1 |
| Nb—Fe | Alt. ID | Species | PI | MW | (mg) | (° C.) | EC50 (M) | IC50 (M) | (Carterra) | (Biacore) | (Octet) |
| MS521_35 | LVGN_S52135 | Mouse | 7.19 | 82465.17 | 0.65 | 66 | 2.4E−11 | 4.9E−10 | 5.30E−09 | ND | 9.09E−09 |
| M320_5 | LVGN_S3205 | Mouse | 7.19 | 82535.25 | 0.45 | 67 | 2.4E−11 | 4.0E−10 | 3.17E−09 | ND | 1.21E−09 |
| MS263_3 | LVGN_S2633 | Mouse | 8.49 | 83266.78 | 1.07 | 66 | 1.3E−09 | ND | ND | ND | ND |
| MS263_21 | LVGN_S26321 | Mouse | 8.33 | 83308.82 | 0.83 | 60 | 2.2E−10 | ND | ND | ND | ND |
| MS263_32 | LVGN_S26332 | Mouse | 7.75 | 81156.21 | 1.15 | 62 | 1.0E−10 | ND | ND | ND | ND |
| M2S521_193 | LVGN_S521193 | Mouse | 8.09 | 82405.21 | 0.31 | 65 | 8.5E−11 | 1.6E−10 | ND | ND | ND |
| M2S521_72 | LVGN_S52172 | Mouse | 7.74 | 82491.43 | 1.16 | 59 | 6.3E−11 | 4.1E−11 | ND | ND | ND |
| M2S320_377 | LVGN_S320377 | Mouse | 8.09 | 82531.31 | 1.56 | 68 | 9.7E−11 | 1.6E−9 | ND | ND | ND |
| M2S320_75 | LVGN_S32075 | Mouse | 7.19 | 82487.16 | 1.87 | 67 | 6.8E−11 | 1.2E−10 | ND | ND | ND |
| M2S521_378 | LVGN_S521378 | Mouse | 7.73 | 82525.39 | 0.92 | 68 | 8.1E−11 | 2.2E−9 | ND | ND | ND |
| M2S521_623 | LVGN_S521623 | Mouse | 7.19 | 82609.3 | 1.28 | 59 | 5.4E−11 | 1.3E−10 | ND | ND | ND |
| HS5_91 | NA | Human | 8.76 | 83572.8 | 1.47 | 65 | 4.2E−11 | ND | ND | 4.80E−08 | 4.79E−08 |
| HS6_111 | NA | Human | 8.77 | 81853.26 | 0.81 | 62 | 6.7E−11 | ND | ND | 1.20E−08 | 1.38E−08 |
| HS6_207 | NA | Human | 8.53 | 82089.36 | 1.39 | 65 | 1.5E−10 | ND | ND | 1.10E−08 | 3.77E−08 |
| HS6_226 | NA | Human | 8.65 | 82195.66 | 1.09 | 68 | 7.0E−11 | ND | ND | 4.20E−09 | 1.61E−08 |
| HS6_106 | NA | Human | 8.66 | 81951.47 | 1.59 | 65 | 4.6E−11 | ND | ND | 4.80E−09 | 9.22E−09 |
| HS5_26 | NA | Human | 8.65 | 83702.89 | 1.31 | 60 | 5.3E−10 | ND | ND | 1.10E−08 | 6.03E−08 |
| HS6_4 | NA | Human | 8.66 | 81899.16 | 1.37 | 64 | 5.8E−10 | ND | ND | 3.12E−07 | 8.43E−08 |
| HAb8_S* | LVGN_S8107 | Human | 7.75 | 82769.74 | 0.97 | 65 | 1.8E−11 | 7.9E−11 | 3.73E−08 | 5.70E−09 | 1.98E−08 |

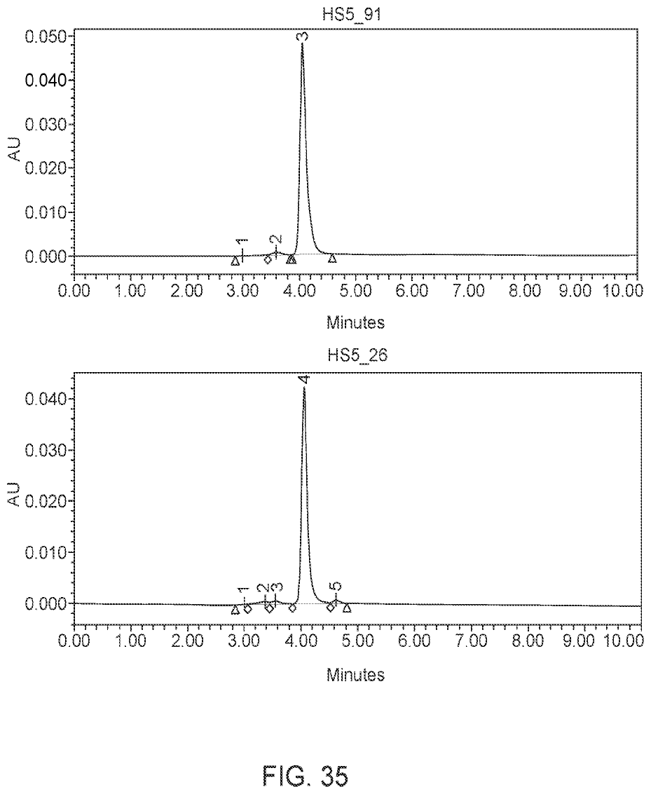

To examine the quality of purified Nb-Fcs, size exclusion chromatography (SEC) analysis was performed. Briefly, 2-10 uL purified Nb-Fc samples were injected into ACQUITY UPLC (Waters) Protein BEH SEC 200, 1.7 μm, 4.6×150 mm column with a flow of 0.3 mL/minute for 10 minutes. A mobile phase of 50 mM Sodium Phosphate, 500 mM NaCl, pH 6.2 was used. A high percentage of Nb-Fcs generated did not exhibit a propensity of aggregation (representative SEC plots on FIG. 35).

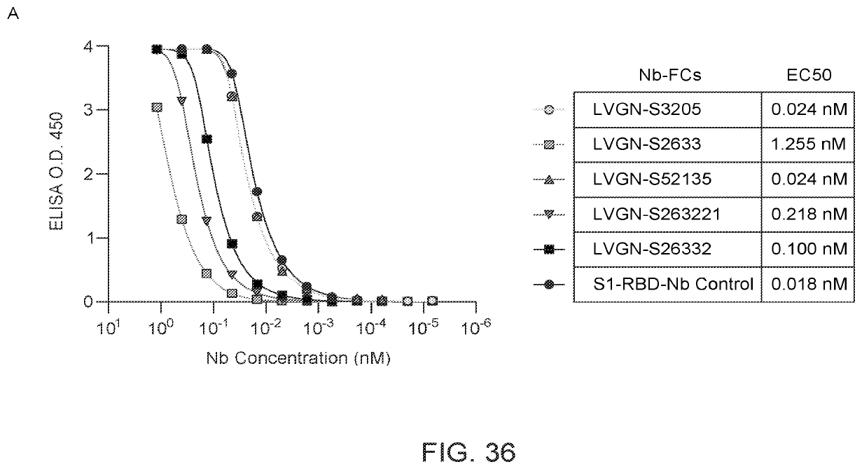

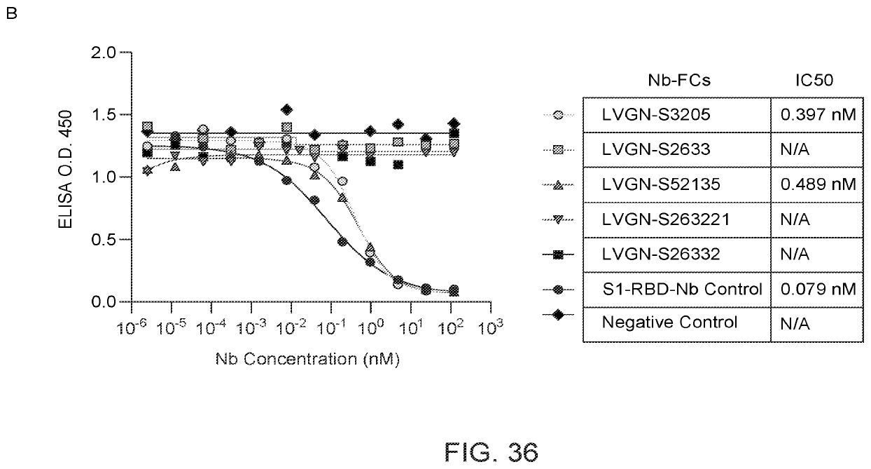

To assess binding affinity of purified SAT Nb-Fc fusions, ELISA assays were conducted with serially diluted protein samples against the SAT antigen. All Nb-Fc tested exhibited an ELISA EC₅₀ in the nanomolar and sub-nanomolar range, comparable to that of a human SAT Nb-Fc control VH-Ab-8 (HAb8-S) (Li et al., Cell, 183:429 (2020)) (FIG. 36A; Table 5). To access their neutralizing potency, a competitive ELISA assay was conducted with COVID-19 Spike-ACE2 Binding Assay Kit (Raybiotech) according to the manufacturer's instructions. A number of Nb-Fcs exhibited potent neutralizing potency against Spike-Ace2 binding, with IC₅₀ comparable to that of HAb8-S (FIG. 36B; Table 5). Interestingly, many of those were identified from secondary screen using the two potent SAT neutralizing nanobodies (LVGN-S3205 and LGVN-S52135) identified from the primary screen, further demonstrating the power of the sequence-driven nanobody discovery pipeline (FIGS. 31 and 37).

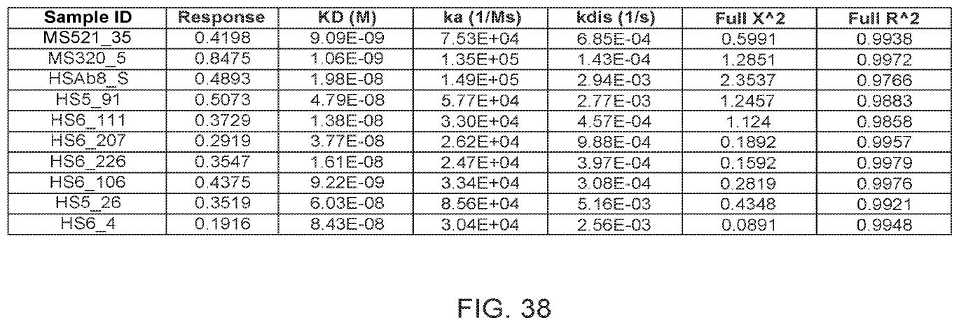

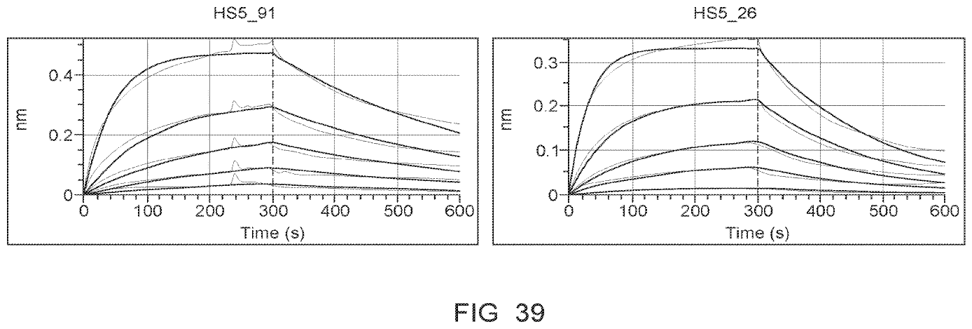

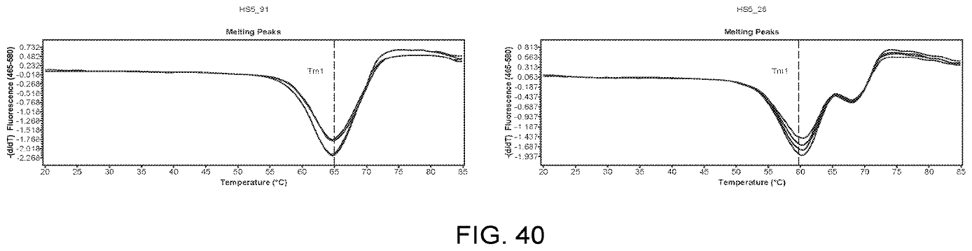

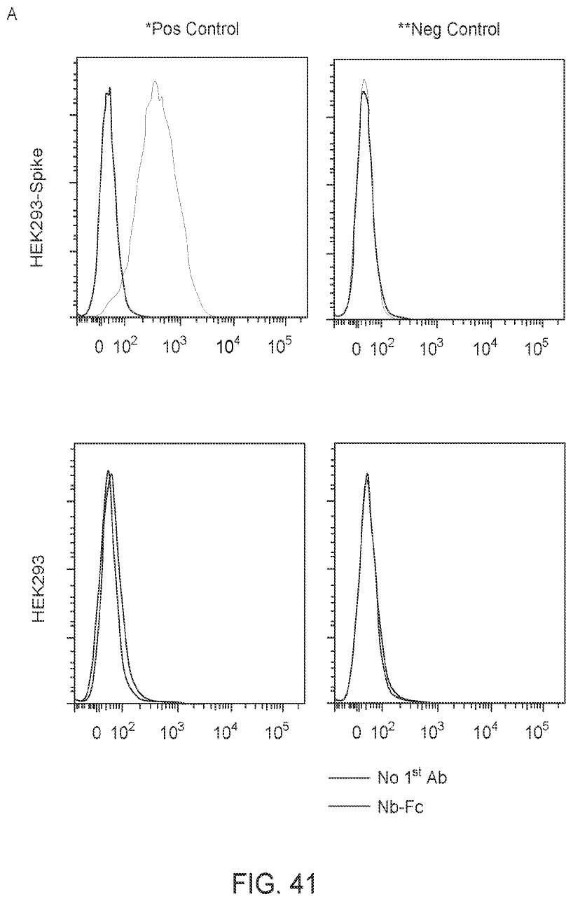

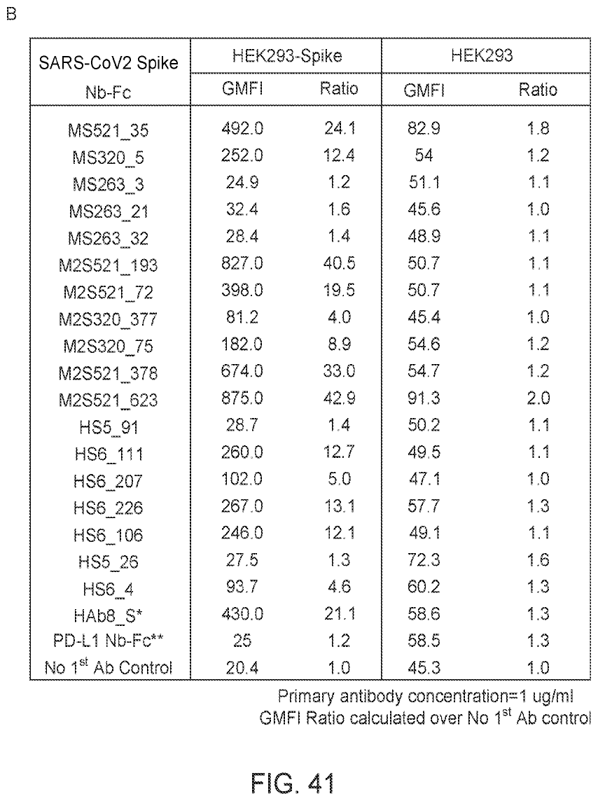

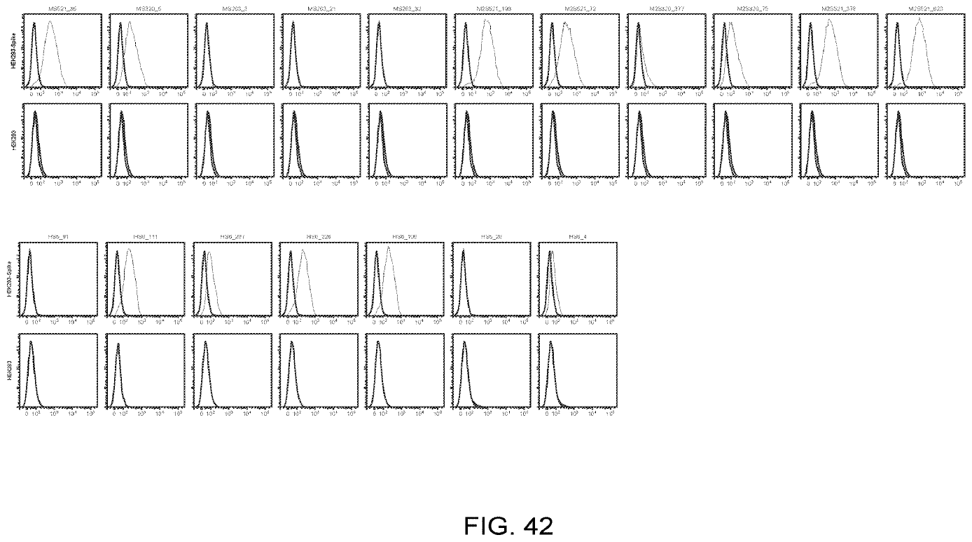

The kinetics of Nb-Fc to SAT binding was analyzed with Surface Plasmon Resonance (SPR) and/or Biolayer Interferometry (BLI) (Table 5). For BLI, binding experiments were performed on the Octet HTX at 25° C. The antibodies were loaded onto Anti-Human Fc Capture (AHC) sensors and then dipped with serial dilutions of antigen (starting at 333 nM, 1:3 dilution, 5 points). Reference sample well (buffer) was used for data analysis. Kinetic constants were calculated using a monovalent (1:1) binding model. Representative kinetics and sensorgrams are shown in FIGS. 38 and 39. Most Nb-Fcs exhibited a single or double digit nanomolar (10⁻⁸-10⁻⁹M) KD, comparable to that of the HAb8-S (Table 5).

To assess thermostability, Differential Scanning Fluorimetry (DSF) was used to measure the melting temperature (Tm) of purified Nb-Fcs. Briefly, Nb-Fcs were mixed with Thermal Shift™ Dye (ThermoFisher) for a final concentration at 1 μg/mL, and a 10 μL/well mixture was transferred into a 384-well plate. The plate was sealed with MicroAmpR Optical Adhesive and loaded onto a Roche LightCyclerR 480 Instrument. Fluorescence signals were collected as the temperature increased from 20° C. to 85° C. at 0.06° C./second. Most purified Nb-Fcs exhibited high thermostability, with an average Tm1=64.28±0.64° C. (PBS, pH7.4) (Table 5). Representative melting curves were shown in FIG. 40.

A FACS based cell binding assay was performed to examine the binding property of Nb-Fcs to Spike proteins on cell surface (FIGS. 41 and 42). HEK293 parental cells and HEK293-Spike cells expressing the SARS-COV-2 Spike(S) protein with an inactivated furin site (293-SARS2-S-dfur, Invivogen #293-cov2-sdf) were incubated with individual Nb-Fcs at 1 μg/mL for 1 hour at 4° C., washed, and then incubated with goat anti-human IgG-Fc coupled to DyLight 594 (ThermoFisher) for 1 hour at 4° C. FACS analysis of these samples was performed on BD LSR II, and geometric mean fluorescence intensities (GMFI) were calculated with FlowJo V10. Twelve out 18 of Nb-Fcs, which bound to SAT in ELISA format, also exhibited cell surface SAT binding above background, with a GMFI ratio between 4.0-42.9 (FIGS. 41 and 42).

TABLE 6
Closely related VH sequences identified using two SARS-COV2 neutralizing
nanobodies, LVGN-S3205 and LGVN-S52135.

| SEQ ID | | |
|---|---|---|
| NO. | Sequence Name | VH Sequences |
| 2 | IGHV1-26/IGHJ4 | EVQLQQSGPELVKPGASVKISCKASGYTFTDYYMNW |
| | | VKQSHGKSLEWIGDINPNNGGTSYNQKFKGKATLTV |
| | | DKSSSTAYMELRSLTSEDSAVYYCAR |
| | | KEYGDYGYAT- - - - - - - - - - DYWGQGTSVTVSS |
| 3 | LVGN-S32075 | EVQLQQSGPELVKPGASVKMSCKASGYTFTDYYMK |
| | | WLKQNHGKSLEWIGDINPNNGDTFYNQKFKGKATLT |
| | | VDTSSSTAYMQLNSLTSEDSAVYYCARKEYGDYGYA |
| | | TDYWGQGTSVTVSS |
| 4 | LVGN-S32062 | EVQLQQSGPELVKPGASVKMSCKASGYTFTDYYVK |
| | | WEKQSHGESLEWIGDINPNNGDTFYNQKFKDKATLT |
| | | VDKSSSTAYMQLNSLTSEDSAVYYCARKEYGNYGY |
| | | AVDYWGQGTSVTVSS |
| 5 | LVGN-S320377 | EVQLQQSGPELVKPGASVKMSCKASGYTFTDYYVK |
| | | WKKQSHGKSLEWIGDINPNNGDTFYNQKFKDKATLT |
| | | VDKSSSTAYMQLNSLTSEDSAVYYCARKEYGNFGYA |
| | | VDYWGQGTSVTVSS |
| 6 | LVGN-S320138 | EVQLQQSGPELVKPGASVKMSCKASGYTFTDYYIKW |
| | | EKQSHGKSLEWIGDINPNNGDIFYNPQFKGRATLTVD |
| | | KSSSTAYMQLNSLTSEDSAVYYCARKEYGDYGYAV |
| | | DYWGQGTSVTVSS |
| 7 | LVGN-S3205 | EVQLQQSGPELVKPGASVKMSCKASGYTFTDYYIKW |
| | | EKQSHGKSLEWIGDINPKNGETFYNQQFKGKATLTV |
| | | DKSSSTAYMQLNSLTSEDSAVYYCARKEYGDYGYA |
| | | VDYWGQGTSVTVSS |
| 8 | LVGN-S52135 | EVQLQQSGPELVKPGASVKISCKASGYTFTDYYMKW |
| | | VKQRHGQSLEWIGNINPTNGDTNYSQNFKGKATLTV |
| | | DKSSTTAYMELRSLTSEDSAVYYCARLEDGYYGYAV |
| | | DYWGQGTSVTVSS |
| 9 | LVGN-S52172 | EVQLQQSGPELVKPGASVKISCKASGYTFTDYYMKW |
| | | AKQSHGKSLEWIGHINPVNGDTSYNQKFKGKATLTV |
| | | DKSSSTVYMELRSLTSEDSAVYYCARLEDGYYGYTM |
| | | DYWGQGTSVTVSS |
| 10 | LVGN-S521193 | EVQLQQSGPELVKPGASVKISCKASGYTFTDYYMKW |
| | | VKQSHGKSLEWIGDINPNNGGARYNQKFKGRATLTV |
| | | DKSSSTAYMELRSLTSEDSAVYYCSRLEDGYYGYAV |
| | | DYWGQGTSVTVSS |
| 11 | LVGN-S521623 | EVQLQQSGPELVKPGASVKISCKASGYTFTDYYMKW |
| | | EKQSHGKSLEWIGDINPNNGGTRYNQKFRGKATLTV |
| | | DKSSSTAYMELRSLTSEDSAVYYCARLEDDYYGYAV |
| | | DYWGQGTSVTVSS |
| 12 | LVGN-S521378 | EVQLQQSGPELVKPGASVKISCKASGYTFTDYYMKW |
| | | VKQSHGKSLEWIGDINPKNGGTNYNQKFKDKATLTV |
| | | DKSSSTAYMELRSLTSEDSAVYYCARLEDGYYGYAI |
| | | DYWSQGTSVTVSS |

### Example 4: Singularity Sapiens VH+ Allelic (SSV6)

This Example describes the design and generation of nucleic acid constructs that can be used to engineer non-human animals (e.g., mice) to have the ability to express extra human variable gene segments.

To expand the immunoglobulin repertoire of the singularity sapiens platform, bioinformatics analysis was conducted to identify additional human VH gene segments with functional alleles and different nucleic acid sequences that result in different amino acid sequences of the encoded antibodies.

The international ImMunoGeneTics information System® (IMGTR) site annotated 53 functional VH domain in the known human population. The singularity sapiens V5 (SSV5) contains 44 functional human VH gene segments from 5 incorporated human immunoglobulin heavy chain (hIGH)-bacterial artificial chromosomes (BACs). Sequence comparisons of the nine human VH gene segments not present in SSV5 with those of the SSV5 VH gene segments revealed six VH gene segments (IGHV1-8*01, IGHV3-9*01, IGHV4-31*03, IGHV4-30-4*01, IGHV4-38-2*02, and IGHV3-43D*04) encoding amino acid sequences having at least two amino acid differences compared with their closest homologous VH domains encoded by human VH gene segments contained in SSV5.

IMGT® annotated five VH gene segments as having ORFs (IGHV3-16, IGHV3-38, IGHV3-38-3, IGHV1-38-4, and IGHV7-81). IGHV3-35 had two alleles with allele 01 annotated as ORF due to an unusual V-heptamer sequence and allele 02 as functional. IGHV8-51-1 had four alleles with alleles 01 and 03 as pseudogenes due to the presence of early stop codons, and alleles 02 and 04 as ORFs due to having unusual V-heptamer sequences. The three alleles of IGHV3-62 (01, 02, and 03) were deemed as pseudogenes, while allele 04 was deemed functional.

All 14 VH gene segments were assembled onto a synthetic array. For the eight VH gene segments with functional alleles, the functional allele (IGHV1-8*01, IGHV3-9*01, IGHV4-31*03, IGHV4-30-4*01, IGHV4-38-2*02, IGHV3-43D*04, IGHV3-35*02, and IGHV3-62*04) was selected to be included on the array. Out of the six remaining VH gene segments having no functional alleles (IGHV3-16, IGHV3-38, IGHV3-38-3, IGHV1-38-4, IGHV8-51-1, and IGHV7-81), the following alleles were selected; IGHV3-16*02, IGHV3-38*02, IGHV3-38-3*01, IGHV1-38-4*01, IGHV8-51-1*02, and IGHV7-81*01. IGHV1-38-4*01 was additionally modified such that the nucleic acid sequence encoded a cysteine residue at position 104 instead of a tyrosine residue.

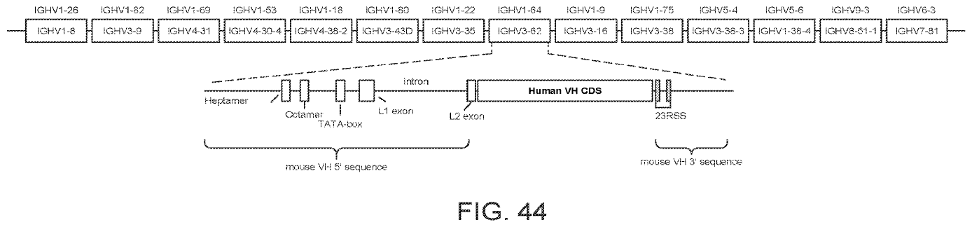

Human VH sequences were mouse-codon optimized. Each VH gene segment on the synthetic hVH+ array had a 5′ upstream sequence containing a VH promoter (250 bp), leader exon 1 and 2, intron, and a 3′ downstream sequence containing the recombination signal sequences (RSS) (100 bp) (FIG. 44). To bypass mutations in the RSS as well as potentially deleterious mutations in the promoter and splicing regions, regulatory elements including promoter, L1, intron, L2, and 3′RSS from a set of murine functional VH gene segments were selected as a framework to drive the expression, splicing and VDJ recombination of human VH gene segments (FIG. 44). 5′ and 3′ sequences of mouse VH gene segments were selected based on the bioinformatic analysis of mouse VH usage rate in the immunoglobulin repertoire from several cohorts of singularity musculus mice that were immunized with several different antigens in multiple campaigns.

### Example 5: Sequences of Exemplary Human VH Gene Segments

IGHV1-8*01
(SEQ ID NO: 18)
CAGGTGCAGCTGGTGCAGTCTGGGGCTGAGGTGAAGAAGCCTGGGGCCTCAGTG
AAGGTCTCCTGCAAGGCTTCTGGATACACCTTCACCAGTTATGATATCAACTGGG
TGCGACAGGCCACTGGACAAGGGCTTGAGTGGATGGGATGGATGAACCCTAACA
GTGGTAACACAGGCTATGCACAGAAGTTCCAGGGCAGAGTCACCATGACCAGGA
ACACCTCCATAAGCACAGCCTACATGGAGCTGAGCAGCCTGAGATCTGAGGACA
CGGCCGTGTATTACTGTGCGAGAGG
codon optimized IGHV1-8*01
(SEQ ID NO: 19)
CAGGTGCAGCTCGTCCAGAGTGGGGCTGAAGTGAAGAAGCCTGGAGCATCTGTG
AAAGTATCCTGCAAAGCGAGTGGCTACACCTTCACCAGCTATGACATCAACTGG
GTGCGGCAGGCAACTGGACAAGGTCTGGAGTGGATGGGCTGGATGAACCCCAAC
AGCGGAAATACTGGCTATGCCCAGAAGTTTCAAGGGCGCGTTACCATGACGAGG
AATACATCCATTTCTACAGCCTACATGGAGCTGAGCTCGTTGCGATCAGAAGATA
CAGCTGTCTATTACTGTGCCAGAGG
IGHV3-9*01
(SEQ ID NO: 20)
GAAGTGCAGCTGGTGGAGTCTGGGGGAGGCTTGGTACAGCCTGGCAGGTCCCTG
AGACTCTCCTGTGCAGCCTCTGGATTCACCTTTGATGATTATGCCATGCACTGGG
TCCGGCAAGCTCCAGGGAAGGGCCTGGAGTGGGTCTCAGGTATTAGTTGGAATA
GTGGTAGCATAGGCTATGCGGACTCTGTGAAGGGCCGATTCACCATCTCCAGAG
ACAACGCCAAGAACTCCCTGTATCTGCAAATGAACAGTCTGAGAGCTGAGGACA
CGGCCTTGTATTACTGTGCAAAAGATA
codon optimized IGHV3-9*01
(SEQ ID NO: 21)
GAAGTGCAGCTGGTGGAGTCTGGAGGAGGGTTGGTTCAGCCAGGAAGATCACTT
CGGCTCAGCTGCGCTGCTAGTGGCTTCACTTTTGATGACTATGCCATGCACTGGG
TAAGGCAAGCTCCTGGGAAAGGCCTGGAGTGGGTCAGCGGGATATCCTGGAACA
GTGGTTCCATTGGCTATGCAGATTCTGTGAAGGGTCGCTTCACCATCTCGAGAGA
CAATGCCAAAAATTCACTCTACCTGCAGATGAACAGCTTACGAGCAGAAGATAC
AGCGCTATACTACTGTGCCAAGGACA
IGHV4-31*03
(SEQ ID NO: 22)
CAGGTGCAGCTGCAGGAGTCGGGCCCAGGACTGGTGAAGCCTTCACAGACCCTG
TCCCTCACCTGCACTGTCTCTGGTGGCTCCATCAGCAGTGGTGGTTACTACTGGA
GCTGGATCCGCCAGCACCCAGGGAAGGGCCTGGAGTGGATTGGGTACATCTATT
ACAGTGGGAGCACCTACTACAACCCGTCCCTCAAGAGTCGAGTTACCATATCAG
TAGACACGTCTAAGAACCAGTTCTCCCTGAAGCTGAGCTCTGTGACTGCCGCGG
ACACGGCCGTGTATTACTGTGCGAGAGA
codon optimized IGHV4-31*03
(SEQ ID NO: 23)
CAAGTCCAGCTGCAGGAGAGTGGACCTGGGCTGGTGAAGCCTAGCCAAACCTTG
AGTCTTACCTGCACTGTGTCGGGTGGCAGCATTAGTTCAGGAGGTTATTACTGGT
CCTGGATCAGACAGCACCCAGGAAAAGGCCTAGAGTGGATTGGCTACATCTATT
ATTCTGGGTCAACGTACTACAACCCCAGCCTGAAGTCCCGGGTCACAATATCTGT
AGACACCAGCAAGAATCAGTTCTCCCTCAAACTCTCATCTGTTACTGCAGCTGAT
ACAGCCGTGTATTACTGTGCCAGGGA
IGHV4-30-4*01
(SEQ ID NO: 24)
CAGGTGCAGCTGCAGGAGTCGGGCCCAGGACTGGTGAAGCCTTCACAGACCCTG
TCCCTCACCTGCACTGTCTCTGGTGGCTCCATCAGCAGTGGTGATTACTACTGGA
GTTGGATCCGCCAGCCCCCAGGGAAGGGCCTGGAGTGGATTGGGTACATCTATT
ACAGTGGGAGCACCTACTACAACCCGTCCCTCAAGAGTCGAGTTACCATATCAG
TAGACACGTCCAAGAACCAGTTCTCCCTGAAGCTGAGCTCTGTGACTGCCGCAG
ACACGGCCGTGTATTACTGTGCCAGAGA
codon optimized IGHV4-30-4*01
(SEQ ID NO: 25)
CAAGTGCAGCTGCAGGAGAGTGGGCCTGGCCTGGTGAAGCCATCCCAAACCCTT
TCGCTCACCTGCACAGTAAGTGGTGGCTCCATATCTTCAGGAGACTATTACTGGA
GCTGGATCAGGCAGCCCCCAGGGAAAGGACTGGAGTGGATTGGCTACATCTACT
ATTCTGGTAGCACGTATTATAATCCTTCCTTGAAAAGCAGAGTTACCATTTCTGT
GGATACTAGCAAGAACCAGTTCTCCCTCAAGCTAAGTTCAGTCACAGCCGCAGA
CACTGCTGTCTACTACTGTGCCCGGGA
IGHV4-38-2*02
(SEQ ID NO: 26)
CAGGTGCAGCTGCAGGAGTCGGGCCCAGGACTGGTGAAGCCTTCGGAGACCCTG
TCCCTCACCTGCACTGTCTCTGGTTACTCCATCAGCAGTGGTTACTACTGGGGCT
GGATCCGGCAGCCCCCAGGGAAGGGGCTGGAGTGGATTGGGAGTATCTATCATA
GTGGGAGCACCTACTACAACCCGTCCCTCAAGAGTCGAGTCACCATATCAGTAG
ACACGTCCAAGAACCAGTTCTCCCTGAAGCTGAGCTCTGTGACCGCCGCAGACA
CGGCCGTGTATTACTGTGCGAGAGA
codon optimized IGHV4-38-2*02
(SEQ ID NO: 27)
CAAGTACAGCTGCAGGAGAGTGGTCCTGGGCTGGTGAAGCCATCAGAAACTCTT
TCACTCACCTGCACTGTTTCAGGCTACAGCATATCCAGTGGCTATTACTGGGGCT
GGATCAGGCAGCCTCCAGGGAAAGGACTAGAGTGGATTGGATCCATCTACCACA
GCGGTAGTACGTATTATAATCCCAGCCTGAAGTCCAGAGTCACCATTTCTGTGGA
CACCAGCAAGAACCAGTTCTCCTTGAAACTCTCTTCTGTCACAGCCGCAGATACA
GCTGTGTACTACTGTGCCCGGGA
IGHV3-43D*04
(SEQ ID NO: 28)
GAAGTGCAGCTGGTGGAGTCTGGGGGAGTCGTGGTACAGCCTGGGGGGTCCCTG
AGACTCTCCTGTGCAGCCTCTGGATTCACCTTTGATGATTATGCCATGCACTGGG
TCCGTCAAGCTCCGGGGAAGGGTCTGGAGTGGGTCTCTCTTATTAGTTGGGATGG
TGGTAGCACATACTATGCAGACTCTGTGAAGGGTCGATTCACCATCTCCAGAGAC
AACAGCAAAAACTCCCTGTATCTGCAAATGAACAGTCTGAGAGCTGAGGACACC
GCCTTGTATTACTGTGCAAAAGATA
codon optimized IGHV3-43D*04
(SEQ ID NO: 29)
GAAGTACAATTGGTGGAGAGTGGCGGTGTTGTCGTGCAGCCTGGAGGTAGTCTC
AGGCTGTCCTGTGCAGCGTCAGGCTTCACTTTTGATGACTATGCCATGCACTGGG
TGCGGCAGGCCCCAGGGAAGGGGCTGGAGTGGGTCTCATTAATTTCCTGGGATG
GAGGCAGCACCTACTATGCAGATTCTGTGAAAGGACGCTTCACGATCTCTAGAG
ACAATTCCAAGAACAGCCTATATCTGCAGATGAACAGCCTTCGAGCTGAAGACA
CAGCTCTCTACTACTGCGCCAAGGACA
IGHV3-35*02
(SEQ ID NO: 30)
GAGGTGCAGCTGGTGGAGTCTGGGGGAGGCTTGGTACAGCCTGGGGGATCCCTG
AGACTCTCCTGTGCAGCCTCTGGATTCACCTTCAGTAACAGTGACATGAACTGGG
TCCATCAGGCTCCAGGAAAGGGGCTGGAGTGGGTATCGGGTGTTAGTTGGAATG
GCAGTAGGACGCACTATGCAGACTCTGTGAAGGGCCAATTCATCATCTCCAGAG
ACAATTCCAGGAACACCCTGTATCTGCAAACGAATAGCCTGAGGGCCGAGGACA
CGGCTGTGTATTACTGTGTGAGAAA
codon optimized IGHV3-35*02
(SEQ ID NO: 31)
GAAGTGCAGCTGGTGGAGAGTGGGGGTGGTCTCGTTCAGCCTGGAGGAAGTCTA
CGGCTTTCCTGTGCCGCGTCAGGCTTTACCTTCAGCAATTCAGACATGAACTGGG
TGCATCAGGCCCCAGGGAAGGGCCTGGAGTGGGTATCTGGAGTCTCTTGGAATG
GCAGCCGTACACACTATGCTGATTCTGTTAAGGGGCAGTTCATCATTTCCCGCGA
TAATTCCAGGAACACACTCTACCTGCAAACCAACAGCTTGCGAGCAGAAGACAC
TGCTGTGTATTACTGCGTCAGAAA
IGHV3-62*04
(SEQ ID NO: 32)
GAGGTGCAGCTGGTGAAGTCTGGAGGAGGCTTGGTACAGCCTGGGGGGTCCCTG
AGACTCTCCTGTGCAGCCTCTGGATTCACCTTCAGTAGCTCTGCTATGCACTGGG
TCCGCCAGGCTCCAAGAAAGGGTTTGGAGTGGGTCTCAGTTATTAGTACAAGTG
GTGATACCGTACTCTACACAGACTCTGTGAAGGGCCGATTCACCATCTCCAGAG
ACAATGCCCAGAATTCACTGTCTCTGCAAATGAACAGCCTGAGAGCCGAGGACA
TGGCTGTGTATTACTGTGTGAAAGA
codon optimized IGHV3-62*04
(SEQ ID NO: 33)
GAAGTACAGCTGGTGAAAAGTGGTGGTGGGCTCGTTCAACCTGGAGGCTCCCTT
CGCCTCAGCTGTGCTGCGAGTGGCTTCACCTTCAGCTCCTCAGCCATGCACTGGG
TTCGGCAGGCACCAAGGAAGGGCCTGGAGTGGGTGTCTGTCATTTCCACTTCTGG
GGACACAGTGCTGTATACAGATAGTGTCAAAGGACGATTTACCATCAGCAGAGA
TAATGCCCAGAACTCATTGTCTCTGCAGATGAACAGCCTAAGAGCAGAGGACAT
GGCTGTGTACTACTGCGTGAAGGA
IGHV3-16*02
(SEQ ID NO: 34)
GAGGTGCAGCTGGTGGAGTCTGGGGGAGGCTTGGTACAGCCTGGGGGGTCCCTG
AGACTCTCCTGTGCAGCCTCTGGATTCACCTTCAGTAACAGTGACATGAACTGGG
CCCGCAAGGCTCCAGGAAAGGGGCTGGAGTGGGTATCGGGTGTTAGTTGGAATG
GCAGTAGGACGCACTATGTGGACTCCGTGAAGCGCCGATTCATCATCTCCAGAG
ACAATTCCAGGAACTCCCTGTATCTGCAAAAGAACAGACGGAGAGCCGAGGACA
TGGCTGTGTATTACTGTGTGAGAAA
codon optimized IGHV3-16*02
(SEQ ID NO: 35)
GAAGTGCAGCTGGTGGAGAGTGGTGGTGGCTTGGTTCAACCAGGAGGCAGCCTA
AGGCTTAGCTGTGCTGCCAGTGGCTTCACATTTTCCAATTCAGATATGAACTGGG
CAAGGAAGGCCCCTGGGAAAGGACTGGAGTGGGTGTCAGGGGTCTCCTGGAATG
GATCCCGTACCCACTATGTGGATTCTGTAAAGCGCCGCTTCATCATTTCTCGGGA
CAACAGCAGAAATTCTCTCTATCTGCAGAAGAACAGAAGACGAGCGGAAGACAT
GGCTGTCTACTACTGCGTTCGGAA
IGHV3-38*02
(SEQ ID NO: 36)
GAGGTGCAGCTGGTGGAGTCTGGGGGAGGCTTGGTACAGCCTAGGGGGTCCCTG
AGACTCTCCTGTGCAGCCTCTGGATTCACCGTCAGTAGCAATGAGATGAGCTGGA
TCCGCCAGGCTCCAGGGAAGGGGCTGGAGTGGGTCTCATCCATTAGTGGTGGTA
GCACATACTACGCAGACTCCAGGAAGGGCAGATTCACCATCTCCAGAGACAATT
CCAAGAACACGCTGTATCTTCAAATGAACAACCTGAGAGCTGAGGGCACGGCCG
TGTATTACTGTGCCAGATATA
codon optimized IGHV3-38*02
(SEQ ID NO: 37)
GAAGTGCAGCTGGTGGAGAGTGGAGGTGGTCTAGTCCAGCCTCGAGGGAGTTTG
AGGCTTAGCTGTGCTGCCTCTGGCTTCACTGTTTCTTCCAATGAGATGTCTTGGAT
CCGGCAAGCCCCAGGGAAGGGCCTGGAGTGGGTGTCCAGCATTTCAGGAGGAA
GCACCTACTATGCAGATTCAAGGAAAGGCCGTTTTACCATATCCAGAGACAACA
GCAAGAATACACTCTACCTGCAGATGAACAACCTCAGAGCAGAAGGGACAGCTG
TATATTACTGCGCCCGCTATA
IGHV3-38-3*01
(SEQ ID NO: 38)
GAGGTGCAGCTGGTGGAGTCTCGGGGAGTCTTGGTACAGCCTGGGGGGTCCCTG
AGACTCTCCTGTGCAGCCTCTGGATTCACCGTCAGTAGCAATGAGATGAGCTGGG
TCCGCCAGGCTCCAGGGAAGGGTCTGGAGTGGGTCTCATCCATTAGTGGTGGTA
GCACATACTACGCAGACTCCAGGAAGGGCAGATTCACCATCTCCAGAGACAATT
CCAAGAACACGCTGCATCTTCAAATGAACAGCCTGAGAGCTGAGGACACGGCTG
TGTATTACTGTAAGAAAGA
codon optimized IGHV3-38-3*01
(SEQ ID NO: 39)
GAAGTGCAGCTAGTGGAGTCAAGAGGTGTTTTGGTCCAGCCTGGAGGAAGTCTC
AGGCTCTCCTGTGCCGCGTCTGGCTTTACTGTATCAAGTAATGAGATGTCCTGGG
TGAGGCAAGCCCCAGGGAAAGGCCTGGAGTGGGTCAGCAGTATTTCTGGTGGCA
GCACCTATTATGCTGACAGCCGGAAAGGGCGCTTCACCATCTCCAGAGACAACA
GCAAGAACACACTGCACCTGCAGATGAATTCTCTTCGAGCAGAAGATACAGCTG
TGTACTACTGCAAGAAGGA
IGHV1-38-4*01
(SEQ ID NO: 40)
CAGGTCCAGCTGGTGCAGTCTTGGGCTGAGGTGAGGAAGTCTGGGGCCTCAGTG
AAAGTCTCCTGTAGTTTTTCTGGGTTTACCATCACCAGCTACGGTATACATTGGG
TGCAACAGTCCCCTGGACAAGGGCTTGAGTGGATGGGATGGATCAACCCTGGCA
ATGGTAGCCCAAGCTATGCCAAGAAGTTTCAGGGCAGATTCACCATGACCAGGG
ACATGTCCACAACCACAGCCTACACAGACCTGAGCAGCCTGACATCTGAGGACA
TGGCTGTGTATTACTGTGCAAGACA
codon optimized IGHV1-38-4*01
(SEQ ID NO: 41)
CAGGTGCAGCTGGTTCAGTCCTGGGCAGAGGTGAGGAAAAGTGGTGCTTCTGTA
AAAGTCTCCTGCAGCTTTTCTGGCTTCACCATAACCAGCTATGGAATTCACTGGG
TCCAGCAGAGCCCCGGGCAAGGGCTGGAGTGGATGGGCTGGATCAACCCTGGAA
ATGGCTCGCCATCCTATGCCAAGAAGTTCCAGGGTCGCTTTACCATGACAAGAG
ATATGTCTACTACGACAGCCTACACAGACCTCTCAAGTCTTACTTCAGAAGACAT
GGCTGTGTACTACTGTGCGCGGCA
IGHV8-51-1*02
(SEQ ID NO: 42)
GAGGCCCAGCTTACAGAGTCTGGGGGAGACTTGGTACACTTAGAGGGGCCCCTG
AGGCTCTCCTGTGCAGCCTCTTGGTTCACCTTCAGTATCTATGAGATTCACTGGGT
TTGCCAGGCCTCAGGGAAGGGGCTGGAATGGGTTGCAGTTATATGGCGTGGTGA
AAGTCATCAATACAATGCAGACTATGTTAGGGGCAGACTCACCACTTCCAGAGA
CAACACCAAGTACATGCTGTACATGCAAATGATCAGCCTGAGAACCCAGAACAT
GGCAGCATTTAACTGTGCAGGAAA
codon optimized IGHV8-51-1*02
(SEQ ID NO: 43)
GAAGCCCAGCTGACAGAAAGTGGAGGAGACCTGGTGCACCTAGAAGGCCCTCTT
CGGCTCAGCTGCGCGGCCTCCTGGTTCACCTTTTCCATATATGAGATCCACTGGG
TCTGCCAAGCTTCTGGGAAAGGCCTGGAGTGGGTGGCTGTAATCTGGAGGGGGG
AGAGCCATCAGTACAATGCAGATTATGTTCGAGGTCGTCTCACAACGTCAAGAG
ACAATACCAAGTACATGCTGTACATGCAGATGATTTCTTTGCGCACTCAGAACAT
GGCTGCCTTCAACTGTGCAGGCAA
IGHV7-81*01
(SEQ ID NO: 44)
CAGGTGCAGCTGGTGCAGTCTGGCCATGAGGTGAAGCAGCCTGGGGCCTCAGTG
AAGGTCTCCTGCAAGGCTTCTGGTTACAGTTTCACCACCTATGGTATGAATTGGG
TGCCACAGGCCCCTGGACAAGGGCTTGAGTGGATGGGATGGTTCAACACCTACA
CTGGGAACCCAACATATGCCCAGGGCTTCACAGGACGGTTTGTCTTCTCCATGGA
CACCTCTGCCAGCACAGCATACCTGCAGATCAGCAGCCTAAAGGCTGAGGACAT
GGCCATGTATTACTGTGCGAGATA
codon optimized IGHV7-81*01
(SEQ ID NO: 45)
CAAGTTCAGCTGGTGCAGAGTGGTCACGAAGTGAAGCAGCCAGGAGCATCTGTC
AAAGTCAGCTGCAAAGCATCAGGCTACAGCTTCACCACTTATGGCATGAACTGG
GTGCCTCAGGCTCCGGGGCAAGGTTTGGAGTGGATGGGCTGGTTCAACACCTAC
ACAGGGAATCCCACCTATGCCCAGGGCTTCACAGGAAGGTTTGTATTTTCCATGG
ATACTTCTGCTTCGACGGCGTATCTGCAGATCTCCAGTCTCAAGGCTGAGGACAT
GGCCATGTACTACTGTGCCAGATG

### Example 6: Exemplary Singularity Sapiens VH+ Arrays

In each nucleic acid sequence, the sequence is annotated with each component indicated by plain text, underlined text, or by bold and underlined text as follows:

```
mouse promoter + mouse L1 exon + mouse intron + mouse L2 exon + human VH + mouse RSS
  1) Mouse VH: IGHV1-26*01 / Human VH: IGHV1-8*01
Nucleic acid sequence present on synthetic array with human VH:
(SEQ ID NO: 46)
TGTATGGGATCCATTTCCTCAGAGAGTTATTGGATTTGGACTAGACTATCCTGCT
GCTTGACCTATGTACCTTTAAGTCCTTCCTCTCCAGCTTTTCTTCATTCGGATTGG
TTATTATATACAAAGTCCCCTGGTCATGAATATGCAAAATACCTAAGTCTATGGT
AGCTAAAAACAGGGATATCAACACCCTGAAAACAACATATGTACAATGTCCTCA
CCACAGACACTGAACACACTGACTCTAACCATGGGATGGAGCTGGATCTTTCTCT
TTCTCCTGTCAGGAACTGCAGGTAAGGGGCTCACCAGTTCCAAATCTGAAGAAA
AGAAATGGCTTGGGATGTCACTGACATCCACTCTGTCTTTCTCTTCACAGGTGTC
CTCTCTCAGGTGCAGCTGGTGCAGTCTGGGGCTGAGGTGAAGAAGCCTGGG
GCCTCAGTGAAGGTCTCCTGCAAGGCTTCTGGATACACCTTCACCAGTTATG
ATATCAACTGGGTGCGACAGGCCACTGGACAAGGGCTTGAGTGGATGGGAT
GGATGAACCCTAACAGTGGTAACACAGGCTATGCACAGAAGTTCCAGGGCA
GAGTCACCATGACCAGGAACACCTCCATAAGCACAGCCTACATGGAGCTGA
GCAGCCTGAGATCTGAGGACACGGCCGTGTATTACTGTGCGAGAGGCACAG
TGCTACAAACACATCCTGAGTGTGTCAGAAACCCTGGAGGTGCAGCAAGCTCCC
TTGGGATTGACAAGACTTAGAGAATAGCCGCTTGCAGACTT
Nucleic acid sequence present on synthetic array with codon optimized human VH:
(SEQ ID NO: 47)
TGTATGGGATCCATTTCCTCAGAGAGTTATTGGATTTGGACTAGACTATCCTGCT
GCTTGACCTATGTACCTTTAAGTCCTTCCTCTCCAGCTTTTCTTCATTCGGATTGG
TTATTATATACAAAGTCCCCTGGTCATGAATATGCAAAATACCTAAGTCTATGGT
AGCTAAAAACAGGGATATCAACACCCTGAAAACAACATATGTACAATGTCCTCA
CCACAGACACTGAACACACTGACTCTAACCATGGGATGGAGCTGGATCTTTCTCT
TTCTCCTGTCAGGAACTGCAGGTAAGGGGCTCACCAGTTCCAAATCTGAAGAAA
AGAAATGGCTTGGGATGTCACTGACATCCACTCTGTCTTTCTCTTCACAGGTGTC
CTCTCTCAGGTGCAGCTCGTCCAGAGTGGGGCTGAAGTGAAGAAGCCTGGA
GCATCTGTGAAAGTATCCTGCAAAGCGAGTGGCTACACCTTCACCAGCTAT
GACATCAACTGGGTGCGGCAGGCAACTGGACAAGGTCTGGAGTGGATGGG
CTGGATGAACCCCAACAGCGGAAATACTGGCTATGCCCAGAAGTTTCAAGG
GCGCGTTACCATGACGAGGAATACATCCATTTCTACAGCCTACATGGAGCT
GAGCTCGTTGCGATCAGAAGATACAGCTGTCTATTACTGTGCCAGAGGCAC
AGTGCTACAAACACATCCTGAGTGTGTCAGAAACCCTGGAGGTGCAGCAAGCTC
CCTTGGGATTGACAAGACTTAGAGAATAGCCGCTTGCAGACTT
  2) Mouse VH: IGHV1-82*01 / Human VH: IGHV3-9*01
Nucleic acid sequence present on synthetic array with human VH:
(SEQ ID NO: 48)
GTTTGGGATGCTTTTCCTCAGGGAGGATTATGACTTGGACCCTAGCATCCTGCTG
CATGACCCATGTGCCTTTTCAGTGCTTTCTCCCTAGTTCTTCTCCAGCTGGACTAG
GTCATTAACTAAGAAATGCACTGCTCATGAATATGCAAATTACTTGAGCCTATGG
TAGTAAATACAGGCATGCCCACACTGTGAAAACAACATATGACTCCTGTCTTCTC
CACAGTCCCAGAACACACTCACTCTAACCATGGAATGGCCTTGTATCTTTCTCTT
CCTCCTGTCAGTAACTGAAGGTAAGGAGCTCAGCAGTTCCAATCTGAAGAGGAG
ATAGGGCCTGAGGTGACAATGACATCCACAATTTCTTTCTCCCGACAGGTGTCCA
CTCCGAAGTGCAGCTGGTGGAGTCTGGGGGAGGCTTGGTACAGCCTGGCAG
GTCCCTGAGACTCTCCTGTGCAGCCTCTGGATTCACCTTTGATGATTATGCC
ATGCACTGGGTCCGGCAAGCTCCAGGGAAGGGCCTGGAGTGGGTCTCAGG
TATTAGTTGGAATAGTGGTAGCATAGGCTATGCGGACTCTGTGAAGGGCCG
ATTCACCATCTCCAGAGACAACGCCAAGAACTCCCTGTATCTGCAAATGAAC
AGTCTGAGAGCTGAGGACACGGCCTTGTATTACTGTGCAAAAGATACACAGT
GTTACAACCACATCCTGAGAGTGTCAGAAACCGTGGAGGAGCAGGAAGCTTCCC
TGGGCCTGAGATGACAGAAAGATTAATCTTTAGACTTGCT
Nucleic acid sequence present on synthetic array with codon optimized human VH:
(SEQ ID NO: 49)
GTTTGGGATGCTTTTCCTCAGGGAGGATTATGACTTGGACCCTAGCATCCTGCTG
CATGACCCATGTGCCTTTTCAGTGCTTTCTCCCTAGTTCTTCTCCAGCTGGACTAG
GTCATTAACTAAGAAATGCACTGCTCATGAATATGCAAATTACTTGAGCCTATGG
TAGTAAATACAGGCATGCCCACACTGTGAAAACAACATATGACTCCTGTCTTCTC
CACAGTCCCAGAACACACTCACTCTAACCATGGAATGGCCTTGTATCTTTCTCTT
CCTCCTGTCAGTAACTGAAGGTAAGGAGCTCAGCAGTTCCAATCTGAAGAGGAG
ATAGGGCCTGAGGTGACAATGACATCCACAATTTCTTTCTCCCGACAGGTGTCCA
CTCCGAAGTGCAGCTGGTGGAGTCTGGAGGAGGGTTGGTTCAGCCAGGAAG
ATCACTTCGGCTCAGCTGCGCTGCTAGTGGCTTCACTTTTGATGACTATGCC
ATGCACTGGGTAAGGCAAGCTCCTGGGAAAGGCCTGGAGTGGGTCAGCGG
GATATCCTGGAACAGTGGTTCCATTGGCTATGCAGATTCTGTGAAGGGTCG
CTTCACCATCTCGAGAGACAATGCCAAAAATTCACTCTACCTGCAGATGAAC
AGCTTACGAGCAGAAGATACAGCGCTATACTACTGTGCCAAGGACACACAG
TGTTACAACCACATCCTGAGAGTGTCAGAAACCGTGGAGGAGCAGGAAGCTTCC
CTGGGCCTGAGATGACAGAAAGATTAATCTTTAGACTTGCT
  3) Mouse VH: IGHV1-69*01 / Human VH: IGHV4-31*03
Nucleic acid sequence present on synthetic array with human VH:
(SEQ ID NO: 50)
ACAAGGTTCAGGAATGAGGTATGGGATTAATTTCATCAGACAGGACAATGACTT
GTGCCTCAGCATCCTGATTTCTGACCCAGGTGTCCCTTCTTCTCCAGCAGGAGTA
GGTGCTTATATAATATGTATCCTGCTCATAAATATGCAAATCCTGTGAATCTACA
GTGGTAAATATAGGGTTGTCTACACCACACAAAAAAGCATAAGATCACTGTTCT
CTCTACAGTTACTGAGCACACAGGACCTCACCATGGGATGGAGCTGTATCATCCT
CTTCTTGGTATCAACAGCTACAGGTAAGGGGCTCACAGGTAAGCAGGCTTGAGA
ACTGGCCATACCTGTGGGTGAAAATGACATCCACTCTCTCTTTCTCTCCACAGGT
GTCCACTCCCAGGTGCAGCTGCAGGAGTCGGGCCCAGGACTGGTGAAGCCT
TCACAGACCCTGTCCCTCACCTGCACTGTCTCTGGTGGCTCCATCAGCAGTG
GTGGTTACTACTGGAGCTGGATCCGCCAGCACCCAGGGAAGGGCCTGGAGT
GGATTGGGTACATCTATTACAGTGGGAGCACCTACTACAACCCGTCCCTCA
AGAGTCGAGTTACCATATCAGTAGACACGTCTAAGAACCAGTTCTCCCTGAA
GCTGAGCTCTGTGACTGCCGCGGACACGGCCGTGTATTACTGTGCGAGAGA
CACAGTGTTGCAACCACATCCTGAGAGTGTCAGAAACCCTGGAGGAGTAGCAAA
CTGCCCTGAGACTGAGGAGACTCAGAGAAGGTTTGCTTGTAGACTT
Nucleic acid sequence present on synthetic array with codon optimized human VH:
(SEQ ID NO: 51)
ACAAGGTTCAGGAATGAGGTATGGGATTAATTTCATCAGACAGGACAATGACTT
GTGCCTCAGCATCCTGATTTCTGACCCAGGTGTCCCTTCTTCTCCAGCAGGAGTA
GGTGCTTATATAATATGTATCCTGCTCATAAATATGCAAATCCTGTGAATCTACA
GTGGTAAATATAGGGTTGTCTACACCACACAAAAAAGCATAAGATCACTGTTCT
CTCTACAGTTACTGAGCACACAGGACCTCACCATGGGATGGAGCTGTATCATCCT
CTTCTTGGTATCAACAGCTACAGGTAAGGGGCTCACAGGTAAGCAGGCTTGAGA
ACTGGCCATACCTGTGGGTGAAAATGACATCCACTCTCTCTTTCTCTCCACAGGT
GTCCACTCCCAAGTCCAGCTGCAGGAGAGTGGACCTGGGCTGGTGAAGCCT
AGCCAAACCTTGAGTCTTACCTGCACTGTGTCGGGTGGCAGCATTAGTTCA
GGAGGTTATTACTGGTCCTGGATCAGACAGCACCCAGGAAAAGGCCTAGAG
TGGATTGGCTACATCTATTATTCTGGGTCAACGTACTACAACCCCAGCCTGA
AGTCCCGGGTCACAATATCTGTAGACACCAGCAAGAATCAGTTCTCCCTCAA
ACTCTCATCTGTTACTGCAGCTGATACAGCCGTGTATTACTGTGCCAGGGAC
ACAGTGTTGCAACCACATCCTGAGAGTGTCAGAAACCCTGGAGGAGTAGCAAAC
TGCCCTGAGACTGAGGAGACTCAGAGAAGGTTTGCTTGTAGACTT
  4) Mouse VH: IGHV1-53*01 / Human VH: IGHV4-30-4*01
Nucleic acid sequence present on synthetic array with human VH:
(SEQ ID NO: 52)
ACAAGGTTCAGGGATGAGGTATGGGATGCATTTCCTTAGACAAGACAAGGACTT
GGGCTTCAGCAACCTGATTCTTGACCCAGATGTCCCTTCTTCTCCAGCAGGAGTA
GGTGCTTATCTAATATGTATCCTGCTCATAAATATGCAAATCCTGTGAATCTACA
GTGGTAAATTTAGGGTTGTCTACACGACACAAAAAAGCATAAGATCACTGTTCTC
TCTACAGTTACTAAGCACACAGGATCTCACCATGGGATGGAGCTGTATCATCCTC
TTTTTGGTAGCAGCAGCTACAGGTAAGGGGCTCACAGTAGCAGGCTTGAGGTCT
GGCCATATACATGGGTGACAATGACATCCACTTTGCCTTTCTCTCCACAGGTGTC
CACTCCCAGGTGCAGCTGCAGGAGTCGGGCCCAGGACTGGTGAAGCCTTCA
CAGACCCTGTCCCTCACCTGCACTGTCTCTGGTGGCTCCATCAGCAGTGGT
GATTACTACTGGAGTTGGATCCGCCAGCCCCCAGGGAAGGGCCTGGAGTGG
ATTGGGTACATCTATTACAGTGGGAGCACCTACTACAACCCGTCCCTCAAGA
GTCGAGTTACCATATCAGTAGACACGTCCAAGAACCAGTTCTCCCTGAAGCT
GAGCTCTGTGACTGCCGCAGACACGGCCGTGTATTACTGTGCCAGAGACAC
AGTGTTGCAACCACATCCTGAGAGTGTCAGAAACCCTGGATGAGTAGCAAACTG
CCCTGAGACTGAGGAGACTCAGAGAAGGTTTGCTTGTAGACTT
Nucleic acid sequence present on synthetic array with codon optimized human VH:
(SEQ ID NO: 53)
ACAAGGTTCAGGGATGAGGTATGGGATGCATTTCCTTAGACAAGACAAGGACTT
GGGCTTCAGCAACCTGATTCTTGACCCAGATGTCCCTTCTTCTCCAGCAGGAGTA
GGTGCTTATCTAATATGTATCCTGCTCATAAATATGCAAATCCTGTGAATCTACA
GTGGTAAATTTAGGGTTGTCTACACGACACAAAAAAGCATAAGATCACTGTTCTC
TCTACAGTTACTAAGCACACAGGATCTCACCATGGGATGGAGCTGTATCATCCTC
TTTTTGGTAGCAGCAGCTACAGGTAAGGGGCTCACAGTAGCAGGCTTGAGGTCT
GGCCATATACATGGGTGACAATGACATCCACTTTGCCTTTCTCTCCACAGGTGTC
CACTCCCAAGTGCAGCTGCAGGAGAGTGGGCCTGGCCTGGTGAAGCCATCC
CAAACCCTTTCGCTCACCTGCACAGTAAGTGGTGGCTCCATATCTTCAGGAG
ACTATTACTGGAGCTGGATCAGGCAGCCCCCAGGGAAAGGACTGGAGTGGA
TTGGCTACATCTACTATTCTGGTAGCACGTATTATAATCCTTCCTTGAAAAG
CAGAGTTACCATTTCTGTGGATACTAGCAAGAACCAGTTCTCCCTCAAGCTA
AGTTCAGTCACAGCCGCAGACACTGCTGTCTACTACTGTGCCCGGGACACA
GTGTTGCAACCACATCCTGAGAGTGTCAGAAACCCTGGATGAGTAGCAAACTGC
CCTGAGACTGAGGAGACTCAGAGAAGGTTTGCTTGTAGACTT
  5) Mouse VH: IGHV1-18*01 / Human VH: IGHV4-38-2*02
Nucleic acid sequence present on synthetic array with human VH:
(SEQ ID NO: 54)
TGAATGGGATCTATTTCTTCAGAGAGTTATTGGATTTGTACTAGACCATCCTACT
GCTTGACCTATGTACCTTTAATTCCTTCCTCTCCAGCTGTTCTCCATTTGGATTGG
TTAGTATATACAAAGTCCCCTGGTCATGACTATGCAAATTACCTAAGTCTATGGT
AGTTAAAAACAGGGATATCAACACCCTGAAAACAACATATGTCCAATGTCCTCT
CCACAGGCACTGAACACACTGACTCTAACCATGGGATGGAGCTGGATCTTTCTCC
TCTTCCTGTCAGGAACTGCAGGTAAGGGGCTCACCATTTCCAAATCTGAAGAAA
AGAAATGGCTTGGGATGTCACTGAAATCCACTCTGTCTTTCTCTTCACAGGTGTC
CTCTCTCAGGTGCAGCTGCAGGAGTCGGGCCCAGGACTGGTGAAGCCTTCG
GAGACCCTGTCCCTCACCTGCACTGTCTCTGGTTACTCCATCAGCAGTGGTT
ACTACTGGGGCTGGATCCGGCAGCCCCCAGGGAAGGGGCTGGAGTGGATT
GGGAGTATCTATCATAGTGGGAGCACCTACTACAACCCGTCCCTCAAGAGT
CGAGTCACCATATCAGTAGACACGTCCAAGAACCAGTTCTCCCTGAAGCTG
AGCTCTGTGACCGCCGCAGACACGGCCGTGTATTACTGTGCGAGAGACACA
GTGCTACAAACACATCCTGAGTGTGTCAGAAACCTTGGAGGTGCAGCAAGCTCC
CTTGGGACTGACAAGACTTAGAGAAGAGTCGCTTGCAGACTT
Nucleic acid sequence present on synthetic array with codon optimized human VH:
(SEQ ID NO: 55)
TGAATGGGATCTATTTCTTCAGAGAGTTATTGGATTTGTACTAGACCATCCTACT
GCTTGACCTATGTACCTTTAATTCCTTCCTCTCCAGCTGTTCTCCATTTGGATTGG
TTAGTATATACAAAGTCCCCTGGTCATGACTATGCAAATTACCTAAGTCTATGGT
AGTTAAAAACAGGGATATCAACACCCTGAAAACAACATATGTCCAATGTCCTCT
CCACAGGCACTGAACACACTGACTCTAACCATGGGATGGAGCTGGATCTTTCTCC
TCTTCCTGTCAGGAACTGCAGGTAAGGGGCTCACCATTTCCAAATCTGAAGAAA
AGAAATGGCTTGGGATGTCACTGAAATCCACTCTGTCTTTCTCTTCACAGGTGTC
CTCTCTCAAGTACAGCTGCAGGAGAGTGGTCCTGGGCTGGTGAAGCCATCA
GAAACTCTTTCACTCACCTGCACTGTTTCAGGCTACAGCATATCCAGTGGCT
ATTACTGGGGCTGGATCAGGCAGCCTCCAGGGAAAGGACTAGAGTGGATTG
GATCCATCTACCACAGCGGTAGTACGTATTATAATCCCAGCCTGAAGTCCAG
AGTCACCATTTCTGTGGACACCAGCAAGAACCAGTTCTCCTTGAAACTCTCT
TCTGTCACAGCCGCAGATACAGCTGTGTACTACTGTGCCCGGGACACAGTGC
TACAAACACATCCTGAGTGTGTCAGAAACCTTGGAGGTGCAGCAAGCTCCCTTG
GGACTGACAAGACTTAGAGAAGAGTCGCTTGCAGACTT
  6) Mouse VH: IGHV1-80*01 / Human VH: IGHV3-43D*04
Nucleic acid sequence present on synthetic array with human VH:
(SEQ ID NO: 56)
GTTTGGGATGCTTTTCCTCAGGGAGGATTATGACTTAGACCCTAGCATCCTGCTG
CCTGACCCATGTGCCTTTTCAGTGCTTTCTCCCTAGTTCTTCTCCAGCTGGACTAG
GTCATTAAGTAAGAAATGCACTGCTCATGAATATGCAAATTACTTGAGCCTATGG
TAGTAAATACAGGCATGCCCACACTGTGAAAACAGCATATGACCCCTGTCTTCTC
CACAGTCCCTGAACACACTGACTCTAACCATGGAATGGCCTTTGATCTTTCTCTT
CCTCCTGTCAGGAACTGCAGGTAAGGGGCTCAGCAGTTCCAATCTGAAGAGGAG
ACAGGGCCTGAGGTGACAATGACATCCACAATGACTTTCTCCCAACAGGTGTCC
AATCCGAAGTGCAGCTGGTGGAGTCTGGGGGAGTCGTGGTACAGCCTGGGG
GGTCCCTGAGACTCTCCTGTGCAGCCTCTGGATTCACCTTTGATGATTATGC
CATGCACTGGGTCCGTCAAGCTCCGGGGAAGGGTCTGGAGTGGGTCTCTCT
TATTAGTTGGGATGGTGGTAGCACATACTATGCAGACTCTGTGAAGGGTCG
ATTCACCATCTCCAGAGACAACAGCAAAAACTCCCTGTATCTGCAAATGAAC
AGTCTGAGAGCTGAGGACACCGCCTTGTATTACTGTGCAAAAGATACACAGT
GTTGCAACCACATCCTGAGTGTGTCAGAAATCCTGGAGGAGCAGGAAGCTTCCC
TGGGCCTGAGATGACAGAAAGATTAATCTTTAGACTTGCT
Nucleic acid sequence present on synthetic array with codon optimized human VH:
(SEQ ID NO: 57)
GTTTGGGATGCTTTTCCTCAGGGAGGATTATGACTTAGACCCTAGCATCCTGCTG
CCTGACCCATGTGCCTTTTCAGTGCTTTCTCCCTAGTTCTTCTCCAGCTGGACTAG
GTCATTAAGTAAGAAATGCACTGCTCATGAATATGCAAATTACTTGAGCCTATGG
TAGTAAATACAGGCATGCCCACACTGTGAAAACAGCATATGACCCCTGTCTTCTC
CACAGTCCCTGAACACACTGACTCTAACCATGGAATGGCCTTTGATCTTTCTCTT
CCTCCTGTCAGGAACTGCAGGTAAGGGGCTCAGCAGTTCCAATCTGAAGAGGAG
ACAGGGCCTGAGGTGACAATGACATCCACAATGACTTTCTCCCAACAGGTGTCC
AATCCGAAGTACAATTGGTGGAGAGTGGCGGTGTTGTCGTGCAGCCTGGAG
GTAGTCTCAGGCTGTCCTGTGCAGCGTCAGGCTTCACTTTTGATGACTATGC
CATGCACTGGGTGCGGCAGGCCCCAGGGAAGGGGCTGGAGTGGGTCTCAT
TAATTTCCTGGGATGGAGGCAGCACCTACTATGCAGATTCTGTGAAAGGAC
GCTTCACGATCTCTAGAGACAATTCCAAGAACAGCCTATATCTGCAGATGAA
CAGCCTTCGAGCTGAAGACACAGCTCTCTACTACTGCGCCAAGGACACACA
GTGTTGCAACCACATCCTGAGTGTGTCAGAAATCCTGGAGGAGCAGGAAGCTTC
CCTGGGCCTGAGATGACAGAAAGATTAATCTTTAGACTTGCT
  7) Mouse VH: IGHV1-22*01 / Human VH: IGHV3-35*02
(SEQ ID NO: 58)
Nucleic acid sequence present on synthetic array with human VH:
TGTATGGGATCCATTTCCTCAGAGAGTTATTGGATTTGGACTAGACCATCCTGCT
GCTTGACCTATGTACCTTTAAGTCCTTCCTCTCCATCTATTCTCCATTTGGATTGG
TTATTATATACAAAGTCCCCTGGTCATGAATATGCAAATTACCTAAGTCTATCGT
AGTTAAAAACAGGGATATCAACATCCTGAAAACAACATATGTCCAATGTCCTCT
CCTCAGACACTGAACACACTGACTCTAACCATGGGATGGAGCTGGATCTTTCTCT
TTCTCCTGTCAGAAACTGCAGGTAAGGGGCTCACCATTTCCAAATCTGAAGAAA
AGAAATGGCTAGGGATGTCACTGACATCCACTCTGTCTTTCTCTTCACAGGTGTC
CTCTCTGAGGTGCAGCTGGTGGAGTCTGGGGGAGGCTTGGTACAGCCTGGG
GGATCCCTGAGACTCTCCTGTGCAGCCTCTGGATTCACCTTCAGTAACAGTG
ACATGAACTGGGTCCATCAGGCTCCAGGAAAGGGGCTGGAGTGGGTATCGG
GTGTTAGTTGGAATGGCAGTAGGACGCACTATGCAGACTCTGTGAAGGGCC
AATTCATCATCTCCAGAGACAATTCCAGGAACACCCTGTATCTGCAAACGAA
TAGCCTGAGGGCCGAGGACACGGCTGTGTATTACTGTGTGAGAAACACAGT
GCTACAAACACATCCTCAGTGTGTCAGAAATCCTGGAGGTGCAGCAAGCTCCCT
TGGGACTGACAAGACTTAGAGAATAGTTGCTTGCAGACGT
Nucleic acid sequence present on synthetic array with codon optimized human VH:
(SEQ ID NO: 59)
TGTATGGGATCCATTTCCTCAGAGAGTTATTGGATTTGGACTAGACCATCCTGCT
GCTTGACCTATGTACCTTTAAGTCCTTCCTCTCCATCTATTCTCCATTTGGATTGG
TTATTATATACAAAGTCCCCTGGTCATGAATATGCAAATTACCTAAGTCTATCGT
AGTTAAAAACAGGGATATCAACATCCTGAAAACAACATATGTCCAATGTCCTCT
CCTCAGACACTGAACACACTGACTCTAACCATGGGATGGAGCTGGATCTTTCTCT
TTCTCCTGTCAGAAACTGCAGGTAAGGGGCTCACCATTTCCAAATCTGAAGAAA
AGAAATGGCTAGGGATGTCACTGACATCCACTCTGTCTTTCTCTTCACAGGTGTC
CTCTCTGAAGTGCAGCTGGTGGAGAGTGGGGGTGGTCTCGTTCAGCCTGGA
GGAAGTCTACGGCTTTCCTGTGCCGCGTCAGGCTTTACCTTCAGCAATTCAG
ACATGAACTGGGTGCATCAGGCCCCAGGGAAGGGCCTGGAGTGGGTATCTG
GAGTCTCTTGGAATGGCAGCCGTACACACTATGCTGATTCTGTTAAGGGGC
AGTTCATCATTTCCCGCGATAATTCCAGGAACACACTCTACCTGCAAACCAA
CAGCTTGCGAGCAGAAGACACTGCTGTGTATTACTGCGTCAGAAACACAGTG
CTACAAACACATCCTCAGTGTGTCAGAAATCCTGGAGGTGCAGCAAGCTCCCTT
GGGACTGACAAGACTTAGAGAATAGTTGCTTGCAGACGT
  8) Mouse VH: IGHV1-64*01 / Human VH: IGHV3-62*04
Nucleic acid sequence present on synthetic array with human VH:
(SEQ ID NO: 60)
AAGGTTCAGGGATGAGATATGAGGGTGAATTTCCACGGACAAGATTAGGACTTG
GGCTTCAGCATCCTGATTCCTGACCCAGGTGTCCCTTCTTCTCCAGCAGGAGTAG
GGGCTCATCTAATATGTATCCTGCTCATGAATATGCAAATCCTCTGAATCTACAT
GGTAAATATAGGGTTGTCTATACCACACACAGAAAAACATGAGATCACAGTTCT
CTCTACAGTTACTGAGCACACAGGACCTCACAATGGGATGGAGCTATATCATCCT
CTTTTTGGTAGCAACAGCTACAGGTAAGGGGCTCACAGTAGCAGGCTTGAGGTC
TGGCCATATACATGGGTGACAATGACATCCACTTTGCCCTTCTCTCCACAGGTGT
CCACTCCGAGGTGCAGCTGGTGAAGTCTGGAGGAGGCTTGGTACAGCCTGG
GGGGTCCCTGAGACTCTCCTGTGCAGCCTCTGGATTCACCTTCAGTAGCTCT
GCTATGCACTGGGTCCGCCAGGCTCCAAGAAAGGGTTTGGAGTGGGTCTCA
GTTATTAGTACAAGTGGTGATACCGTACTCTACACAGACTCTGTGAAGGGC
CGATTCACCATCTCCAGAGACAATGCCCAGAATTCACTGTCTCTGCAAATGA
ACAGCCTGAGAGCCGAGGACATGGCTGTGTATTACTGTGTGAAAGACACAG
TGTTGCAACCACATCCTGAGAGTGTCTGAAAACCTGGAGGGGTAACAAACTGCC
CTGGGACTTAGGAGACTCAGAGAAAGTTTGCTTGTAGACTT
Nucleic acid sequence present on synthetic array with codon optimized human VH:
(SEQ ID NO: 61)
AAGGTTCAGGGATGAGATATGAGGGTGAATTTCCACGGACAAGATTAGGACTTG
GGCTTCAGCATCCTGATTCCTGACCCAGGTGTCCCTTCTTCTCCAGCAGGAGTAG
GGGCTCATCTAATATGTATCCTGCTCATGAATATGCAAATCCTCTGAATCTACAT
GGTAAATATAGGGTTGTCTATACCACACACAGAAAAACATGAGATCACAGTTCT
CTCTACAGTTACTGAGCACACAGGACCTCACAATGGGATGGAGCTATATCATCCT
CTTTTTGGTAGCAACAGCTACAGGTAAGGGGCTCACAGTAGCAGGCTTGAGGTC
TGGCCATATACATGGGTGACAATGACATCCACTTTGCCCTTCTCTCCACAGGTGT
CCACTCCGAAGTACAGCTGGTGAAAAGTGGTGGTGGGCTCGTTCAACCTGG
AGGCTCCCTTCGCCTCAGCTGTGCTGCGAGTGGCTTCACCTTCAGCTCCTCA
GCCATGCACTGGGTTCGGCAGGCACCAAGGAAGGGCCTGGAGTGGGTGTC
TGTCATTTCCACTTCTGGGGACACAGTGCTGTATACAGATAGTGTCAAAGGA
CGATTTACCATCAGCAGAGATAATGCCCAGAACTCATTGTCTCTGCAGATGA
ACAGCCTAAGAGCAGAGGACATGGCTGTGTACTACTGCGTGAAGGACACAG
TGTTGCAACCACATCCTGAGAGTGTCTGAAAACCTGGAGGGGTAACAAACTGCC
CTGGGACTTAGGAGACTCAGAGAAAGTTTGCTTGTAGACTT
  9) Mouse VH: IGHV1-9*01 / Human VH: IGHV3-16*02
Nucleic acid sequence present on synthetic array with human VH:
(SEQ ID NO: 62)
AGCTTACCAAAAATGTAGGAAAAGTATTTTTTCAGAGAAGATTAGGACATTGAC
CAGAGCATCCTGATACCTGACGCATGTGCCTACTGTCCAGTAGTTCTCCTGCTCA
ATTAGGTTCTTATCTAAGAAGTTAATGCTCATGAATATGCAAATTACTTGAGTCT
ATGGCAGTAAATACAGGGGTGACCACATGCAGAAAACAGCATATGATCAGTGTC
CTCTCCAAAGTCCTTGAACATAGACTCTAACCATGGAATGGACCTGGGTCTTTCT
CTTCCTCCTGTCAGTAACTGCAGGTAAGGGGCTCACCATTTTCAAATCTGAAGAA
GAGACAGAGCTTGAGGTGACAATGACAACCACTCTGCCTTTCTCTCCACAGGTGT
CCACTCCGAGGTGCAGCTGGTGGAGTCTGGGGGAGGCTTGGTACAGCCTGG
GGGGTCCCTGAGACTCTCCTGTGCAGCCTCTGGATTCACCTTCAGTAACAG
TGACATGAACTGGGCCCGCAAGGCTCCAGGAAAGGGGCTGGAGTGGGTAT
CGGGTGTTAGTTGGAATGGCAGTAGGACGCACTATGTGGACTCCGTGAAGC
GCCGATTCATCATCTCCAGAGACAATTCCAGGAACTCCCTGTATCTGCAAAA
GAACAGACGGAGAGCCGAGGACATGGCTGTGTATTACTGTGTGAGAAACAC
AGTGTTGTAACCACATCCTGAGTGTGTCAGAAACTCTGGAGGAGCAGCAAGCTG
CCCTGGGTCTGAAATATCAGAAAAGGCTAACATTTAGACTTCC
Nucleic acid sequence present on synthetic array with codon optimized human VH:
(SEQ ID NO: 63)
AGCTTACCAAAAATGTAGGAAAAGTATTTTTTCAGAGAAGATTAGGACATTGAC
CAGAGCATCCTGATACCTGACGCATGTGCCTACTGTCCAGTAGTTCTCCTGCTCA
ATTAGGTTCTTATCTAAGAAGTTAATGCTCATGAATATGCAAATTACTTGAGTCT
ATGGCAGTAAATACAGGGGTGACCACATGCAGAAAACAGCATATGATCAGTGTC
CTCTCCAAAGTCCTTGAACATAGACTCTAACCATGGAATGGACCTGGGTCTTTCT
CTTCCTCCTGTCAGTAACTGCAGGTAAGGGGCTCACCATTTTCAAATCTGAAGAA
GAGACAGAGCTTGAGGTGACAATGACAACCACTCTGCCTTTCTCTCCACAGGTGT
CCACTCCGAAGTGCAGCTGGTGGAGAGTGGTGGTGGCTTGGTTCAACCAGG
AGGCAGCCTAAGGCTTAGCTGTGCTGCCAGTGGCTTCACATTTTCCAATTCA
GATATGAACTGGGCAAGGAAGGCCCCTGGGAAAGGACTGGAGTGGGTGTC
AGGGGTCTCCTGGAATGGATCCCGTACCCACTATGTGGATTCTGTAAAGCG
CCGCTTCATCATTTCTCGGGACAACAGCAGAAATTCTCTCTATCTGCAGAAG
AACAGAAGACGAGCGGAAGACATGGCTGTCTACTACTGCGTTCGGAACACA
GTGTTGTAACCACATCCTGAGTGTGTCAGAAACTCTGGAGGAGCAGCAAGCTGC
CCTGGGTCTGAAATATCAGAAAAGGCTAACATTTAGACTTCC
10) Mouse VH: IGHV1-75*01 / Human VH: IGHV3-38*02
Nucleic acid sequence present on synthetic array with human VH:
(SEQ ID NO: 64)
AAAGTGTTTGGGATACATTTCCTCAGAGAGGATTAGGACTTGGACCTGAGCTGCT
TGACCCAGGTGCCTTTTCAGTCCTTCCTCTCCAGTTTTTCTCTAGATGGACTAGGT
CCTTAACTAAAAAATGCACTGCTTGTGAATATGCAAATCACCCACGTCTATGGCA
GTAAATACATGGATGTCCACAGCCTGAAAACAACCTATGATCAGTGTCCTCTCTA
CACAGTCCCTGACGACACTGACTCTAACCATGGGATGGAGCTGGATCTTTCTCTT
CCTCCTGTCAGGAACTGCAGGTAAGGGAATTAATTCCAAATCTCACCAATTCCAA
ATCTGAAGTGGAGACAGGGTCTGAGGTGATAATGACATCCACAGTGCCTTTCTTT
CCATAGGTGTCCATTGCGAGGTGCAGCTGGTGGAGTCTGGGGGAGGCTTGGT
ACAGCCTAGGGGGTCCCTGAGACTCTCCTGTGCAGCCTCTGGATTCACCGT
CAGTAGCAATGAGATGAGCTGGATCCGCCAGGCTCCAGGGAAGGGGCTGG
AGTGGGTCTCATCCATTAGTGGTGGTAGCACATACTACGCAGACTCCAGGA
AGGGCAGATTCACCATCTCCAGAGACAATTCCAAGAACACGCTGTATCTTCA
AATGAACAACCTGAGAGCTGAGGGCACGGCCGTGTATTACTGTGCCAGATA
TACACAGTGTTACAACCACATCCTGAGTGTGTCAGAAACCCTGGAGGAGCAGGA
AGCTGCACTGGGACTGAGATGACAGAAAGATTAATCCTTAGACTTTCT
Nucleic acid sequence present on synthetic array with codon optimized human VH:
(SEQ ID NO: 65)
AAAGTGTTTGGGATACATTTCCTCAGAGAGGATTAGGACTTGGACCTGAGCTGCT
TGACCCAGGTGCCTTTTCAGTCCTTCCTCTCCAGTTTTTCTCTAGATGGACTAGGT
CCTTAACTAAAAAATGCACTGCTTGTGAATATGCAAATCACCCACGTCTATGGCA
GTAAATACATGGATGTCCACAGCCTGAAAACAACCTATGATCAGTGTCCTCTCTA
CACAGTCCCTGACGACACTGACTCTAACCATGGGATGGAGCTGGATCTTTCTCTT
CCTCCTGTCAGGAACTGCAGGTAAGGGAATTAATTCCAAATCTCACCAATTCCAA
ATCTGAAGTGGAGACAGGGTCTGAGGTGATAATGACATCCACAGTGCCTTTCTTT
CCATAGGTGTCCATTGCGAAGTGCAGCTGGTGGAGAGTGGAGGTGGTCTAGT
CCAGCCTCGAGGGAGTTTGAGGCTTAGCTGTGCTGCCTCTGGCTTCACTGT
TTCTTCCAATGAGATGTCTTGGATCCGGCAAGCCCCAGGGAAGGGCCTGGA
GTGGGTGTCCAGCATTTCAGGAGGAAGCACCTACTATGCAGATTCAAGGAA
AGGCCGTTTTACCATATCCAGAGACAACAGCAAGAATACACTCTACCTGCA
GATGAACAACCTCAGAGCAGAAGGGACAGCTGTATATTACTGCGCCCGCTA
TACACAGTGTTACAACCACATCCTGAGTGTGTCAGAAACCCTGGAGGAGCAGGA
AGCTGCACTGGGACTGAGATGACAGAAAGATTAATCCTTAGACTTTCT
11) Mouse VH: IGHV5-4*01 / Human VH: IGHV3-38-3*01
Nucleic acid sequence present on synthetic array with human VH:
(SEQ ID NO: 66)
GAAACTAAAATGTGTTTCAAATGCTGTAAATAAAATCTGGTTTTTTGATGCCTTA
TATATCTGTTATCATCAGTGGCATCAGCCTAGGTCCAACTCCGGAGCATGGTATA
GCAGGAAGACATGCAAATAAGTCTTCTCTGTGCCCATGAAAAACACCTCGGCCC
TGACCCTGCAGCTCTGACAGAGGAGGCCGGTCCTGGATTCGAGTTCCTCACATTC
AGTGATGAGCACTGAACACGGACCCCTCACCATGAACTTCGGGCTCAGCTTGAT
TTTCCTTGTCCTTGTTTTAAAAGGTAATTTATTGAGAAGAGATGACATCTGTTGTA
TGCACATGAGACAGAGAAAAATTGTTGTTTGTTTTGTTAGTGACAGTTTTCCAAC
CAGTATTCTCTGTTTGCAGGTGTCCAGTGTGAGGTGCAGCTGGTGGAGTCTCG
GGGAGTCTTGGTACAGCCTGGGGGGTCCCTGAGACTCTCCTGTGCAGCCTC
TGGATTCACCGTCAGTAGCAATGAGATGAGCTGGGTCCGCCAGGCTCCAGG
GAAGGGTCTGGAGTGGGTCTCATCCATTAGTGGTGGTAGCACATACTACGC
AGACTCCAGGAAGGGCAGATTCACCATCTCCAGAGACAATTCCAAGAACAC
GCTGCATCTTCAAATGAACAGCCTGAGAGCTGAGGACACGGCTGTGTATTA
CTGTAAGAAAGACACAATGAGGAAATGTTACTGTGAGCTCAAACTAAAACCTCC
TGCAGAGCACCCAAGACCAGCAGGGGGCGGAGAGAGCATAGTAATTTGGAAAT
TTGCA
Nucleic acid sequence present on synthetic array with codon optimized human VH:
(SEQ ID NO: 67)
GAAACTAAAATGTGTTTCAAATGCTGTAAATAAAATCTGGTTTTTTGATGCCTTA
TATATCTGTTATCATCAGTGGCATCAGCCTAGGTCCAACTCCGGAGCATGGTATA
GCAGGAAGACATGCAAATAAGTCTTCTCTGTGCCCATGAAAAACACCTCGGCCC
TGACCCTGCAGCTCTGACAGAGGAGGCCGGTCCTGGATTCGAGTTCCTCACATTC
AGTGATGAGCACTGAACACGGACCCCTCACCATGAACTTCGGGCTCAGCTTGAT
TTTCCTTGTCCTTGTTTTAAAAGGTAATTTATTGAGAAGAGATGACATCTGTTGTA
TGCACATGAGACAGAGAAAAATTGTTGTTTGTTTTGTTAGTGACAGTTTTCCAAC
CAGTATTCTCTGTTTGCAGGTGTCCAGTGTGAAGTGCAGCTAGTGGAGTCAAG
AGGTGTTTTGGTCCAGCCTGGAGGAAGTCTCAGGCTCTCCTGTGCCGCGTC
TGGCTTTACTGTATCAAGTAATGAGATGTCCTGGGTGAGGCAAGCCCCAGG
GAAAGGCCTGGAGTGGGTCAGCAGTATTTCTGGTGGCAGCACCTATTATGC
TGACAGCCGGAAAGGGCGCTTCACCATCTCCAGAGACAACAGCAAGAACAC
ACTGCACCTGCAGATGAATTCTCTTCGAGCAGAAGATACAGCTGTGTACTAC
TGCAAGAAGGACACAATGAGGAAATGTTACTGTGAGCTCAAACTAAAACCTCCT
GCAGAGCACCCAAGACCAGCAGGGGGCGGAGAGAGCATAGTAATTTGGAAATT
TGCA
12) Mouse VH: IGHV5-6*01 / Human VH: IGHV1-38-4*01
Nucleic acid sequence present on synthetic array with human VH:
(SEQ ID NO: 68)
GAAACTACAATATGTTTCAAATGCTGTAACTGAAATCTGGTTTTTTGATGCTTAT
ATCTGGTATCATCAGTGACTTCAGATTTAGTCCAACCCCAGAGCATGGTATAGCA
GGAAGACATGCAAATAAGTCTTCTCTGTGCCCATGAAAAACACCTCGGCCCTGA
CCCTGCAGCTCTGACAGAGGAGGCCGGTCCTGGATTCGATTCCCAGTTCCTCACA
TTCAGTCAGCACTGAACACGGACCCCTCACCATGAACTTCGGGCTCAGCTTGATT
TTCCTTGCCCTTATTTTAAAAGGTAATTTATTGAGAAGAGATGACATCTCTTGTAT
GCACGTGAGAGAGAAAAATTGTTTTGTTAGTGACAGTTTTCCAACCAGTATTCTC
TGTTTGCAGGTGTCCAGTGTCAGGTCCAGCTGGTGCAGTCTTGGGCTGAGGT
GAGGAAGTCTGGGGCCTCAGTGAAAGTCTCCTGTAGTTTTTCTGGGTTTAC
CATCACCAGCTACGGTATACATTGGGTGCAACAGTCCCCTGGACAAGGGCT
TGAGTGGATGGGATGGATCAACCCTGGCAATGGTAGCCCAAGCTATGCCAA
GAAGTTTCAGGGCAGATTCACCATGACCAGGGACATGTCCACAACCACAGC
CTACACAGACCTGAGCAGCCTGACATCTGAGGACATGGCTGTGTATTACTG
TGCAAGACACACAATGAGGAAATGTTACTGTGAGCTCAAACTAAAACCTCCTGC
AGAGCACCCAGGACCAGCAGGGGGCGCAGAGAGCACATGGAGTTCTGATTCAC
AG
Nucleic acid sequence present on synthetic array with codon optimized human VH:
(SEQ ID NO: 69)
GAAACTACAATATGTTTCAAATGCTGTAACTGAAATCTGGTTTTTTGATGCTTAT
ATCTGGTATCATCAGTGACTTCAGATTTAGTCCAACCCCAGAGCATGGTATAGCA
GGAAGACATGCAAATAAGTCTTCTCTGTGCCCATGAAAAACACCTCGGCCCTGA
CCCTGCAGCTCTGACAGAGGAGGCCGGTCCTGGATTCGATTCCCAGTTCCTCACA
TTCAGTCAGCACTGAACACGGACCCCTCACCATGAACTTCGGGCTCAGCTTGATT
TTCCTTGCCCTTATTTTAAAAGGTAATTTATTGAGAAGAGATGACATCTCTTGTAT
GCACGTGAGAGAGAAAAATTGTTTTGTTAGTGACAGTTTTCCAACCAGTATTCTC
TGTTTGCAGGTGTCCAGTGTCAGGTGCAGCTGGTTCAGTCCTGGGCAGAGGT
GAGGAAAAGTGGTGCTTCTGTAAAAGTCTCCTGCAGCTTTTCTGGCTTCACC
ATAACCAGCTATGGAATTCACTGGGTCCAGCAGAGCCCCGGGCAAGGGCTG
GAGTGGATGGGCTGGATCAACCCTGGAAATGGCTCGCCATCCTATGCCAAG
AAGTTCCAGGGTCGCTTTACCATGACAAGAGATATGTCTACTACGACAGCCT
ACACAGACCTCTCAAGTCTTACTTCAGAAGACATGGCTGTGTACTACTGTGC
GCGGCACACAATGAGGAAATGTTACTGTGAGCTCAAACTAAAACCTCCTGCAGA
GCACCCAGGACCAGCAGGGGGCGCAGAGAGCACATGGAGTTCTGATTCACAG
13) Mouse VH: IGHV9-3*01 / Human VH: IGHV8-51-1*02
Nucleic acid sequence present on synthetic array with human VH:
(SEQ ID NO: 70)
CTCAGAGGACAGCACAATCAGCATGAACTAATAAACATCAGCTAAGATAAGGCA
CAGGCTCAGCTATATAGGGTCAGGGATCTTTGTAAATCTGATTGTGTATCCAGTC
TAGTTCAATGTGACTTAGGAAGGCCAGTCATATGCAAATCTAGAGAAGACTTTA
GAGTAGAAATCTGAGGCTCACCTCACATACCAGCAAGGGAGTGACCAGTTAGTC
TTAAGGCACCACTGAGCCCAAGTCTTAGACATCATGGGTTGGCTGTGGAACTTGC
TATTCCTGATGGCAGCTGCCCAAAGTAAGACATCAGAAAAAAAGAGTTCCAAGG
GGAATTGAAGCAGTTCCATGAATACTCACCTTCCTGTGTTCTTTTCACAGGTGCC
CAAGCAGAGGCCCAGCTTACAGAGTCTGGGGGAGACTTGGTACACTTAGAG
GGGCCCCTGAGGCTCTCCTGTGCAGCCTCTTGGTTCACCTTCAGTATCTATG
AGATTCACTGGGTTTGCCAGGCCTCAGGGAAGGGGCTGGAATGGGTTGCAG
TTATATGGCGTGGTGAAAGTCATCAATACAATGCAGACTATGTTAGGGGCA
GACTCACCACTTCCAGAGACAACACCAAGTACATGCTGTACATGCAAATGAT
CAGCCTGAGAACCCAGAACATGGCAGCATTTAACTGTGCAGGAAACACAGT
GTGAAAACCACATCCTGAGGGTGTCAAAAACCATGAGGAGAAGGTGGTTCAGCT
GTGTCCAGAAGCAACCAGAGGAAACATTCTCTCCTTGGTG
Nucleic acid sequence present on synthetic array with codon optimized human VH:
(SEQ ID NO: 71)
CTCAGAGGACAGCACAATCAGCATGAACTAATAAACATCAGCTAAGATAAGGCA
CAGGCTCAGCTATATAGGGTCAGGGATCTTTGTAAATCTGATTGTGTATCCAGTC
TAGTTCAATGTGACTTAGGAAGGCCAGTCATATGCAAATCTAGAGAAGACTTTA
GAGTAGAAATCTGAGGCTCACCTCACATACCAGCAAGGGAGTGACCAGTTAGTC
TTAAGGCACCACTGAGCCCAAGTCTTAGACATCATGGGTTGGCTGTGGAACTTGC
TATTCCTGATGGCAGCTGCCCAAAGTAAGACATCAGAAAAAAAGAGTTCCAAGG
GGAATTGAAGCAGTTCCATGAATACTCACCTTCCTGTGTTCTTTTCACAGGTGCC
CAAGCAGAAGCCCAGCTGACAGAAAGTGGAGGAGACCTGGTGCACCTAGAA
GGCCCTCTTCGGCTCAGCTGCGCGGCCTCCTGGTTCACCTTTTCCATATATG
AGATCCACTGGGTCTGCCAAGCTTCTGGGAAAGGCCTGGAGTGGGTGGCTG
TAATCTGGAGGGGGGAGAGCCATCAGTACAATGCAGATTATGTTCGAGGTC
GTCTCACAACGTCAAGAGACAATACCAAGTACATGCTGTACATGCAGATGA
TTTCTTTGCGCACTCAGAACATGGCTGCCTTCAACTGTGCAGGCAACACAGT
GTGAAAACCACATCCTGAGGGTGTCAAAAACCATGAGGAGAAGGTGGTTCAGCT
GTGTCCAGAAGCAACCAGAGGAAACATTCTCTCCTTGGTG
14) Mouse VH: IGHV6-3*01 / Human VH: IGHV7-81*01
Nucleic acid sequence present on synthetic array with human VH:
(SEQ ID NO: 72)
TTAGTAAATGATATACTGATGTTGTAATGCCACTTTTCCTTTACTATTTCTCAGAT
ATCTATCTGCATTATTATTTGAGTCTTCTAAGGCTCCCACCCTGAGCTCATGATAT
AGCACACAGAAATGCAAATCATATTTCCCTTTGATTATGAATACCAGCCCTGAGA
CTCTAAAGCTCTGACAGAGGCACCTAACTGTGGACTCACAAGTCTTTCCCTTCAG
TGACCAACACGGACACAGAACATTCACCATGGACTTGAGACTGAGCTGTGCTTTT
ATTATTGTTCTTTTAAAAGGTAATTCATAGATAATAAGAGATATTGAGTGTGTGA
GTGGACGGGAGTGAGGGAACACTGAATATTTTGACGGCTTACTGAGAGGGTTCT
CTGTGTTTTCAGGGGTCCAGAGTCAGGTGCAGCTGGTGCAGTCTGGCCATGA
GGTGAAGCAGCCTGGGGCCTCAGTGAAGGTCTCCTGCAAGGCTTCTGGTTA
CAGTTTCACCACCTATGGTATGAATTGGGTGCCACAGGCCCCTGGACAAGG
GCTTGAGTGGATGGGATGGTTCAACACCTACACTGGGAACCCAACATATGC
CCAGGGCTTCACAGGACGGTTTGTCTTCTCCATGGACACCTCTGCCAGCAC
AGCATACCTGCAGATCAGCAGCCTAAAGGCTGAGGACATGGCCATGTATTA
CTGTGCGAGATACACAGTGAGAAGTCTTCATTGTGAGTCTAGACACAAACTTAC
CCAAAGGAGCTCTCAGTACCAGCAGGGGGAGCACAGTGACAATCGAATCCATAA
ATGG
Nucleic acid sequence present on synthetic array with codon optimized human VH:
(SEQ ID NO: 73)
TTAGTAAATGATATACTGATGTTGTAATGCCACTTTTCCTTTACTATTTCTCAGAT
ATCTATCTGCATTATTATTTGAGTCTTCTAAGGCTCCCACCCTGAGCTCATGATAT
AGCACACAGAAATGCAAATCATATTTCCCTTTGATTATGAATACCAGCCCTGAGA
CTCTAAAGCTCTGACAGAGGCACCTAACTGTGGACTCACAAGTCTTTCCCTTCAG
TGACCAACACGGACACAGAACATTCACCATGGACTTGAGACTGAGCTGTGCTTTT
ATTATTGTTCTTTTAAAAGGTAATTCATAGATAATAAGAGATATTGAGTGTGTGA
GTGGACGGGAGTGAGGGAACACTGAATATTTTGACGGCTTACTGAGAGGGTTCT
CTGTGTTTTCAGGGGTCCAGAGTCAAGTTCAGCTGGTGCAGAGTGGTCACGAA
GTGAAGCAGCCAGGAGCATCTGTCAAAGTCAGCTGCAAAGCATCAGGCTAC
AGCTTCACCACTTATGGCATGAACTGGGTGCCTCAGGCTCCGGGGCAAGGT
TTGGAGTGGATGGGCTGGTTCAACACCTACACAGGGAATCCCACCTATGCC
CAGGGCTTCACAGGAAGGTTTGTATTTTCCATGGATACTTCTGCTTCGACGG
CGTATCTGCAGATCTCCAGTCTCAAGGCTGAGGACATGGCCATGTACTACT
GTGCCAGATGCACAGTGAGAAGTCTTCATTGTGAGTCTAGACACAAACTTACCC
AAAGGAGCTCTCAGTACCAGCAGGGGGAGCACAGTGACAATCGAATCCATAAAT
GG
```

### Example 7: Polypeptide Sequences Encoded by Exemplary VH Gene Segments

IGHV1-8*01 encoded amino acid sequence
(SEQ ID NO: 74)
QVQLVQSGAEVKKPGASVKVSCKASGYTFTSYDINWVRQATGQGLEWMG
WMNPNSGNTGYAQKFQGRVTMTRNTSISTAYMELSSLRSEDTAVYYCAR
IGHV3-9*01 encoded amino acid sequence
(SEQ ID NO: 75)
EVQLVESGGGLVQPGRSLRLSCAASGFTFDDYAMHWVRQAPGKGLEWVS
GISWNSGSIGYADSVKGRFTISRDNAKNSLYLQMNSLRAEDTALYYCAKD
IGHV4-31*03 encoded amino acid sequence
(SEQ ID NO: 76)
QVQLQESGPGLVKPSQTLSLTCTVSGGSISSGGYYWSWIRQHPGKGLEW
IGYIYYSGSTYYNPSLKSRVTISVDTSKNQFSLKLSSVTAADTAVYYCA
R
IGHV4-30-4*01 encoded amino acid sequence
(SEQ ID NO: 77)
QVQLQESGPGLVKPSQTLSLTCTVSGGSISSGDYYWSWIRQPPGKGLEW
IGYIYYSGSTYYNPSLKSRVTISVDTSKNQFSLKLSSVTAADTAVYYCA
R
IGHV4-38-2*02 encoded amino acid sequence
(SEQ ID NO: 78)
QVQLQESGPGLVKPSETLSLTCTVSGYSISSGYYWGWIRQPPGKGLEWI
GSIYHSGSTYYNPSLKSRVTISVDTSKNQFSLKLSSVTAADTAVYYCAR
IGHV3-43D*04 encoded amino acid sequence
(SEQ ID NO: 79)
EVQLVESGGVVVQPGGSLRLSCAASGFTFDDYAMHWVRQAPGKGLEWVS
LISWDGGSTYYADSVKGRFTISRDNSKNSLYLQMNSLRAEDTALYYCAK
D
IGHV3-35*02 encoded amino acid sequence
(SEQ ID NO: 80)
EVQLVESGGGLVQPGGSLRLSCAASGFTFSNSDMNWVHQAPGKGLEWVS
GVSWNGSRTHYADSVKGQFIISRDNSRNTLYLQTNSLRAEDTAVYYCVR
IGHV3-62*04 encoded amino acid sequence
(SEQ ID NO: 81)
EVQLVKSGGGLVQPGGSLRLSCAASGFTFSSSAMHWVRQAPRKGLEWVS
VISTSGDTVLYTDSVKGRFTISRDNAQNSLSLQMNSLRAEDMAVYYCVK
IGHV3-16*02 encoded amino acid sequence
(SEQ ID NO: 82)
EVQLVESGGGLVQPGGSLRLSCAASGFTFSNSDMNWARKAPGKGLEWVS
GVSWNGSRTHYVDSVKRRFIISRDNSRNSLYLQKNRRRAEDMAVYYCVR
IGHV3-38*02 encoded amino acid sequence
(SEQ ID NO: 83)
EVQLVESGGGLVQPRGSLRLSCAASGFTVSSNEMSWIRQAPGKGLEWVS
SISGGSTYYADSRKGRFTISRDNSKNTLYLQMNNLRAEGTAVYYCARY
IGHV3-38-3*01 encoded amino acid sequence
(SEQ ID NO: 84)
EVQLVESRGVLVQPGGSLRLSCAASGFTVSSNEMSWVRQAPGKGLEWVS
SISGGSTYYADSRKGRFTISRDNSKNTLHLQMNSLRAEDTAVYYCKK
IGHV1-38-4*01 encoded amino acid sequence
(SEQ ID NO: 85)
QVQLVQSWAEVRKSGASVKVSCSFSGFTITSYGIHWVQQSPGQGLEWMG
WINPGNGSPSYAKKFQGRFTMTRDMSTTTAYTDLSSLTSEDMAVYYCAR
IGHV8-51-1*02 encoded amino acid sequence
(SEQ ID NO: 86)
EAQLTESGGDLVHLEGPLRLSCAASWFTFSIYEIHWVCQASGKGLEWVA
VIWRGESHQYNADYVRGRLTTSRDNTKYMLYMQMISLRTQNMAAFNCAG
IGHV7-81*01 encoded amino acid sequence
(SEQ ID NO: 87)
QVQLVQSGHEVKQPGASVKVSCKASGYSFTTYGMNWVPQAPGQGLEWMG
WENTYTGNPTYAQGFTGRFVFSMDTSASTAYLQISSLKAEDMAMYYCAR

### Example 8: Singularity Sapiens V6 Mice Expressing Extra Human Variable Gene Segments

This Example describes the design and generation of mice that express extra VH gene segments (e.g., extra human VH gene segments) that can be used to expand the VH domain diversity of antibodies such as heavy chain only antibodies.

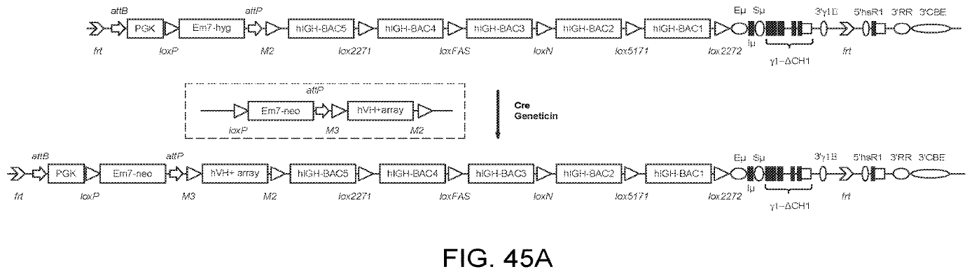

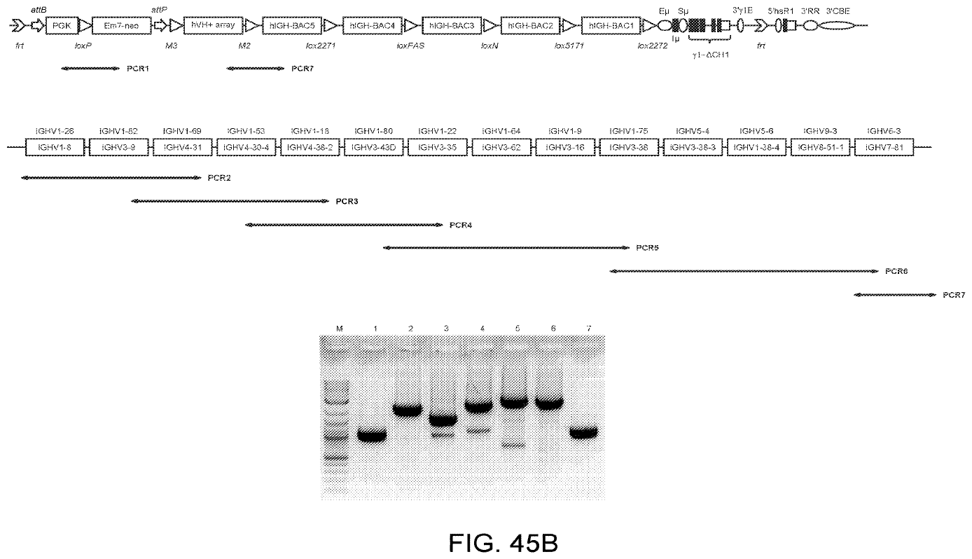

An array containing three lox sequences (LoxP, M2, and M3) and a neomycin cassette is integrated into the singularity sapiens SSV5 (generated with either hIGH-BAC5 or hIGH-BAC5*) allele via recombination mediated cassette exchange (RMCE) to generate SSV6 (FIG. 45).

Addition of these 14 VH domains into SSV5 to generate SSV6 further enhances the immunoglobulin repertoire diversity from 44 to 58 functional VH gene segments (Table 7).

TABLE 7
Exemplary human VH domains added in SSV6 and their closest
VH homologs in SSV5 and their amino acid differences.

| | Closest SSV5 | Amino Acid |
|---|---|---|
| Human VH domain: | Functional Homolog | Differences |
| IGHV1-8*01 | IGHV1-2*04 | 12 |
| IGHV3-9*01 | IGHV3-43*01 | 11 |
| IGHV4-31*03 | IGHV4-30-4*01 | 2 |
| IGHV4-30-4*01 | IGHV4-61*01 | 4 |
| IGHV4-38-2*02 | IGHV4-39*01 | 5 |
| IGHV3-43D*04 | IGHV3-43*01 | 2 |
| IGHV3-35*02 | IGHV3-23*04 | 18 |
| IGHV3-62*04 | IGHV3-48*03 | 16 |
| IGHV3-16*02 | IGHV3-48*03 | 12 |
| IGHV3-38*02 | IGHV3-53*02 | 10 |
| IGHV3-38-3*01 | IGHV3-53*02 | 11 |
| IGHV1-38-4*01 | IGHV1-46*01 | 25 |
| IGHV8-51-1*02 | IGHV3-33*01 | 38 |
| IGHV7-81*01 | IGHV7-4-1*01 | 14 |

### Example 9: Additional Singularity-Based Non-Human Animals: VHHs and VNARs

### Generation of the Singularity Sapacos Allelic Series

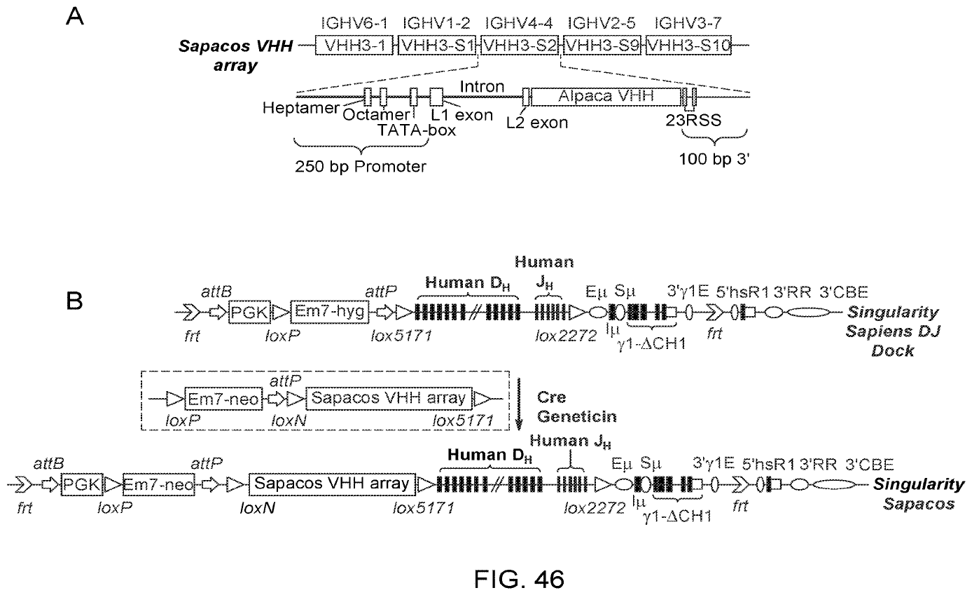

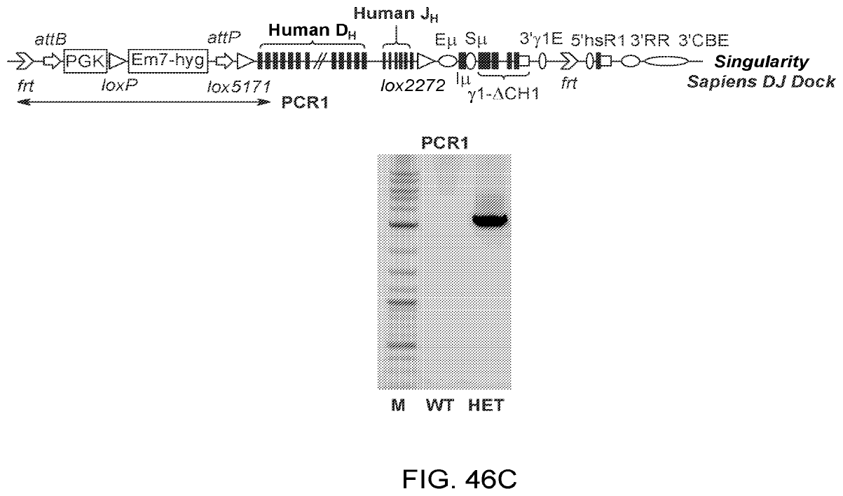

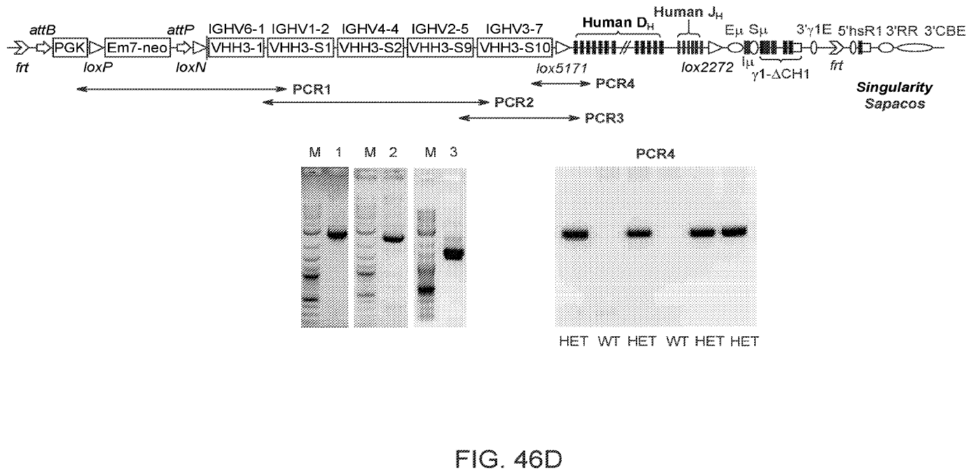

The capability of the Singularity platform was expanded by constructing a synthetic array (Sapacos VHH) containing 5 known VHHs of the alpaca (Vicugna pacos) (Achour et al., J. Immunol., 181 (3): 2001-2009 (2008)). An individual VHH element was grafted onto the framework of a selected human VH component consisting of the about 250 bp human promoter containing regulatory elements (e.g., TATA-box, octamer, and heptamer) involved in VH transcription, human leader exon 1, human intron 1 and human leader exon 2 in the upstream, and human recombination signal sequences (RSS) in the downstream (FIG. 46A). Specific human VHs were selected based on their high utilization rates in humans and humanized rodent models (e.g., rats and mice). The Sapacos VHH array was designed to contain flanking disparate lox elements to facilitate its targeted integration into the Singularity Sapiens IgH locus and was inserted into the Singularity Sapiens DJ dock allele via RMCE (FIG. 46B-46D). Mice generated from this genetic engineering are referred to as Singularity Sapacos mice and are assessed for their ability to produce alpaca-human-mouse chimeric heavy chain only antibodies (e.g., alpaca-human-mouse chimeric heavy chain IgG1-ΔCH1 antibodies).

The alpaca-human-mouse chimeric heavy chain only antibodies produced from the Singularity Sapacos mice will have the naturally optimized nanobody properties of alpaca and access the additional immune diversity via the human D and J elements, enabling rapid humanization of alpaca VHHs for therapeutic applications in humans. The Sapacos VHH array can be readily expanded via repeated rounds of RMCE-mediated integration of arrays containing additional V_{H}Hs of alpaca and other camelid species.

### Sequence of Singularity Sapacos Array (L1 and L2 are Underlined, Alpaca V_{H}H Sequence is Bold and Underline, RSS is Italic)

1) Human VH: IGHV6-1; Alpaca V_{H}H substitution: VHH3-1
(SEQ ID NO: 88)
GGGCCCTGCCTCTGAGCTCCTCTTTGCATCCAATCTGCTGAAGAACATGGCTCTA
GGGAAACCCAGTTGTAGACCTGAGGGCCCCGGCTCTTCAATGAGCCATCTCCGT
CCCGGGGCCTTATATCAGCAAGTGACGCACACAGGCAAATGCCAGGGTGTGGTT
TCCTGTTTAAATGTAGCCTCCCCCGCTGCAGAACTGCAGAGCCTGCTGAATTCTG
GCTGACCAGGGCAGTCACCAGAGCTCCAGACAATGTCTGTCTCCTTCCTCATCTT
CCTGCCCGTGCTGGGCCTCCCATGGGGTCAGTGTCAGGGAGATGCCGTATTCACA
GCAGCATTCACAGACTGAGGGGTGTTTCACTTTGCTGTTTCCTTTTGTCTCCAGGT
GTCCTGTCACAGGTGCAGCTGGTGGAGTCAGGTGGAGGATTAGTTCAGGCT
GGAGGTTCTCTTCGACTATCCTGCGCGGCCAGTGGGCGCACCTTCAGCTCC
TATGCCATGGGCTGGTTTCGTCAAGCACCTGGGAAGGAGAGGGAATTTGTG
GCAGCCATTTCCTGGTCAGGTGGCAGCACATACTATGCAGACTCTGTAAAA
GGGCGGTTTACCATAAGTAGAGACAATGCCAAGAACACTGTGTACCTGCAG
ATGAACAGCTTGAAACCAGAAGATACAGCTGTCTATTACTGTGCTGCTCACA
GTGAGGGGAAGTCAGTGTGAGCCCAGACACAAACCTCCCTGCAGGGATGCTCAGGA
CCCCAGAAGGCACCCAGCACTACCAGCGCAGGGCCCAGAC
2) Human VH: IGHV1-2; Alpaca V_{H}H substitution: VHH3-S1
(SEQ ID NO: 89)
GGGGACACACATCATTAAACAAGGATTGGGACAGGGACTTCAGCGTCCCACTGT
TGCATGGCCCATAAATTATGTGTGTTCTCTTTCTCATCTTGGATCAAGTCTAGAGC
TATGAAATAGTATCCCTCATGAATATGCAAATAACCTGAGATTTACTGAAGTAAA
TACAGATCTGTCCTGTGCCCTGAGAGCATCACCCAGCAACCACATCTGTCCTCTA
GAGAATCCCCTGAGAGCTCCGTTCCTCACCATGGACTGGACCTGGAGGATCCTCT
TCTTGGTGGCAGCAGCCACAGGTAAGAGGCTCCCTAGTCCCAGTGATGAGAAAG
AGATTGAGTCCAGTCCAGGGAGATCTCATCCACTTCTGTGTTCTCTCCACAGGAG
CCCACTCCCAGGTGCAGCTGGTGGAGAGCGGGGGTGGGTTAGTACAACCAG
GTGGCTCCCTACGACTTAGTTGCGCTGCAAGTGGCAGCATCTTCTCCATAAA
CGCCATGGGCTGGTACCGGCAAGCCCCTGGGAAGCAGCGTGAACTGGTGG
CAGCCATCACTTCTGGAGGCTCTACGAACTATGCAGACTCCGTTAAAGGAC
GCTTCACCATTTCAAGAGATAATGCCAAGAACACAGTGTATCTGCAGATGAA
TTCATTGAAACCCGAGGATACTGCAGTCTACTACTGTAATGCTCACAGTGTGA
AAACCCACATCCTGAGGGTGTCAGAAACCCCAGGGAGGAGGCAGCTGTGCTGGGG
CTGAGAAATGAAAGGGATTACTATTTTTAATGTTG
3) Human VH: IGHV4-4; Alpaca V_{H}H substitution: VHH3-S2
(SEQ ID NO: 90)
GGGCATGGCTAGTTGAGGCCCCAGGAAGAGAACTGAGTTCTCAAAGGGCAAAGC
AAGCATCCTCATCCCAGGGCGAGCCTAAAAGACTGGGGCCTCCCTCATCCCTTTT
CACCTCTTTATACAAAGGCACCACCTACATGCAAATCCTCACTTAGGCACCCACA
GGAAACCACCACACATTTCCTTAAATTCAGGGTCCAGCTCACATGGGAAATACTT
TCTGAGAGTCATGGACCTCCTGCACAAGAACATGAAACACCTGTGGTTCTTCCTC
CTCCTGGTGGCAGCTCCCAGATGTGAGTGTCTCAAGGCTGCAGACATGGGGGTA
TGGGAGGTGCCTCTGATCCCAGGGCTCACTGTGGGTCTCTCTGTTCACAGGGGTC
CTGTCTCAGGTGCAGCTGGTGGAGTCTGGTGGGGGCCTCGTCCAACCAGGA
GGCTCACTCCGACTTTCCTGTGCTGCTTCAGGATCGATCTTTAGTATAAATG
CCATGGGCTGGTACAGGCAGGCCCCTGGGAAGCAGAGAGAGCTGGTTGCT
GCCATCAACACTGGAGGTGGCAGCACCTATTATGCAGATTCTGTGAAAGGG
CGTTTTACCATTTCCCGGGACAATGCAAAGAACACACTGTATCTACAAATGA
ACAGCTTAAAAAGTGAAGGAACAGCTGTCTACTACTGCGCAGCCCACAGTGA
GGGGAGGTGAGTGTGAGCCCAGACACAAACCTCCCTGCAGGGAGGCGGAGGGGAC
CGGCGCAGGTGCTGCTCAGAGCCAGCAGGGGGCGCGC
4) Human VH: IGHV2-5; Alpaca V_{H}H substitution: VHH3-S9
(SEQ ID NO: 91)
TGACTTCTGCAAAGACTTCTACTCAGAATCTACTTGCCCAGCCTTAGATTAATGC
CATCTGAATTACACTGATCATGTTACTATCACTGCTCCTCACCACAGATGCAACA
CCCTCCTGAGTCCTGAAACCTGACTCCATCCCATAGAGTAGGGCACAGATGAGG
GGAATGCAAATCTCCACCAGCTCCACCCTCCTCTGGGTTGAAAAAGCCGAGCAC
AGGTCCCAGCTCAGTGACTCCTGTGCCCCACCATGGACACACTTTGCTCCACGCT
CCTGCTGCTGACCATCCCTTCATGTGAGTGCTGTGGTCAGGGACTCCTTCACGGG
TGAAACATCAGTTTTCTTGTTTGTGGGCTTCATCTTCTTATGCTTTCTCCACAGGG
GTCTTGTCCCAAGTCCAGCTGGTGGAGTCTGGTGGTGGCCTTGTACAGGCAG
GAGGGTCACTTCGGCACAGCTGTGCTGCGTCTGGACTCACCTTTGGATCCT
ATGCCATGGGCTGGTACAGGCAGGCCCCCGGGAAAGAACGAGAGCTGGTT
GCTGCCATCAGCAGTGGGGGCAGTACGTATTATGCAGACTCTGTCAAAGGC
CGCTTCACCATATCCAGAGATAATGCCAAGAACACTCTCTACCTGCAGATGA
ACAGCCTGAAGCCTGAAGGAACAGCTGTGTACTACTGCAATGCACACAAAGA
CACAGCCCAGGGCACCTCCTGTACAAAAACCCAGGCTGCTTCTCATTGGTGCTCCCT
CCCCACCTCTGCAGAACAGGAAAGTGCAGCTGAGA
5) Human VH: IGHV3-7; Alpaca V_{H}H substitution: VHH3-S10
(SEQ ID NO: 92)
ACAGCATATTTTCCAAATACCATCATTGTCAGCAAACTTCTGCAGAGCACCGTCT
TCTTATATGGGTACAGCCTATTCCTCCAGCATCCCACTAGAGCTTCTTATATAGTA
GGAGACATGCAAATAGGGCCCTCCCTCTACTGATGAAAACCAACCCAACCCTGA
CCCTGCAGGTCTCAGAGAGGAGCCTTAGCCCTGGACTCCAAGGCCTTTCCACTTG
GTGATCAGCACTGAGCACAGAGGACTCACCATGGAGTTGGGGCTGAGCTGGGTT
TTCCTTGTTGCTATTTTAGAAGGTGATTCATGGAAAACTAGGAAGATTGAGTGTG
TGTGGATATGAGTGTGAGAAACAGTGGATTTGTGTGGCAGTTTCTGACCTTGGTG
TCTCTTTGTTTGCAGGTGTCCAGTGTCAGGTGCAGCTGGTGGAGTCAGGAGGT
GGTTTGGTTCAAGCAGGGGGCAGTTTACGACACAGCTGTGCTGCTTCTGGG
CTCACTTTTGGAAGCTATGCCATGGGCTGGTATCGGCAAGCGCCAGGGAAA
GAAAGAGAACTTGTTGCAGCCATAAGCAGTGGTGGCTCAACCTACTATGCA
GACTCTGTGAAAGGACGCTTCACCATCTCCAGGGATAATGCCAAGAACACA
GTCTACCTGCAGATGAACAGCCTGAAGCCTGAAGGAACAGCTGTGTCCTAC
TGCAATGCTCACAGTGAGGGGAAGTCAGTGTGAGCCCAGACACAAACCTCCCTGCA
GGGGTCCCTTGGGACCACCAGGGGGCGACAGGGCATTGAGCACGGGGCTGTCT

### Generation of the Singularity Savnars Allelic Series

Cartilaginous fish (e.g., sharks, skates, and rays) produce heavy chain antibodies from a special class of immunoglobulin known as variable new antigen receptor (VNAR) (Greenberg et al., Nature, 374 (6518): 168-73 (1995)).

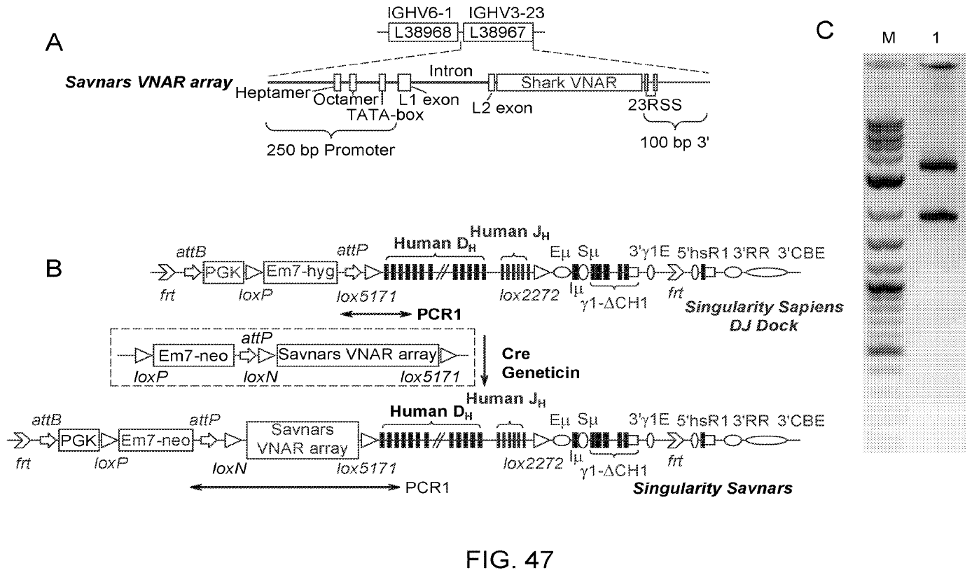

The capability of the Singularity platform was expanded by constructing a synthetic shark VNAR array. The individual VNAR element selected from the germline sequences of the nurse shark (Ginglymostoma cirratum) was grafted onto the framework of a selected human VH component that included the about 250 bp upstream human promoter containing regulatory elements (e.g., TATA-box, octamer, and heptamer) involved in VH transcription, human leader exon 1, human intron 1 and human leader exon 2 in the upstream, and human recombination signal sequences (RSS) in the downstream (FIG. 47A). The VNAR array dubbed Savnars are synthesized and are inserted into the Singularity Sapiens DJ allele via RMCE (FIGS. 47B, 47C). Mice that are generated from this genetic engineering are referred to as Singularity Savnars mice and are assessed for the capacity to produce shark-human-mouse chimeric heavy chain antibodies (e.g., shark-human-mouse chimeric heavy chain IgG1-ΔCH1 antibodies).

The shark-human-mouse chimeric heavy chain antibodies produced from the Singularity Savnars mice can have the superior biophysical properties of VNARs and benefit the additional immune diversity of the human D and J elements, enabling rapid humanization for therapeutic applications in humans. The immune repertoire of Singularity Savnars mice can be readily expanded via repeated rounds of RMCE-mediated integration of arrays containing additional VNARs from other shark species.

### Sequence of Singularity Savnars Array (Alpaca V_{H}H Sequence is Bold and Underline)

1) Human VH: IGHV6-1; Nurse shark VNAR substitution: L38968
(SEQ ID NO: 93)
GGGCCCTGCCTCTGAGCTCCTCTTTGCATCCAATCTGCTGAAGAACATGGCTCTA
GGGAAACCCAGTTGTAGACCTGAGGGCCCCGGCTCTTCAATGAGCCATCTCCGT
CCCGGGGCCTTATATCAGCAAGTGACGCACACAGGCAAATGCCAGGGTGTGGTT
TCCTGTTTAAATGTAGCCTCCCCCGCTGCAGAACTGCAGAGCCTGCTGAATTCTG
GCTGACCAGGGCAGTCACCAGAGCTCCAGACAATGTCTGTCTCCTTCCTCATCTT
CCTGCCCGTGCTGGGCCTCCCATGGGGTCAGTGTCAGGGAGATGCCGTATTCACA
GCAGCATTCACAGACTGAGGGGTGTTTCACTTTGCTGTTTCCTTTTGTCTCCAGGT
GTCCTGTCAGCCAGGGTGGACCAGACACCTCGGAGTGTTACCAAGGAGACA
GGAGAATCACTCACCATCAACTGTGTGCTTCGAGATGCTTCCTATGCATTGG
GTTCTACGTGCTGGTACAGGAAGAAGTCTGGGTCAACAAATGAAGAGAGCA
TAAGCAAAGGAGGCAGATATGTGGAGACTGTCAACAGCGGCTCCAAAAGTT
TCTCCCTGAGAATTAATGACCTGACTGTAGAAGATGGTGGCACCTACCGCT
GTGGGGTCCACAGTGAGGGGAAGTCAGTGTGAGCCCAGACACAAACCTCCCTGCA
GGGATGCTCAGGACCCCAGAAGGCACCCAGCACTACCAGCGCAGGGCCCAGAC
2) Human VH: IGHV3-23; Nurse shark VNAR substitution: L38967
(SEQ ID NO: 94)
AGCACAATTTCCCAATGCTTTCAATATCACAGATCTCCCCGAGGACATTCTGACA
TGCTCTGAGCCCCACTATCTCCAAAGGCCTCTCACCCCAGAGCTTACTATATAGT
AGGAGATATGCAAATAGAGCCCTCCGTCTGCTGATGAAAACCAGCCCAGCCCTG
ACCCTGCAGCTCTGAGAGAGGAGCCCAGCCCTGGGATTTTCAGGTGTTTTCATTT
GGTGATCAGGACTGAACAGAGAGAACTCACCATGGAGTTTGGGCTGAGCTGGCT
TTTTCTTGTGGCTATTTTAAAAGGTAATTCATGGAGAAATAGAAAAATTGAGTGT
GAATGGATAAGAGTGAGAGAAACAGTGGATACGTGTGGCAGTTTCTGACCAGGG
TTTCTTTTTGTTTGCAGGTGTCCAGTGTGCTCGAGTAGACCAGACTCCTAAGAC
TATCACCAAGGAGACAGGAGAATCACTCACCATCAACTGTGTTCTTAGAGAT
ACGTCCTATGCATTGGGGTCCACCTACTGGTACAGGAAGAAGCTGGGCTCT
ACAAATGAGGAAAGCATTTCAAAAGGTGGGCGGTATGTGGAGACTGTCAAC
AGTGGAAGCAAAAGTCTATCTCTGCGTATAAATGGCCTGAAGGTGGAAGAC
AGCTGGACATACCGCTGCAAAGCCACAGTGAGGGGAAGTCATTGTGAGCCCAGA
CACAAACCTCCCTGCAGGAACGATGGGGGGGAAATCAGCGGCAGGGGGCGCTCA
GGACCCGCTGATCAGA

### Example 10: Singularity Sapiens TCR Allelic Series

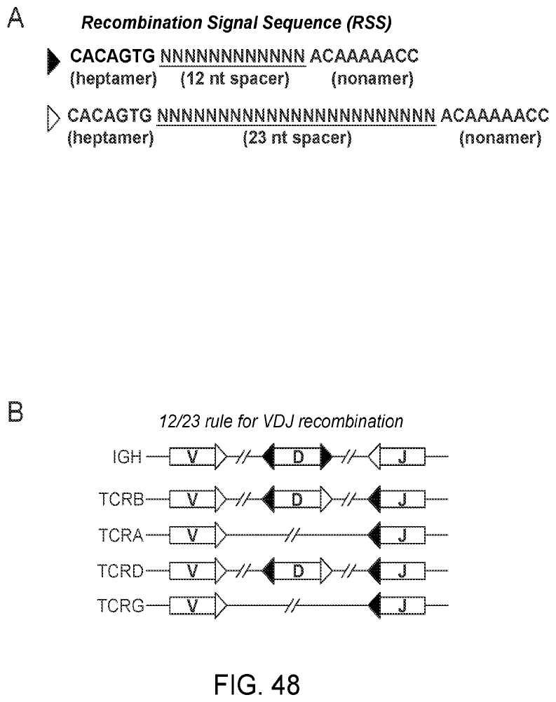

This Example describes the design and generation of nucleic acid constructs that can be used to generate mice expressing a TCR variable domain gene segment with Ig constant domains. TCR is a membrane surface heterodimeric protein consisting of pairs of either α and β subunits or γ and δ subunits. TCR associates with CD3 proteins and recognizes specific peptide bound within the MHC complex (pMHC) via the variable domains of either αβ or γδ. Analogous to VDJ recombination seen in the generation of the heavy chains and light chains of B-cell antibodies, formation of distinct αβ and γδ TCRs is driven by RAG1/2-mediated recombination of discrete V, D, and J gene segments to assemble either the β or δ chains and recombination of V and J gene segments to assemble either the α or γ chains (FIGS. 48A-48B).

A human TCRβ variable domain gene segment repertoire is engineered into a Singularity mouse platform to express antibody-like molecules containing (a) a TCRβ variable domain and (b) IgG Hinge, IgG CH2 and IgG CH3 domains.

One approach was designed to utilize V, D, and J gene segments of TCRB to drive VDJ recombination. A hTCRB-VDJ-BAC1 was engineered to contain one TCRB D gene segment (TRBD1), six TCRB J gene segments (TRBJ1-1, TRBJ1-2, TRBJ1-3, TRBJ1-4, TRBJ1-5, and TRBJ1-6), and the first seven TCRB V gene segments (TRBV20-1, TRBV23-1, TRBV24-1, TRBV25-1, TRBV27, TRBV28, and TRBV29-1 (FIG. 49A). Another approach was designed to utilize only V gene segments of TCRB to undergo VDJ recombination with D and J gene segments of IGH. A hTCRB-V-BAC1 was engineered to contain only the first seven TCRB V segments (TTRBV20-1, TRBV23-1, TRBV24-1, TRBV25-1, TRBV27, TRBV28, and TRBV29-1) (FIG. 50A).

TABLE 8
List of TCRB BACs and their corresponding
chromosome coordinates.

| | Source BAC Chromosome | |
|---|---|---|
| Source BAC | coordinates | Engineered BAC |
| CH17-318M5 | chr7: 142618756-142824109 | hTCRB-VDJ-BAC1 |
| CH17-44P21 | chr7: 142479113-142695716 | hTCRB-VDJ-BAC2 |
| CH17-366L11 | chr7: 142298700-142498385 | hTCRB-VDJ-BAC3 |
| CH17-318M5 | chr7: 142618756-142824109 | hTCRB-V-BAC1 |
| CH17-44P21 | chr7: 142479113-142695716 | hTCRB-V-BAC2 |
| CH17-366L11 | chr7: 142298700-142498385 | hTCRB-V-BAC3 |

### Example 11. Singularity Sapiens TCR Mice Expressing Human TCR B VDJ Gene Segments with Nucleic Acid Encoding Mouse Ig Constant Domains

This Example describes generation of mice expressing human TCRB variable domain gene segments with mouse IgG CH2 and CH3 nucleic acid to produce antibody-like molecules that include (1) a mouse IgG Hinge, mouse IgG CH2 domain and a mouse IgG CH3 domain, and (2) a human TCRB variable domain. These antibody-like molecules also lack mouse IgG CH1 domains.

A BAC1 (CH17-318M5) containing one TCRB D gene segment (TRBD1), six TCR J gene segments (TRBJ1-1, TRBJ1-2, TRBJ1-3, TRBJ1-4, TRBJ1-5, and TRBJ1-6), and the first seven TCR V gene segments (T TRBV20-1, TRBV23-1, TRBV24-1, TRBV25-1, TRBV27, TRBV28, and TRBV29-1) was engineered to contain three lox sites (LoxP, Lox5171, and Lox2272) and a hygromycin selection cassette (FIG. 49B). The engineered TCRB-VDJ-BAC1 was integrated into the mouse Igh locus of ES cells harboring the Singularity Hyperdock allele via RMCE (FIG. 49B). Using this approach, we were able to generate Singularity Sapiens TCR ES cells and F1 mice in turn, in which each TCRB V gene segment was confirmed by PCR and Sanger sequencing (FIGS. 49C and 49D).

The rest of the TCRB V gene segments are incorporated with two other engineered VDJ BACs-hTCRB-VDJ-BAC2 and hTCRB-VDJ-BAC3 (Table 8) using sequential RMCE.

A similar approach is used to expand the immune repertoire of the Singularity Sapiens TCR platform by adding V gene segments from TCRA, TCRD and/or TCRG.

### Example 12. Singularity Sapiens TCR Mice Expressing Human TCR B V Gene Segments with Nucleic Acid Encoding Human Ig D and J Domains and with Nucleic Acid Encoding Mouse Constant Domains

This Example describes generation of mice expressing human TCRB variable domain gene segments with human IgG D and J gene segments and with mouse IgG CH2 and CH3 nucleic acid to produce antibody-like molecules that include (1) a mouse IgG CH2 domain and a mouse IgG CH3 domain, (2) a human TCRB variable domain, and (3) human IgG D and J domains. These antibody-like molecules also lack mouse IgG CH1 domains.

A BAC1 (CH17-318M5; FIG. 50A) containing the first seven human TCR V segments (TRBV20-1, TRBV23-1, TRBV24-1, TRBV25-1, TRBV27, TRBV28, and TRBV29-1) is engineered to contain three lox sites (LoxP, LoxN, and Lox5171) and a neomycin selection cassette (FIG. 50B). The engineered TCRB-V-BAC1 is integrated into the mouse Igh locus of ES cells harboring the singularity DJ dock allele via RMCE (FIG. 50B). The rest of the TCRB V gene segments are incorporated using two other V BACs-hTCRB-V-BAC2 and hTCRB-V-BAC3 using sequential RMCE.

A similar approach is used to expand the immune repertoire of the singularity TCR platform by adding V gene segments from TCRA, TCRD and/or TCRG.

### Example 13: Constructs for Producing Antibody-Like Molecules Having Amino Acid Sequences of a ¹⁰FN3 Polypeptide

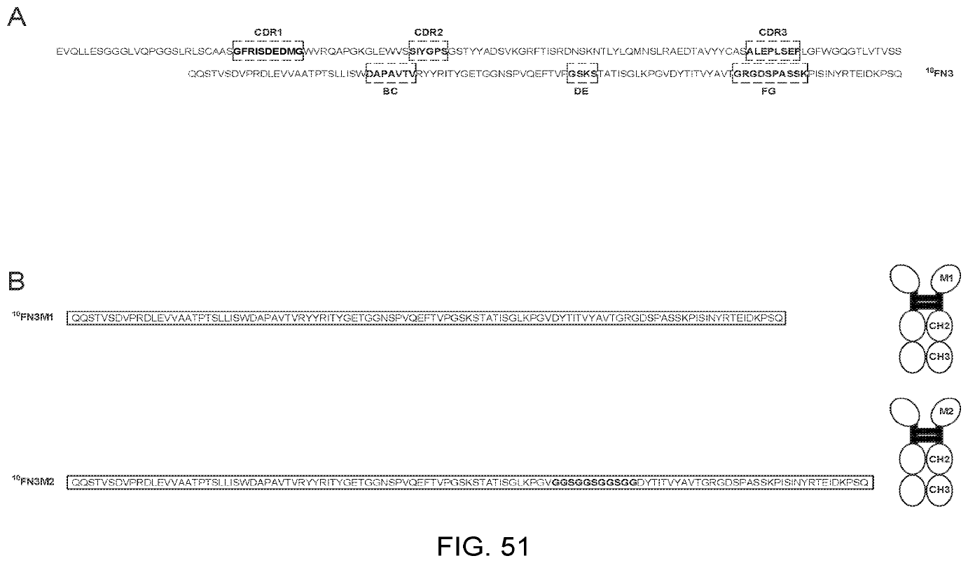

¹⁰FN3 has three exposed loops termed BC, DE, and FG that correspond to the three CDRs of antibody variable domain (FIG. 51A). To test the secretability of ¹⁰FN3 when expressed as a part of the IgG heavy chain, two constructs of ¹⁰FN3, 10FN3M1 and ¹⁰FN3M2, were synthesized. Each contained a C-terminal Fc and N-terminal signal peptide. ¹⁰FN3M1 has the same amino acid sequence as that of ¹⁰FN3, while an 11 amino acid spacer was added in ¹⁰FN3M2 to test ¹⁰FN3 tolerance to altered framework (FIG. 51B).

The synthetic constructs were cloned into plasmids, which were then transfected into CHO cells for transient expression. Both ¹⁰FN3 construct expressed robustly in CHO cells, and each protein was efficiently secreted to the media. In addition, the secreted Fc fusion monobodies exhibited excellent solubility (Table 9), with ¹⁰FN3M1 being superior to ¹⁰FN3M2.

TABLE 9
Analytical results of ¹⁰FN3M1 and ¹⁰FN3M2 purified from a 30 mL culture of CHO cells.

| | Estimated | Reducing | Non- | | | Endotoxin | | Expression |
|---|---|---|---|---|---|---|---|---|
| ¹⁰FN3 | Amount | Molecular | reducing | SEC-214 | SEC-280 | level | Concentration | yield |
| construct | (mg) | Weight (MW) | MW | nm | nm | (EU/) | (mg/mL) | (mg/L) |
| ¹⁰FN3M1 | 10.66 | 37 kDa | 74 kDa | 99.587% | 99.609% | <1 | 2.03 | 355.33 |
| ¹⁰FN3M2 | 2.55 | 38 kDa | 76 kDa | 56.309% | 56.331% | <1 | 0.92 | 85 |

Next, ¹⁰FN3 was engineered to test its ability to act as Ig frameworks. Based on alignment of its amino acids with that of the antibody variable region (FIG. 51A), ¹⁰FN3 was split into two segments: a “VM” segment and a “JM” segment, leaving out the FG loop region (FIG. 52A). A 23RSS sequence was appended to the 3′ end of the “VM” segment and “JM” segment to enable RAG1/2-mediated VDJ recombination. The “VM” and “JM” segments were synthesized separately (FIG. 52A). The “VM” construct was mouse-codon optimized and placed within a mouse VH scaffold containing a 250 bp upstream promoter of a mouse VH (IGHV1-26), leader exon 1, intron 1, and leader exon 2, and 100 bp downstream sequence containing the 23RSS sequence (FIG. 52B).

### Example 14: Exemplary ¹⁰FN3 Domains

Amino acid sequence of an exemplary first ¹⁰FN3
amino acid sequence for a VM1 domain:
(SEQ ID NO: C24)
QQSTVSDVPRDLEVVAATPTSLLISWDAPAVTVRYYRITYGETGGNSPV
QEFTVPGSKSTATISGLKPGVDYTITVYAVT
Amino acid sequence of an exemplary second ¹⁰FN3
amino acid sequence for a VM1 domain:
(SEQ ID NO: C26)
QQSTVSDVPRDLEVVAATPTSLLISWDAPAVTVRYYRITYGETGGNSPV
QEFTVPGSKSTATISGLKPGVGGSGGSGGSGGDYTITVYAVT
Amino acid sequence of an exemplary first ¹⁰FN3
amino acid sequence for a JM domain:
(SEQ ID NO: C25)
PISINYRTEIDKP
Amino acid sequence of an exemplary second ¹⁰FN3
amino acid sequence for a JM domain:
(SEQ ID NO: C27)
PISINYRTEIDKPSQ

### Example 15: Singularity Sapiens Monobody Mice for Producing Antibody-Like Molecules Having Amino Acid Sequences of a ¹⁰FN3 Polypeptide

This Example describes generation of mice that can express (a) a first amino acid sequence of a FN3 polypeptide (e.g., a ¹⁰FN3 polypeptide), (b) a human Ig variable D domain, (c) a second amino acid sequence of a FN3 polypeptide (e.g., a ¹⁰FN3 polypeptide), and (d) a mouse Ig CH2 domain and a mouse Ig CH3 domain (e.g., endogenous Ig CH2 and/or CH3 domains). Such mice can be used to generate miniaturized single domain monobodies that are more stable and function intracellularly as antibody-like molecules.

Two constructs are introduced into mouse ES cells harboring the Singularity Sapiens DJ dock containing all human IGHD and IGHJ segments (FIG. 53). First, an ¹⁰FN3JM construct is used to swap out the human IGHJ segments via CRISPR-Cas9. Next, the construct containing ¹⁰FN3VM1, three lox sites (LoxP, LoxN, and Lox5171), and a neomycin selection cassette is integrated upstream of the human IGHD gene segments via RMCE. Using this approach, we were able to generate Singularity Sapiens Monobody ES cell clones for chimera generation (FIG. 53).

Such generated mice are used to produce antibody-like molecules having a stable scaffold of ¹⁰FN3VM, random human IGHDs for enhanced CDR3 diversity, and ¹⁰FN3J joined to the constant region of CH1 truncated IgG1. Additional antibody-like molecule diversity is driven by junctional diversification during VDJ recombination and somatic hypermutation during in vivo affinity maturation.

### Example 16: Modified Human JH Domains for Improved Solubility and Stability

This Example describes the design and generation of nucleic acid constructs that can be used to generate mice that can express particular modified human JH domains with mouse Ig constant domains.

To examine whether substitution of tryptophan within human JH domains with an amino acid other than tryptophan improves solubility and stability of single domain antibodies, two fully human sdAbs generated from the singularity platform (HS5_91 and HS6_106) were selected for protein engineering to produce two sets of nanobody-Fc fusions in which the tryptophan within framework region 4 (W103), which is encoded by a human JH domain, was substituted for various amino acids (W2X). Analysis of the size exclusion chromatography (SEC) profiles and the protein yields indicated that substitution with any of aspartic acid (D), glutamic acid (E), or proline (P) led to severe aggregation and low protein yield. By contrast, substitution with any of arginine (R), lysine (K), tyrosine (Y), serine(S), threonine (T), histidine (H), or glutamine (Q) resulted in high levels of expression of Nb-Fcs that were less prone to aggregation, and thus may increase nanobody solubility and stability (Table 10).

TABLE 10
Biophysical analytic results of two human nanobodies (HS5_91 and HS6_106)
in which the W103 residue was substituted with indicated amino acids.

| | | Non- | | | | Expression |
|---|---|---|---|---|---|---|
| Nb-Fc W2X | Reducing | reducing | SEC-214 | SEC-280 | Concentration | yield |
| mutants | MW | MW | nm | nm | (mg/mL) | (mg/L) |
| HS5_91_W_R | 40 kDa | 80 kDa | 91.764% | 92.196% | 0.65 | 91 |
| HS5_91_W_K | 40 kDa | 80 kDa | 91.667% | 92.048% | 0.61 | 86 |
| HS5_91_W_D | 40 kDa | 80 kDa | 1.587% | 1.404% | 0.17 | 12.67 |
| HS5_91_W_E | 40 kDa | 80 kDa | 37.867% | 38.604% | 0.27 | 25 |
| HS5_91_W_Y | 40 kDa | 80 kDa | 49.447% | 49.203% | 1.21 | 151.4 |
| HS5_91_W_S | 40 kDa | 80 kDa | 79.895% | 79.522% | 1.19 | 164 |
| HS5_91_W_T | 40 kDa | 80 kDa | 89.076% | 88.739% | 1.2 | 162 |
| HS5_91_W_H | 40 kDa | 80 kDa | 88.287% | 88.908% | 1.09 | 153 |
| HS5_91_W_P | 40 kDa | 80 kDa | 3.442% | 3.984% | 0.19 | 3 |
| HS5_91_W_Q | 40 kDa | 80 kDa | 87.313% | 87.116% | 0.64 | 85 |
| HS6_106_W_R | 39 kDa | 78 kDa | 80.150% | 80.082% | 0.8 | 100 |
| HS6_106_W_K | 39 kDa | 78 kDa | 62.267% | 63.248% | 0.63 | 87 |
| HS6_106_W_D | 39 kDa | 78 kDa | 7.414% | 7.056% | 0.29 | 10.33 |
| HS6_106_W_E | 39 kDa | 78 kDa | 24.811% | 25.270% | 0.12 | 14 |
| HS6_106_W_Y | 39 kDa | 78 kDa | 95.120% | 95.855% | 1.23 | 185 |
| HS6_106_W_S | 39 kDa | 78 kDa | 53.890% | 55.002% | 1.19 | 134 |
| HS6_106_W_T | 39 kDa | 78 kDa | 70.105% | 70.135% | 1.47 | 202.33 |
| HS6_106_W_H | 39 kDa | 78 kDa | 62.972% | 63.927% | 1.27 | 172 |
| HS6_106_W_P | 39 kDa | 78 kDa | 24.420% | 24.745% | 0.3 | 30 |
| HS6_106_W_Q | 39 kDa | 78 kDa | 70.134% | 70.962% | 0.78 | 107.4 |

The W103 residue is encoded in the six human J segments. Analysis of human HcAbs generated from the Singularity Sapiens mice revealed preferential usage of IGHJ3 and IGHJ4. A synthetic construct (Syn JH-W2X) was designed to substitute W103 residue in the IGHJ3 and IGHJ4 gene segments with R and H, respectively (FIG. 54A).

### Example 17: Exemplary Modified Human JH Gene Segments

Wild-type human JH3 (IGHJ3*02)
Nucleic acid sequence of wild-type JH3 gene
segment
(SEQ ID NO: 107)
GATGCTTTTGATATCTGGGGCCAAGGGACAATGGTCACCGTCTCTTCAG
Amino acid sequence of wild-type JH3 domain
(SEQ ID NO: 108)
DAFDIWGQGTMVTVSS
Wild-type human JH4 (IGHJ4*02)
Nucleic acid sequence of wild-type JH4 gene
segment
(SEQ ID NO: 109)
TACTTTGACTACTGGGGCCAGGGAACCCTGGTCACCGTCTCCTCAG
Amino acid sequence of wild-type JH4 domain
(SEQ ID NO: 110)
YFDYWGQGTLVTVSS
Examples of modified human JH3
Nucleic acid sequence of modified JH3 gene segment
(SEQ ID NO: 111)
GATGCTTTTGATATCNNNGGCCAAGGGACAATGGTCACCGTCTCTTCAG
Amino acid sequence of modified JH3 domain
(SEQ ID NO: 105)
DAFDIXGQGTMVTVSS
Examples of modified human JH4
Nucleic acid sequence of modified JH4 gene segment
(SEQ ID NO: 112)
TACTTTGACTACNNNGGCCAGGGAACCCTGGTCACCGTCTCCTCAG
Amino acid sequence of modified JH4 domain
(SEQ ID NO: 106)
YFDYXGQGTLVTVSS

For each of the above examples, “NNN” in the nucleic acid sequence can be any codon other than a stop codon or a codon encoding tryptophan, and “X” in the amino acid sequence can be any amino acid other than tryptophan. In some cases, the “X” in the amino acid sequence can be arginine (R), lysine (K), tyrosine (Y), serine(S), threonine (T), histidine (H), or glutamine (Q).

Syn-JH-W2X Construct:
(SEQ ID NO: 113)
AGCCCCCAGCCCCACAGGCCCCCTACCAGCCGCAGGGTTTTGGCTGAGC
TGAGAACCACTGTGCTAACTGGGGACACAGTGATTGGCAGCTCTACAAA
AACCATGCTCCCCCGGGACCCCGGGCTGTGGGTTTCTGTAGCCCCTGGC
TCAGGGCTGACTCACCGTGGCTGAATACTTCCAGCACTGGGGCCAGGGC
ACCCTGGTCACCGTCTCCTCAGGTGAGTCTGCTGTCTGGGGATAGCGGG
GAGCCAGGTGTACTGGGCCAGGCAAGGGCTTTGGCTTCAGACTTGGGGA
CAGGTGCTCAGCAAAGGAGGTCGGCAGGAGGGCGGAGGGTGTGTTTTTG
TATGGGAGAAGCAGGAGGGCAGAGGCTGTGCTACTGGTACTTCGATCTC
TGGGGCCGTGGCACCCTGGTCACTGTCTCCTCAGGTGAGTCCCACTGCA
GCCCCCTCCCAGTCTTCTCTGTCCAGGCACCAGGCCAGGTATCTGGGGT
CTGCAGCCGGCCTGGGTCTGGCCTGAGGCCACACCAGCTGCCATCCCTG
GGGTCTCCGCCATGGGCTGCATGCCAGAGCCCTGCTGTCACTTAGCCCT
GGGGCCAGCTGGAGCCCCCAAGGACAGGCAGGGACCCCGCTGGGCTTCA
GCCCCGTCAGGGACCCTCCACAGGTAGCAAGCAGGCCGAGGGCAGGGAC
GGGAAGGAGAAGTTGTGGGCAGAGCCTGGGCTGGGGCTGGGCGCTGGCT
GTTCATGTGCCGGGGACCAGGCCTGCGCTTTAGTGTGGCTACAAGTGCT
TGGAGCACTGGGGCCAGGGCAGCCCGGCCACCGTCTCCCTGGGAACGTC
ACCCCTCCCTGCCTGGGTCTCAGCCCGGGGGTCTGTGTGGCTGGGGACA
GGGACGCCGGCTGCCTCTGCTCTGTGCTTGGGCCATGTGACCCATTCGA
GTGTCCTGCACGGGCACAGGTTTATGTCTGGGCAGGAACAGGGACTGTG
TCCCTGTGTGATGCTTTTGATATCNNNGGCCAAGGGACAATGGTCACCG
TCTCTTCAGGTAAGATGGCTTTCCTTCTGCCTCCTTTCTCTGGGCCCAG
CGTCCTCTGTCCTGGAGCTGGGAGATAATGTCCGGGGGCTCCTTGGTCT
GCGCTGGGCCATGTGGGGCCCTCCGGGGCTCCTTCTCCGGCTGTTTGGG
ACCACGTTCAGCAGAAGGCCTTTCTTTGGGAACTGGGACTCTGCTGCTG
GGGCAAAGGGTGGGCAGAGTCATGCTTGTGCTGGGGACAAAATGACCTT
GGGACACGGGGCTGGCTGCCACGGCCGGCCCGGGACAGTCGGAGAGTCA
GGTTTTTGTGCACCCCTTAATGGGGCCTCCCACAATGTGACTACTTTGA
CTACNNNGGCCAGGGAACCCTGGTCACCGTCTCCTCAGGTGAGTCCTCA
CAACCTCTCTCCTGCTTTAACTCTGAAGGGTTTTGCTGCATTTTTGGGG
GGAAATAAGCGTGCTGGGTCTCCTGCCAAGAGAGCCCCGGAGCAGCCTG
GGGGGCTCAGGAGGATGCCCTGAGGCAACAGCGGCCACACAGACGAGGG
GCAAGGGCTCCAGATGCTCCTTCCTCCTGAGCCCAGCAGCACGGGTCTC
TCTGTGGCCAGGGCCACCCTGGGCCTCTGGGGTCCAATGTCCAACAACC
CCCGGGCCCTCCCCGGGCTCAGTCTGAGAGGGTCCCAGGGACTTAGCGG
GGTGCCAGTTCTTGCCTGGGGTCCTGGCATTGTTGTCACAATGTGACAA
CTGGTTCGACCCCTGGGGCCAGGGAACCCTGGTCACCGTCTCCTCAGGT
GAGTCCTCACCACCCCCTCTCTGAGTCCACTTAGGGAGACTCAGCTTGC
CAGGGTCTCAGGGTCAGAGTCTTGGAGGCATTTTGGAGGTCAGGAAAGA
AAGCTGGGGAGAGGGACCCTTCGAATGGGAACCCAGCCTGTCCTCCCCA
AGTCCGGCCACAGATGTCGGCAGCTGGGGGGCTCCTTCGGCTGGTCTGG
GGTGACCTCTCTCCGCTTCACCTGGAGCATTCTCAGGGGCTGTCGTGAT
GATTGCGTGGTGGGACTCTGTCCCGCTCCAAGGCACCCGCTCTCTGGGA
CGGGTGCCCCCCGGGGTTTTTGGACTCCTGGGGGTGACTTAGCAGCCGT
CTGCTTGCAGTTGGACTTCCCAGGCCGACAGTGGTCTGGCTTCTGAGGG
GTCAGGCCAGAATGTGGGGTACGTGGGAGGCCAGCAGAGGGTTCCATGA
GAAGGGCAGGACAGGGCCACGGACAGTCAGCTTCCATGTGACGCCCGGA
GACAGAAGGTCTCTGGGTGGCTGGGTTTTTGTGGGGTGAGGATGGACAT
TCTGCCATTGTGAT

For each of the above sequences, the first section of bold and underlined text corresponds to IGHJ3 with the codon for W103 indicated by NNN, and the second section of bold and underlined text corresponds to IGHJ4 with the codon for W103 indicated by NNN.

### Example 18: Mice Expressing Modified Human JH Domains with Mouse Ig Constant Domains

This Example describes the generation of mice that can express particular modified human JH domains with mouse Ig constant domains.

The Syn-JH-W2X construct is introduced into ES cells harboring the Singularity Sapiens allele (e.g., SSV5) via CRISPR-Cas9 to replace the endogenous IGHJ3-IGHJ4 (FIG. 54B). Other similar Syn JH-W2X constructs are generated to examine other substitutions including substitution of W103 residue in the IGHJ3 and/or IGHJ4 gene segments with serine(S), threonine (T), lysine (K), and/or tyrosine (Y). Each similar construct is introduced into ES cells harboring the Singularity Sapiens allele via CRISPR-Cas9 as described for the Syn-JH-W2X construct. Engineered ES lines are used to produce mice that generate HcAbs that lack tryptophan residues in the human JH domain and a) lack tryptophan residues in the human JH domain, (b) have improved stability and/or solubility, and/or (c) have improved pH-responsiveness and/or plasma half-life.

### Example 19: Modified FR2 Regions of Human VH Domains for Improved Solubility and Stability

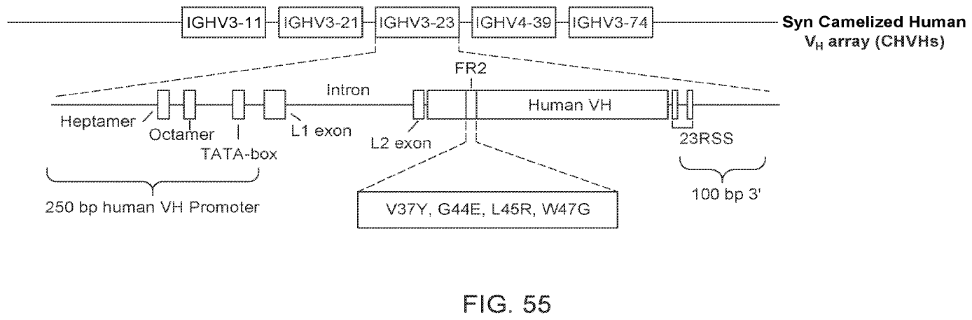

This Example describes the design of a nucleic acid construct that can be used to generate mice that can express particular modified human VH segments together with mouse Ig constant domains. To further improve the biochemical properties of nanobodies generated from the singularity platform, five human germline VH gene segments (IGHV3-11, IGHV3-21, IGHV3-23, IGHV4-39, and IGHV3-74 gene segments) were selected, and the nucleic acid encoding the VH domain of each of these was modified such that the amino acid residues corresponding to positions 37, 44, 45, and 47 within the FR2 of each of these VH domains were changed to Y, E, R, and G, respectively (FIG. 55). Each VH gene segment contained its own regulatory elements including a 250 bp upstream promoter, leader exon 1 and 2, intron, and 100 bp downstream sequence containing the 23RSS recombination signal sequences. The modified amino acid sequences of these five VH domains are listed in Example 22.

### Example 20: Exemplary Sequences of Modified Human VH Domains

In each of the nucleic acid sequences shown in this Example, the modified nucleotide codons are in bold and underlined text. The sequences correspond to the most highly utilized mouse codons for the modified amino acids.

1) Modified IGHV3-11
(SEQ ID NO: 114)
GTTATATTTTCCTGAGAGATAGGATTACCTCCAGTGTTTTCCGGGACCC
TCTCATCTGCTCTGGGCACTGCCCTCTCCTCCAGCGTCCCACTAGAGCT
TGCTATATAGTAGGAGACATGCAAATAGGGCCCTCCCTCTGCTGATAAA
AACCAGCCGAGCCCAGACCCTGCAGCTCTGGGAGAAGAGCCCCAGCCCC
AGAATTCCCAGGAGTTTCCATTCGGTGATCAGCACTGAACACAGAGGAC
TCACCATGGAGTTTGGGCTGAGCTGGGTTTTCCTTGTTGCTATTATAAA
AGGTGATTTATGGAGAACTAGAGACATTGAGTGGACGTGAGTGAGATAA
GCAGTGAATATATGTGGCAGTTTCTGACCAGGTTGTCTCTGTGTTTGCA
GGTGTCCAGTGTCAGGTGCAGCTGGTGGAGTCTGGGGGAGGCTTGGTCA
AGCCTGGAGGGTCCCTGAGACTCTCCTGTGCAGCCTCTGGATTCACCTT
CAGTGACTACTACATGAGCTGGTACCGCCAGGCTCCAGGGAAGGAGAGG
GAGGGCGTTTCATACATTAGTAGTAGTAGTAGTTACACAAACTACGCAG
ACTCTGTGAAGGGCCGATTCACCATCTCCAGAGACAACGCCAAGAACTC
ACTGTATCTGCAAATGAACAGCCTGAGAGCCGAGGACACGGCTGTGTAT
TACTGTGCGAGAGACACAGTGAGGGGAGGTCAGTGTGAGCCCAGACACA
AACCTCCCTGCAGGGGTCCCCAGGACCACCAGGGGGCGCCCGGGACACT
GTGCACGGGGCTGTCT
2) Modified IGHV3-21
(SEQ ID NO: 115)
GACAGCATATTTTCCAAATACCATCGTCAGCAAACATCTGCAGGGCACC
GTCTTATTATCTGGGTACAGCCTATTCCTCCAGCGTCCCACCCTAGAGC
TTGTTATATAGTAGGAGATATGCAAATAGGGACCTCCCTCTACTGATGA
AAACCAACCGAACCCTGACCCTGCAGCTCTGAGAGAGGAGCCTTAGCCC
TGGATTCCAAGGCCTATCCACTTGGTGATCAGCACTGAGCACCGAGGAT
TCACCATGGAACTGGGGCTCCGCTGGGTTTTCCTTGTTGCTATTTTAGA
AGGTGAATCATGGAAAAGTAGAGAGATTTAGTGTGTGTGGATATGAGTG
AGAGAAACGGTGGATGTGTGTGACAGTTTCTGACCAATGTCTCTCTGTT
TGCAGGTGTCCAGTGTGAGGTGCAGCTGGTGGAGTCTGGGGGAGGCCTG
GTCAAGCCTGGGGGGTCCCTGAGACTCTCCTGTGCAGCCTCTGGATTCA
CCTTCAGTAGCTATAGCATGAACTGGTACCGCCAGGCTCCAGGGAAGGA
GAGGGAGGGCGTCTCATCCATTAGTAGTAGTAGTAGTTACATATACTAC
GCAGACTCAGTGAAGGGCCGATTCACCATCTCCAGAGACAACGCCAAGA
ACTCACTGTATCTGCAAATGAACAGCCTGAGAGCCGAGGACACGGCTGT
GTATTACTGTGCGAGAGACACAGTGAGGGGAAGTCAGTGTGAGCCCAGA
CACAAACCTCCCTGCAGGGGTCCCCAGGACCACCAGGGGGCGCCCGGGA
CACTGTGCACGGGGCTGTCT
3) Modified IGHV3-23
(SEQ ID NO: 116)
AGCACAATTTCCCAATGCTTTCAATATCACAGATCTCCCCGAGGACATT
CTGACATGCTCTGAGCCCCACTATCTCCAAAGGCCTCTCACCCCAGAGC
TTACTATATAGTAGGAGATATGCAAATAGAGCCCTCCGTCTGCTGATGA
AAACCAGCCCAGCCCTGACCCTGCAGCTCTGAGAGAGGAGCCCAGCCCT
GGGATTTTCAGGTGTTTTCATTTGGTGATCAGGACTGAACAGAGAGAAC
TCACCATGGAGTTTGGGCTGAGCTGGCTTTTTCTTGTGGCTATTTTAAA
AGGTAATTCATGGAGAAATAGAAAAATTGAGTGTGAATGGATAAGAGTG
AGAGAAACAGTGGATACGTGTGGCAGTTTCTGACCAGGGTTTCTTTTTG
TTTGCAGGTGTCCAGTGTGAGGTGCAGCTGGTGGAGTCTGGGGGAGGCT
TGGTACAGCCTGGGGGGTCCCTGAGACTCTCCTGTGCAGCCTCTGGATT
CACCTTTAGCAGCTATGCCATGAGCTGGTACCGCCAGGCTCCAGGGAAG
GAGAGGGAGGGCGTCTCAGCTATTAGTGGTAGTGGTGGTAGCACATACT
ACGCAGACTCCGTGAAGGGCCGGTTCACCATCTCCAGAGACAATTCCAA
GAACACGCTGTATCTGCAAATGAACAGCCTGAGAGCCGAGGACACGGCC
GTATATTACTGTGCGAAAGACACAGTGAGGGGAAGTCATTGTGAGCCCA
GACACAAACCTCCCTGCAGGAACGATGGGGGGGAAATCAGCGGCAGGGG
GCGCTCAGGACCCGCTGATCAG
4) Modified IGHV4-39
(SEQ ID NO: 117)
TGGGCTTGGAGAGGGGAGGCCCCAAGAAGAGAACTGAGTTCTCAAAGGG
CACAGCCAGCATTCTCCTCCCAGGGTGAGCTCAAAAGACTGGCGCCTCT
CTCATCCCTTTTCACTGCTCCGTACAAACGCACCACCCCCATGCAAATC
CTCACTTAGGCGCCCACAGGAAGCCACCACACATTTCCTTAAATTCAGG
TCCAACTCATAAGGGAAATGCTTTCTGAGAGTCATGGATCTCATGTGCA
AGAAAATGAAGCACCTGTGGTTCTTCCTCCTGCTGGTGGCGGCTCCCAG
ATGTGAGTGTTTCTAGGATGCAGACATGGAGATATGGGAGGCTGCCTCT
GATCCCAGGGCTCACTGTGGGTTTTTCTGTTCACAGGGGTCCTGTCCCA
GCTGCAGCTGCAGGAGTCGGGCCCAGGACTGGTGAAGCCTTCGGAGACC
CTGTCCCTCACCTGCACTGTCTCTGGTGGCTCCATCAGCAGTAGTAGTT
ACTACTGGGGCTGGTACCGCCAGCCCCCAGGGAAGGAGAGGGAGGGCAT
TGGGAGTATCTATTATAGTGGGAGCACCTACTACAACCCGTCCCTCAAG
AGTCGAGTCACCATATCCGTAGACACGTCCAAGAACCAGTTCTCCCTGA
AGCTGAGCTCTGTGACCGCCGCAGACACGGCTGTGTATTACTGTGCGAG
ACACACAGTGAGGGGAGGTGAGTGTGAGCCCAGACAAAAACCTCCCTGC
AGGGAGGCTGAGGGGGGGGCGCAGGTGCAGCTCAGGGCCAGCAGGGGGC
GTGC
5) Modified IGHV3-74
(SEQ ID NO: 118)
TTCATAATAAGCACAATTTCTCAAATCCCATTGTTGTCACCCATCTTCC
TCAGGACACTTTCATCTGCCCTGGGTCCTGCTCTTTCTTCAGGTGTCTC
ACCCCAGAGCTTGATATATAGTAGGAGACATGCAAATAGGGCCCTCACT
CTGCTGAAGAAAACCAGCCCTGCAGCTCTGGGAGAGGAGCCCCAGCCCT
GGGATTCCCAGCTGTTTCTGCTTGCTGATCAGGACTGCACACAGAGAAC
TCACCATGGAGTTTGGGCTGAGCTGGGTTTTCCTTGTTGCTATTTTAAA
AGGTGATTCATGGAGAACTGGAGATATGGAGTGTGAATGGACATGAGTG
AGATAAGCAGTGGATGTGTGTGGCAGTTTCTGACCAGGGTGTCTCTGTG
TTTGCAGGTGTCCAGTGTGAGGTGCAGCTGGTGGAGTCCGGGGGAGGCT
TAGTTCAGCCTGGGGGGTCCCTGAGACTCTCCTGTGCAGCCTCTGGATT
CACCTTCAGTAGCTACTGGATGCACTGGTACCGCCAAGCTCCAGGGAAG
GAGAGGGTGGGCGTCTCACGTATTAATAGTGATGGGAGTAGCACAAGCT
ACGCGGACTCCGTGAAGGGCCGATTCACCATCTCCAGAGACAACGCCAA
GAACACGCTGTATCTGCAAATGAACAGTCTGAGAGCCGAGGACACGGCT
GTGTATTACTGTGCAAGAGACACAGTGAGGGGAAGTCAATGTGAGCCCA
GACACAAACCTCGCTGCAGGGGCATCTGAGACCACGAGGGGGTGTCCTG
GGCCCTGTGAACTGGGCTGCTC

In each of the amino acid sequences shown in this Example, the modified residues are in bold and underlined text in the following order: amino acid position 37, amino acid position 44, amino acid position 45, and amino acid position 47. In some cases, any of the following human Ig VH domains can be modified to have only one of the four depicted substitutions, to have only two of the four depicted substitutions, to have only three of the four depicted substitutions.

1) Polypeptide encoded by a modified IGHV3-11
(SEQ ID NO: 119)
QVQLVESGGGLVKPGGSLRLSCAASGFTFSDYYMSWYRQAPGKEREGVSY
ISSSSSYTNYADSVKGRFTISRDNAKNSLYLQMNSLRAEDTAVYYCAR
2) Polypeptide encoded by a modified IGHV3-21
(SEQ ID NO: 120)
EVQLVESGGGLVKPGGSLRLSCAASGFTFSSYSMNWYRQAPGKEREGVSS
ISSSSSYIYYADSVKGRFTISRDNAKNSLYLQMNSLRAEDTAVYYCAR
3) Polypeptide encoded by a modified IGHV3-23
(SEQ ID NO: 121)
EVQLVESGGGLVQPGGSLRLSCAASGFTFSSYAMSWYRQAPGKEREGVSA
ISGSGGSTYYADSVKGRFTISRDNSKNTLYLQMNSLRAEDTAVYYCAK
4) Polypeptide encoded by a modified IGHV4-39
(SEQ ID NO: 122)
QLQLQESGPGLVKPSETLSLTCTVSGGSISSSSYYWGWYRQPPGKEREGI
GSIYYSGSTYYNPSLKSRVTISVDTSKNQFSLKLSSVTAADTAVYYCAR
5) Polypeptide encoded by a modified IGHV3-74
(SEQ ID NO: 123)
EVQLVESGGGLVQPGGSLRLSCAASGFTFSSYWMHWYRQAPGKERVGVSR
INSDGSSTSYADSVKGRFTISRDNAKNTLYLQMNSLRAEDTAVYYCAR

### Example 21: Mice Expressing Modified Human VH Domains

This Example describes generation of mice that can express particular human VH domains together with mouse Ig constant domains.

The three lox sequences (LoxP, LoxN, and Lox5171) and a neomycin selection cassette are added to a construct containing the modified VH gene segments described in Example 22. The construct was introduced into ES cells harboring the Singularity Sapiens DJ dock allele via recombination-mediated cassette exchange (RMCE) integration (FIG. 56).

### Example 22: Constructs for In Vivo Affinity Maturation of Input Nanobody Domains

This Example describes the design and generation of nucleic acid constructs containing nucleic acid encoding an input nanobody domain that can be used for the generation of cells (e.g., ES cells) that can be used to make a chimeric non-human animal having the ability to promote in vivo affinity maturation of the input nanobody domain.

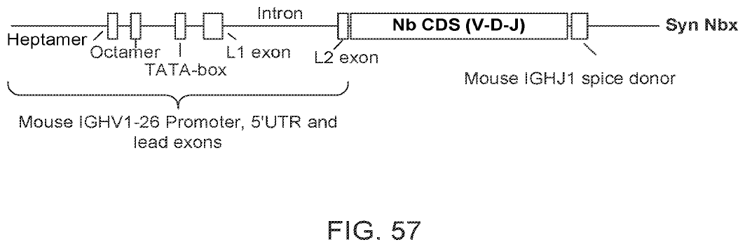

Candidate antibodies are selected from a list of binders. A synthetic construct is designed to contain the 250 bp promoter of a mouse IGHV1-26 and its 5′ UTR, L1 exon, intron, and L2 exon sequences fused in-frame to the coding sequence of the preassembled heavy chain variable region (e.g., V, D, and J domains) for a particular selected antibody, followed by the splice donor sequence downstream of mouse IGHJ1 (FIG. 57).

### Example 23: In Vivo Affinity Maturation of Input Nanobody Domains

This Example describes the design and generation of chimeric mice that can be used for in vivo affinity maturation that occurs during the development and maturation of B cells.

Three lox sites (LoxP, Lox5171, and Lox2272) and a hygromycin selection cassette are added to the synthetic construct. The construct with the lox sites and neomycin selection cassette is integrated into mouse ES cells containing the homozygous Singularity Hyperdock allele at the endogenous Igh locus via recombination mediated cassette exchange (RMCE) (FIG. 58). The engineered ES cells are injected into B-cell deficient embryos such as those from Rag2 knock out mice or IgH knock out mice to produce high percentage chimeras, which are immunized with the same antigen recognized by the input nanobody domain.

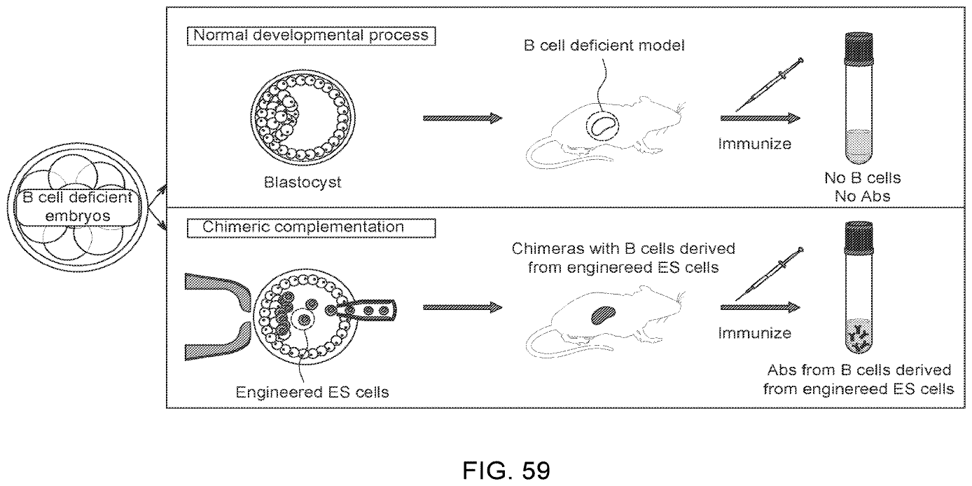

Since no mature B cells are produced from an embryo from either a Rag2 knock out mouse or an IgH knock out mouse, the humoral immune responses of the resulting chimeric mouse are mediated by heavy chain only antibodies derived from the integrated construct encoding the input nanobody domain, eliminating the need for lengthy breeding (FIG. 59). This “Trojan mouse” approach is broadly applicable for generating chimeric cohorts directly from engineered ES cells carrying IgH mutations for immunization

Binders resulting from in vivo affinity maturation are identified from next generation sequencing-based screens and are assessed for improved affinity and/or biophysical properties.

### Example 24: Additional Embodiments

### Embodiment A1

A non-human animal, wherein the genome of said non-human animal comprises an immunoglobulin heavy chain (IgH) allele, wherein said IgH allele comprises an endogenous nucleic acid encoding a CH2 or CH3 domain of an IgG subclass, wherein said IgH allele lacks nucleic acid encoding at least a portion of an endogenous CH1 domain of said IgG subclass or at least a portion of an endogenous regulatory element that drives expression of said endogenous CH1 domain, wherein said IgH allele comprises one or more Ig VH gene segments selected from the group consisting of an Ig VH gene segment that encodes the amino acid sequence set forth in SEQ ID NO:74, an Ig VH gene segment that encodes the amino acid sequence set forth in SEQ ID NO:75, an Ig VH gene segment that encodes the amino acid sequence set forth in SEQ ID NO:76, an Ig VH gene segment that encodes the amino acid sequence set forth in SEQ ID NO:77, an Ig VH gene segment that encodes the amino acid sequence set forth in SEQ ID NO:78, an Ig VH gene segment that encodes the amino acid sequence set forth in SEQ ID NO:79, an Ig VH gene segment that encodes the amino acid sequence set forth in SEQ ID NO:80, an Ig VH gene segment that encodes the amino acid sequence set forth in SEQ ID NO:81, an Ig VH gene segment that encodes the amino acid sequence set forth in SEQ ID NO:82, an Ig VH gene segment that encodes the amino acid sequence set forth in SEQ ID NO:83, an Ig VH gene segment that encodes the amino acid sequence set forth in SEQ ID NO:84, an Ig VH gene segment that encodes the amino acid sequence set forth in SEQ ID NO:85, an Ig VH gene segment that encodes the amino acid sequence set forth in SEQ ID NO:86, and an Ig VH gene segment that encodes the amino acid sequence set forth in SEQ ID NO:87, wherein said non-human animal is capable of producing an antibody, wherein said antibody comprises said CH2 or CH3 domain of said IgG subclass and a variable domain comprising the amino acid sequence encoded by one of said Ig VH gene segments and lacks said CH1 domain.

### Embodiment A2

The non-human animal of Embodiment A1, wherein said IgH allele lacks endogenous nucleic acid encoding at least a portion of an IgM constant domain, endogenous nucleic acid encoding at least a portion of an IgD constant domain, endogenous nucleic acid encoding at least a portion of an IgE constant domain, or endogenous nucleic acid encoding at least a portion of an IgA constant domain.

### Embodiment A3

The non-human animal of any one of said Embodiments A1-A2, wherein said IgH allele of said non-human animal comprises endogenous nucleic acid encoding said CH2 domain and said CH3 domain of said IgG subclass.

### Embodiment A4

The non-human animal of any one of Embodiments A1-A3, wherein said IgH allele of said non-human animal comprises endogenous nucleic acid encoding a hinge domain of said IgG subclass.

### Embodiment A5

The non-human animal of any one of Embodiments A1-A4, wherein said IgG subclass is an IgG2 subclass.

### Embodiment A6

The non-human animal of any one of Embodiments A1-A5, wherein said IgG subclass is an IgG2a, IgG2b, IgG2c, IgG3, or IgG4 subclass.

### Embodiment A7

The non-human animal of any one of Embodiments A1-A6, wherein said IgG subclass is an IgG1 subclass.

### Embodiment A8

The non-human animal of Embodiment A7, wherein said IgH allele lacks endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain.

### Embodiment A9

The non-human animal of Embodiment A7, wherein said IgH allele lacks endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain.

### Embodiment A10

The non-human animal of Embodiment A7, wherein said IgH allele lacks endogenous nucleic acid encoding each of the IgG2 constant domains, endogenous nucleic acid encoding each of the IgG3 constant domains, or endogenous nucleic acid encoding each of the IgG4 constant domains.

### Embodiment A11

The non-human animal of Embodiment A7, wherein said IgH allele lacks endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains.

### Embodiment A12

The non-human animal of any one of Embodiments A1-A11, wherein said IgH allele lacks endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain.

### Embodiment A13

The non-human animal of any one of Embodiments A1-A11, wherein said IgH allele lacks endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains.

### Embodiment A14

The non-human animal of any one of Embodiments A1-A13, wherein said IgH allele lacks endogenous nucleic acid encoding each of the IgM constant domains.

### Embodiment A15

The non-human animal of any one of Embodiments A1-A14, wherein said IgH allele lacks endogenous nucleic acid encoding each of the IgD constant domains.

### Embodiment A16

The non-human animal of any one of Embodiments A1-A15, wherein said IgH allele lacks endogenous nucleic acid encoding each of the IgE constant domains.

### Embodiment A17

The non-human animal of any one of Embodiments A1-A16, wherein said IgH allele lacks endogenous nucleic acid encoding IgA CH1 and CH2 constant domains.

### Embodiment A18

The non-human animal of any one of Embodiments A1-A17, wherein said IgH allele lacks nucleic acid encoding said endogenous CH1 domain.

### Embodiment A19

The non-human animal of any one of Embodiments A1-A18, wherein said IgH allele lacks said endogenous regulatory element.

### Embodiment A20

The non-human animal of any one of Embodiments A1-A19, wherein said IgH allele comprises an endogenous Eμ.

### Embodiment A21

The non-human animal of Embodiment A20, wherein the first nucleic acid sequence encoding a full length CH2 domain downstream of said endogenous Eμ is nucleic acid encoding an IgG CH2 domain.

### Embodiment A22

The non-human animal of Embodiment A20, wherein the first nucleic acid sequence encoding a full length CH2 domain downstream of said endogenous Eμ is nucleic acid encoding an IgG1 CH2 domain.

### Embodiment A23

The non-human animal of any one of Embodiments A1-A22, wherein said IgH allele comprises an endogenous Sμ, an endogenous Iμ promoter, an endogenous Iμ exon, or a combination thereof.

### Embodiment A24

The non-human animal of Embodiment A23, wherein the first nucleic acid sequence encoding a full length CH2 domain downstream of said endogenous Sμ, said endogenous Iμ promoter, or said endogenous Iμ exon is nucleic acid encoding an IgG CH2 domain.

### Embodiment A25

The non-human animal of Embodiment A23, wherein the first nucleic acid sequence encoding a full length CH2 domain downstream of said endogenous Sμ, said endogenous Iμ promoter, or said endogenous Iμ exon is nucleic acid encoding an IgG1 CH2 domain.

### Embodiment A26

The non-human animal of any one of Embodiments A1-A25, wherein said IgH allele comprises an endogenous 3′γ1E.

### Embodiment A27

The non-human animal of Embodiment A26, wherein said IgH allele lacks endogenous nucleic acid encoding a full length CH2 domain downstream of said endogenous 3′γ1E.

### Embodiment A28

The non-human animal of any one of Embodiments A1-A27, wherein said IgH allele comprises an endogenous 5′hsR1.

### Embodiment A29

The non-human animal of Embodiment A28, wherein the first nucleic acid sequence encoding a full length CH2 domain upstream of said endogenous 5′hsR1 is nucleic acid encoding an IgG CH2 domain.

### Embodiment A30

The non-human animal of Embodiment A28, wherein the first nucleic acid sequence encoding a full length CH2 domain upstream of said endogenous 5′hsR1 is nucleic acid encoding an IgG1 CH2 domain.

### Embodiment A31

The non-human animal of any one of Embodiments A1-A30, wherein said IgH allele comprises an endogenous 3′RR.

### Embodiment A32

The non-human animal of Embodiment A31, wherein the first nucleic acid sequence encoding a full length CH2 domain upstream of said endogenous 3′RR is nucleic acid encoding an IgG CH2 domain.

### Embodiment A33

The non-human animal of Embodiment A31, wherein the first nucleic acid sequence encoding a full length CH2 domain upstream of said endogenous 3′RR is nucleic acid encoding an IgG1 CH2 domain.

### Embodiment A34

The non-human animal of any one of Embodiments A1-A33, wherein said IgH allele comprises an endogenous 3′CBE.

### Embodiment A35

The non-human animal of Embodiment A34, wherein the first nucleic acid sequence encoding a full length CH2 domain upstream of said endogenous 3′CBE is nucleic acid encoding an IgG CH2 domain.

### Embodiment A36

The non-human animal of Embodiment A34, wherein the first nucleic acid sequence encoding a full length CH2 domain upstream of said endogenous 3′CBE is nucleic acid encoding an IgG1 CH2 domain.

### Embodiment A37

The non-human animal of any one of Embodiments A1-A36, wherein at least one allele of said genome lacks at least a portion of the endogenous Ig heavy chain variable region.

### Embodiment A38

The non-human animal of any one of Embodiments A1-A36, wherein at least one allele of said genome lacks all the exons of the endogenous Ig heavy chain variable region.

### Embodiment A39

The non-human animal of any one of Embodiments A1-A36, wherein both alleles of said genome lack all the exons of the endogenous Ig heavy chain variable region.

### Embodiment A40

The non-human animal of any one of Embodiments A1-A36, wherein neither allele of said genome comprises an endogenous exon of an Ig heavy chain variable region.

### Embodiment A41

The non-human animal of any one of Embodiments A1-A40, wherein said IgH allele comprises, in addition to said one or more Ig VH gene segments selected from said group, exogenous nucleic acid encoding one or more human Ig VH gene segments.

### Embodiment A42

The non-human animal of Embodiment A41, wherein said IgH allele comprises three or more human Ig VH gene segments.

### Embodiment A43

The non-human animal of Embodiment A41, wherein said IgH allele comprises 26 or more human Ig VH gene segments.

### Embodiment A44

The non-human animal of Embodiment A41, wherein said IgH allele comprises 65 or more human Ig VH gene segments.

### Embodiment A45

The non-human animal of Embodiment A41, wherein said IgH allele comprises 85 or more human Ig VH gene segments.

### Embodiment A46

The non-human animal of Embodiment A41, wherein said IgH allele comprises 129 human Ig VH gene segments.

### Embodiment A47

The non-human animal of Embodiment A41, wherein said IgH allele comprises at least 44 functional human Ig VH gene segments.

### Embodiment A48

The non-human animal of Embodiment A41, wherein said IgH allele comprises at least 58 functional human Ig VH gene segments.

### Embodiment A49

The non-human animal of any one of Embodiments A1-A48, wherein said IgH allele comprises 13 or more human Ig DH gene segments.

### Embodiment A50

The non-human animal of any one of Embodiments A1-A48, wherein said IgH allele comprises 27 human Ig DH gene segments.

### Embodiment A51

The non-human animal of any one of Embodiments A1-A50, wherein said IgH allele comprises three or more human Ig VH gene segments.

### Embodiment A52

The non-human animal of any one of Embodiments A1-A51, wherein said IgH allele comprises 9 human Ig VH gene segments.

### Embodiment A53

The non-human animal of any one of Embodiments A1-A40, wherein said IgH allele comprises 129 or more human Ig VH gene segments, 27 or more human Ig DH gene segments, and 9 or more human Ig VH gene segments.

### Embodiment A54

The non-human animal of any one of Embodiments A1-A53, wherein said IgH allele comprises two or more of said Ig VH gene segments selected from said group.

### Embodiment A55

The non-human animal of any one of Embodiments A1-A53, wherein said IgH allele comprises three or more of said Ig VH gene segments selected from said group.

### Embodiment A56

The non-human animal of any one of Embodiments A1-A53, wherein said IgH allele comprises four or more of said Ig VH gene segments selected from said group.

### Embodiment A57

The non-human animal of any one of Embodiments A1-A53, wherein said IgH allele comprises five or more of said Ig VH gene segments selected from said group.

### Embodiment A58

The non-human animal of any one of Embodiments A1-A53, wherein said IgH allele comprises six or more of said Ig VH gene segments selected from said group.

### Embodiment A59

The non-human animal of any one of Embodiments A1-A53, wherein said IgH allele comprises seven or more of said Ig VH gene segments selected from said group.

### Embodiment A60

The non-human animal of any one of Embodiments A1-A53, wherein said IgH allele comprises eight or more of said Ig VH gene segments selected from said group.

### Embodiment A61

The non-human animal of any one of Embodiments A1-A53, wherein said IgH allele comprises nine or more of said Ig VH gene segments selected from said group.

### Embodiment A62

The non-human animal of any one of Embodiments A1-A53, wherein said IgH allele comprises ten or more of said Ig VH gene segments selected from said group.

### Embodiment A63

The non-human animal of any one of Embodiments A1-A53, wherein said IgH allele comprises 11 or more of said Ig VH gene segments selected from said group.

### Embodiment A64

The non-human animal of any one of Embodiments A1-A53, wherein said IgH allele comprises 12 or more of said Ig VH gene segments selected from said group.

### Embodiment A65

The non-human animal of any one of Embodiments A1-A53, wherein said IgH allele comprises 13 or more of said Ig VH gene segments selected from said group.

### Embodiment A66

The non-human animal of any one of Embodiments A1-A53, wherein said IgH allele comprises an Ig VH gene segment that encodes the amino acid sequence set forth in SEQ ID NO:74, an Ig VH gene segment that encodes the amino acid sequence set forth in SEQ ID NO:75, an Ig VH gene segment that encodes the amino acid sequence set forth in SEQ ID NO:76, an Ig VH gene segment that encodes the amino acid sequence set forth in SEQ ID NO:77, an Ig VH gene segment that encodes the amino acid sequence set forth in SEQ ID NO:78, an Ig VH gene segment that encodes the amino acid sequence set forth in SEQ ID NO:79, an Ig VH gene segment that encodes the amino acid sequence set forth in SEQ ID NO:80, an Ig VH gene segment that encodes the amino acid sequence set forth in SEQ ID NO:81, an Ig VH gene segment that encodes the amino acid sequence set forth in SEQ ID NO:82, an Ig VH gene segment that encodes the amino acid sequence set forth in SEQ ID NO:83, an Ig VH gene segment that encodes the amino acid sequence set forth in SEQ ID NO:84, an Ig VH gene segment that encodes the amino acid sequence set forth in SEQ ID NO:85, an Ig VH gene segment that encodes the amino acid sequence set forth in SEQ ID NO:86, and an Ig VH gene segment that encodes the amino acid sequence set forth in SEQ ID NO:87.

### Embodiment A67

The non-human animal of any one of Embodiments A1-A66, wherein said IgH allele comprises an endogenous regulatory element for each of said one or more Ig VH gene segments.

### Embodiment A668

The non-human animal of Embodiment A67, wherein said endogenous regulatory element is an endogenous promoter sequence of said non-human animal.

### Embodiment A69

The non-human animal of any one of Embodiments A1-A68, wherein said IgH allele comprises an endogenous exon encoding a leader sequence for each of said one or more Ig VH gene segments.

### Embodiment A70

The non-human animal of Embodiment A69, wherein said endogenous exon is an endogenous L1 exon of said non-human animal.

### Embodiment A71

The non-human animal of Embodiment A69, wherein said endogenous exon is an endogenous L2 exon of said non-human animal.

### Embodiment A72

The non-human animal of Embodiment A69, wherein said IgH allele comprises endogenous L1 and L2 exons encoding a leader sequence for each of said one or more Ig VH gene segments.

### Embodiment A73

The non-human animal of any one of Embodiments A1-A72, wherein said IgH allele comprises at least one exogenous recombinase site recognition nucleic acid sequence.

### Embodiment A74

The non-human animal of Embodiment A73, wherein said at least one exogenous recombinase site recognition nucleic acid sequence is located upstream of said endogenous nucleic acid encoding said CH2 or CH3 domain of said IgG subclass.

### Embodiment A75

The non-human animal of any one of Embodiments A73-A74, wherein said IgH allele comprises one, two, three, four, five, six, seven, eight, nine, or ten different exogenous recombinase site recognition nucleic acid sequences.

### Embodiment A76

The non-human animal of any one of Embodiments A73-A75, wherein said IgH allele comprises at least three different exogenous recombinase site recognition nucleic acid sequences.

### Embodiment A77

The non-human animal of any one of Embodiments A73-A75, wherein said IgH allele comprises at least five different exogenous recombinase site recognition nucleic acid sequences.

### Embodiment A78

The non-human animal of any one of Embodiments A75-A77, wherein each of said different exogenous recombinase site recognition nucleic acid sequences is located less than 2.5 Mb upstream of an endogenous Eμ.

### Embodiment A79

The non-human animal of any one of Embodiments A75-A77, wherein each of said different exogenous recombinase site recognition nucleic acid sequences is located less than 2.0 Mb, less than 1.5 Mb, less than 1.0 Mb, less than 500 kb, or less than 250 kb upstream of an endogenous Eμ.

### Embodiment A80

The non-human animal of any one of Embodiments A75-A77, wherein each of said different exogenous recombinase site recognition nucleic acid sequences is located less than 200 kb, less than 100 kb, less than 50 kb, less than 25 kb, or less than 10 kb upstream of an endogenous Eμ.

### Embodiment A81

The non-human animal of any one of Embodiments A1-A80, wherein said non-human animal is a mouse or rat.

### Embodiment A82

The non-human animal of any one of Embodiments A1-A80, wherein said non-human animal is a mouse.

### Embodiment A83

A method of producing a non-human animal of any one of Embodiments A1-A82, wherein said method comprises (a) introducing exogenous nucleic acid comprising one or more Ig VH gene segments selected from the group consisting of an Ig VH gene segment that encodes the amino acid sequence set forth in SEQ ID NO:74, an Ig VH gene segment that encodes the amino acid sequence set forth in SEQ ID NO:75, an Ig VH gene segment that encodes the amino acid sequence set forth in SEQ ID NO:76, an Ig VH gene segment that encodes the amino acid sequence set forth in SEQ ID NO:77, an Ig VH gene segment that encodes the amino acid sequence set forth in SEQ ID NO:78, an Ig VH gene segment that encodes the amino acid sequence set forth in SEQ ID NO:79, an Ig VH gene segment that encodes the amino acid sequence set forth in SEQ ID NO:80, an Ig VH gene segment that encodes the amino acid sequence set forth in SEQ ID NO:81, an Ig VH gene segment that encodes the amino acid sequence set forth in SEQ ID NO:82, an Ig VH gene segment that encodes the amino acid sequence set forth in SEQ ID NO:83, an Ig VH gene segment that encodes the amino acid sequence set forth in SEQ ID NO:84, an Ig VH gene segment that encodes the amino acid sequence set forth in SEQ ID NO:85, an Ig VH gene segment that encodes the amino acid sequence set forth in SEQ ID NO:86, and an Ig VH gene segment that encodes the amino acid sequence set forth in SEQ ID NO:87 into an endogenous IgH locus in a stem cell of a non-human animal; (b) implanting said stem cell into a blastocyst; (c) implanting said blastocyst into a pseudo-pregnant non-human animal to obtain a chimeric non-human animal; (d) crossing said chimeric non-human animal to a wild-type non-human animal to produce offspring; (e) screening the offspring for heterozygosity; and (f) identifying a founder non-human animal carrying said one or more Ig VH gene segments.

### Embodiment A84

The method of Embodiment A83, wherein said stem cell is an embryonic stem cell.

### Embodiment A85

A method of producing an antibody in a non-human animal of any one of Embodiments A1-A82, wherein said method comprises administering an antigen to said non-human animal, wherein one or more B cells in said non-human animal produce an antibody comprising a variable domain encoded by one of said Ig VH gene segments.

### Embodiment A86

The method of Embodiment A85, wherein said antibody is a single domain antibody (sdAb).

### Embodiment A87

The method of Embodiment A85 or A86, wherein said antibody comprises an amino acid sequence selected from SEQ ID NOs: 74-87.

### Embodiment A88

A method of obtaining a B cell that produces an antibody capable of binding to an antigen, wherein said method comprises:
(a) administering said antigen to a non-human animal of any one of Embodiments A1-A82, wherein one or more B cells in said non-human animal produce said antibody, and wherein said antibody comprises a variable domain comprising the amino acid sequence encoded by one of said Ig VH gene segments, and
(b) isolating said one or more B cells from said non-human animal.

### Embodiment A89

The method of Embodiment A88, wherein said antibody is a single domain antibody (sdAb).

### Embodiment A90

The method of Embodiment A88 or A89, wherein said antibody comprises an amino acid sequence selected from SEQ ID NOs: 74-87.

### Embodiment B1

A non-human animal, wherein the genome of said non-human animal comprises an immunoglobulin heavy chain (IgH) allele, wherein said IgH allele comprises an endogenous nucleic acid encoding a CH2 or CH3 domain of an IgG subclass, wherein said IgH allele lacks nucleic acid encoding at least a portion of an endogenous CH1 domain of said IgG subclass or at least a portion of an endogenous regulatory element that drives expression of said endogenous CH1 domain, wherein said IgH allele comprises nucleic acid encoding one or more T cell receptor (TCR) variable domains, wherein said non-human animal is capable of producing an antibody-like molecule, wherein said antibody-like molecule comprises said CH2 or CH3 domain of said IgG subclass and one of said TCR variable domains and lacks said CH1 domain.

### Embodiment B2

The non-human animal of Embodiment B1, wherein said IgH allele lacks endogenous nucleic acid encoding at least a portion of an IgM constant domain, endogenous nucleic acid encoding at least a portion of an IgD constant domain, endogenous nucleic acid encoding at least a portion of an IgE constant domain, or endogenous nucleic acid encoding at least a portion of an IgA constant domain.

### Embodiment B3

The non-human animal of any one of said Embodiments B1-B2, wherein said IgH allele of said non-human animal comprises endogenous nucleic acid encoding said CH2 domain and said CH3 domain of said IgG subclass.

### Embodiment B4

The non-human animal of any one of Embodiments B1-B3, wherein said IgH allele of said non-human animal comprises endogenous nucleic acid encoding a hinge domain of said IgG subclass.

### Embodiment B5

The non-human animal of any one of Embodiments B1-B4, wherein said IgG subclass is an IgG2 subclass.

### Embodiment B6

The non-human animal of any one of Embodiments B1-B5, wherein said IgG subclass is an IgG2a, IgG2b, IgG2c, IgG3, or IgG4 subclass.

### Embodiment B7

The non-human animal of any one of Embodiments B1-B6, wherein said IgG subclass is an IgG1 subclass.

### Embodiment B8

The non-human animal of Embodiment B7, wherein said IgH allele lacks endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain.

### Embodiment B9

The non-human animal of Embodiment B7, wherein said IgH allele lacks endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain.

### Embodiment B10

The non-human animal of Embodiment B7, wherein said IgH allele lacks endogenous nucleic acid encoding each of the IgG2 constant domains, endogenous nucleic acid encoding each of the IgG3 constant domains, or endogenous nucleic acid encoding each of the IgG4 constant domains.

### Embodiment B11

The non-human animal of Embodiment B7, wherein said IgH allele lacks endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains.

### Embodiment B12

The non-human animal of any one of Embodiments B1-B11, wherein said IgH allele lacks endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain.

### Embodiment B13

The non-human animal of any one of Embodiments B1-B11, wherein said IgH allele lacks endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains.

### Embodiment B14

The non-human animal of any one of Embodiments B1-B13, wherein said IgH allele lacks endogenous nucleic acid encoding each of the IgM constant domains.

### Embodiment B15

The non-human animal of any one of Embodiments B1-B14, wherein said IgH allele lacks endogenous nucleic acid encoding each of the IgD constant domains.

### Embodiment B16

The non-human animal of any one of Embodiments B1-B15, wherein said IgH allele lacks endogenous nucleic acid encoding each of the IgE constant domains.

### Embodiment B17

The non-human animal of any one of Embodiments B1-B16, wherein said IgH allele lacks endogenous nucleic acid encoding IgA CH1 and CH2 constant domains.

### Embodiment B18

The non-human animal of any one of Embodiments B1-B17, wherein said IgH allele lacks nucleic acid encoding said endogenous CH1 domain.

### Embodiment B19

The non-human animal of any one of Embodiments B1-B18, wherein said IgH allele lacks said endogenous regulatory element.

### Embodiment B20

The non-human animal of any one of Embodiments B1-B19, wherein said IgH allele comprises an endogenous Eμ.

### Embodiment B21

The non-human animal of Embodiment B20, wherein the first nucleic acid sequence encoding a full length CH2 domain downstream of said endogenous Eμ is nucleic acid encoding an IgG CH2 domain.

### Embodiment B22

The non-human animal of Embodiment B20, wherein the first nucleic acid sequence encoding a full length CH2 domain downstream of said endogenous Eμ is nucleic acid encoding an IgG1 CH2 domain.

### Embodiment B23

The non-human animal of any one of Embodiments B1-B22, wherein said IgH allele comprises an endogenous Sμ, an endogenous Iμ promoter, an endogenous Iμ exon, or a combination thereof.

### Embodiment B24

The non-human animal of Embodiment B23, wherein the first nucleic acid sequence encoding a full length CH2 domain downstream of said endogenous Sμ, said endogenous Iμ promoter, or said endogenous Iμ exon is nucleic acid encoding an IgG CH2 domain.

### Embodiment B25

The non-human animal of Embodiment B23, wherein the first nucleic acid sequence encoding a full length CH2 domain downstream of said endogenous Sμ, said endogenous Iμ promoter, or said endogenous Iμ exon is nucleic acid encoding an IgG1 CH2 domain.

### Embodiment B26

The non-human animal of any one of Embodiments B1-B25, wherein said IgH allele comprises an endogenous 3′γ1E.

### Embodiment B27

The non-human animal of Embodiment B26, wherein said IgH allele lacks endogenous nucleic acid encoding a full length CH2 domain downstream of said endogenous 3′γ1E.

### Embodiment B28

The non-human animal of any one of Embodiments B1-B27, wherein said IgH allele comprises an endogenous 5′hsR1.

### Embodiment B29

The non-human animal of Embodiment B28, wherein the first nucleic acid sequence encoding a full length CH2 domain upstream of said endogenous 5′hsR1 is nucleic acid encoding an IgG CH2 domain.

### Embodiment B30

The non-human animal of Embodiment B28, wherein the first nucleic acid sequence encoding a full length CH2 domain upstream of said endogenous 5′hsR1 is nucleic acid encoding an IgG1 CH2 domain.

### Embodiment B31

The non-human animal of any one of Embodiments B1-B30, wherein said IgH allele comprises an endogenous 3′RR.

### Embodiment B32

The non-human animal of Embodiment B31, wherein the first nucleic acid sequence encoding a full length CH2 domain upstream of said endogenous 3′RR is nucleic acid encoding an IgG CH2 domain.

### Embodiment B33

The non-human animal of Embodiment B31, wherein the first nucleic acid sequence encoding a full length CH2 domain upstream of said endogenous 3′RR is nucleic acid encoding an IgG1 CH2 domain.

### Embodiment B34

The non-human animal of any one of Embodiments B1-B33, wherein said IgH allele comprises an endogenous 3′CBE.

### Embodiment B35

The non-human animal of Embodiment B34, wherein the first nucleic acid sequence encoding a full length CH2 domain upstream of said endogenous 3′CBE is nucleic acid encoding an IgG CH2 domain.

### Embodiment B36

The non-human animal of Embodiment B34, wherein the first nucleic acid sequence encoding a full length CH2 domain upstream of said endogenous 3′CBE is nucleic acid encoding an IgG1 CH2 domain.

### Embodiment B37

The non-human animal of any one of Embodiments B1-B36, wherein at least one allele of said genome lacks at least a portion of the endogenous Ig heavy chain variable region.

### Embodiment B38

The non-human animal of any one of Embodiments B1-B36, wherein at least one allele of said genome lacks all the exons of the endogenous Ig heavy chain variable region.

### Embodiment B39

The non-human animal of any one of Embodiments B1-B36, wherein both alleles of said genome lack all the exons of the endogenous Ig heavy chain variable region.

### Embodiment B40

The non-human animal of any one of Embodiments B1-B36, wherein neither allele of said genome comprises an endogenous exon of an Ig heavy chain variable region.

### Embodiment B41

The non-human animal of any one of Embodiments B1-B40, wherein said IgH allele comprises, in addition to said nucleic acid encoding one or more TCR variable domains, exogenous nucleic acid encoding one or more human Ig VH gene segments.

### Embodiment B42

The non-human animal of Embodiment B41, wherein said IgH allele comprises three or more human Ig VH gene segments.

### Embodiment B43

The non-human animal of Embodiment B41, wherein said IgH allele comprises 26 or more human Ig VH gene segments.

### Embodiment B44

The non-human animal of Embodiment B41, wherein said IgH allele comprises 65 or more human Ig VH gene segments.

### Embodiment B45

The non-human animal of Embodiment B41, wherein said IgH allele comprises 85 or more human Ig VH gene segments.

### Embodiment B46

The non-human animal of Embodiment B41, wherein said IgH allele comprises 129 human Ig VH gene segments.

### Embodiment B47

The non-human animal of any one of Embodiments B1-B46, wherein said IgH allele comprises 13 or more human Ig DH gene segments.

### Embodiment B48

The non-human animal of any one of Embodiments B1-B46, wherein said IgH allele comprises 27 human Ig DH gene segments.

### Embodiment B49

The non-human animal of any one of Embodiments B1-B48, wherein said IgH allele comprises three or more human Ig VH gene segments.

### Embodiment B50

The non-human animal of any one of Embodiments B1-B49, wherein said IgH allele comprises 9 human Ig VH gene segments.

### Embodiment B51

The non-human animal of any one of Embodiments B1-B40, wherein said IgH allele comprises 126 or more human Ig VH gene segments, 27 or more human Ig DH gene segments, and 9 or more human Ig VH gene segments.

### Embodiment B52

The non-human animal of any one of Embodiments B1-B51, wherein said one or more TCR variable domains are TCR Vα domains.

### Embodiment B53

The non-human animal of Embodiment B52, wherein said one or more TCR variable domains comprises five or more TCR Vα domains.

### Embodiment B54

The non-human animal of any one of Embodiments B52-B53, wherein said IgH allele comprises nucleic acid encoding one or more TCR Jα domains.

### Embodiment B55

The non-human animal of any one of Embodiments B1-B51, wherein said one or more TCR variable domains are TCR Vβ domains.

### Embodiment B56

The non-human animal of Embodiment B55, wherein said one or more TCR variable domains comprises five or more TCR Vβ domains.

### Embodiment B57

The non-human animal of any one of Embodiments B55-B56, wherein said IgH allele comprises nucleic acid encoding one or more TCR Dβ domains.

### Embodiment B58

The non-human animal of any one of Embodiments B55-B57, wherein said IgH allele comprises nucleic acid encoding one or more TCR Jβ domains.

### Embodiment B59

The non-human animal of any one of Embodiments B1-B51, wherein said one or more TCR variable domains are TCR Vy domains.

### Embodiment B60

The non-human animal of Embodiment B59, wherein said one or more TCR variable domains comprises five or more TCR Vy domains.

### Embodiment B61

The non-human animal of any one of Embodiments B59-B60, wherein said IgH allele comprises nucleic acid encoding one or more TCR Jγ domains.

### Embodiment B62

The non-human animal of any one of Embodiments B1-B51, wherein said one or more TCR variable domains are TCR Vô domains.

### Embodiment B63

The non-human animal of Embodiment B62, wherein said one or more TCR variable domains comprises five or more TCR Vô domains.

### Embodiment B64

The non-human animal of any one of Embodiments B62-B63, wherein said IgH allele comprises nucleic acid encoding one or more TCR Dô domains.

### Embodiment B65

The non-human animal of any one of Embodiments B62-B64, wherein said IgH allele comprises nucleic acid encoding one or more TCR Jβ domains.

### Embodiment B66

The non-human animal of any one of Embodiments B1-B65, wherein said one or more TCR variable domains are human TCR variable domains.

### Embodiment B67

The non-human animal of any one of Embodiments B57 and B64, wherein said one or more TCR Dβ or Dδ domains are human TCR Dβ or Dδ domains.

### Embodiment B68

The non-human animal of any one of Embodiments B54, B58, B61, and B65, wherein said one or more TCR Jα, Jβ, Jγ, or Jδ domains are human TCR Jα, Jβ, Jγ, or Jδ domains.

### Embodiment B69

The non-human animal of any one of Embodiments B1-B53, B55, B56, B59, B60, B62, B63, and B66, wherein said IgH allele lacks nucleic acid encoding TCR D or J domains.

### Embodiment B70

The non-human animal of any one of Embodiments B1-B53, B55, B56, B59, B60, B62, B63, and B66, wherein said IgH allele lacks nucleic acid encoding TCR D and J domains.

### Embodiment B71

The non-human animal of any one of Embodiments B1-B70, wherein said IgH allele comprises at least one exogenous recombinase site recognition nucleic acid sequence.

### Embodiment B72

The non-human animal of Embodiment B71, wherein said at least one exogenous recombinase site recognition nucleic acid sequence is located upstream of said endogenous nucleic acid encoding said CH2 or CH3 domain of said IgG subclass.

### Embodiment B73

The non-human animal of any one of Embodiments B71-B72, wherein said IgH allele comprises one, two, three, four, five, six, seven, eight, nine, or ten different exogenous recombinase site recognition nucleic acid sequences.

### Embodiment B74

The non-human animal of any one of Embodiments B71-B73, wherein said IgH allele comprises at least three different exogenous recombinase site recognition nucleic acid sequences.

### Embodiment B75

The non-human animal of any one of Embodiments B71-B73, wherein said IgH allele comprises at least five different exogenous recombinase site recognition nucleic acid sequences.

### Embodiment B76

The non-human animal of any one of Embodiments B73-B75, wherein each of said different exogenous recombinase site recognition nucleic acid sequences is located less than 2.5 Mb upstream of an endogenous Eμ.

### Embodiment B77

The non-human animal of any one of Embodiments B73-B75, wherein each of said different exogenous recombinase site recognition nucleic acid sequences is located less than 2.0 Mb, less than 1.5 Mb, less than 1.0 Mb, less than 500 kb, or less than 250 kb upstream of an endogenous Eμ.

### Embodiment B78

The non-human animal of any one of Embodiments B73-B75, wherein each of said different exogenous recombinase site recognition nucleic acid sequences is located less than 200 kb, less than 100 kb, less than 50 kb, less than 25 kb, or less than 10 kb upstream of an endogenous Eμ.

### Embodiment B79

The non-human animal of any one of Embodiments B1-B78, wherein said non-human animal is a mouse or rat.

### Embodiment B80

The non-human animal of any one of Embodiments B1-B78, wherein said non-human animal is a mouse.

### Embodiment B81

A method of producing a non-human animal of any one of Embodiments B1-B80, wherein said method comprises (a) introducing exogenous nucleic acid encoding one or more TCR variable domains into an endogenous IgH locus in a stem cell of a non-human animal; (b) implanting said stem cell into a blastocyst; (c) implanting said blastocyst into a pseudo-pregnant non-human animal to obtain a chimeric non-human animal; (d) crossing said chimeric non-human animal to a wild-type non-human animal to produce offspring; (e) screening the offspring for heterozygosity; and (f) identifying a founder non-human animal carrying said exogenous nucleic acid.

### Embodiment B82

The method of Embodiment B81, wherein said stem cell is an embryonic stem cell.

### Embodiment B83

A method of producing an antibody-like molecule in a non-human animal of any one of Embodiments B1-B80, wherein said method comprises administering an antigen to said non-human animal, wherein one or more B cells in said non-human animal produce an antibody-like molecule comprising one of said one or more TCR variable domains.

### Embodiment B84

The method of Embodiment B83, wherein said antibody-like molecule is a single chain antibody-like molecule.

### Embodiment B85

A method of obtaining a B cell that produces an antibody-like molecule capable of binding to an antigen, wherein said method comprises:
(a) administering said antigen to a non-human animal of any one of Embodiments B1-B80, wherein one or more B cells in said non-human animal produce said antibody-like molecule, and wherein said antibody-like molecule comprises one of said one or more TCR variable domains, and
(b) isolating said one or more B cells from said non-human animal.

### Embodiment B86

The method of Embodiment B85, wherein said antibody-like molecule is a single chain antibody-like molecule.

### Embodiment C1

A non-human animal, wherein the genome of said non-human animal comprises an immunoglobulin heavy chain (IgH) allele, wherein said IgH allele comprises an endogenous nucleic acid encoding a CH2 or CH3 domain of an IgG subclass, wherein said IgH allele lacks nucleic acid encoding at least a portion of an endogenous CH1 domain of said IgG subclass or at least a portion of an endogenous regulatory element that drives expression of said endogenous CH1 domain, wherein said IgH allele comprises nucleic acid encoding a first amino acid sequence of a FN3 polypeptide, wherein said IgH allele comprises nucleic acid encoding one or more human Ig variable D domains, wherein said IgH allele comprises nucleic acid encoding a second amino acid sequence of a FN3 polypeptide, wherein said non-human animal is capable of producing an antibody-like molecule, wherein said antibody-like molecule comprises said CH2 or CH3 domain of said IgG subclass, said first amino acid sequence, one of said human Ig variable D domains, and said second amino acid sequence and lacks said CH1 domain.

### Embodiment C2

The non-human animal of Embodiment C1, wherein said IgH allele lacks endogenous nucleic acid encoding at least a portion of an IgM constant domain, endogenous nucleic acid encoding at least a portion of an IgD constant domain, endogenous nucleic acid encoding at least a portion of an IgE constant domain, or endogenous nucleic acid encoding at least a portion of an IgA constant domain.

### Embodiment C3

The non-human animal of any one of said Embodiments C1-C₂, wherein said IgH allele of said non-human animal comprises endogenous nucleic acid encoding said CH2 domain and said CH3 domain of said IgG subclass.

### Embodiment C4

The non-human animal of any one of Embodiments C₁-C₃, wherein said IgH allele of said non-human animal comprises endogenous nucleic acid encoding a hinge domain of said IgG subclass.

### Embodiment C5

The non-human animal of any one of Embodiments C₁-C₄, wherein said IgG subclass is an IgG2 subclass.

### Embodiment C6

The non-human animal of any one of Embodiments C₁-C₅, wherein said IgG subclass is an IgG2a, IgG2b, IgG2c, IgG3, or IgG4 subclass.

### Embodiment C7

The non-human animal of any one of Embodiments C₁-C₆, wherein said IgG subclass is an IgG1 subclass.

### Embodiment C8

The non-human animal of Embodiment C7, wherein said IgH allele lacks endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain.

### Embodiment C9

The non-human animal of Embodiment C7, wherein said IgH allele lacks endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain.

### Embodiment C10

The non-human animal of Embodiment C7, wherein said IgH allele lacks endogenous nucleic acid encoding each of the IgG2 constant domains, endogenous nucleic acid encoding each of the IgG3 constant domains, or endogenous nucleic acid encoding each of the IgG4 constant domains.

### Embodiment C11

The non-human animal of Embodiment C7, wherein said IgH allele lacks endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains.

### Embodiment C12

The non-human animal of any one of Embodiments C1-C11, wherein said IgH allele lacks endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain.

### Embodiment C13

The non-human animal of any one of Embodiments C1-C11, wherein said IgH allele lacks endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains.

### Embodiment C14

The non-human animal of any one of Embodiments C1-C13, wherein said IgH allele lacks endogenous nucleic acid encoding each of the IgM constant domains.

### Embodiment C15

The non-human animal of any one of Embodiments C1-C14, wherein said IgH allele lacks endogenous nucleic acid encoding each of the IgD constant domains.

### Embodiment C16

The non-human animal of any one of Embodiments C1-C15, wherein said IgH allele lacks endogenous nucleic acid encoding each of the IgE constant domains.

### Embodiment C17

The non-human animal of any one of Embodiments C1-C16, wherein said IgH allele lacks endogenous nucleic acid encoding IgA CH1 and CH2 constant domains.

### Embodiment C18

The non-human animal of any one of Embodiments C1-C17, wherein said IgH allele lacks nucleic acid encoding said endogenous CH1 domain.

### Embodiment C19

The non-human animal of any one of Embodiments C1-C18, wherein said IgH allele lacks said endogenous regulatory element.

### Embodiment C20

The non-human animal of any one of Embodiments C1-C19, wherein said IgH allele comprises an endogenous Eμ.

### Embodiment C21

The non-human animal of Embodiment C20, wherein the first nucleic acid sequence encoding a full length CH2 domain downstream of said endogenous Eμ is nucleic acid encoding an IgG CH2 domain.

### Embodiment C22

The non-human animal of Embodiment C20, wherein the first nucleic acid sequence encoding a full length CH2 domain downstream of said endogenous Eμ is nucleic acid encoding an IgG1 CH2 domain.

### Embodiment C23

The non-human animal of any one of Embodiments C1-C22, wherein said IgH allele comprises an endogenous Sμ, an endogenous Iμ promoter, an endogenous Iμ exon, or a combination thereof.

### Embodiment C24

The non-human animal of Embodiment C23, wherein the first nucleic acid sequence encoding a full length CH2 domain downstream of said endogenous Sμ, said endogenous Iμ promoter, or said endogenous Iμ exon is nucleic acid encoding an IgG CH2 domain.

### Embodiment C25

The non-human animal of Embodiment C23, wherein the first nucleic acid sequence encoding a full length CH2 domain downstream of said endogenous Sμ, said endogenous Iμ promoter, or said endogenous Iμ exon is nucleic acid encoding an IgG1 CH2 domain.

### Embodiment C26

The non-human animal of any one of Embodiments C1-C25, wherein said IgH allele comprises an endogenous 3′γ1E.

### Embodiment C27

The non-human animal of Embodiment C26, wherein said IgH allele lacks endogenous nucleic acid encoding a full length CH2 domain downstream of said endogenous 3′γ1E.

### Embodiment C28

The non-human animal of any one of Embodiments C1-C27, wherein said IgH allele comprises an endogenous 5′hsR1.

### Embodiment C29

The non-human animal of Embodiment C28, wherein the first nucleic acid sequence encoding a full length CH2 domain upstream of said endogenous 5′hsR1 is nucleic acid encoding an IgG CH2 domain.

### Embodiment C30

The non-human animal of Embodiment C28, wherein the first nucleic acid sequence encoding a full length CH2 domain upstream of said endogenous 5′hsR1 is nucleic acid encoding an IgG1 CH2 domain.

### Embodiment C31

The non-human animal of any one of Embodiments C1-C30, wherein said IgH allele comprises an endogenous 3′RR.

### Embodiment C32

The non-human animal of Embodiment C31, wherein the first nucleic acid sequence encoding a full length CH2 domain upstream of said endogenous 3′RR is nucleic acid encoding an IgG CH2 domain.

### Embodiment C33

The non-human animal of Embodiment C31, wherein the first nucleic acid sequence encoding a full length CH2 domain upstream of said endogenous 3′RR is nucleic acid encoding an IgG1 CH2 domain.

### Embodiment C34

The non-human animal of any one of Embodiments C1-C33, wherein said IgH allele comprises an endogenous 3′CBE.

### Embodiment C35

The non-human animal of Embodiment C34, wherein the first nucleic acid sequence encoding a full length CH2 domain upstream of said endogenous 3′CBE is nucleic acid encoding an IgG CH2 domain.

### Embodiment C36

The non-human animal of Embodiment C34, wherein the first nucleic acid sequence encoding a full length CH2 domain upstream of said endogenous 3′CBE is nucleic acid encoding an IgG1 CH2 domain.

### Embodiment C37

The non-human animal of any one of Embodiments C1-C36, wherein at least one allele of said genome lacks at least a portion of the endogenous Ig heavy chain variable region.

### Embodiment C38

The non-human animal of any one of Embodiments C1-C36, wherein at least one allele of said genome lacks all the exons of the endogenous Ig heavy chain variable region.

### Embodiment C39

The non-human animal of any one of Embodiments C1-C36, wherein both alleles of said genome lack all the exons of the endogenous Ig heavy chain variable region.

### Embodiment C40

The non-human animal of any one of Embodiments C1-C36, wherein neither allele of said genome comprises an endogenous exon of an Ig heavy chain variable region.

### Embodiment C41

The non-human animal of any one of Embodiments C1-C40, wherein said FN3 polypeptide is a human ¹⁰FN3 polypeptide.

### Embodiment C42

The non-human animal of any one of Embodiments C1-C41, wherein said first amino acid sequence comprises the amino acid sequence set forth in any one of SEQ ID NOs:C24 and C26.

### Embodiment C43

The non-human animal of any one of Embodiments C1-C42, wherein said second amino acid sequence comprises the amino acid sequence set forth in any one of SEQ ID NOs:C25 and C27.

### Embodiment C44

The non-human animal of any one of Embodiments C1-C43, wherein said IgH allele comprises nucleic acid encoding two or more human Ig variable D domains.

### Embodiment C45

The non-human animal of any one of Embodiments C1-C43, wherein said IgH allele comprises nucleic acid encoding three or more human Ig variable D domains.

### Embodiment C46

The non-human animal of any one of Embodiments C1-C43, wherein said IgH allele comprises nucleic acid encoding four or more human Ig variable D domains.

### Embodiment C47

The non-human animal of any one of Embodiments C1-C43, wherein said IgH allele comprises nucleic acid encoding five or more human Ig variable D domains.

### Embodiment C48

The non-human animal of any one of Embodiments C1-C43, wherein said IgH allele comprises nucleic acid encoding six or more human Ig variable D domains.

### Embodiment C49

The non-human animal of any one of Embodiments C1-C43, wherein said IgH allele comprises nucleic acid encoding seven or more human Ig variable D domains.

### Embodiment C50

The non-human animal of any one of Embodiments C1-C43, wherein said IgH allele comprises nucleic acid encoding eight or more human Ig variable D domains.

### Embodiment C51

The non-human animal of any one of Embodiments C1-C50, wherein said IgH allele comprises at least one exogenous recombinase site recognition nucleic acid sequence.

### Embodiment C52

The non-human animal of Embodiment C51, wherein said at least one exogenous recombinase site recognition nucleic acid sequence is located upstream of said endogenous nucleic acid encoding said CH2 or CH3 domain of said IgG subclass.

### Embodiment C53

The non-human animal of any one of Embodiments C51-C52, wherein said IgH allele comprises one, two, three, four, five, six, seven, eight, nine, or ten different exogenous recombinase site recognition nucleic acid sequences.

### Embodiment C54

The non-human animal of any one of Embodiments C51-C53, wherein said IgH allele comprises at least three different exogenous recombinase site recognition nucleic acid sequences.

### Embodiment C55

The non-human animal of any one of Embodiments C51-C53, wherein said IgH allele comprises at least five different exogenous recombinase site recognition nucleic acid sequences.

### Embodiment C56

The non-human animal of any one of Embodiments C53-C55, wherein each of said different exogenous recombinase site recognition nucleic acid sequences is located less than 2.5 Mb upstream of an endogenous Eμ.

### Embodiment C57

The non-human animal of any one of Embodiments C53-C55, wherein each of said different exogenous recombinase site recognition nucleic acid sequences is located less than 2.0 Mb, less than 1.5 Mb, less than 1.0 Mb, less than 500 kb, or less than 250 kb upstream of an endogenous Eμ.

### Embodiment C58

The non-human animal of any one of Embodiments C53-C55, wherein each of said different exogenous recombinase site recognition nucleic acid sequences is located less than 200 kb, less than 100 kb, less than 50 kb, less than 25 kb, or less than 10 kb upstream of an endogenous Eμ.

### Embodiment C59

The non-human animal of any one of Embodiments C1-C58, wherein said non-human animal is a mouse or rat.

### Embodiment C60

The non-human animal of any one of Embodiments C1-C58, wherein said non-human animal is a mouse.

### Embodiment C61

A method of producing a non-human animal of any one of Embodiments C1-C60, wherein said method comprises either:
(a1) introducing exogenous nucleic acid encoding said first amino acid sequence of said FN3 polypeptide into an endogenous IgH locus in a stem cell of a non-human animal whose genome comprises an endogenous IgH locus comprising said nucleic acid encoding one or more human Ig variable D domains and nucleic acid encoding said second amino acid sequence of said FN3 polypeptide; (b1) implanting said stem cell into a blastocyst; (c1) implanting said blastocyst into a pseudo-pregnant non-human animal to obtain a chimeric non-human animal; (d1) crossing said chimeric non-human animal to a non-human animal to produce offspring; (e1) screening the offspring for heterozygosity; and (f1) identifying a founder non-human animal carrying said exogenous nucleic acid; or
(a2) introducing exogenous nucleic acid encoding said second amino acid sequence of said FN3 polypeptide into an endogenous IgH locus in a stem cell of a non-human animal whose genome comprises an endogenous IgH locus comprising said nucleic acid encoding one or more human Ig variable D domains and nucleic acid encoding said first amino acid sequence of said FN3 polypeptide; (b1) implanting said stem cell into a blastocyst; (c1) implanting said blastocyst into a pseudo-pregnant non-human animal to obtain a chimeric non-human animal; (d1) crossing said chimeric non-human animal to a non-human animal to produce offspring; (e1) screening the offspring for heterozygosity; and (f1) identifying a founder non-human animal carrying said exogenous nucleic acid.

### Embodiment C62

The method of Embodiment C61, wherein said stem cell is an embryonic stem cell.

### Embodiment C63

A method of producing an antibody-like molecule in a non-human animal of any one of Embodiments C1-C60, wherein said method comprises administering an antigen to said non-human animal, wherein one or more B cells in said non-human animal produce an antibody-like molecule comprising said first amino acid sequence, one of said human Ig variable D domains, and said second amino acid sequence.

### Embodiment C64

The method of Embodiment C63, wherein said antibody-like molecule is a single chain antibody-like molecule.

### Embodiment C65

A method of obtaining a B cell that produces an antibody-like molecule capable of binding to an antigen, wherein said method comprises:
(a) administering said antigen to a non-human animal of any one of Embodiments C1-C60, wherein one or more B cells in said non-human animal produce said antibody-like molecule, and wherein said antibody-like molecule comprises said first amino acid sequence, one of said human Ig variable D domains, and said second amino acid sequence, and
(b) isolating said one or more B cells from said non-human animal.

### Embodiment C66

The method of Embodiment C65, wherein said antibody-like molecule is a single chain antibody-like molecule.

### Embodiment D1

A non-human animal, wherein the genome of said non-human animal comprises an immunoglobulin heavy chain (IgH) allele, wherein said IgH allele comprises an endogenous nucleic acid encoding a CH2 or CH3 domain of an IgG subclass, wherein said IgH allele lacks nucleic acid encoding at least a portion of an endogenous CH1 domain of said IgG subclass or at least a portion of an endogenous regulatory element that drives expression of said endogenous CH1 domain, wherein said IgH allele comprises one or more modified human Ig JH gene segments that do not encode for at least one naturally-occurring tryptophan residue, wherein said non-human animal is capable of producing an antibody, wherein said antibody (a) comprises said CH2 or CH3 domain of said IgG subclass, (b) comprises a variable region comprising a JH domain comprising the amino acid sequence encoded by one of said modified human Ig JH gene segments, and (c) lacks said CH1 domain.

### Embodiment D2

The non-human animal of Embodiment D1, wherein said IgH allele lacks endogenous nucleic acid encoding at least a portion of an IgM constant domain, endogenous nucleic acid encoding at least a portion of an IgD constant domain, endogenous nucleic acid encoding at least a portion of an IgE constant domain, or endogenous nucleic acid encoding at least a portion of an IgA constant domain.

### Embodiment D3

The non-human animal of any one of said Embodiments D1-D2, wherein said IgH allele of said non-human animal comprises endogenous nucleic acid encoding said CH2 domain and said CH3 domain of said IgG subclass.

### Embodiment D4

The non-human animal of any one of Embodiments D1-D3, wherein said IgH allele of said non-human animal comprises endogenous nucleic acid encoding a hinge domain of said IgG subclass.

### Embodiment D5

The non-human animal of any one of Embodiments D1-D4, wherein said IgG subclass is an IgG2 subclass.

### Embodiment D6

The non-human animal of any one of Embodiments D1-D5, wherein said IgG subclass is an IgG2a, IgG2b, IgG2c, IgG3, or IgG4 subclass.

### Embodiment D7

The non-human animal of any one of Embodiments D1-D6, wherein said IgG subclass is an IgG1 subclass.

### Embodiment D8

The non-human animal of Embodiment D7, wherein said IgH allele lacks endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain.

### Embodiment D9

The non-human animal of Embodiment D7, wherein said IgH allele lacks endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain.

### Embodiment D10

The non-human animal of Embodiment D7, wherein said IgH allele lacks endogenous nucleic acid encoding each of the IgG2 constant domains, endogenous nucleic acid encoding each of the IgG3 constant domains, or endogenous nucleic acid encoding each of the IgG4 constant domains.

### Embodiment D11

The non-human animal of Embodiment D7, wherein said IgH allele lacks endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains.

### Embodiment D12

The non-human animal of any one of Embodiments D1-D11, wherein said IgH allele lacks endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain.

### Embodiment D13

The non-human animal of any one of Embodiments D1-D11, wherein said IgH allele lacks endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains.

### Embodiment D14

The non-human animal of any one of Embodiments D1-D13, wherein said IgH allele lacks endogenous nucleic acid encoding each of the IgM constant domains.

### Embodiment D15

The non-human animal of any one of Embodiments D1-D14, wherein said IgH allele lacks endogenous nucleic acid encoding each of the IgD constant domains.

### Embodiment D16

The non-human animal of any one of Embodiments D1-D15, wherein said IgH allele lacks endogenous nucleic acid encoding each of the IgE constant domains.

### Embodiment D17

The non-human animal of any one of Embodiments D1-D16, wherein said IgH allele lacks endogenous nucleic acid encoding IgA CH1 and CH2 constant domains.

### Embodiment D18

The non-human animal of any one of Embodiments D1-D17, wherein said IgH allele lacks nucleic acid encoding said endogenous CH1 domain.

### Embodiment D19

The non-human animal of any one of Embodiments D1-D18, wherein said IgH allele lacks said endogenous regulatory element.

### Embodiment D20

The non-human animal of any one of Embodiments D1-D19, wherein said IgH allele comprises an endogenous Eμ.

### Embodiment D21

The non-human animal of Embodiment D20, wherein the first nucleic acid sequence encoding a full length CH2 domain downstream of said endogenous Eμ is nucleic acid encoding an IgG CH2 domain.

### Embodiment D22

The non-human animal of Embodiment D20, wherein the first nucleic acid sequence encoding a full length CH2 domain downstream of said endogenous Eμ is nucleic acid encoding an IgG1 CH2 domain.

### Embodiment D23

The non-human animal of any one of Embodiments D1-D22, wherein said IgH allele comprises an endogenous Sμ, an endogenous Iμ promoter, an endogenous Iμ exon, or a combination thereof.

### Embodiment D24

The non-human animal of Embodiment D23, wherein the first nucleic acid sequence encoding a full length CH2 domain downstream of said endogenous Sμ, said endogenous Iμ promoter, or said endogenous Iμ exon is nucleic acid encoding an IgG CH2 domain.

### Embodiment D25

The non-human animal of Embodiment D23, wherein the first nucleic acid sequence encoding a full length CH2 domain downstream of said endogenous Sμ, said endogenous Iμ promoter, or said endogenous Iμ exon is nucleic acid encoding an IgG1 CH2 domain.

### Embodiment D26

The non-human animal of any one of Embodiments D1-D25, wherein said IgH allele comprises an endogenous 3′γ1E.

### Embodiment D27

The non-human animal of Embodiment D26, wherein said IgH allele lacks endogenous nucleic acid encoding a full length CH2 domain downstream of said endogenous 3′γ1E.

### Embodiment D28

The non-human animal of any one of Embodiments D1-D27, wherein said IgH allele comprises an endogenous 5′hsR1.

### Embodiment D29

The non-human animal of Embodiment D28, wherein the first nucleic acid sequence encoding a full length CH2 domain upstream of said endogenous 5′hsR1 is nucleic acid encoding an IgG CH2 domain.

### Embodiment D30

The non-human animal of Embodiment D28, wherein the first nucleic acid sequence encoding a full length CH2 domain upstream of said endogenous 5′hsR1 is nucleic acid encoding an IgG1 CH2 domain.

### Embodiment D31

The non-human animal of any one of Embodiments D1-D30, wherein said IgH allele comprises an endogenous 3′RR.

### Embodiment D32

The non-human animal of Embodiment D31, wherein the first nucleic acid sequence encoding a full length CH2 domain upstream of said endogenous 3′RR is nucleic acid encoding an IgG CH2 domain.

### Embodiment D33

The non-human animal of Embodiment D31, wherein the first nucleic acid sequence encoding a full length CH2 domain upstream of said endogenous 3′RR is nucleic acid encoding an IgG1 CH2 domain.

### Embodiment D34

The non-human animal of any one of Embodiments D1-D33, wherein said IgH allele comprises an endogenous 3′CBE.

### Embodiment D35

The non-human animal of Embodiment D34, wherein the first nucleic acid sequence encoding a full length CH2 domain upstream of said endogenous 3′CBE is nucleic acid encoding an IgG CH2 domain.

### Embodiment D36

The non-human animal of Embodiment D34, wherein the first nucleic acid sequence encoding a full length CH2 domain upstream of said endogenous 3′CBE is nucleic acid encoding an IgG1 CH2 domain.

### Embodiment D37

The non-human animal of any one of Embodiments D1-D36, wherein at least one allele of said genome lacks at least a portion of the endogenous Ig heavy chain variable region.

### Embodiment D38

The non-human animal of any one of Embodiments D1-D36, wherein at least one allele of said genome lacks all the exons of the endogenous Ig heavy chain variable region.

### Embodiment D39

The non-human animal of any one of Embodiments D1-D36, wherein both alleles of said genome lack all the exons of the endogenous Ig heavy chain variable region.

### Embodiment D40

The non-human animal of any one of Embodiments D1-D36, wherein neither allele of said genome comprises an endogenous exon of an Ig heavy chain variable region.

### Embodiment D41

The non-human animal of any one of Embodiments D1-D40, wherein said IgH allele comprises exogenous nucleic acid encoding one or more human Ig VH gene segments.

### Embodiment D42

The non-human animal of Embodiment D41, wherein said IgH allele comprises three or more human Ig VH gene segments.

### Embodiment D43

The non-human animal of Embodiment D41, wherein said IgH allele comprises 26 or more human Ig VH gene segments.

### Embodiment D44

The non-human animal of Embodiment D41, wherein said IgH allele comprises 65 or more human Ig VH gene segments.

### Embodiment D45

The non-human animal of Embodiment D41, wherein said IgH allele comprises 85 or more human Ig VH gene segments.

### Embodiment D46

The non-human animal of Embodiment D41, wherein said IgH allele comprises 129 human Ig VH gene segments.

### Embodiment D47

The non-human animal of any one of Embodiments D1-D46, wherein said IgH allele comprises 13 or more human Ig DH gene segments.

### Embodiment D48

The non-human animal of any one of Embodiments D1-D46, wherein said IgH allele comprises 27 human Ig DH gene segments.

### Embodiment D49

The non-human animal of any one of Embodiments D1-D48, wherein said IgH allele comprises three or more of said modified human Ig JH gene segments.

### Embodiment D50

The non-human animal of any one of Embodiments D1-D49, wherein said IgH allele comprises five or more of said modified human Ig JH gene segments.

### Embodiment D51

The non-human animal of any one of Embodiments D1-D40, wherein said IgH allele comprises 129 or more human Ig VH gene segments, 27 or more human Ig DH gene segments, and 6 or more of said modified human Ig JH gene segments.

### Embodiment D52

The non-human animal of any one of Embodiments D1-D51, wherein said one or more modified human Ig JH gene segments are selected from the group consisting of a modified human Ig JH gene segment that encodes an Ig JH domain having the amino acid sequence set forth in SEQ ID NO:105 and a modified human Ig JH gene segment that encodes an Ig JH domain having the amino acid sequence set forth in SEQ ID NO: 106, wherein the “X” residue within each sequence identifier is an amino acid other than tryptophan.

### Embodiment D53

The non-human animal of Embodiment D52, wherein the “X” residue within each sequence identifier is arginine (R), lysine (K), tyrosine (Y), serine(S), threonine (T), histidine (H), or glutamine (Q).

### Embodiment D54

The non-human animal of Embodiment D52, wherein the “X” residue within each sequence identifier is arginine (R), lysine (K), tyrosine (Y), serine(S), threonine (T), or glutamine (Q).

### Embodiment D55

The non-human animal of Embodiment D52, wherein the “X” residue within each sequence identifier is arginine (R), lysine (K), serine(S), threonine (T), histidine (H), or glutamine (Q).

### Embodiment D56

The non-human animal of Embodiment D52, wherein the “X” residue within each sequence identifier is arginine (R), tyrosine (Y), serine(S), threonine (T), histidine (H), or glutamine (Q).

### Embodiment D57

The non-human animal of Embodiment D52, wherein the “X” residue within each sequence identifier is tyrosine (Y), serine(S), or threonine (T).

### Embodiment D58

The non-human animal of Embodiment D52, wherein the “X” residue within each sequence identifier is tyrosine (Y) or serine(S).

### Embodiment D59

The non-human animal of Embodiment D52, wherein the “X” residue within each sequence identifier is arginine (R).

### Embodiment D60

The non-human animal of Embodiment D52, wherein the “X” residue within each sequence identifier is lysine (K).

### Embodiment D61

The non-human animal of Embodiment D52, wherein the “X” residue within each sequence identifier is tyrosine (Y).

### Embodiment D62

The non-human animal of Embodiment D52, wherein the “X” residue within each sequence identifier is serine(S).

### Embodiment D63

The non-human animal of Embodiment D52, wherein the “X” residue within each sequence identifier is threonine (T).

### Embodiment D64

The non-human animal of Embodiment D52, wherein the “X” residue within each sequence identifier is histidine (H).

### Embodiment D65

The non-human animal of Embodiment D52, wherein the “X” residue within each sequence identifier is glutamine (Q).

### Embodiment D66

The non-human animal of any one of Embodiments D1-D65, wherein said IgH allele comprises at least one exogenous recombinase site recognition nucleic acid sequence.

### Embodiment D67

The non-human animal of Embodiment D66, wherein said at least one exogenous recombinase site recognition nucleic acid sequence is located upstream of said endogenous nucleic acid encoding said CH2 or CH3 domain of said IgG subclass.

### Embodiment D68

The non-human animal of any one of Embodiments D66-D67, wherein said IgH allele comprises one, two, three, four, five, six, seven, eight, nine, or ten different exogenous recombinase site recognition nucleic acid sequences.

### Embodiment D69

The non-human animal of any one of Embodiments D66-D68, wherein said IgH allele comprises at least three different exogenous recombinase site recognition nucleic acid sequences.

### Embodiment D70

The non-human animal of any one of Embodiments D66-D68, wherein said IgH allele comprises at least five different exogenous recombinase site recognition nucleic acid sequences.

### Embodiment D71

The non-human animal of any one of Embodiments D66-D70, wherein each of said different exogenous recombinase site recognition nucleic acid sequences is located less than 2.5 Mb upstream of an endogenous Eμ.

### Embodiment D72

The non-human animal of any one of Embodiments D66-D70, wherein each of said different exogenous recombinase site recognition nucleic acid sequences is located less than 2.0 Mb, less than 1.5 Mb, less than 1.0 Mb, less than 500 kb, or less than 250 kb upstream of an endogenous Eμ.

### Embodiment D73

The non-human animal of any one of Embodiments D66-D70, wherein each of said different exogenous recombinase site recognition nucleic acid sequences is located less than 200 kb, less than 100 kb, less than 50 kb, less than 25 kb, or less than 10 kb upstream of an endogenous Eμ.

### Embodiment D74

The non-human animal of any one of Embodiments D1-D73, wherein said non-human animal is a mouse or rat.

### Embodiment D75

The non-human animal of any one of Embodiments D1-D73, wherein said non-human animal is a mouse.

### Embodiment D76

A method of producing a non-human animal of any one of Embodiments D1-D75, wherein said method comprises (a) introducing exogenous nucleic acid comprising one or more modified human Ig JH gene segments that do not encode for at least one naturally-occurring tryptophan residue, into an endogenous IgH locus in a stem cell of a non-human animal; (b) implanting said stem cell into a blastocyst; (c) implanting said blastocyst into a pseudo-pregnant non-human animal to obtain a chimeric non-human animal; (d) crossing said chimeric non-human animal to a wild-type non-human animal to produce offspring; (e) screening the offspring for heterozygosity; and (f) identifying a founder non-human animal carrying said one or more modified human Ig JH gene segments.

### Embodiment D77

The method of Embodiment D76, wherein said stem cell is an embryonic stem cell.

### Embodiment D78

A method of producing an antibody in a non-human animal of any one of Embodiments D1-D75, wherein said method comprises administering an antigen to said non-human animal, wherein one or more B cells in said non-human animal produce an antibody comprising a JH domain encoded by one of said modified human Ig JH gene segments.

### Embodiment D79

The method of Embodiment D78, wherein said antibody is a single domain antibody (sdAb).

### Embodiment D80

A method of obtaining a B cell that produces an antibody capable of binding to an antigen, wherein said method comprises:
(a) administering said antigen to a non-human animal of any one of Embodiments D1-D75, wherein one or more B cells in said non-human animal produce said antibody, and wherein said antibody comprises a JH domain encoded by one of said modified human Ig JH gene segments, and
(b) isolating said one or more B cells from said non-human animal.

### Embodiment D81

The method of Embodiment D80, wherein said antibody is a single domain antibody (sdAb).

### Embodiment D82

The method of any one of Embodiments D1-D81, wherein said one or more modified human Ig JH gene segments do not encode for any naturally-occurring tryptophan residues.

### Embodiment E1

A non-human animal, wherein the genome of said non-human animal comprises an immunoglobulin heavy chain (IgH) allele, wherein said IgH allele comprises an endogenous nucleic acid encoding a CH2 or CH3 domain of an IgG subclass, wherein said IgH allele lacks nucleic acid encoding at least a portion of an endogenous CH1 domain of said IgG subclass or at least a portion of an endogenous regulatory element that drives expression of said endogenous CH1 domain, wherein said IgH allele comprises one or more Ig VH gene segments selected from the group consisting of an Ig VH gene segment that encodes the amino acid sequence set forth in SEQ ID NO:119, an Ig VH gene segment that encodes the amino acid sequence set forth in SEQ ID NO:120, an Ig VH gene segment that encodes the amino acid sequence set forth in SEQ ID NO: 121, an Ig VH gene segment that encodes the amino acid sequence set forth in SEQ ID NO:122, and an Ig VH gene segment that encodes the amino acid sequence set forth in SEQ ID NO:123, wherein said non-human animal is capable of producing an antibody, wherein said antibody (a) comprises said CH2 or CH3 domain of said IgG subclass and a variable domain comprising the amino acid sequence encoded by one of said Ig VH gene segments and (b) lacks said CH1 domain.

### Embodiment E2

The non-human animal of Embodiment E1, wherein said IgH allele lacks endogenous nucleic acid encoding at least a portion of an IgM constant domain, endogenous nucleic acid encoding at least a portion of an IgD constant domain, endogenous nucleic acid encoding at least a portion of an IgE constant domain, or endogenous nucleic acid encoding at least a portion of an IgA constant domain.

### Embodiment E3

The non-human animal of any one of said Embodiments E1-E2, wherein said IgH allele of said non-human animal comprises endogenous nucleic acid encoding said CH2 domain and said CH3 domain of said IgG subclass.

### Embodiment E4

The non-human animal of any one of Embodiments E1-E3, wherein said IgH allele of said non-human animal comprises endogenous nucleic acid encoding a hinge domain of said IgG subclass.

### Embodiment E5

The non-human animal of any one of Embodiments E1-E4, wherein said IgG subclass is an IgG2 subclass.

### Embodiment E6

The non-human animal of any one of Embodiments E1-E5, wherein said IgG subclass is an IgG2a, IgG2b, IgG2c, IgG3, or IgG4 subclass.

### Embodiment E7

The non-human animal of any one of Embodiments E1-E6, wherein said IgG subclass is an IgG1 subclass.

### Embodiment E8

The non-human animal of Embodiment E7, wherein said IgH allele lacks endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain.

### Embodiment E9

The non-human animal of Embodiment E7, wherein said IgH allele lacks endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain.

### Embodiment E10

The non-human animal of Embodiment E7, wherein said IgH allele lacks endogenous nucleic acid encoding each of the IgG2 constant domains, endogenous nucleic acid encoding each of the IgG3 constant domains, or endogenous nucleic acid encoding each of the IgG4 constant domains.

### Embodiment E11

The non-human animal of Embodiment E7, wherein said IgH allele lacks endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains.

### Embodiment E12

The non-human animal of any one of Embodiments E1-E11, wherein said IgH allele lacks endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain.

### Embodiment E13

The non-human animal of any one of Embodiments E1-E11, wherein said IgH allele lacks endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains.

### Embodiment E14

The non-human animal of any one of Embodiments E1-E13, wherein said IgH allele lacks endogenous nucleic acid encoding each of the IgM constant domains.

### Embodiment E15

The non-human animal of any one of Embodiments E1-E14, wherein said IgH allele lacks endogenous nucleic acid encoding each of the IgD constant domains.

### Embodiment E16

The non-human animal of any one of Embodiments E1-E15, wherein said IgH allele lacks endogenous nucleic acid encoding each of the IgE constant domains.

### Embodiment E17

The non-human animal of any one of Embodiments E1-E16, wherein said IgH allele lacks endogenous nucleic acid encoding IgA CH1 and CH2 constant domains.

### Embodiment E18

The non-human animal of any one of Embodiments E1-E17, wherein said IgH allele lacks nucleic acid encoding said endogenous CH1 domain.

### Embodiment E19

The non-human animal of any one of Embodiments E1-E18, wherein said IgH allele lacks said endogenous regulatory element.

### Embodiment E20

The non-human animal of any one of Embodiments E1-E19, wherein said IgH allele comprises an endogenous Eμ.

### Embodiment E21

The non-human animal of Embodiment E20, wherein the first nucleic acid sequence encoding a full length CH2 domain downstream of said endogenous Eμ is nucleic acid encoding an IgG CH2 domain.

### Embodiment E22

The non-human animal of Embodiment E20, wherein the first nucleic acid sequence encoding a full length CH2 domain downstream of said endogenous Eμ is nucleic acid encoding an IgG1 CH2 domain.

### Embodiment E23

The non-human animal of any one of Embodiments E1-E22, wherein said IgH allele comprises an endogenous Sμ, an endogenous Iμ promoter, an endogenous Iμ exon, or a combination thereof.

### Embodiment E24

The non-human animal of Embodiment E23, wherein the first nucleic acid sequence encoding a full length CH2 domain downstream of said endogenous Sμ, said endogenous Iμ promoter, or said endogenous Iμ exon is nucleic acid encoding an IgG CH2 domain.

### Embodiment E25

The non-human animal of Embodiment E23, wherein the first nucleic acid sequence encoding a full length CH2 domain downstream of said endogenous Sμ, said endogenous Iμ promoter, or said endogenous Iμ exon is nucleic acid encoding an IgG1 CH2 domain.

### Embodiment E26

The non-human animal of any one of Embodiments E1-E25, wherein said IgH allele comprises an endogenous 3′γ1E.

### Embodiment E27

The non-human animal of Embodiment E26, wherein said IgH allele lacks endogenous nucleic acid encoding a full length CH2 domain downstream of said endogenous 3′γ1E.

### Embodiment E28

The non-human animal of any one of Embodiments E1-E27, wherein said IgH allele comprises an endogenous 5′hsR1.

### Embodiment E29

The non-human animal of Embodiment E28, wherein the first nucleic acid sequence encoding a full length CH2 domain upstream of said endogenous 5′hsR1 is nucleic acid encoding an IgG CH2 domain.

### Embodiment E30

The non-human animal of Embodiment E28, wherein the first nucleic acid sequence encoding a full length CH2 domain upstream of said endogenous 5′hsR1 is nucleic acid encoding an IgG1 CH2 domain.

### Embodiment E31

The non-human animal of any one of Embodiments E1-E30, wherein said IgH allele comprises an endogenous 3′RR.

### Embodiment E32

The non-human animal of Embodiment E31, wherein the first nucleic acid sequence encoding a full length CH2 domain upstream of said endogenous 3′RR is nucleic acid encoding an IgG CH2 domain.

### Embodiment E33

The non-human animal of Embodiment E31, wherein the first nucleic acid sequence encoding a full length CH2 domain upstream of said endogenous 3′RR is nucleic acid encoding an IgG1 CH2 domain.

### Embodiment E34

The non-human animal of any one of Embodiments E1-E33, wherein said IgH allele comprises an endogenous 3′CBE.

### Embodiment E35

The non-human animal of Embodiment E34, wherein the first nucleic acid sequence encoding a full length CH2 domain upstream of said endogenous 3′CBE is nucleic acid encoding an IgG CH2 domain.

### Embodiment E36

The non-human animal of Embodiment E34, wherein the first nucleic acid sequence encoding a full length CH2 domain upstream of said endogenous 3′CBE is nucleic acid encoding an IgG1 CH2 domain.

### Embodiment E37

The non-human animal of any one of Embodiments E1-E36, wherein at least one allele of said genome lacks at least a portion of the endogenous Ig heavy chain variable region.

### Embodiment E38

The non-human animal of any one of Embodiments E1-E36, wherein at least one allele of said genome lacks all the exons of the endogenous Ig heavy chain variable region.

### Embodiment E39

The non-human animal of any one of Embodiments E1-E36, wherein both alleles of said genome lack all the exons of the endogenous Ig heavy chain variable region.

### Embodiment E40

The non-human animal of any one of Embodiments E1-E36, wherein neither allele of said genome comprises an endogenous exon of an Ig heavy chain variable region.

### Embodiment E41

The non-human animal of any one of Embodiments E1-E40, wherein said IgH allele comprises, in addition to said one or more Ig VH gene segments selected from said group, exogenous nucleic acid encoding one or more human Ig VH gene segments.

### Embodiment E42

The non-human animal of Embodiment E41, wherein said IgH allele comprises three or more human Ig VH gene segments.

### Embodiment E43

The non-human animal of Embodiment E41, wherein said IgH allele comprises 26 or more human Ig VH gene segments.

### Embodiment E44

The non-human animal of Embodiment E41, wherein said IgH allele comprises 65 or more human Ig VH gene segments.

### Embodiment E45

The non-human animal of Embodiment E41, wherein said IgH allele comprises 85 or more human Ig VH gene segments.

### Embodiment E46

The non-human animal of Embodiment E41, wherein said IgH allele comprises 129 human Ig VH gene segments.

### Embodiment E47

The non-human animal of any one of Embodiments E1-E46, wherein said IgH allele comprises 13 or more human Ig DH gene segments.

### Embodiment E48

The non-human animal of any one of Embodiments E1-E46, wherein said IgH allele comprises 27 human Ig DH gene segments.

### Embodiment E49

The non-human animal of any one of Embodiments E1-E48, wherein said IgH allele comprises three or more human Ig JH gene segments.

### Embodiment E50

The non-human animal of any one of Embodiments E1-E49, wherein said IgH allele comprises 9 human Ig JH gene segments.

### Embodiment E51

The non-human animal of any one of Embodiments E1-E40, wherein said IgH allele comprises 129 or more human Ig VH gene segments, 27 or more human Ig DH gene segments, and 9 or more human Ig JH gene segments.

### Embodiment E52

The non-human animal of any one of Embodiments E1-E51, wherein said IgH allele comprises two or more of said Ig VH gene segments selected from said group.

### Embodiment E53

The non-human animal of any one of Embodiments E1-E51, wherein said IgH allele comprises three or more of said Ig VH gene segments selected from said group.

### Embodiment E54

The non-human animal of any one of Embodiments E1-E51, wherein said IgH allele comprises four or more of said Ig VH gene segments selected from said group.

### Embodiment E55

The non-human animal of any one of Embodiments E1-E51, wherein said IgH allele comprises said Ig VH gene segment that encodes the amino acid sequence set forth in SEQ ID NO:119, said Ig VH gene segment that encodes the amino acid sequence set forth in SEQ ID NO: 120, said Ig VH gene segment that encodes the amino acid sequence set forth in SEQ ID NO: 121, said Ig VH gene segment that encodes the amino acid sequence set forth in SEQ ID NO: 122, and said Ig VH gene segment that encodes the amino acid sequence set forth in SEQ ID NO:123.

### Embodiment E56

The non-human animal of any one of Embodiments E1-E55, wherein said IgH allele comprises an endogenous regulatory element for each of said one or more Ig VH gene segments.

### Embodiment E57

The non-human animal of Embodiment E56, wherein said endogenous regulatory element is an endogenous promoter sequence of said non-human animal.

### Embodiment E58

The non-human animal of any one of Embodiments E1-E57, wherein said IgH allele comprises an endogenous exon encoding a leader sequence for each of said one or more Ig VH gene segments.

### Embodiment E59

The non-human animal of Embodiment E58, wherein said endogenous exon is an endogenous L1 exon of said non-human animal.

### Embodiment E60

The non-human animal of Embodiment E58, wherein said endogenous exon is an endogenous L2 exon of said non-human animal.

### Embodiment E61

The non-human animal of Embodiment E58, wherein said IgH allele comprises endogenous L1 and L2 exons encoding a leader sequence for each of said one or more Ig VH gene segments.

### Embodiment E62

The non-human animal of any one of Embodiments E1-E61, wherein said IgH allele comprises at least one exogenous recombinase site recognition nucleic acid sequence.

### Embodiment E63

The non-human animal of Embodiment E62, wherein said at least one exogenous recombinase site recognition nucleic acid sequence is located upstream of said endogenous nucleic acid encoding said CH2 or CH3 domain of said IgG subclass.

### Embodiment E64

The non-human animal of any one of Embodiments E62-E63, wherein said IgH allele comprises one, two, three, four, five, six, seven, eight, nine, or ten different exogenous recombinase site recognition nucleic acid sequences.

### Embodiment E65

The non-human animal of any one of Embodiments E62-E64, wherein said IgH allele comprises at least three different exogenous recombinase site recognition nucleic acid sequences.

### Embodiment E66

The non-human animal of any one of Embodiments E62-E64, wherein said IgH allele comprises at least five different exogenous recombinase site recognition nucleic acid sequences.

### Embodiment E67

The non-human animal of any one of Embodiments E62-E64, wherein each of said different exogenous recombinase site recognition nucleic acid sequences is located less than 2.5 Mb upstream of an endogenous Eμ.

### Embodiment E68

The non-human animal of any one of Embodiments E62-E64, wherein each of said different exogenous recombinase site recognition nucleic acid sequences is located less than 2.0 Mb, less than 1.5 Mb, less than 1.0 Mb, less than 500 kb, or less than 250 kb upstream of an endogenous Eμ.

### Embodiment E69

The non-human animal of any one of Embodiments E62-E64, wherein each of said different exogenous recombinase site recognition nucleic acid sequences is located less than 200 kb, less than 100 kb, less than 50 kb, less than 25 kb, or less than 10 kb upstream of an endogenous Eμ.

### Embodiment E70

The non-human animal of any one of Embodiments E1-E69, wherein said non-human animal is a mouse or rat.

### Embodiment E71

The non-human animal of any one of Embodiments E1-E69, wherein said non-human animal is a mouse.

### Embodiment E72

A method of producing a non-human animal of any one of Embodiments E1-E71, wherein said method comprises (a) introducing exogenous nucleic acid comprising one or more Ig VH gene segments selected from the group consisting of an Ig VH gene segment that encodes the amino acid sequence set forth in SEQ ID NO:119, an Ig VH gene segment that encodes the amino acid sequence set forth in SEQ ID NO: 120, an Ig VH gene segment that encodes the amino acid sequence set forth in SEQ ID NO:121, an Ig VH gene segment that encodes the amino acid sequence set forth in SEQ ID NO:122, and an Ig VH gene segment that encodes the amino acid sequence set forth in SEQ ID NO: 123 into an endogenous IgH locus in a stem cell of a non-human animal; (b) implanting said stem cell into a blastocyst; (c) implanting said blastocyst into a pseudo-pregnant non-human animal to obtain a chimeric non-human animal; (d) crossing said chimeric non-human animal to a wild-type non-human animal to produce offspring; (e) screening the offspring for heterozygosity; and (f) identifying a founder non-human animal carrying said one or more Ig VH gene segments.

### Embodiment E73

The method of Embodiment E72, wherein said stem cell is an embryonic stem cell.

### Embodiment E74

A method of producing an antibody in a non-human animal of any one of Embodiments E1-E71, wherein said method comprises administering an antigen to said non-human animal, wherein one or more B cells in said non-human animal produce an antibody comprising a variable domain encoded by one of said Ig VH gene segments.

### Embodiment E75

The method of Embodiment E74, wherein said antibody is a single domain antibody (sdAb).

### Embodiment E76

The method of Embodiment E74 or E75, wherein said antibody comprises an amino acid sequence selected from SEQ ID NOs: 119-123.

### Embodiment E77

A method of obtaining a B cell that produces an antibody capable of binding to an antigen, wherein said method comprises:
(a) administering said antigen to a non-human animal of any one of Embodiments E1-E71, wherein one or more B cells in said non-human animal produce said antibody, and wherein said antibody comprises a variable domain comprising the amino acid sequence encoded by one of said Ig VH gene segments, and
(b) isolating said one or more B cells from said non-human animal.

### Embodiment E78

The method of Embodiment E77, wherein said antibody is a single domain antibody (sdAb).

### Embodiment E79

The method of Embodiment E77 or E78, wherein said antibody comprises an amino acid sequence selected from SEQ ID NOs: 119-123.

### Embodiment F1

A chimeric mouse generated from a mouse embryo comprising at least two different mouse cells having different genomes, wherein the genome of a first cell of said at least two different mouse cells comprises a genomic modification that prevents cells derived from said first cell from producing endogenous mouse immunoglobulins, wherein the genome of a second cell of said at least two different mouse cells comprises an IgH locus comprising a nucleic acid sequence encoding an input nanobody domain, wherein said chimeric mouse comprises cells originating from said first cell and cells originating from said second cell, wherein said chimeric mouse produces heavy chain only antibodies containing said input nanobody domain and optionally one or more variants of said heavy chain only antibody that underwent affinity maturation within said chimeric mouse, and wherein said chimeric mouse does not produce endogenous mouse immunoglobulins.

### Embodiment F2

The chimeric mouse of Embodiment F1, wherein said genomic modification of said first cell comprises a homozygous disruption of a nucleic acid sequence encoding a recombination activating gene 2 (Rag2) polypeptide.

### Embodiment F3

The chimeric mouse of Embodiment F1, wherein said genomic modification of said first cell comprises a homozygous disruption of a regulatory nucleic acid sequence that regulates expression of a Rag2 polypeptide.

### Embodiment F4

The chimeric mouse of Embodiment F1, wherein said genomic modification of said first cell comprises a homozygous disruption of nucleic acid encoding immunoglobulin (Ig) domains needed for expression of endogenous mouse antibodies.

### Embodiment F5

The chimeric mouse of Embodiment F1, wherein said genomic modification of said first cell comprises a homozygous disruption of a regulatory nucleic acid sequence that regulates expression of endogenous mouse antibodies.

### Embodiment F6

The chimeric mouse of any one of Embodiments F1-F5, wherein said IgH locus comprises an endogenous nucleic acid encoding a CH2 or CH3 domain of an IgG subclass, wherein said IgH locus lacks nucleic acid encoding at least a portion of an endogenous CH1 domain of said IgG subclass or at least a portion of an endogenous regulatory element that drives expression of said endogenous CH1 domain, wherein said heavy chain only antibody (a) comprises said CH2 or CH3 domain of said IgG subclass and (b) lacks said CH1 domain.

### Embodiment F7

The chimeric mouse of Embodiment F6, wherein said IgH locus comprises endogenous nucleic acid encoding a hinge, said CH2 domain, and said CH3 domain of said IgG subclass.

### Embodiment F8

The chimeric mouse of any one of Embodiments F6-F7, wherein said IgH locus comprises endogenous nucleic acid encoding a hinge domain of said IgG subclass.

### Embodiment F9

The chimeric mouse of any one of Embodiments F6-F8, wherein said IgG subclass is an IgG2 subclass.

### Embodiment F10

The chimeric mouse of any one of Embodiments F6-F8, wherein said IgG subclass is an IgG2a, IgG2b, IgG2c, IgG3, or IgG4 subclass.

### Embodiment F11

The chimeric mouse of any one of Embodiments F6-F8, wherein said IgG subclass is an IgG1 subclass.

### Embodiment F12

The chimeric mouse of Embodiment F11, wherein said IgH locus lacks endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain.

### Embodiment F13

The chimeric mouse of Embodiment F11, wherein said IgH locus lacks endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain.

### Embodiment F14

The chimeric mouse of Embodiment F11, wherein said IgH locus lacks endogenous nucleic acid encoding each of the IgG2 constant domains, endogenous nucleic acid encoding each of the IgG3 constant domains, or endogenous nucleic acid encoding each of the IgG4 constant domains.

### Embodiment F15

The chimeric mouse of Embodiment F11, wherein said IgH locus lacks endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains.

### Embodiment F16

The chimeric mouse of any one of Embodiments F1-F15, wherein said IgH locus lacks endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains.

### Embodiment F17

The chimeric mouse of any one of Embodiments F1-F16, wherein said IgH locus lacks endogenous nucleic acid encoding each of the IgM constant domains.

### Embodiment F18

The chimeric mouse of any one of Embodiments F1-F17, wherein said IgH locus lacks endogenous nucleic acid encoding each of the IgD constant domains.

### Embodiment F19

The chimeric mouse of any one of Embodiments F1-F18, wherein said IgH locus lacks endogenous nucleic acid encoding each of the IgE constant domains.

### Embodiment F20

The chimeric mouse of any one of Embodiments F1-F19, wherein said IgH locus lacks endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain.

### Embodiment F21

The chimeric mouse of any one of Embodiments F1-F20, wherein said IgH locus lacks endogenous nucleic acid encoding IgA CH1 and CH2 constant domains.

### Embodiment F22

The chimeric mouse of any one of Embodiments F1-F21, wherein said IgH locus lacks nucleic acid encoding said endogenous CH1 domain.

### Embodiment F23

The chimeric mouse of any one of Embodiments F1-F22, wherein said IgH locus lacks said endogenous regulatory element.

### Embodiment F24

The chimeric mouse of any one of Embodiments F1-F23, wherein said IgH locus comprises an endogenous Eμ.

### Embodiment F25

The chimeric mouse of Embodiment F24, wherein the first nucleic acid sequence encoding a full length CH2 domain downstream of said endogenous Eμ is nucleic acid encoding an IgG CH2 domain.

### Embodiment F26

The chimeric mouse of Embodiment F25, wherein the first nucleic acid sequence encoding a full length CH2 domain downstream of said endogenous Eμ is nucleic acid encoding an IgG1 CH2 domain.

### Embodiment F27

The chimeric mouse of any one of Embodiments F1-F26, wherein said IgH locus comprises an endogenous Sμ, an endogenous Iμ promoter, an endogenous Iμ exon, or a combination thereof.

### Embodiment F28

The chimeric mouse of Embodiment F27, wherein the first nucleic acid sequence encoding a full length CH2 domain downstream of said endogenous Sμ, said endogenous Iμ promoter, or said endogenous Iμ exon is nucleic acid encoding an IgG CH2 domain.

### Embodiment F29

The chimeric mouse of Embodiment F27, wherein the first nucleic acid sequence encoding a full length CH2 domain downstream of said endogenous Sμ, said endogenous Iμ promoter, or said endogenous Iμ exon is nucleic acid encoding an IgG1 CH2 domain.

### Embodiment F30

The chimeric mouse of any one of Embodiments F1-F29, wherein said IgH locus comprises an endogenous 3′γ1E.

### Embodiment F31

The chimeric mouse of Embodiment F30, wherein said IgH locus lacks endogenous nucleic acid encoding a full length CH2 domain downstream of said endogenous 3′γ1E.

### Embodiment F32

The chimeric mouse of any one of Embodiments F1-F31, wherein said IgH locus comprises an endogenous 5′hsR1.

### Embodiment F33

The chimeric mouse of Embodiment F32, wherein the first nucleic acid sequence encoding a full length CH2 domain upstream of said endogenous 5′hsR1 is nucleic acid encoding an IgG CH2 domain.

### Embodiment F34

The chimeric mouse of Embodiment F32, wherein the first nucleic acid sequence encoding a full length CH2 domain upstream of said endogenous 5′hsR1 is nucleic acid encoding an IgG1 CH2 domain.

### Embodiment F35

The chimeric mouse of any one of Embodiments F1-F34, wherein said IgH locus comprises an endogenous 3′RR.

### Embodiment F36

The chimeric mouse of Embodiment F35, wherein the first nucleic acid sequence encoding a full length CH2 domain upstream of said endogenous 3′RR is nucleic acid encoding an IgG CH2 domain.

### Embodiment F37

The chimeric mouse of Embodiment F35, wherein the first nucleic acid sequence encoding a full length CH2 domain upstream of said endogenous 3′RR is nucleic acid encoding an IgG1 CH2 domain.

### Embodiment F38

The chimeric mouse of any one of Embodiments F1-F37, wherein said IgH locus comprises an endogenous 3′CBE.

### Embodiment F39

The chimeric mouse of Embodiment F38, wherein the first nucleic acid sequence encoding a full length CH2 domain upstream of said endogenous 3′CBE is nucleic acid encoding an IgG CH2 domain.

### Embodiment F40

The chimeric mouse of Embodiment F38, wherein the first nucleic acid sequence encoding a full length CH2 domain upstream of said endogenous 3′CBE is nucleic acid encoding an IgG1 CH2 domain.

### Embodiment F41

The chimeric mouse of any one of Embodiments F1-F40, wherein at least one allele of said genome of said second cell lacks at least a portion of the endogenous Ig heavy chain variable region.

### Embodiment F42

The chimeric mouse of any one of Embodiments F1-F40, wherein at least one allele of said genome of said second cell lacks all the exons of the endogenous Ig heavy chain variable region.

### Embodiment F43

The chimeric mouse of any one of Embodiments F1-F40, wherein both alleles of said genome of said second cell lack all the exons of the endogenous Ig heavy chain variable region.

### Embodiment F44

The chimeric mouse of any one of Embodiments F1-F40, wherein neither allele of said genome of said second cell comprises an endogenous exon of an Ig heavy chain variable region.

### Embodiment F45

The chimeric mouse of any one of Embodiments F1-F40, wherein one allele of said genome of said second cell comprises said IgH locus.

### Embodiment F46

The chimeric mouse of any one of Embodiments F1-F40, wherein one allele of said genome of said second cell comprises said IgH locus, and the other allele of said genome of said second cell comprises said IgH locus with the exception that it does not comprise said nucleic acid sequence encoding said input nanobody domain.

### Embodiment F47

The chimeric mouse of any one of Embodiments F1-F46, wherein said genomic modification of said first cell comprises a homozygous disruption of a nucleic acid sequence encoding a Rag1 polypeptide.

### Embodiment F48

The chimeric mouse of any one of Embodiments F1-F46, wherein said genomic modification of said first cell comprises a homozygous disruption of a regulatory nucleic acid sequence that regulates expression of a Rag1 polypeptide.

### Embodiment F49

A method for making a chimeric mouse, wherein said method comprises developing said chimeric mouse within the uterus of a surrogate female mouse from an implanted mouse embryo comprising at least two different mouse cells having different genomes, wherein the genome of a first cell of said at least two different mouse cells comprises a genomic modification that prevents cells derived from said first cell from producing endogenous mouse immunoglobulins, wherein the genome of a second cell of said at least two different mouse cells comprises an IgH locus comprising a nucleic acid sequence encoding an input nanobody domain, wherein said chimeric mouse comprises cells originating from said first cell and cells originating from said second cell, wherein said chimeric mouse produces heavy chain only antibodies containing said input nanobody domain and optionally one or more variants of said heavy chain only antibodies that underwent affinity maturation within said chimeric mouse, and wherein said chimeric mouse does not produce endogenous mouse immunoglobulins.

### Embodiment F50

The method of Embodiment F49, wherein said genomic modification of said first cell comprises a homozygous disruption of a nucleic acid sequence encoding a recombination activating gene 2 (Rag2) polypeptide.

### Embodiment F51

The method of Embodiment F49, wherein said genomic modification of said first cell comprises a homozygous disruption of a regulatory nucleic acid sequence that regulates expression of a Rag2 polypeptide

### Embodiment F52

The method of Embodiment F49, wherein said genomic modification of said first cell comprises a homozygous disruption of nucleic acid encoding immunoglobulin (Ig) domains needed for expression of endogenous mouse antibodies.

### Embodiment F53

The method of Embodiment F49, wherein said genomic modification of said first cell comprises a homozygous disruption of a regulatory nucleic acid sequence that regulates expression of endogenous mouse antibodies.

### Embodiment F54

The method of any one of Embodiments F49-F53, wherein said IgH locus comprises an endogenous nucleic acid encoding a CH2 or CH3 domain of an IgG subclass, wherein said IgH locus lacks nucleic acid encoding at least a portion of an endogenous CH1 domain of said IgG subclass or at least a portion of an endogenous regulatory element that drives expression of said endogenous CH1 domain, wherein said heavy chain only antibody (a) comprises said CH2 or CH3 domain of said IgG subclass and (b) lacks said CH1 domain.

### Embodiment F55

The method of Embodiment F54, wherein said IgH locus comprises endogenous nucleic acid encoding a hinge, said CH2 domain, and said CH3 domain of said IgG subclass.

### Embodiment F56

The method of any one of Embodiments F54-F55, wherein said IgH locus comprises endogenous nucleic acid encoding a hinge domain of said IgG subclass.

### Embodiment F57

The method of any one of Embodiments F54-F56, wherein said IgG subclass is an IgG2 subclass.

### Embodiment F58

The method of any one of Embodiments F54-F56, wherein said IgG subclass is an IgG2a, IgG2b, IgG2c, IgG3, or IgG4 subclass.

### Embodiment F59

The method of any one of Embodiments F54-F56, wherein said IgG subclass is an IgG1 subclass.

### Embodiment F60

The method of Embodiment F59, wherein said IgH locus lacks endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain.

### Embodiment F61

The method of Embodiment F59, wherein said IgH locus lacks endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain.

### Embodiment F62

The method of Embodiment F59, wherein said IgH locus lacks endogenous nucleic acid encoding each of the IgG2 constant domains, endogenous nucleic acid encoding each of the IgG3 constant domains, or endogenous nucleic acid encoding each of the IgG4 constant domains.

### Embodiment F63

The method of Embodiment F59, wherein said IgH locus lacks endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains.

### Embodiment F64

The method of any one of Embodiments F49-F63, wherein said IgH locus lacks endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains.

### Embodiment F65

The method of any one of Embodiments F49-F64, wherein said IgH locus lacks endogenous nucleic acid encoding each of the IgM constant domains.

### Embodiment F66

The method of any one of Embodiments F49-F65, wherein said IgH locus lacks endogenous nucleic acid encoding each of the IgD constant domains.

### Embodiment F67

The method of any one of Embodiments F49-F66, wherein said IgH locus lacks endogenous nucleic acid encoding each of the IgE constant domains.

### Embodiment F68

The method of any one of Embodiments F49-F67, wherein said IgH locus lacks endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain.

### Embodiment F69

The method of any one of Embodiments F49-F68, wherein said IgH locus lacks endogenous nucleic acid encoding IgA CH1 and CH2 constant domains.

### Embodiment F70

The method of any one of Embodiments F49-F69, wherein said IgH locus lacks nucleic acid encoding said endogenous CH1 domain.

### Embodiment F71

The method of any one of Embodiments F49-F70, wherein said IgH locus lacks said endogenous regulatory element.

### Embodiment F72

The method of any one of Embodiments F49-F71, wherein said IgH locus comprises an endogenous Eμ.

### Embodiment F73

The method of Embodiment F72, wherein the first nucleic acid sequence encoding a full length CH2 domain downstream of said endogenous Eμ is nucleic acid encoding an IgG CH2 domain.

### Embodiment F74

The method of Embodiment F72, wherein the first nucleic acid sequence encoding a full length CH2 domain downstream of said endogenous Eμ is nucleic acid encoding an IgG1 CH2 domain.

### Embodiment F75

The method of any one of Embodiments F49-F74, wherein said IgH locus comprises an endogenous Sμ, an endogenous Iμ promoter, an endogenous Iμ exon, or a combination thereof.

### Embodiment F76

The method of Embodiment F75, wherein the first nucleic acid sequence encoding a full length CH2 domain downstream of said endogenous Sμ, said endogenous Iμ promoter, or said endogenous Iμ exon is nucleic acid encoding an IgG CH2 domain.

### Embodiment F77

The method of Embodiment F75, wherein the first nucleic acid sequence encoding a full length CH2 domain downstream of said endogenous Sμ, said endogenous Iμ promoter, or said endogenous Iμ exon is nucleic acid encoding an IgG1 CH2 domain.

### Embodiment F78

The method of any one of Embodiments F49-F77, wherein said IgH locus comprises an endogenous 3′γ1E.

### Embodiment F79

The method of Embodiment F78, wherein said IgH locus lacks endogenous nucleic acid encoding a full length CH2 domain downstream of said endogenous 3′γ1E.

### Embodiment F80

The method of any one of Embodiments F49-F79, wherein said IgH locus comprises an endogenous 5′hsR1.

### Embodiment F81

The method of Embodiment F80, wherein the first nucleic acid sequence encoding a full length CH2 domain upstream of said endogenous 5′hsR1 is nucleic acid encoding an IgG CH2 domain.

### Embodiment F82

The method of Embodiment F80, wherein the first nucleic acid sequence encoding a full length CH2 domain upstream of said endogenous 5′hsR1 is nucleic acid encoding an IgG1 CH2 domain.

### Embodiment F83

The method of any one of Embodiments F49-F82, wherein said IgH locus comprises an endogenous 3′RR.

### Embodiment F84

The method of Embodiment F83, wherein the first nucleic acid sequence encoding a full length CH2 domain upstream of said endogenous 3′RR is nucleic acid encoding an IgG CH2 domain.

### Embodiment F85

The method of Embodiment F83, wherein the first nucleic acid sequence encoding a full length CH2 domain upstream of said endogenous 3′RR is nucleic acid encoding an IgG1 CH2 domain.

### Embodiment F86

The method of any one of Embodiments F49-F85, wherein said IgH locus comprises an endogenous 3′CBE.

### Embodiment F87

The method of Embodiment F86, wherein the first nucleic acid sequence encoding a full length CH2 domain upstream of said endogenous 3′CBE is nucleic acid encoding an IgG CH2 domain.

### Embodiment F88

The method of Embodiment F86, wherein the first nucleic acid sequence encoding a full length CH2 domain upstream of said endogenous 3′CBE is nucleic acid encoding an IgG1 CH2 domain.

### Embodiment F89

The method of any one of Embodiments F49-F88, wherein at least one allele of said genome of said second cell lacks at least a portion of the endogenous Ig heavy chain variable region.

### Embodiment F90

The method of any one of Embodiments F49-F88, wherein at least one allele of said genome of said second cell lacks all the exons of the endogenous Ig heavy chain variable region.

### Embodiment F91

The method of any one of Embodiments F49-F88, wherein both alleles of said genome of said second cell lack all the exons of the endogenous Ig heavy chain variable region.

### Embodiment F92

The method of any one of Embodiments F49-F88, wherein neither allele of said genome of said second cell comprises an endogenous exon of an Ig heavy chain variable region.

### Embodiment F93

The method of any one of Embodiments F49-F88, wherein one allele of said genome of said second cell comprises said IgH locus.

### Embodiment F94

The method of any one of Embodiments F49-F88, wherein one allele of said genome of said second cell comprises said IgH locus, and the other allele of said genome of said second cell comprises said IgH locus with the exception that it does not comprise said nucleic acid sequence encoding said input nanobody domain.

### Embodiment F95

The method of any one of Embodiments F49-F94, wherein said genomic modification of said first cell comprises a homozygous disruption of a nucleic acid sequence encoding a Rag1 polypeptide.

### Embodiment F96

The method of any one of Embodiments F49-F94, wherein said genomic modification of said first cell comprises a homozygous disruption of a regulatory nucleic acid sequence that regulates expression of a Rag1 polypeptide.

### Embodiment F97

A method for obtaining a variant of a heavy chain only antibody containing an input nanobody domain that underwent in vivo affinity maturation, wherein said method comprises:
(a) administering an antigen recognized by a nanobody containing said input nanobody domain or an antibody containing said input nanobody domain to a chimeric mouse generated from a mouse embryo comprising at least two different mouse cells having different genomes, wherein the genome of a first cell of said at least two different mouse cells comprises a genomic modification that prevents cells derived from said first cell from producing endogenous mouse immunoglobulins, wherein the genome of a second cell of said at least two different mouse cells comprises an IgH locus comprising a nucleic acid sequence encoding said input nanobody domain, wherein said chimeric mouse comprises cells originating from said first cell and cells originating from said second cell, wherein said chimeric mouse produces heavy chain only antibodies containing said input nanobody domain and one or more variants of said heavy chain only antibody containing said input nanobody domain that underwent affinity maturation within said chimeric mouse, wherein said chimeric mouse does not produce endogenous mouse immunoglobulins, and
(b) obtaining at least one of said one or more variants or a B cell producing at least one of said one or more variants from said chimeric mouse, thereby obtaining said variant of said heavy chain only antibody containing said input nanobody domain that underwent in vivo affinity maturation.

### Embodiment F98

The method of Embodiment F97, wherein said genomic modification of said first cell comprises a homozygous disruption of a nucleic acid sequence encoding a Rag2 polypeptide.

### Embodiment F99

The method of Embodiment F97, wherein said genomic modification of said first cell comprises a homozygous disruption of a regulatory nucleic acid sequence that regulates expression of a Rag2 polypeptide

### Embodiment F100

The method of Embodiment F97, wherein said genomic modification of said first cell comprises a homozygous disruption of nucleic acid encoding immunoglobulin (Ig) domains needed for expression of endogenous mouse antibodies.

### Embodiment F101

The method of Embodiment F97, wherein said genomic modification of said first cell comprises a homozygous disruption of a regulatory nucleic acid sequence that regulates expression of endogenous mouse antibodies.

### Embodiment F102

The method of any one of Embodiments F97-F101, wherein said IgH locus comprises an endogenous nucleic acid encoding a CH2 or CH3 domain of an IgG subclass, wherein said IgH locus lacks nucleic acid encoding at least a portion of an endogenous CH1 domain of said IgG subclass or at least a portion of an endogenous regulatory element that drives expression of said endogenous CH1 domain, wherein said heavy chain only antibody (a) comprises said CH2 or CH3 domain of said IgG subclass and (b) lacks said CH1 domain.

### Embodiment F103

The method of Embodiment F102, wherein said IgH locus comprises endogenous nucleic acid encoding a hinge, said CH2 domain, and said CH3 domain of said IgG subclass.

### Embodiment F104

The method of any one of Embodiments F102-F103, wherein said IgH locus comprises endogenous nucleic acid encoding a hinge domain of said IgG subclass.

### Embodiment F105

The method of any one of Embodiments F102-F104, wherein said IgG subclass is an IgG2 subclass.

### Embodiment F106

The method of any one of Embodiments F102-F104, wherein said IgG subclass is an IgG2a, IgG2b, IgG2c, IgG3, or IgG4 subclass.

### Embodiment F107

The method of any one of Embodiments F102-F104, wherein said IgG subclass is an IgG1 subclass.

### Embodiment F108

The method of Embodiment F107, wherein said IgH locus lacks endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain.

### Embodiment F109

The method of Embodiment F107, wherein said IgH locus lacks endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain.

### Embodiment F110

The method of Embodiment F107, wherein said IgH locus lacks endogenous nucleic acid encoding each of the IgG2 constant domains, endogenous nucleic acid encoding each of the IgG3 constant domains, or endogenous nucleic acid encoding each of the IgG4 constant domains.

### Embodiment F111

The method of Embodiment F107, wherein said IgH locus lacks endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains.

### Embodiment F112

The method of any one of Embodiments F97-F111, wherein said IgH locus lacks endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains.

### Embodiment F113

The method of any one of Embodiments F97-F112, wherein said IgH locus lacks endogenous nucleic acid encoding each of the IgM constant domains.

### Embodiment F114

The method of any one of Embodiments F97-F113, wherein said IgH locus lacks endogenous nucleic acid encoding each of the IgD constant domains.

### Embodiment F115

The method of any one of Embodiments F97-F114, wherein said IgH locus lacks endogenous nucleic acid encoding each of the IgE constant domains.

### Embodiment F116

The method of any one of Embodiments F97-F115, wherein said IgH locus lacks endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain.

### Embodiment F117

The method of any one of Embodiments F97-F116, wherein said IgH locus lacks endogenous nucleic acid encoding IgA CH1 and CH2 constant domains.

### Embodiment F118

The method of any one of Embodiments F97-F117, wherein said IgH locus lacks nucleic acid encoding said endogenous CH1 domain.

### Embodiment F119

The method of any one of Embodiments F97-F118, wherein said IgH locus lacks said endogenous regulatory element.

### Embodiment F120

The method of any one of Embodiments F95-F119, wherein said IgH locus comprises an endogenous Eμ.

### Embodiment F121

The method of Embodiment F120, wherein the first nucleic acid sequence encoding a full length CH2 domain downstream of said endogenous Eμ is nucleic acid encoding an IgG CH2 domain.

### Embodiment F122

The method of Embodiment F120, wherein the first nucleic acid sequence encoding a full length CH2 domain downstream of said endogenous Eμ is nucleic acid encoding an IgG1 CH2 domain.

### Embodiment F123

The method of any one of Embodiments F97-F122, wherein said IgH locus comprises an endogenous Sμ, an endogenous Iμ promoter, an endogenous Iμ exon, or a combination thereof.

### Embodiment F124

The method of Embodiment F123, wherein the first nucleic acid sequence encoding a full length CH2 domain downstream of said endogenous Sμ, said endogenous Iμ promoter, or said endogenous Iμ exon is nucleic acid encoding an IgG CH2 domain.

### Embodiment F125

The method of Embodiment F123, wherein the first nucleic acid sequence encoding a full length CH2 domain downstream of said endogenous Sμ, said endogenous Iμ promoter, or said endogenous Iμ exon is nucleic acid encoding an IgG1 CH2 domain.

### Embodiment F126

The method of any one of Embodiments F97-F125, wherein said IgH locus comprises an endogenous 3′γ1E.

### Embodiment F127

The method of Embodiment F126, wherein said IgH locus lacks endogenous nucleic acid encoding a full length CH2 domain downstream of said endogenous 3′γ1E.

### Embodiment F128

The method of any one of Embodiments F97-F127, wherein said IgH locus comprises an endogenous 5′hsR1.

### Embodiment F129

The method of Embodiment F128, wherein the first nucleic acid sequence encoding a full length CH2 domain upstream of said endogenous 5′hsR1 is nucleic acid encoding an IgG CH2 domain.

### Embodiment F130

The method of Embodiment F128, wherein the first nucleic acid sequence encoding a full length CH2 domain upstream of said endogenous 5′hsR1 is nucleic acid encoding an IgG1 CH2 domain.

### Embodiment F131

The method of any one of Embodiments F97-F130, wherein said IgH locus comprises an endogenous 3′RR.

### Embodiment F132

The method of Embodiment F131, wherein the first nucleic acid sequence encoding a full length CH2 domain upstream of said endogenous 3′RR is nucleic acid encoding an IgG CH2 domain.

### Embodiment F133

The method of Embodiment F131, wherein the first nucleic acid sequence encoding a full length CH2 domain upstream of said endogenous 3′RR is nucleic acid encoding an IgG1 CH2 domain.

### Embodiment F134

The method of any one of Embodiments F97-F133, wherein said IgH locus comprises an endogenous 3′CBE.

### Embodiment F135

The method of Embodiment F134, wherein the first nucleic acid sequence encoding a full length CH2 domain upstream of said endogenous 3′CBE is nucleic acid encoding an IgG CH2 domain.

### Embodiment F136

The method of Embodiment F134, wherein the first nucleic acid sequence encoding a full length CH2 domain upstream of said endogenous 3′CBE is nucleic acid encoding an IgG1 CH2 domain.

### Embodiment F137

The method of any one of Embodiments F97-F136, wherein at least one allele of said genome of said second cell lacks at least a portion of the endogenous Ig heavy chain variable region.

### Embodiment F138

The method of any one of Embodiments F97-F136, wherein at least one allele of said genome of said second cell lacks all the exons of the endogenous Ig heavy chain variable region.

### Embodiment F139

The method of any one of Embodiments F97-F136, wherein both alleles of said genome of said second cell lack all the exons of the endogenous Ig heavy chain variable region.

### Embodiment F140

The method of any one of Embodiments F97-F136, wherein neither allele of said genome of said second cell comprises an endogenous exon of an Ig heavy chain variable region.

### Embodiment F141

The method of any one of Embodiments F97-F136, wherein one allele of said genome of said second cell comprises said IgH locus.

### Embodiment F142

The method of any one of Embodiments F97-F136, wherein one allele of said genome of said second cell comprises said IgH locus, and the other allele of said genome of said second cell comprises said IgH locus with the exception that it does not comprise said nucleic acid sequence encoding said input nanobody domain.

### Embodiment F143

The method of any one of Embodiments F97-F142, wherein said genomic modification of said first cell comprises a homozygous disruption of a nucleic acid sequence encoding a Rag1 polypeptide.

### Embodiment F144

The method of any one of Embodiments F97-F142, wherein said genomic modification of said first cell comprises a homozygous disruption of a regulatory nucleic acid sequence that regulates expression of a Rag1 polypeptide.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-12. (canceled)

13. A non-human animal, wherein the genome of said non-human animal comprises an immunoglobulin heavy chain (IgH) allele, wherein said IgH allele comprises an endogenous nucleic acid encoding a CH2 or CH3 domain of an IgG subclass, wherein said IgH allele lacks nucleic acid encoding at least a portion of an endogenous CH1 domain of said IgG subclass or at least a portion of an endogenous regulatory element that drives expression of said endogenous CH1 domain, wherein said IgH allele comprises one or more modified human Ig JH gene segments that do not encode for at least one naturally-occurring tryptophan residue, wherein said non-human animal is capable of producing an antibody, wherein said antibody (a) comprises said CH2 or CH3 domain of said IgG subclass, (b) comprises a variable region comprising a JH domain comprising the amino acid sequence encoded by one of said modified human Ig JH gene segments, and (c) lacks said CH1 domain.

14. (canceled)

15. A method of producing an antibody in a non-human animal of claim 13, wherein said method comprises administering an antigen to said non-human animal, wherein one or more B cells in said non-human animal produce an antibody comprising a JH domain encoded by one of said modified human Ig JH gene segments.

16. A method of obtaining a B cell that produces an antibody capable of binding to an antigen, wherein said method comprises:
(a) administering said antigen to a non-human animal of claim 13, wherein one or more B cells in said non-human animal produce said antibody, and wherein said antibody comprises a JH domain encoded by one of said modified human Ig JH gene segments, and
(b) isolating said one or more B cells from said non-human animal.

17-23. (canceled)

24. The non-human animal of claim 13, wherein said IgH allele lacks endogenous nucleic acid encoding at least a portion of an IgM constant domain, endogenous nucleic acid encoding at least a portion of an IgD constant domain, endogenous nucleic acid encoding at least a portion of an IgE constant domain, or endogenous nucleic acid encoding at least a portion of an IgA constant domain.

25. The non-human animal of claim 13, wherein said IgH allele of said non-human animal comprises endogenous nucleic acid encoding said CH2 domain and said CH3 domain of said IgG subclass.

26. The non-human animal of claim 13, wherein said IgH allele of said non-human animal comprises endogenous nucleic acid encoding a hinge domain of said IgG subclass.

27. The non-human animal of claim 13, wherein said IgH allele lacks endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain.

28. The non-human animal of claim 13, wherein said IgH allele lacks endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains.

29. The non-human animal of claim 13, wherein said IgH allele lacks nucleic acid encoding said endogenous CH1 domain.

30. The non-human animal of claim 13, wherein said IgH allele lacks said endogenous regulatory element.

31. The non-human animal of claim 13, wherein said IgH allele comprises an endogenous Eμ.

32. The non-human animal of claim 31, wherein the first nucleic acid sequence encoding a full length CH2 domain downstream of said endogenous Eμ is nucleic acid encoding an IgG CH2 domain.

33. The non-human animal of claim 20, wherein the first nucleic acid sequence encoding a full length CH2 domain downstream of said endogenous Eμ is nucleic acid encoding an IgG1 CH2 domain.

34. The non-human animal of claim 13, wherein said IgH allele comprises an endogenous Sμ, an endogenous Iμ promoter, an endogenous Iμ exon, or a combination thereof.

35. The non-human animal of claim 34, wherein the first nucleic acid sequence encoding a full length CH2 domain downstream of said endogenous Sμ, said endogenous Iμ promoter, or said endogenous Iμ exon is nucleic acid encoding an IgG CH2 domain.

36. The non-human animal of claim 35, wherein the first nucleic acid sequence encoding a full length CH2 domain downstream of said endogenous Sμ, said endogenous Iμ promoter, or said endogenous Iμ exon is nucleic acid encoding an IgG1 CH2 domain.

37. The non-human animal of claim 13, wherein said IgH allele comprises an endogenous 3′γ1E.

38. The non-human animal of claim 26, wherein said IgH allele lacks endogenous nucleic acid encoding a full length CH2 domain downstream of said endogenous 3′γ1E.

39. The non-human animal of claim 13, wherein said IgH allele comprises an endogenous 5′hsR1.

40. The non-human animal of claim 28, wherein the first nucleic acid sequence encoding a full length CH2 domain upstream of said endogenous 5′hsR1 is nucleic acid encoding an IgG CH2 domain.


SEQUENCE LISTING (177 sequences)

SEQ ID NO: 1 - DNA, 47 bp, synthetic construct
  source 1..47: mol_type = other DNA
gacaatggtcaccgtctcttcagtgcccagggattgtggttgtaagc

SEQ ID NO: 2 - protein, 121 aa, synthetic construct
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCARKEYGDYGYATDYWGQGTSVTVSS

SEQ ID NO: 3 - protein, 121 aa, synthetic construct
EVQLQQSGPELVKPGASVKMSCKASGYTFTDYYMKWLKQNHGKSLEWIGDINPNNGDTFYNQKFKGKATLTVDTSSSTAYMQLNSLTSEDSAVYYCARKEYGDYGYATDYWGQGTSVTVSS

SEQ ID NO: 4 - protein, 121 aa, synthetic construct
EVQLQQSGPELVKPGASVKMSCKASGYTFTDYYVKWEKQSHGESLEWIGDINPNNGDTFYNQKFKDKATLTVDKSSSTAYMQLNSLTSEDSAVYYCARKEYGNYGYAVDYWGQGTSVTVSS

SEQ ID NO: 5 - protein, 121 aa, synthetic construct
EVQLQQSGPELVKPGASVKMSCKASGYTFTDYYVKWKKQSHGKSLEWIGDINPNNGDTFYNQKFKDKATLTVDKSSSTAYMQLNSLTSEDSAVYYCARKEYGNFGYAVDYWGQGTSVTVSS

SEQ ID NO: 6 - protein, 121 aa, synthetic construct
EVQLQQSGPELVKPGASVKMSCKASGYTFTDYYIKWEKQSHGKSLEWIGDINPNNGDIFYNPQFKGRATLTVDKSSSTAYMQLNSLTSEDSAVYYCARKEYGDYGYAVDYWGQGTSVTVSS

SEQ ID NO: 7 - protein, 121 aa, synthetic construct
EVQLQQSGPELVKPGASVKMSCKASGYTFTDYYIKWEKQSHGKSLEWIGDINPKNGETFYNQQFKGKATLTVDKSSSTAYMQLNSLTSEDSAVYYCARKEYGDYGYAVDYWGQGTSVTVSS

SEQ ID NO: 8 - protein, 121 aa, synthetic construct
EVQLQQSGPELVKPGASVKISCKASGYTFTDYYMKWVKQRHGQSLEWIGNINPTNGDTNYSQNFKGKATLTVDKSSTTAYMELRSLTSEDSAVYYCARLEDGYYGYAVDYWGQGTSVTVSS

SEQ ID NO: 9 - protein, 121 aa, synthetic construct
EVQLQQSGPELVKPGASVKISCKASGYTFTDYYMKWAKQSHGKSLEWIGHINPVNGDTSYNQKFKGKATLTVDKSSSTVYMELRSLTSEDSAVYYCARLEDGYYGYTMDYWGQGTSVTVSS

SEQ ID NO: 10 - protein, 121 aa, synthetic construct
EVQLQQSGPELVKPGASVKISCKASGYTFTDYYMKWVKQSHGKSLEWIGDINPNNGGARYNQKFKGRATLTVDKSSSTAYMELRSLTSEDSAVYYCSRLEDGYYGYAVDYWGQGTSVTVSS

SEQ ID NO: 11 - protein, 121 aa, synthetic construct
EVQLQQSGPELVKPGASVKISCKASGYTFTDYYMKWEKQSHGKSLEWIGDINPNNGGTRYNQKFRGKATLTVDKSSSTAYMELRSLTSEDSAVYYCARLEDDYYGYAVDYWGQGTSVTVSS

SEQ ID NO: 12 - protein, 121 aa, synthetic construct
EVQLQQSGPELVKPGASVKISCKASGYTFTDYYMKWVKQSHGKSLEWIGDINPKNGGTNYNQKFKDKATLTVDKSSSTAYMELRSLTSEDSAVYYCARLEDGYYGYAIDYWSQGTSVTVSS

SEQ ID NO: 13 - DNA, 52 bp, synthetic construct
  source 1..52: mol_type = other DNA
ctacactctttccctacacgacgctcttccgatctnnnnnnnnnnnnggggg

SEQ ID NO: 14 - DNA, 35 bp, synthetic construct
  source 1..35: mol_type = other DNA
ctacactctttccctacacgacgctcttccgatct

SEQ ID NO: 15 - DNA, 22 bp, synthetic construct
  source 1..22: mol_type = other DNA
ggtggttgtgcaggccctcatg

SEQ ID NO: 16 - DNA, 23 bp, synthetic construct
  source 1..23: mol_type = other DNA
ccatggagttagtttgggcagca

SEQ ID NO: 17 - DNA, 23 bp, synthetic construct
  source 1..23: mol_type = other DNA
caaggcttacaaccacaatccct

SEQ ID NO: 18 - DNA, 296 bp, Homo sapiens
  source 1..296: mol_type = genomic DNA
caggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagagg

SEQ ID NO: 19 - DNA, 296 bp, synthetic construct
  source 1..296: mol_type = other DNA
caggtgcagctcgtccagagtggggctgaagtgaagaagcctggagcatctgtgaaagtatcctgcaaagcgagtggctacaccttcaccagctatgacatcaactgggtgcggcaggcaactggacaaggtctggagtggatgggctggatgaaccccaacagcggaaatactggctatgcccagaagtttcaagggcgcgttaccatgacgaggaatacatccatttctacagcctacatggagctgagctcgttgcgatcagaagatacagctgtctattactgtgccagagg

SEQ ID NO: 20 - DNA, 298 bp, Homo sapiens
  source 1..298: mol_type = genomic DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagata

SEQ ID NO: 21 - DNA, 298 bp, synthetic construct
  source 1..298: mol_type = other DNA
gaagtgcagctggtggagtctggaggagggttggttcagccaggaagatcacttcggctcagctgcgctgctagtggcttcacttttgatgactatgccatgcactgggtaaggcaagctcctgggaaaggcctggagtgggtcagcgggatatcctggaacagtggttccattggctatgcagattctgtgaagggtcgcttcaccatctcgagagacaatgccaaaaattcactctacctgcagatgaacagcttacgagcagaagatacagcgctatactactgtgccaaggaca

SEQ ID NO: 22 - DNA, 299 bp, Homo sapiens
  source 1..299: mol_type = genomic DNA
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagaga

SEQ ID NO: 23 - DNA, 299 bp, synthetic construct
  source 1..299: mol_type = other DNA
caagtccagctgcaggagagtggacctgggctggtgaagcctagccaaaccttgagtcttacctgcactgtgtcgggtggcagcattagttcaggaggttattactggtcctggatcagacagcacccaggaaaaggcctagagtggattggctacatctattattctgggtcaacgtactacaaccccagcctgaagtcccgggtcacaatatctgtagacaccagcaagaatcagttctccctcaaactctcatctgttactgcagctgatacagccgtgtattactgtgccaggga

SEQ ID NO: 24 - DNA, 299 bp, Homo sapiens
  source 1..299: mol_type = genomic DNA
caggtgcagctgcaggagtcgggcccaggactggtgaagccttcacagaccctgtccctcacctgcactgtctctggtggctccatcagcagtggtgattactactggagttggatccgccagcccccagggaagggcctggagtggattgggtacatctattacagtgggagcacctactacaacccgtccctcaagagtcgagttaccatatcagtagacacgtccaagaaccagttctccctgaagctgagctctgtgactgccgcagacacggccgtgtattactgtgccagaga

SEQ ID NO: 25 - DNA, 299 bp, synthetic construct
  source 1..299: mol_type = other DNA
caagtgcagctgcaggagagtgggcctggcctggtgaagccatcccaaaccctttcgctcacctgcacagtaagtggtggctccatatcttcaggagactattactggagctggatcaggcagcccccagggaaaggactggagtggattggctacatctactattctggtagcacgtattataatccttccttgaaaagcagagttaccatttctgtggatactagcaagaaccagttctccctcaagctaagttcagtcacagccgcagacactgctgtctactactgtgcccggga

SEQ ID NO: 26 - DNA, 296 bp, Homo sapiens
  source 1..296: mol_type = genomic DNA
caggtgcagctgcaggagtcgggcccaggactggtgaagccttcggagaccctgtccctcacctgcactgtctctggttactccatcagcagtggttactactggggctggatccggcagcccccagggaaggggctggagtggattgggagtatctatcatagtgggagcacctactacaacccgtccctcaagagtcgagtcaccatatcagtagacacgtccaagaaccagttctccctgaagctgagctctgtgaccgccgcagacacggccgtgtattactgtgcgagaga

SEQ ID NO: 27 - DNA, 296 bp, synthetic construct
  source 1..296: mol_type = other DNA
caagtacagctgcaggagagtggtcctgggctggtgaagccatcagaaactctttcactcacctgcactgtttcaggctacagcatatccagtggctattactggggctggatcaggcagcctccagggaaaggactagagtggattggatccatctaccacagcggtagtacgtattataatcccagcctgaagtccagagtcaccatttctgtggacaccagcaagaaccagttctccttgaaactctcttctgtcacagccgcagatacagctgtgtactactgtgcccggga

SEQ ID NO: 28 - DNA, 298 bp, Homo sapiens
  source 1..298: mol_type = genomic DNA
gaagtgcagctggtggagtctgggggagtcgtggtacagcctggggggtccctgagactctcctgtgcagcctctggattcacctttgatgattatgccatgcactgggtccgtcaagctccggggaagggtctggagtgggtctctcttattagttgggatggtggtagcacatactatgcagactctgtgaagggtcgattcaccatctccagagacaacagcaaaaactccctgtatctgcaaatgaacagtctgagagctgaggacaccgccttgtattactgtgcaaaagata

SEQ ID NO: 29 - DNA, 298 bp, Homo sapiens
  source 1..298: mol_type = genomic DNA
gaagtacaattggtggagagtggcggtgttgtcgtgcagcctggaggtagtctcaggctgtcctgtgcagcgtcaggcttcacttttgatgactatgccatgcactgggtgcggcaggccccagggaaggggctggagtgggtctcattaatttcctgggatggaggcagcacctactatgcagattctgtgaaaggacgcttcacgatctctagagacaattccaagaacagcctatatctgcagatgaacagccttcgagctgaagacacagctctctactactgcgccaaggaca

SEQ ID NO: 30 - DNA, 296 bp, Homo sapiens
  source 1..296: mol_type = genomic DNA
gaggtgcagctggtggagtctgggggaggcttggtacagcctgggggatccctgagactctcctgtgcagcctctggattcaccttcagtaacagtgacatgaactgggtccatcaggctccaggaaaggggctggagtgggtatcgggtgttagttggaatggcagtaggacgcactatgcagactctgtgaagggccaattcatcatctccagagacaattccaggaacaccctgtatctgcaaacgaatagcctgagggccgaggacacggctgtgtattactgtgtgagaaa

SEQ ID NO: 31 - DNA, 296 bp, synthetic construct
  source 1..296: mol_type = other DNA
gaagtgcagctggtggagagtgggggtggtctcgttcagcctggaggaagtctacggctttcctgtgccgcgtcaggctttaccttcagcaattcagacatgaactgggtgcatcaggccccagggaagggcctggagtgggtatctggagtctcttggaatggcagccgtacacactatgctgattctgttaaggggcagttcatcatttcccgcgataattccaggaacacactctacctgcaaaccaacagcttgcgagcagaagacactgctgtgtattactgcgtcagaaa

SEQ ID NO: 32 - DNA, 296 bp, Homo sapiens
  source 1..296: mol_type = genomic DNA
gaggtgcagctggtgaagtctggaggaggcttggtacagcctggggggtccctgagactctcctgtgcagcctctggattcaccttcagtagctctgctatgcactgggtccgccaggctccaagaaagggtttggagtgggtctcagttattagtacaagtggtgataccgtactctacacagactctgtgaagggccgattcaccatctccagagacaatgcccagaattcactgtctctgcaaatgaacagcctgagagccgaggacatggctgtgtattactgtgtgaaaga

SEQ ID NO: 33 - DNA, 296 bp, synthetic construct
  source 1..296: mol_type = other DNA
gaagtacagctggtgaaaagtggtggtgggctcgttcaacctggaggctcccttcgcctcagctgtgctgcgagtggcttcaccttcagctcctcagccatgcactgggttcggcaggcaccaaggaagggcctggagtgggtgtctgtcatttccacttctggggacacagtgctgtatacagatagtgtcaaaggacgatttaccatcagcagagataatgcccagaactcattgtctctgcagatgaacagcctaagagcagaggacatggctgtgtactactgcgtgaagga

SEQ ID NO: 34 - DNA, 296 bp, Homo sapiens
  source 1..296: mol_type = genomic DNA
gaggtgcagctggtggagtctgggggaggcttggtacagcctggggggtccctgagactctcctgtgcagcctctggattcaccttcagtaacagtgacatgaactgggcccgcaaggctccaggaaaggggctggagtgggtatcgggtgttagttggaatggcagtaggacgcactatgtggactccgtgaagcgccgattcatcatctccagagacaattccaggaactccctgtatctgcaaaagaacagacggagagccgaggacatggctgtgtattactgtgtgagaaa

SEQ ID NO: 35 - DNA, 296 bp, synthetic construct
  source 1..296: mol_type = other DNA
gaagtgcagctggtggagagtggtggtggcttggttcaaccaggaggcagcctaaggcttagctgtgctgccagtggcttcacattttccaattcagatatgaactgggcaaggaaggcccctgggaaaggactggagtgggtgtcaggggtctcctggaatggatcccgtacccactatgtggattctgtaaagcgccgcttcatcatttctcgggacaacagcagaaattctctctatctgcagaagaacagaagacgagcggaagacatggctgtctactactgcgttcggaa

SEQ ID NO: 36 - DNA, 292 bp, Homo sapiens
  source 1..292: mol_type = genomic DNA
gaggtgcagctggtggagtctgggggaggcttggtacagcctagggggtccctgagactctcctgtgcagcctctggattcaccgtcagtagcaatgagatgagctggatccgccaggctccagggaaggggctggagtgggtctcatccattagtggtggtagcacatactacgcagactccaggaagggcagattcaccatctccagagacaattccaagaacacgctgtatcttcaaatgaacaacctgagagctgagggcacggccgtgtattactgtgccagatata

SEQ ID NO: 37 - DNA, 292 bp, synthetic construct
  source 1..292: mol_type = other DNA
gaagtgcagctggtggagagtggaggtggtctagtccagcctcgagggagtttgaggcttagctgtgctgcctctggcttcactgtttcttccaatgagatgtcttggatccggcaagccccagggaagggcctggagtgggtgtccagcatttcaggaggaagcacctactatgcagattcaaggaaaggccgttttaccatatccagagacaacagcaagaatacactctacctgcagatgaacaacctcagagcagaagggacagctgtatattactgcgcccgctata

SEQ ID NO: 38 - DNA, 290 bp, Homo sapiens
  source 1..290: mol_type = genomic DNA
gaggtgcagctggtggagtctcggggagtcttggtacagcctggggggtccctgagactctcctgtgcagcctctggattcaccgtcagtagcaatgagatgagctgggtccgccaggctccagggaagggtctggagtgggtctcatccattagtggtggtagcacatactacgcagactccaggaagggcagattcaccatctccagagacaattccaagaacacgctgcatcttcaaatgaacagcctgagagctgaggacacggctgtgtattactgtaagaaaga

SEQ ID NO: 39 - DNA, 290 bp, synthetic construct
  source 1..290: mol_type = other DNA
gaagtgcagctagtggagtcaagaggtgttttggtccagcctggaggaagtctcaggctctcctgtgccgcgtctggctttactgtatcaagtaatgagatgtcctgggtgaggcaagccccagggaaaggcctggagtgggtcagcagtatttctggtggcagcacctattatgctgacagccggaaagggcgcttcaccatctccagagacaacagcaagaacacactgcacctgcagatgaattctcttcgagcagaagatacagctgtgtactactgcaagaagga

SEQ ID NO: 40 - DNA, 296 bp, Homo sapiens
  source 1..296: mol_type = genomic DNA
caggtccagctggtgcagtcttgggctgaggtgaggaagtctggggcctcagtgaaagtctcctgtagtttttctgggtttaccatcaccagctacggtatacattgggtgcaacagtcccctggacaagggcttgagtggatgggatggatcaaccctggcaatggtagcccaagctatgccaagaagtttcagggcagattcaccatgaccagggacatgtccacaaccacagcctacacagacctgagcagcctgacatctgaggacatggctgtgtattactgtgcaagaca

SEQ ID NO: 41 - DNA, 296 bp, synthetic construct
  source 1..296: mol_type = other DNA
caggtgcagctggttcagtcctgggcagaggtgaggaaaagtggtgcttctgtaaaagtctcctgcagcttttctggcttcaccataaccagctatggaattcactgggtccagcagagccccgggcaagggctggagtggatgggctggatcaaccctggaaatggctcgccatcctatgccaagaagttccagggtcgctttaccatgacaagagatatgtctactacgacagcctacacagacctctcaagtcttacttcagaagacatggctgtgtactactgtgcgcggca

SEQ ID NO: 42 - DNA, 296 bp, Homo sapiens
  source 1..296: mol_type = genomic DNA
gaggcccagcttacagagtctgggggagacttggtacacttagaggggcccctgaggctctcctgtgcagcctcttggttcaccttcagtatctatgagattcactgggtttgccaggcctcagggaaggggctggaatgggttgcagttatatggcgtggtgaaagtcatcaatacaatgcagactatgttaggggcagactcaccacttccagagacaacaccaagtacatgctgtacatgcaaatgatcagcctgagaacccagaacatggcagcatttaactgtgcaggaaa

SEQ ID NO: 43 - DNA, 296 bp, synthetic construct
  source 1..296: mol_type = other DNA
gaagcccagctgacagaaagtggaggagacctggtgcacctagaaggccctcttcggctcagctgcgcggcctcctggttcaccttttccatatatgagatccactgggtctgccaagcttctgggaaaggcctggagtgggtggctgtaatctggaggggggagagccatcagtacaatgcagattatgttcgaggtcgtctcacaacgtcaagagacaataccaagtacatgctgtacatgcagatgatttctttgcgcactcagaacatggctgccttcaactgtgcaggcaa

SEQ ID NO: 44 - DNA, 296 bp, Homo sapiens
  source 1..296: mol_type = genomic DNA
caggtgcagctggtgcagtctggccatgaggtgaagcagcctggggcctcagtgaaggtctcctgcaaggcttctggttacagtttcaccacctatggtatgaattgggtgccacaggcccctggacaagggcttgagtggatgggatggttcaacacctacactgggaacccaacatatgcccagggcttcacaggacggtttgtcttctccatggacacctctgccagcacagcatacctgcagatcagcagcctaaaggctgaggacatggccatgtattactgtgcgagata

SEQ ID NO: 45 - DNA, 296 bp, synthetic construct
  source 1..296: mol_type = other DNA
caagttcagctggtgcagagtggtcacgaagtgaagcagccaggagcatctgtcaaagtcagctgcaaagcatcaggctacagcttcaccacttatggcatgaactgggtgcctcaggctccggggcaaggtttggagtggatgggctggttcaacacctacacagggaatcccacctatgcccagggcttcacaggaaggtttgtattttccatggatacttctgcttcgacggcgtatctgcagatctccagtctcaaggctgaggacatggccatgtactactgtgccagatg

SEQ ID NO: 46 - DNA, 786 bp, synthetic construct
  source 1..786: mol_type = other DNA
tgtatgggatccatttcctcagagagttattggatttggactagactatcctgctgcttgacctatgtacctttaagtccttcctctccagcttttcttcattcggattggttattatatacaaagtcccctggtcatgaatatgcaaaatacctaagtctatggtagctaaaaacagggatatcaacaccctgaaaacaacatatgtacaatgtcctcaccacagacactgaacacactgactctaaccatgggatggagctggatctttctctttctcctgtcaggaactgcaggtaaggggctcaccagttccaaatctgaagaaaagaaatggcttgggatgtcactgacatccactctgtctttctcttcacaggtgtcctctctcaggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagaggcacagtgctacaaacacatcctgagtgtgtcagaaaccctggaggtgcagcaagctcccttgggattgacaagacttagagaatagccgcttgcagactt

SEQ ID NO: 47 - DNA, 786 bp, synthetic construct
  source 1..786: mol_type = other DNA
tgtatgggatccatttcctcagagagttattggatttggactagactatcctgctgcttgacctatgtacctttaagtccttcctctccagcttttcttcattcggattggttattatatacaaagtcccctggtcatgaatatgcaaaatacctaagtctatggtagctaaaaacagggatatcaacaccctgaaaacaacatatgtacaatgtcctcaccacagacactgaacacactgactctaaccatgggatggagctggatctttctctttctcctgtcaggaactgcaggtaaggggctcaccagttccaaatctgaagaaaagaaatggcttgggatgtcactgacatccactctgtctttctcttcacaggtgtcctctctcaggtgcagctcgtccagagtggggctgaagtgaagaagcctggagcatctgtgaaagtatcctgcaaagcgagtggctacaccttcaccagctatgacatcaactgggtgcggcaggcaactggacaaggtctggagtggatgggctggatgaaccccaacagcggaaatactggctatgcccagaagtttcaagggcgcgttaccatgacgaggaatacatccatttctacagcctacatggagctgagctcgttgcgatcagaagatacagctgtctattactgtgccagaggcacagtgctacaaacacatcctgagtgtgtcagaaaccctggaggtgcagcaagctcccttgggattgacaagacttagagaatagccgcttgcagactt

SEQ ID NO: 48 - DNA, 787 bp, synthetic construct
  source 1..787: mol_type = other DNA
gtttgggatgcttttcctcagggaggattatgacttggaccctagcatcctgctgcatgacccatgtgccttttcagtgctttctccctagttcttctccagctggactaggtcattaactaagaaatgcactgctcatgaatatgcaaattacttgagcctatggtagtaaatacaggcatgcccacactgtgaaaacaacatatgactcctgtcttctccacagtcccagaacacactcactctaaccatggaatggccttgtatctttctcttcctcctgtcagtaactgaaggtaaggagctcagcagttccaatctgaagaggagatagggcctgaggtgacaatgacatccacaatttctttctcccgacaggtgtccactccgaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatacacagtgttacaaccacatcctgagagtgtcagaaaccgtggaggagcaggaagcttccctgggcctgagatgacagaaagattaatctttagacttgct

SEQ ID NO: 49 - DNA, 787 bp, synthetic construct
  source 1..787: mol_type = other DNA
gtttgggatgcttttcctcagggaggattatgacttggaccctagcatcctgctgcatgacccatgtgccttttcagtgctttctccctagttcttctccagctggactaggtcattaactaagaaatgcactgctcatgaatatgcaaattacttgagcctatggtagtaaatacaggcatgcccacactgtgaaaacaacatatgactcctgtcttctccacagtcccagaacacactcactctaaccatggaatggccttgtatctttctcttcctcctgtcagtaactgaaggtaaggagctcagcagttccaatctgaagaggagatagggcctgaggtgacaatgacatccacaatttctttctcccgacaggtgtccactccgaagtgcagctggtggagtctggaggagggttggttcagccaggaagatcacttcggctcagctgcgctgctagtggcttcacttttgatgactatgccatgcactgggtaaggcaagctcctgggaaaggcctggagtgggtcagcgggatatcctggaacagtggttccattggctatgcagattctgtgaagggtcgcttcaccatctcgagagacaatgccaaaaattcactctacctgcagatgaacagcttacgagcagaagatacagcgctatactactgtgccaaggacacacagtgttacaaccacatcctgagagtgtcagaaaccgtggaggagcaggaagcttccctgggcctgagatgacagaaagattaatctttagacttgct

SEQ ID NO: 50 - DNA, 790 bp, synthetic construct
  source 1..790: mol_type = other DNA
acaaggttcaggaatgaggtatgggattaatttcatcagacaggacaatgacttgtgcctcagcatcctgatttctgacccaggtgtcccttcttctccagcaggagtaggtgcttatataatatgtatcctgctcataaatatgcaaatcctgtgaatctacagtggtaaatatagggttgtctacaccacacaaaaaagcataagatcactgttctctctacagttactgagcacacaggacctcaccatgggatggagctgtatcatcctcttcttggtatcaacagctacaggtaaggggctcacaggtaagcaggcttgagaactggccatacctgtgggtgaaaatgacatccactctctctttctctccacaggtgtccactcccaggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagagacacagtgttgcaaccacatcctgagagtgtcagaaaccctggaggagtagcaaactgccctgagactgaggagactcagagaaggtttgcttgtagactt

SEQ ID NO: 51 - DNA, 790 bp, synthetic construct
  source 1..790: mol_type = other DNA
acaaggttcaggaatgaggtatgggattaatttcatcagacaggacaatgacttgtgcctcagcatcctgatttctgacccaggtgtcccttcttctccagcaggagtaggtgcttatataatatgtatcctgctcataaatatgcaaatcctgtgaatctacagtggtaaatatagggttgtctacaccacacaaaaaagcataagatcactgttctctctacagttactgagcacacaggacctcaccatgggatggagctgtatcatcctcttcttggtatcaacagctacaggtaaggggctcacaggtaagcaggcttgagaactggccatacctgtgggtgaaaatgacatccactctctctttctctccacaggtgtccactcccaagtccagctgcaggagagtggacctgggctggtgaagcctagccaaaccttgagtcttacctgcactgtgtcgggtggcagcattagttcaggaggttattactggtcctggatcagacagcacccaggaaaaggcctagagtggattggctacatctattattctgggtcaacgtactacaaccccagcctgaagtcccgggtcacaatatctgtagacaccagcaagaatcagttctccctcaaactctcatctgttactgcagctgatacagccgtgtattactgtgccagggacacagtgttgcaaccacatcctgagagtgtcagaaaccctggaggagtagcaaactgccctgagactgaggagactcagagaaggtttgcttgtagactt

SEQ ID NO: 52 - DNA, 788 bp, synthetic construct
  source 1..788: mol_type = other DNA
acaaggttcagggatgaggtatgggatgcatttccttagacaagacaaggacttgggcttcagcaacctgattcttgacccagatgtcccttcttctccagcaggagtaggtgcttatctaatatgtatcctgctcataaatatgcaaatcctgtgaatctacagtggtaaatttagggttgtctacacgacacaaaaaagcataagatcactgttctctctacagttactaagcacacaggatctcaccatgggatggagctgtatcatcctctttttggtagcagcagctacaggtaaggggctcacagtagcaggcttgaggtctggccatatacatgggtgacaatgacatccactttgcctttctctccacaggtgtccactcccaggtgcagctgcaggagtcgggcccaggactggtgaagccttcacagaccctgtccctcacctgcactgtctctggtggctccatcagcagtggtgattactactggagttggatccgccagcccccagggaagggcctggagtggattgggtacatctattacagtgggagcacctactacaacccgtccctcaagagtcgagttaccatatcagtagacacgtccaagaaccagttctccctgaagctgagctctgtgactgccgcagacacggccgtgtattactgtgccagagacacagtgttgcaaccacatcctgagagtgtcagaaaccctggatgagtagcaaactgccctgagactgaggagactcagagaaggtttgcttgtagactt

SEQ ID NO: 53 - DNA, 788 bp, synthetic construct
  source 1..788: mol_type = other DNA
acaaggttcagggatgaggtatgggatgcatttccttagacaagacaaggacttgggcttcagcaacctgattcttgacccagatgtcccttcttctccagcaggagtaggtgcttatctaatatgtatcctgctcataaatatgcaaatcctgtgaatctacagtggtaaatttagggttgtctacacgacacaaaaaagcataagatcactgttctctctacagttactaagcacacaggatctcaccatgggatggagctgtatcatcctctttttggtagcagcagctacaggtaaggggctcacagtagcaggcttgaggtctggccatatacatgggtgacaatgacatccactttgcctttctctccacaggtgtccactcccaagtgcagctgcaggagagtgggcctggcctggtgaagccatcccaaaccctttcgctcacctgcacagtaagtggtggctccatatcttcaggagactattactggagctggatcaggcagcccccagggaaaggactggagtggattggctacatctactattctggtagcacgtattataatccttccttgaaaagcagagttaccatttctgtggatactagcaagaaccagttctccctcaagctaagttcagtcacagccgcagacactgctgtctactactgtgcccgggacacagtgttgcaaccacatcctgagagtgtcagaaaccctggatgagtagcaaactgccctgagactgaggagactcagagaaggtttgcttgtagactt

SEQ ID NO: 54 - DNA, 786 bp, synthetic construct
  source 1..786: mol_type = other DNA
tgaatgggatctatttcttcagagagttattggatttgtactagaccatcctactgcttgacctatgtacctttaattccttcctctccagctgttctccatttggattggttagtatatacaaagtcccctggtcatgactatgcaaattacctaagtctatggtagttaaaaacagggatatcaacaccctgaaaacaacatatgtccaatgtcctctccacaggcactgaacacactgactctaaccatgggatggagctggatctttctcctcttcctgtcaggaactgcaggtaaggggctcaccatttccaaatctgaagaaaagaaatggcttgggatgtcactgaaatccactctgtctttctcttcacaggtgtcctctctcaggtgcagctgcaggagtcgggcccaggactggtgaagccttcggagaccctgtccctcacctgcactgtctctggttactccatcagcagtggttactactggggctggatccggcagcccccagggaaggggctggagtggattgggagtatctatcatagtgggagcacctactacaacccgtccctcaagagtcgagtcaccatatcagtagacacgtccaagaaccagttctccctgaagctgagctctgtgaccgccgcagacacggccgtgtattactgtgcgagagacacagtgctacaaacacatcctgagtgtgtcagaaaccttggaggtgcagcaagctcccttgggactgacaagacttagagaagagtcgcttgcagactt

SEQ ID NO: 55 - DNA, 786 bp, synthetic construct
  source 1..786: mol_type = other DNA
tgaatgggatctatttcttcagagagttattggatttgtactagaccatcctactgcttgacctatgtacctttaattccttcctctccagctgttctccatttggattggttagtatatacaaagtcccctggtcatgactatgcaaattacctaagtctatggtagttaaaaacagggatatcaacaccctgaaaacaacatatgtccaatgtcctctccacaggcactgaacacactgactctaaccatgggatggagctggatctttctcctcttcctgtcaggaactgcaggtaaggggctcaccatttccaaatctgaagaaaagaaatggcttgggatgtcactgaaatccactctgtctttctcttcacaggtgtcctctctcaagtacagctgcaggagagtggtcctgggctggtgaagccatcagaaactctttcactcacctgcactgtttcaggctacagcatatccagtggctattactggggctggatcaggcagcctccagggaaaggactagagtggattggatccatctaccacagcggtagtacgtattataatcccagcctgaagtccagagtcaccatttctgtggacaccagcaagaaccagttctccttgaaactctcttctgtcacagccgcagatacagctgtgtactactgtgcccgggacacagtgctacaaacacatcctgagtgtgtcagaaaccttggaggtgcagcaagctcccttgggactgacaagacttagagaagagtcgcttgcagactt

SEQ ID NO: 56 - DNA, 787 bp, synthetic construct
  source 1..787: mol_type = other DNA
gtttgggatgcttttcctcagggaggattatgacttagaccctagcatcctgctgcctgacccatgtgccttttcagtgctttctccctagttcttctccagctggactaggtcattaagtaagaaatgcactgctcatgaatatgcaaattacttgagcctatggtagtaaatacaggcatgcccacactgtgaaaacagcatatgacccctgtcttctccacagtccctgaacacactgactctaaccatggaatggcctttgatctttctcttcctcctgtcaggaactgcaggtaaggggctcagcagttccaatctgaagaggagacagggcctgaggtgacaatgacatccacaatgactttctcccaacaggtgtccaatccgaagtgcagctggtggagtctgggggagtcgtggtacagcctggggggtccctgagactctcctgtgcagcctctggattcacctttgatgattatgccatgcactgggtccgtcaagctccggggaagggtctggagtgggtctctcttattagttgggatggtggtagcacatactatgcagactctgtgaagggtcgattcaccatctccagagacaacagcaaaaactccctgtatctgcaaatgaacagtctgagagctgaggacaccgccttgtattactgtgcaaaagatacacagtgttgcaaccacatcctgagtgtgtcagaaatcctggaggagcaggaagcttccctgggcctgagatgacagaaagattaatctttagacttgct

SEQ ID NO: 57 - DNA, 787 bp, synthetic construct
  source 1..787: mol_type = other DNA
gtttgggatgcttttcctcagggaggattatgacttagaccctagcatcctgctgcctgacccatgtgccttttcagtgctttctccctagttcttctccagctggactaggtcattaagtaagaaatgcactgctcatgaatatgcaaattacttgagcctatggtagtaaatacaggcatgcccacactgtgaaaacagcatatgacccctgtcttctccacagtccctgaacacactgactctaaccatggaatggcctttgatctttctcttcctcctgtcaggaactgcaggtaaggggctcagcagttccaatctgaagaggagacagggcctgaggtgacaatgacatccacaatgactttctcccaacaggtgtccaatccgaagtacaattggtggagagtggcggtgttgtcgtgcagcctggaggtagtctcaggctgtcctgtgcagcgtcaggcttcacttttgatgactatgccatgcactgggtgcggcaggccccagggaaggggctggagtgggtctcattaatttcctgggatggaggcagcacctactatgcagattctgtgaaaggacgcttcacgatctctagagacaattccaagaacagcctatatctgcagatgaacagccttcgagctgaagacacagctctctactactgcgccaaggacacacagtgttgcaaccacatcctgagtgtgtcagaaatcctggaggagcaggaagcttccctgggcctgagatgacagaaagattaatctttagacttgct

SEQ ID NO: 58 - DNA, 786 bp, synthetic construct
  source 1..786: mol_type = other DNA
tgtatgggatccatttcctcagagagttattggatttggactagaccatcctgctgcttgacctatgtacctttaagtccttcctctccatctattctccatttggattggttattatatacaaagtcccctggtcatgaatatgcaaattacctaagtctatcgtagttaaaaacagggatatcaacatcctgaaaacaacatatgtccaatgtcctctcctcagacactgaacacactgactctaaccatgggatggagctggatctttctctttctcctgtcagaaactgcaggtaaggggctcaccatttccaaatctgaagaaaagaaatggctagggatgtcactgacatccactctgtctttctcttcacaggtgtcctctctgaggtgcagctggtggagtctgggggaggcttggtacagcctgggggatccctgagactctcctgtgcagcctctggattcaccttcagtaacagtgacatgaactgggtccatcaggctccaggaaaggggctggagtgggtatcgggtgttagttggaatggcagtaggacgcactatgcagactctgtgaagggccaattcatcatctccagagacaattccaggaacaccctgtatctgcaaacgaatagcctgagggccgaggacacggctgtgtattactgtgtgagaaacacagtgctacaaacacatcctcagtgtgtcagaaatcctggaggtgcagcaagctcccttgggactgacaagacttagagaatagttgcttgcagacgt

SEQ ID NO: 59 - DNA, 786 bp, synthetic construct
  source 1..786: mol_type = other DNA
tgtatgggatccatttcctcagagagttattggatttggactagaccatcctgctgcttgacctatgtacctttaagtccttcctctccatctattctccatttggattggttattatatacaaagtcccctggtcatgaatatgcaaattacctaagtctatcgtagttaaaaacagggatatcaacatcctgaaaacaacatatgtccaatgtcctctcctcagacactgaacacactgactctaaccatgggatggagctggatctttctctttctcctgtcagaaactgcaggtaaggggctcaccatttccaaatctgaagaaaagaaatggctagggatgtcactgacatccactctgtctttctcttcacaggtgtcctctctgaagtgcagctggtggagagtgggggtggtctcgttcagcctggaggaagtctacggctttcctgtgccgcgtcaggctttaccttcagcaattcagacatgaactgggtgcatcaggccccagggaagggcctggagtgggtatctggagtctcttggaatggcagccgtacacactatgctgattctgttaaggggcagttcatcatttcccgcgataattccaggaacacactctacctgcaaaccaacagcttgcgagcagaagacactgctgtgtattactgcgtcagaaacacagtgctacaaacacatcctcagtgtgtcagaaatcctggaggtgcagcaagctcccttgggactgacaagacttagagaatagttgcttgcagacgt

SEQ ID NO: 60 - DNA, 785 bp, synthetic construct
  source 1..785: mol_type = other DNA
aaggttcagggatgagatatgagggtgaatttccacggacaagattaggacttgggcttcagcatcctgattcctgacccaggtgtcccttcttctccagcaggagtaggggctcatctaatatgtatcctgctcatgaatatgcaaatcctctgaatctacatggtaaatatagggttgtctataccacacacagaaaaacatgagatcacagttctctctacagttactgagcacacaggacctcacaatgggatggagctatatcatcctctttttggtagcaacagctacaggtaaggggctcacagtagcaggcttgaggtctggccatatacatgggtgacaatgacatccactttgcccttctctccacaggtgtccactccgaggtgcagctggtgaagtctggaggaggcttggtacagcctggggggtccctgagactctcctgtgcagcctctggattcaccttcagtagctctgctatgcactgggtccgccaggctccaagaaagggtttggagtgggtctcagttattagtacaagtggtgataccgtactctacacagactctgtgaagggccgattcaccatctccagagacaatgcccagaattcactgtctctgcaaatgaacagcctgagagccgaggacatggctgtgtattactgtgtgaaagacacagtgttgcaaccacatcctgagagtgtctgaaaacctggaggggtaacaaactgccctgggacttaggagactcagagaaagtttgcttgtagactt

SEQ ID NO: 61 - DNA, 785 bp, synthetic construct
  source 1..785: mol_type = other DNA
aaggttcagggatgagatatgagggtgaatttccacggacaagattaggacttgggcttcagcatcctgattcctgacccaggtgtcccttcttctccagcaggagtaggggctcatctaatatgtatcctgctcatgaatatgcaaatcctctgaatctacatggtaaatatagggttgtctataccacacacagaaaaacatgagatcacagttctctctacagttactgagcacacaggacctcacaatgggatggagctatatcatcctctttttggtagcaacagctacaggtaaggggctcacagtagcaggcttgaggtctggccatatacatgggtgacaatgacatccactttgcccttctctccacaggtgtccactccgaagtacagctggtgaaaagtggtggtgggctcgttcaacctggaggctcccttcgcctcagctgtgctgcgagtggcttcaccttcagctcctcagccatgcactgggttcggcaggcaccaaggaagggcctggagtgggtgtctgtcatttccacttctggggacacagtgctgtatacagatagtgtcaaaggacgatttaccatcagcagagataatgcccagaactcattgtctctgcagatgaacagcctaagagcagaggacatggctgtgtactactgcgtgaaggacacagtgttgcaaccacatcctgagagtgtctgaaaacctggaggggtaacaaactgccctgggacttaggagactcagagaaagtttgcttgtagactt

SEQ ID NO: 62 - DNA, 786 bp, synthetic construct
  source 1..786: mol_type = other DNA
agcttaccaaaaatgtaggaaaagtattttttcagagaagattaggacattgaccagagcatcctgatacctgacgcatgtgcctactgtccagtagttctcctgctcaattaggttcttatctaagaagttaatgctcatgaatatgcaaattacttgagtctatggcagtaaatacaggggtgaccacatgcagaaaacagcatatgatcagtgtcctctccaaagtccttgaacatagactctaaccatggaatggacctgggtctttctcttcctcctgtcagtaactgcaggtaaggggctcaccattttcaaatctgaagaagagacagagcttgaggtgacaatgacaaccactctgcctttctctccacaggtgtccactccgaggtgcagctggtggagtctgggggaggcttggtacagcctggggggtccctgagactctcctgtgcagcctctggattcaccttcagtaacagtgacatgaactgggcccgcaaggctccaggaaaggggctggagtgggtatcgggtgttagttggaatggcagtaggacgcactatgtggactccgtgaagcgccgattcatcatctccagagacaattccaggaactccctgtatctgcaaaagaacagacggagagccgaggacatggctgtgtattactgtgtgagaaacacagtgttgtaaccacatcctgagtgtgtcagaaactctggaggagcagcaagctgccctgggtctgaaatatcagaaaaggctaacatttagacttcc

SEQ ID NO: 63 - DNA, 786 bp, synthetic construct
  source 1..786: mol_type = other DNA
agcttaccaaaaatgtaggaaaagtattttttcagagaagattaggacattgaccagagcatcctgatacctgacgcatgtgcctactgtccagtagttctcctgctcaattaggttcttatctaagaagttaatgctcatgaatatgcaaattacttgagtctatggcagtaaatacaggggtgaccacatgcagaaaacagcatatgatcagtgtcctctccaaagtccttgaacatagactctaaccatggaatggacctgggtctttctcttcctcctgtcagtaactgcaggtaaggggctcaccattttcaaatctgaagaagagacagagcttgaggtgacaatgacaaccactctgcctttctctccacaggtgtccactccgaagtgcagctggtggagagtggtggtggcttggttcaaccaggaggcagcctaaggcttagctgtgctgccagtggcttcacattttccaattcagatatgaactgggcaaggaaggcccctgggaaaggactggagtgggtgtcaggggtctcctggaatggatcccgtacccactatgtggattctgtaaagcgccgcttcatcatttctcgggacaacagcagaaattctctctatctgcagaagaacagaagacgagcggaagacatggctgtctactactgcgttcggaacacagtgttgtaaccacatcctgagtgtgtcagaaactctggaggagcagcaagctgccctgggtctgaaatatcagaaaaggctaacatttagacttcc

SEQ ID NO: 64 - DNA, 795 bp, synthetic construct
  source 1..795: mol_type = other DNA
aaagtgtttgggatacatttcctcagagaggattaggacttggacctgagctgcttgacccaggtgccttttcagtccttcctctccagtttttctctagatggactaggtccttaactaaaaaatgcactgcttgtgaatatgcaaatcacccacgtctatggcagtaaatacatggatgtccacagcctgaaaacaacctatgatcagtgtcctctctacacagtccctgacgacactgactctaaccatgggatggagctggatctttctcttcctcctgtcaggaactgcaggtaagggaattaattccaaatctcaccaattccaaatctgaagtggagacagggtctgaggtgataatgacatccacagtgcctttctttccataggtgtccattgcgaggtgcagctggtggagtctgggggaggcttggtacagcctagggggtccctgagactctcctgtgcagcctctggattcaccgtcagtagcaatgagatgagctggatccgccaggctccagggaaggggctggagtgggtctcatccattagtggtggtagcacatactacgcagactccaggaagggcagattcaccatctccagagacaattccaagaacacgctgtatcttcaaatgaacaacctgagagctgagggcacggccgtgtattactgtgccagatatacacagtgttacaaccacatcctgagtgtgtcagaaaccctggaggagcaggaagctgcactgggactgagatgacagaaagattaatccttagactttct

SEQ ID NO: 65 - DNA, 795 bp, synthetic construct
  source 1..795: mol_type = other DNA
aaagtgtttgggatacatttcctcagagaggattaggacttggacctgagctgcttgacccaggtgccttttcagtccttcctctccagtttttctctagatggactaggtccttaactaaaaaatgcactgcttgtgaatatgcaaatcacccacgtctatggcagtaaatacatggatgtccacagcctgaaaacaacctatgatcagtgtcctctctacacagtccctgacgacactgactctaaccatgggatggagctggatctttctcttcctcctgtcaggaactgcaggtaagggaattaattccaaatctcaccaattccaaatctgaagtggagacagggtctgaggtgataatgacatccacagtgcctttctttccataggtgtccattgcgaagtgcagctggtggagagtggaggtggtctagtccagcctcgagggagtttgaggcttagctgtgctgcctctggcttcactgtttcttccaatgagatgtcttggatccggcaagccccagggaagggcctggagtgggtgtccagcatttcaggaggaagcacctactatgcagattcaaggaaaggccgttttaccatatccagagacaacagcaagaatacactctacctgcagatgaacaacctcagagcagaagggacagctgtatattactgcgcccgctatacacagtgttacaaccacatcctgagtgtgtcagaaaccctggaggagcaggaagctgcactgggactgagatgacagaaagattaatccttagactttct

SEQ ID NO: 66 - DNA, 804 bp, synthetic construct
  source 1..804: mol_type = other DNA
gaaactaaaatgtgtttcaaatgctgtaaataaaatctggttttttgatgccttatatatctgttatcatcagtggcatcagcctaggtccaactccggagcatggtatagcaggaagacatgcaaataagtcttctctgtgcccatgaaaaacacctcggccctgaccctgcagctctgacagaggaggccggtcctggattcgagttcctcacattcagtgatgagcactgaacacggacccctcaccatgaacttcgggctcagcttgattttccttgtccttgttttaaaaggtaatttattgagaagagatgacatctgttgtatgcacatgagacagagaaaaattgttgtttgttttgttagtgacagttttccaaccagtattctctgtttgcaggtgtccagtgtgaggtgcagctggtggagtctcggggagtcttggtacagcctggggggtccctgagactctcctgtgcagcctctggattcaccgtcagtagcaatgagatgagctgggtccgccaggctccagggaagggtctggagtgggtctcatccattagtggtggtagcacatactacgcagactccaggaagggcagattcaccatctccagagacaattccaagaacacgctgcatcttcaaatgaacagcctgagagctgaggacacggctgtgtattactgtaagaaagacacaatgaggaaatgttactgtgagctcaaactaaaacctcctgcagagcacccaagaccagcagggggcggagagagcatagtaatttggaaatttgca

SEQ ID NO: 67 - DNA, 804 bp, synthetic construct
  source 1..804: mol_type = other DNA
gaaactaaaatgtgtttcaaatgctgtaaataaaatctggttttttgatgccttatatatctgttatcatcagtggcatcagcctaggtccaactccggagcatggtatagcaggaagacatgcaaataagtcttctctgtgcccatgaaaaacacctcggccctgaccctgcagctctgacagaggaggccggtcctggattcgagttcctcacattcagtgatgagcactgaacacggacccctcaccatgaacttcgggctcagcttgattttccttgtccttgttttaaaaggtaatttattgagaagagatgacatctgttgtatgcacatgagacagagaaaaattgttgtttgttttgttagtgacagttttccaaccagtattctctgtttgcaggtgtccagtgtgaagtgcagctagtggagtcaagaggtgttttggtccagcctggaggaagtctcaggctctcctgtgccgcgtctggctttactgtatcaagtaatgagatgtcctgggtgaggcaagccccagggaaaggcctggagtgggtcagcagtatttctggtggcagcacctattatgctgacagccggaaagggcgcttcaccatctccagagacaacagcaagaacacactgcacctgcagatgaattctcttcgagcagaagatacagctgtgtactactgcaagaaggacacaatgaggaaatgttactgtgagctcaaactaaaacctcctgcagagcacccaagaccagcagggggcggagagagcatagtaatttggaaatttgca

SEQ ID NO: 68 - DNA, 801 bp, synthetic construct
  source 1..801: mol_type = other DNA
gaaactacaatatgtttcaaatgctgtaactgaaatctggttttttgatgcttatatctggtatcatcagtgacttcagatttagtccaaccccagagcatggtatagcaggaagacatgcaaataagtcttctctgtgcccatgaaaaacacctcggccctgaccctgcagctctgacagaggaggccggtcctggattcgattcccagttcctcacattcagtcagcactgaacacggacccctcaccatgaacttcgggctcagcttgattttccttgcccttattttaaaaggtaatttattgagaagagatgacatctcttgtatgcacgtgagagagaaaaattgttttgttagtgacagttttccaaccagtattctctgtttgcaggtgtccagtgtcaggtccagctggtgcagtcttgggctgaggtgaggaagtctggggcctcagtgaaagtctcctgtagtttttctgggtttaccatcaccagctacggtatacattgggtgcaacagtcccctggacaagggcttgagtggatgggatggatcaaccctggcaatggtagcccaagctatgccaagaagtttcagggcagattcaccatgaccagggacatgtccacaaccacagcctacacagacctgagcagcctgacatctgaggacatggctgtgtattactgtgcaagacacacaatgaggaaatgttactgtgagctcaaactaaaacctcctgcagagcacccaggaccagcagggggcgcagagagcacatggagttctgattcacag

SEQ ID NO: 69 - DNA, 801 bp, synthetic construct
  source 1..801: mol_type = other DNA
gaaactacaatatgtttcaaatgctgtaactgaaatctggttttttgatgcttatatctggtatcatcagtgacttcagatttagtccaaccccagagcatggtatagcaggaagacatgcaaataagtcttctctgtgcccatgaaaaacacctcggccctgaccctgcagctctgacagaggaggccggtcctggattcgattcccagttcctcacattcagtcagcactgaacacggacccctcaccatgaacttcgggctcagcttgattttccttgcccttattttaaaaggtaatttattgagaagagatgacatctcttgtatgcacgtgagagagaaaaattgttttgttagtgacagttttccaaccagtattctctgtttgcaggtgtccagtgtcaggtgcagctggttcagtcctgggcagaggtgaggaaaagtggtgcttctgtaaaagtctcctgcagcttttctggcttcaccataaccagctatggaattcactgggtccagcagagccccgggcaagggctggagtggatgggctggatcaaccctggaaatggctcgccatcctatgccaagaagttccagggtcgctttaccatgacaagagatatgtctactacgacagcctacacagacctctcaagtcttacttcagaagacatggctgtgtactactgtgcgcggcacacaatgaggaaatgttactgtgagctcaaactaaaacctcctgcagagcacccaggaccagcagggggcgcagagagcacatggagttctgattcacag

SEQ ID NO: 70 - DNA, 783 bp, synthetic construct
  source 1..783: mol_type = other DNA
ctcagaggacagcacaatcagcatgaactaataaacatcagctaagataaggcacaggctcagctatatagggtcagggatctttgtaaatctgattgtgtatccagtctagttcaatgtgacttaggaaggccagtcatatgcaaatctagagaagactttagagtagaaatctgaggctcacctcacataccagcaagggagtgaccagttagtcttaaggcaccactgagcccaagtcttagacatcatgggttggctgtggaacttgctattcctgatggcagctgcccaaagtaagacatcagaaaaaaagagttccaaggggaattgaagcagttccatgaatactcaccttcctgtgttcttttcacaggtgcccaagcagaggcccagcttacagagtctgggggagacttggtacacttagaggggcccctgaggctctcctgtgcagcctcttggttcaccttcagtatctatgagattcactgggtttgccaggcctcagggaaggggctggaatgggttgcagttatatggcgtggtgaaagtcatcaatacaatgcagactatgttaggggcagactcaccacttccagagacaacaccaagtacatgctgtacatgcaaatgatcagcctgagaacccagaacatggcagcatttaactgtgcaggaaacacagtgtgaaaaccacatcctgagggtgtcaaaaaccatgaggagaaggtggttcagctgtgtccagaagcaaccagaggaaacattctctccttggtg

SEQ ID NO: 71 - DNA, 783 bp, synthetic construct
  source 1..783: mol_type = other DNA
ctcagaggacagcacaatcagcatgaactaataaacatcagctaagataaggcacaggctcagctatatagggtcagggatctttgtaaatctgattgtgtatccagtctagttcaatgtgacttaggaaggccagtcatatgcaaatctagagaagactttagagtagaaatctgaggctcacctcacataccagcaagggagtgaccagttagtcttaaggcaccactgagcccaagtcttagacatcatgggttggctgtggaacttgctattcctgatggcagctgcccaaagtaagacatcagaaaaaaagagttccaaggggaattgaagcagttccatgaatactcaccttcctgtgttcttttcacaggtgcccaagcagaagcccagctgacagaaagtggaggagacctggtgcacctagaaggccctcttcggctcagctgcgcggcctcctggttcaccttttccatatatgagatccactgggtctgccaagcttctgggaaaggcctggagtgggtggctgtaatctggaggggggagagccatcagtacaatgcagattatgttcgaggtcgtctcacaacgtcaagagacaataccaagtacatgctgtacatgcagatgatttctttgcgcactcagaacatggctgccttcaactgtgcaggcaacacagtgtgaaaaccacatcctgagggtgtcaaaaaccatgaggagaaggtggttcagctgtgtccagaagcaaccagaggaaacattctctccttggtg

SEQ ID NO: 72 - DNA, 805 bp, synthetic construct
  source 1..805: mol_type = other DNA
ttagtaaatgatatactgatgttgtaatgccacttttcctttactatttctcagatatctatctgcattattatttgagtcttctaaggctcccaccctgagctcatgatatagcacacagaaatgcaaatcatatttccctttgattatgaataccagccctgagactctaaagctctgacagaggcacctaactgtggactcacaagtctttcccttcagtgaccaacacggacacagaacattcaccatggacttgagactgagctgtgcttttattattgttcttttaaaaggtaattcatagataataagagatattgagtgtgtgagtggacgggagtgagggaacactgaatattttgacggcttactgagagggttctctgtgttttcaggggtccagagtcaggtgcagctggtgcagtctggccatgaggtgaagcagcctggggcctcagtgaaggtctcctgcaaggcttctggttacagtttcaccacctatggtatgaattgggtgccacaggcccctggacaagggcttgagtggatgggatggttcaacacctacactgggaacccaacatatgcccagggcttcacaggacggtttgtcttctccatggacacctctgccagcacagcatacctgcagatcagcagcctaaaggctgaggacatggccatgtattactgtgcgagatacacagtgagaagtcttcattgtgagtctagacacaaacttacccaaaggagctctcagtaccagcagggggagcacagtgacaatcgaatccataaatgg

SEQ ID NO: 73 - DNA, 805 bp, synthetic construct
  source 1..805: mol_type = other DNA
ttagtaaatgatatactgatgttgtaatgccacttttcctttactatttctcagatatctatctgcattattatttgagtcttctaaggctcccaccctgagctcatgatatagcacacagaaatgcaaatcatatttccctttgattatgaataccagccctgagactctaaagctctgacagaggcacctaactgtggactcacaagtctttcccttcagtgaccaacacggacacagaacattcaccatggacttgagactgagctgtgcttttattattgttcttttaaaaggtaattcatagataataagagatattgagtgtgtgagtggacgggagtgagggaacactgaatattttgacggcttactgagagggttctctgtgttttcaggggtccagagtcaagttcagctggtgcagagtggtcacgaagtgaagcagccaggagcatctgtcaaagtcagctgcaaagcatcaggctacagcttcaccacttatggcatgaactgggtgcctcaggctccggggcaaggtttggagtggatgggctggttcaacacctacacagggaatcccacctatgcccagggcttcacaggaaggtttgtattttccatggatacttctgcttcgacggcgtatctgcagatctccagtctcaaggctgaggacatggccatgtactactgtgccagatgcacagtgagaagtcttcattgtgagtctagacacaaacttacccaaaggagctctcagtaccagcagggggagcacagtgacaatcgaatccataaatgg

SEQ ID NO: 74 - protein, 98 aa, synthetic construct
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAR

SEQ ID NO: 75 - protein, 99 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKD

SEQ ID NO: 76 - protein, 99 aa, synthetic construct
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCAR

SEQ ID NO: 77 - protein, 99 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAR

SEQ ID NO: 78 - protein, 98 aa, synthetic construct
QVQLQESGPGLVKPSETLSLTCTVSGYSISSGYYWGWIRQPPGKGLEWIGSIYHSGSTYYNPSLKSRVTISVDTSKNQFSLKLSSVTAADTAVYYCAR

SEQ ID NO: 79 - protein, 99 aa, synthetic construct
EVQLVESGGVVVQPGGSLRLSCAASGFTFDDYAMHWVRQAPGKGLEWVSLISWDGGSTYYADSVKGRFTISRDNSKNSLYLQMNSLRAEDTALYYCAKD

SEQ ID NO: 80 - protein, 98 aa, synthetic construct
EVQLVESGGGLVQPGGSLRLSCAASGFTFSNSDMNWVHQAPGKGLEWVSGVSWNGSRTHYADSVKGQFIISRDNSRNTLYLQTNSLRAEDTAVYYCVR

SEQ ID NO: 81 - protein, 98 aa, synthetic construct
EVQLVKSGGGLVQPGGSLRLSCAASGFTFSSSAMHWVRQAPRKGLEWVSVISTSGDTVLYTDSVKGRFTISRDNAQNSLSLQMNSLRAEDMAVYYCVK

SEQ ID NO: 82 - protein, 98 aa, synthetic construct
EVQLVESGGGLVQPGGSLRLSCAASGFTFSNSDMNWARKAPGKGLEWVSGVSWNGSRTHYVDSVKRRFIISRDNSRNSLYLQKNRRRAEDMAVYYCVR

SEQ ID NO: 83 - protein, 97 aa, synthetic construct
EVQLVESGGGLVQPRGSLRLSCAASGFTVSSNEMSWIRQAPGKGLEWVSSISGGSTYYADSRKGRFTISRDNSKNTLYLQMNNLRAEGTAVYYCARY

SEQ ID NO: 84 - protein, 96 aa, synthetic construct
EVQLVESRGVLVQPGGSLRLSCAASGFTVSSNEMSWVRQAPGKGLEWVSSISGGSTYYADSRKGRFTISRDNSKNTLHLQMNSLRAEDTAVYYCKK

SEQ ID NO: 85 - protein, 98 aa, synthetic construct
QVQLVQSWAEVRKSGASVKVSCSFSGFTITSYGIHWVQQSPGQGLEWMGWINPGNGSPSYAKKFQGRFTMTRDMSTTTAYTDLSSLTSEDMAVYYCAR

SEQ ID NO: 86 - protein, 98 aa, synthetic construct
EAQLTESGGDLVHLEGPLRLSCAASWFTFSIYEIHWVCQASGKGLEWVAVIWRGESHQYNADYVRGRLTTSRDNTKYMLYMQMISLRTQNMAAFNCAG

SEQ ID NO: 87 - protein, 98 aa, synthetic construct
QVQLVQSGHEVKQPGASVKVSCKASGYSFTTYGMNWVPQAPGQGLEWMGWFNTYTGNPTYAQGFTGRFVFSMDTSASTAYLQISSLKAEDMAMYYCAR

SEQ ID NO: 88 - DNA, 787 bp, synthetic construct
  source 1..787: mol_type = other DNA
gggccctgcctctgagctcctctttgcatccaatctgctgaagaacatggctctagggaaacccagttgtagacctgagggccccggctcttcaatgagccatctccgtcccggggccttatatcagcaagtgacgcacacaggcaaatgccagggtgtggtttcctgtttaaatgtagcctcccccgctgcagaactgcagagcctgctgaattctggctgaccagggcagtcaccagagctccagacaatgtctgtctccttcctcatcttcctgcccgtgctgggcctcccatggggtcagtgtcagggagatgccgtattcacagcagcattcacagactgaggggtgtttcactttgctgtttccttttgtctccaggtgtcctgtcacaggtgcagctggtggagtcaggtggaggattagttcaggctggaggttctcttcgactatcctgcgcggccagtgggcgcaccttcagctcctatgccatgggctggtttcgtcaagcacctgggaaggagagggaatttgtggcagccatttcctggtcaggtggcagcacatactatgcagactctgtaaaagggcggtttaccataagtagagacaatgccaagaacactgtgtacctgcagatgaacagcttgaaaccagaagatacagctgtctattactgtgctgctcacagtgaggggaagtcagtgtgagcccagacacaaacctccctgcagggatgctcaggaccccagaaggcacccagcactaccagcgcagggcccagac

SEQ ID NO: 89 - DNA, 783 bp, synthetic construct
  source 1..783: mol_type = other DNA
ggggacacacatcattaaacaaggattgggacagggacttcagcgtcccactgttgcatggcccataaattatgtgtgttctctttctcatcttggatcaagtctagagctatgaaatagtatccctcatgaatatgcaaataacctgagatttactgaagtaaatacagatctgtcctgtgccctgagagcatcacccagcaaccacatctgtcctctagagaatcccctgagagctccgttcctcaccatggactggacctggaggatcctcttcttggtggcagcagccacaggtaagaggctccctagtcccagtgatgagaaagagattgagtccagtccagggagatctcatccacttctgtgttctctccacaggagcccactcccaggtgcagctggtggagagcgggggtgggttagtacaaccaggtggctccctacgacttagttgcgctgcaagtggcagcatcttctccataaacgccatgggctggtaccggcaagcccctgggaagcagcgtgaactggtggcagccatcacttctggaggctctacgaactatgcagactccgttaaaggacgcttcaccatttcaagagataatgccaagaacacagtgtatctgcagatgaattcattgaaacccgaggatactgcagtctactactgtaatgctcacagtgtgaaaacccacatcctgagggtgtcagaaaccccagggaggaggcagctgtgctggggctgagaaatgaaagggattactatttttaatgttg

SEQ ID NO: 90 - DNA, 783 bp, synthetic construct
  source 1..783: mol_type = other DNA
gggcatggctagttgaggccccaggaagagaactgagttctcaaagggcaaagcaagcatcctcatcccagggcgagcctaaaagactggggcctccctcatcccttttcacctctttatacaaaggcaccacctacatgcaaatcctcacttaggcacccacaggaaaccaccacacatttccttaaattcagggtccagctcacatgggaaatactttctgagagtcatggacctcctgcacaagaacatgaaacacctgtggttcttcctcctcctggtggcagctcccagatgtgagtgtctcaaggctgcagacatgggggtatgggaggtgcctctgatcccagggctcactgtgggtctctctgttcacaggggtcctgtctcaggtgcagctggtggagtctggtgggggcctcgtccaaccaggaggctcactccgactttcctgtgctgcttcaggatcgatctttagtataaatgccatgggctggtacaggcaggcccctgggaagcagagagagctggttgctgccatcaacactggaggtggcagcacctattatgcagattctgtgaaagggcgttttaccatttcccgggacaatgcaaagaacacactgtatctacaaatgaacagcttaaaaagtgaaggaacagctgtctactactgcgcagcccacagtgaggggaggtgagtgtgagcccagacacaaacctccctgcagggaggcggaggggaccggcgcaggtgctgctcagagccagcagggggcgcgc

SEQ ID NO: 91 - DNA, 784 bp, synthetic construct
  source 1..784: mol_type = other DNA
tgacttctgcaaagacttctactcagaatctacttgcccagccttagattaatgccatctgaattacactgatcatgttactatcactgctcctcaccacagatgcaacaccctcctgagtcctgaaacctgactccatcccatagagtagggcacagatgaggggaatgcaaatctccaccagctccaccctcctctgggttgaaaaagccgagcacaggtcccagctcagtgactcctgtgccccaccatggacacactttgctccacgctcctgctgctgaccatcccttcatgtgagtgctgtggtcagggactccttcacgggtgaaacatcagttttcttgtttgtgggcttcatcttcttatgctttctccacaggggtcttgtcccaagtccagctggtggagtctggtggtggccttgtacaggcaggagggtcacttcggcacagctgtgctgcgtctggactcacctttggatcctatgccatgggctggtacaggcaggcccccgggaaagaacgagagctggttgctgccatcagcagtgggggcagtacgtattatgcagactctgtcaaaggccgcttcaccatatccagagataatgccaagaacactctctacctgcagatgaacagcctgaagcctgaaggaacagctgtgtactactgcaatgcacacaaagacacagcccagggcacctcctgtacaaaaacccaggctgcttctcattggtgctccctccccacctctgcagaacaggaaagtgcagctgaga

SEQ ID NO: 92 - DNA, 801 bp, synthetic construct
  source 1..801: mol_type = other DNA
acagcatattttccaaataccatcattgtcagcaaacttctgcagagcaccgtcttcttatatgggtacagcctattcctccagcatcccactagagcttcttatatagtaggagacatgcaaatagggccctccctctactgatgaaaaccaacccaaccctgaccctgcaggtctcagagaggagccttagccctggactccaaggcctttccacttggtgatcagcactgagcacagaggactcaccatggagttggggctgagctgggttttccttgttgctattttagaaggtgattcatggaaaactaggaagattgagtgtgtgtggatatgagtgtgagaaacagtggatttgtgtggcagtttctgaccttggtgtctctttgtttgcaggtgtccagtgtcaggtgcagctggtggagtcaggaggtggtttggttcaagcagggggcagtttacgacacagctgtgctgcttctgggctcacttttggaagctatgccatgggctggtatcggcaagcgccagggaaagaaagagaacttgttgcagccataagcagtggtggctcaacctactatgcagactctgtgaaaggacgcttcaccatctccagggataatgccaagaacacagtctacctgcagatgaacagcctgaagcctgaaggaacagctgtgtcctactgcaatgctcacagtgaggggaagtcagtgtgagcccagacacaaacctccctgcaggggtcccttgggaccaccagggggcgacagggcattgagcacggggctgtct

SEQ ID NO: 93 - DNA, 748 bp, synthetic construct
  source 1..748: mol_type = other DNA
gggccctgcctctgagctcctctttgcatccaatctgctgaagaacatggctctagggaaacccagttgtagacctgagggccccggctcttcaatgagccatctccgtcccggggccttatatcagcaagtgacgcacacaggcaaatgccagggtgtggtttcctgtttaaatgtagcctcccccgctgcagaactgcagagcctgctgaattctggctgaccagggcagtcaccagagctccagacaatgtctgtctccttcctcatcttcctgcccgtgctgggcctcccatggggtcagtgtcagggagatgccgtattcacagcagcattcacagactgaggggtgtttcactttgctgtttccttttgtctccaggtgtcctgtcagccagggtggaccagacacctcggagtgttaccaaggagacaggagaatcactcaccatcaactgtgtgcttcgagatgcttcctatgcattgggttctacgtgctggtacaggaagaagtctgggtcaacaaatgaagagagcataagcaaaggaggcagatatgtggagactgtcaacagcggctccaaaagtttctccctgagaattaatgacctgactgtagaagatggtggcacctaccgctgtggggtccacagtgaggggaagtcagtgtgagcccagacacaaacctccctgcagggatgctcaggaccccagaaggcacccagcactaccagcgcagggcccagac

SEQ ID NO: 94 - DNA, 765 bp, synthetic construct
  source 1..765: mol_type = other DNA
agcacaatttcccaatgctttcaatatcacagatctccccgaggacattctgacatgctctgagccccactatctccaaaggcctctcaccccagagcttactatatagtaggagatatgcaaatagagccctccgtctgctgatgaaaaccagcccagccctgaccctgcagctctgagagaggagcccagccctgggattttcaggtgttttcatttggtgatcaggactgaacagagagaactcaccatggagtttgggctgagctggctttttcttgtggctattttaaaaggtaattcatggagaaatagaaaaattgagtgtgaatggataagagtgagagaaacagtggatacgtgtggcagtttctgaccagggtttctttttgtttgcaggtgtccagtgtgctcgagtagaccagactcctaagactatcaccaaggagacaggagaatcactcaccatcaactgtgttcttagagatacgtcctatgcattggggtccacctactggtacaggaagaagctgggctctacaaatgaggaaagcatttcaaaaggtgggcggtatgtggagactgtcaacagtggaagcaaaagtctatctctgcgtataaatggcctgaaggtggaagacagctggacataccgctgcaaagccacagtgaggggaagtcattgtgagcccagacacaaacctccctgcaggaacgatgggggggaaatcagcggcagggggcgctcaggacccgctgatcaga

SEQ ID NO: 95 - protein, 120 aa, Homo sapiens
EVQLLESGGGLVQPGGSLRLSCAASGFRISDEDMGWVRQAPGKGLEVWSSIYGPSGSTYYADSVKGRFTISRDNSKNTLYLQMNSLRAEDTAVYYCASALEPLSEPLGFWGQGTLVTVSS

SEQ ID NO: 96 - protein, 105 aa, synthetic construct
QQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQ

SEQ ID NO: 97 - protein, 116 aa, synthetic construct
QQSTVSDVPRDLEVVAATPTSLLISWDAPAVTVRYYRITYGETGGNSPVQEFTVPGSKSTATISGLKPGVGGSGGSGGSGGDYTITVYAVTGRGDSPASSKPISINYRTEIDKPSQ

SEQ ID NO: 98 - DNA, 28 bp, synthetic construct
  source 1..28: mol_type = other DNA
cacagtgnnnnnnnnnnnnacaaaaacc

SEQ ID NO: 99 - DNA, 39 bp, synthetic construct
  source 1..39: mol_type = other DNA
cacagtgnnnnnnnnnnnnnnnnnnnnnnnacaaaaacc

SEQ ID NO: 100 - protein, 104 aa, synthetic construct
QQSTVSDVPRDLEWAATPTSLLISWDAPAVTVRYYRITYGETGGNSPVQEFTVPGSKSTATISGLKPGVDYTITVYAVTGRGDSPASSKPISINYRTEIDKPSQ

SEQ ID NO: 101 - protein, 80 aa, synthetic construct
QQSTVSDVPRDLEVVAATPTSLLISWDAPAVTVRYYRITYGETGGNSPVQEFTVPGSKSTATISGLKPGVDYTITVYAVT

SEQ ID NO: 102 - protein, 13 aa, synthetic construct
PISINYRTEIDKP

SEQ ID NO: 103 - protein, 91 aa, synthetic construct
QQSTVSDVPRDLEVVAATPTSLLISWDAPAVTVRYYRITYGETGGNSPVQEFTVPGSKSTATISGLKPGVGGSGGSGGSGGDYTITVYAVT

SEQ ID NO: 104 - protein, 15 aa, synthetic construct
PISINYRTEIDKPSQ

SEQ ID NO: 105 - protein, 16 aa, synthetic construct
  VARIANT 6: note = any amino acid other than tryptophan
DAFDIXGQGTMVTVSS

SEQ ID NO: 106 - protein, 15 aa, synthetic construct
  VARIANT 5: note = any amino acid other than tryptophan
YFDYXGQGTLVTVSS

SEQ ID NO: 107 - DNA, 49 bp, Homo sapiens
  source 1..49: mol_type = genomic DNA
gatgcttttgatatctggggccaagggacaatggtcaccgtctcttcag

SEQ ID NO: 108 - protein, 16 aa, Homo sapiens
DAFDIWGQGTMVTVSS

SEQ ID NO: 109 - DNA, 46 bp, Homo sapiens
  source 1..46: mol_type = genomic DNA
tactttgactactggggccagggaaccctggtcaccgtctcctcag

SEQ ID NO: 110 - protein, 15 aa, Homo sapiens
YFDYWGQGTLVTVSS

SEQ ID NO: 111 - DNA, 49 bp, synthetic construct
  source 1..49: mol_type = other DNA
  misc_feature 16..18: product = any codon other than a stop codon or a codon encoding tryptophan
gatgcttttgatatcnnnggccaagggacaatggtcaccgtctcttcag

SEQ ID NO: 112 - DNA, 46 bp, synthetic construct
  source 1..46: mol_type = other DNA
  misc_feature 13..15: product = any codon other than a stop codon or a codon encoding tryptophan
tactttgactacnnnggccagggaaccctggtcaccgtctcctcag

SEQ ID NO: 113 - DNA, 2366 bp, synthetic construct
  source 1..2366: mol_type = other DNA
agcccccagccccacaggccccctaccagccgcagggttttggctgagctgagaaccactgtgctaactggggacacagtgattggcagctctacaaaaaccatgctcccccgggaccccgggctgtgggtttctgtagcccctggctcagggctgactcaccgtggctgaatacttccagcactggggccagggcaccctggtcaccgtctcctcaggtgagtctgctgtctggggatagcggggagccaggtgtactgggccaggcaagggctttggcttcagacttggggacaggtgctcagcaaaggaggtcggcaggagggcggagggtgtgtttttgtatgggagaagcaggagggcagaggctgtgctactggtacttcgatctctggggccgtggcaccctggtcactgtctcctcaggtgagtcccactgcagccccctcccagtcttctctgtccaggcaccaggccaggtatctggggtctgcagccggcctgggtctggcctgaggccacaccagctgccatccctggggtctccgccatgggctgcatgccagagccctgctgtcacttagccctggggccagctggagcccccaaggacaggcagggaccccgctgggcttcagccccgtcagggaccctccacaggtagcaagcaggccgagggcagggacgggaaggagaagttgtgggcagagcctgggctggggctgggcgctggctgttcatgtgccggggaccaggcctgcgctttagtgtggctacaagtgcttggagcactggggccagggcagcccggccaccgtctccctgggaacgtcacccctccctgcctgggtctcagcccgggggtctgtgtggctggggacagggacgccggctgcctctgctctgtgcttgggccatgtgacccattcgagtgtcctgcacgggcacaggtttatgtctgggcaggaacagggactgtgtccctgtgtgatgcttttgatatcnnnggccaagggacaatggtcaccgtctcttcaggtaagatggctttccttctgcctcctttctctgggcccagcgtcctctgtcctggagctgggagataatgtccgggggctccttggtctgcgctgggccatgtggggccctccggggctccttctccggctgtttgggaccacgttcagcagaaggcctttctttgggaactgggactctgctgctggggcaaagggtgggcagagtcatgcttgtgctggggacaaaatgaccttgggacacggggctggctgccacggccggcccgggacagtcggagagtcaggtttttgtgcaccccttaatggggcctcccacaatgtgactactttgactacnnnggccagggaaccctggtcaccgtctcctcaggtgagtcctcacaacctctctcctgctttaactctgaagggttttgctgcatttttggggggaaataagcgtgctgggtctcctgccaagagagccccggagcagcctggggggctcaggaggatgccctgaggcaacagcggccacacagacgaggggcaagggctccagatgctccttcctcctgagcccagcagcacgggtctctctgtggccagggccaccctgggcctctggggtccaatgtccaacaacccccgggccctccccgggctcagtctgagagggtcccagggacttagcggggtgccagttcttgcctggggtcctggcattgttgtcacaatgtgacaactggttcgacccctggggccagggaaccctggtcaccgtctcctcaggtgagtcctcaccaccccctctctgagtccacttagggagactcagcttgccagggtctcagggtcagagtcttggaggcattttggaggtcaggaaagaaagctggggagagggacccttcgaatgggaacccagcctgtcctccccaagtccggccacagatgtcggcagctggggggctccttcggctggtctggggtgacctctctccgcttcacctggagcattctcaggggctgtcgtgatgattgcgtggtgggactctgtcccgctccaaggcacccgctctctgggacgggtgccccccggggtttttggactcctgggggtgacttagcagccgtctgcttgcagttggacttcccaggccgacagtggtctggcttctgaggggtcaggccagaatgtggggtacgtgggaggccagcagagggttccatgagaagggcaggacagggccacggacagtcagcttccatgtgacgcccggagacagaaggtctctgggtggctgggtttttgtggggtgaggatggacattctgccattgtgat

SEQ ID NO: 114 - DNA, 800 bp, synthetic construct
  source 1..800: mol_type = other DNA
gttatattttcctgagagataggattacctccagtgttttccgggaccctctcatctgctctgggcactgccctctcctccagcgtcccactagagcttgctatatagtaggagacatgcaaatagggccctccctctgctgataaaaaccagccgagcccagaccctgcagctctgggagaagagccccagccccagaattcccaggagtttccattcggtgatcagcactgaacacagaggactcaccatggagtttgggctgagctgggttttccttgttgctattataaaaggtgatttatggagaactagagacattgagtggacgtgagtgagataagcagtgaatatatgtggcagtttctgaccaggttgtctctgtgtttgcaggtgtccagtgtcaggtgcagctggtggagtctgggggaggcttggtcaagcctggagggtccctgagactctcctgtgcagcctctggattcaccttcagtgactactacatgagctggtaccgccaggctccagggaaggagagggagggcgtttcatacattagtagtagtagtagttacacaaactacgcagactctgtgaagggccgattcaccatctccagagacaacgccaagaactcactgtatctgcaaatgaacagcctgagagccgaggacacggctgtgtattactgtgcgagagacacagtgaggggaggtcagtgtgagcccagacacaaacctccctgcaggggtccccaggaccaccagggggcgcccgggacactgtgcacggggctgtct

SEQ ID NO: 115 - DNA, 804 bp, synthetic construct
  source 1..804: mol_type = other DNA
gacagcatattttccaaataccatcgtcagcaaacatctgcagggcaccgtcttattatctgggtacagcctattcctccagcgtcccaccctagagcttgttatatagtaggagatatgcaaatagggacctccctctactgatgaaaaccaaccgaaccctgaccctgcagctctgagagaggagccttagccctggattccaaggcctatccacttggtgatcagcactgagcaccgaggattcaccatggaactggggctccgctgggttttccttgttgctattttagaaggtgaatcatggaaaagtagagagatttagtgtgtgtggatatgagtgagagaaacggtggatgtgtgtgacagtttctgaccaatgtctctctgtttgcaggtgtccagtgtgaggtgcagctggtggagtctgggggaggcctggtcaagcctggggggtccctgagactctcctgtgcagcctctggattcaccttcagtagctatagcatgaactggtaccgccaggctccagggaaggagagggagggcgtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagacacagtgaggggaagtcagtgtgagcccagacacaaacctccctgcaggggtccccaggaccaccagggggcgcccgggacactgtgcacggggctgtct

SEQ ID NO: 116 - DNA, 806 bp, synthetic construct
  source 1..806: mol_type = other DNA
agcacaatttcccaatgctttcaatatcacagatctccccgaggacattctgacatgctctgagccccactatctccaaaggcctctcaccccagagcttactatatagtaggagatatgcaaatagagccctccgtctgctgatgaaaaccagcccagccctgaccctgcagctctgagagaggagcccagccctgggattttcaggtgttttcatttggtgatcaggactgaacagagagaactcaccatggagtttgggctgagctggctttttcttgtggctattttaaaaggtaattcatggagaaatagaaaaattgagtgtgaatggataagagtgagagaaacagtggatacgtgtggcagtttctgaccagggtttctttttgtttgcaggtgtccagtgtgaggtgcagctggtggagtctgggggaggcttggtacagcctggggggtccctgagactctcctgtgcagcctctggattcacctttagcagctatgccatgagctggtaccgccaggctccagggaaggagagggagggcgtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagacacagtgaggggaagtcattgtgagcccagacacaaacctccctgcaggaacgatgggggggaaatcagcggcagggggcgctcaggacccgctgatcag

SEQ ID NO: 117 - DNA, 789 bp, synthetic construct
  source 1..789: mol_type = other DNA
tgggcttggagaggggaggccccaagaagagaactgagttctcaaagggcacagccagcattctcctcccagggtgagctcaaaagactggcgcctctctcatcccttttcactgctccgtacaaacgcaccacccccatgcaaatcctcacttaggcgcccacaggaagccaccacacatttccttaaattcaggtccaactcataagggaaatgctttctgagagtcatggatctcatgtgcaagaaaatgaagcacctgtggttcttcctcctgctggtggcggctcccagatgtgagtgtttctaggatgcagacatggagatatgggaggctgcctctgatcccagggctcactgtgggtttttctgttcacaggggtcctgtcccagctgcagctgcaggagtcgggcccaggactggtgaagccttcggagaccctgtccctcacctgcactgtctctggtggctccatcagcagtagtagttactactggggctggtaccgccagcccccagggaaggagagggagggcattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagacacacagtgaggggaggtgagtgtgagcccagacaaaaacctccctgcagggaggctgagggggcgggcgcaggtgcagctcagggccagcagggggcgtgc

SEQ ID NO: 118 - DNA, 806 bp, synthetic construct
  source 1..806: mol_type = other DNA
ttcataataagcacaatttctcaaatcccattgttgtcacccatcttcctcaggacactttcatctgccctgggtcctgctctttcttcaggtgtctcaccccagagcttgatatatagtaggagacatgcaaatagggccctcactctgctgaagaaaaccagccctgcagctctgggagaggagccccagccctgggattcccagctgtttctgcttgctgatcaggactgcacacagagaactcaccatggagtttgggctgagctgggttttccttgttgctattttaaaaggtgattcatggagaactggagatatggagtgtgaatggacatgagtgagataagcagtggatgtgtgtggcagtttctgaccagggtgtctctgtgtttgcaggtgtccagtgtgaggtgcagctggtggagtccgggggaggcttagttcagcctggggggtccctgagactctcctgtgcagcctctggattcaccttcagtagctactggatgcactggtaccgccaagctccagggaaggagagggtgggcgtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagagacacagtgaggggaagtcaatgtgagcccagacacaaacctcgctgcaggggcatctgagaccacgagggggtgtcctgggccctgtgaactgggctgctc

SEQ ID NO: 119 - protein, 98 aa, synthetic construct
QVQLVESGGGLVKPGGSLRLSCAASGFTFSDYYMSWYRQAPGKEREGVSYISSSSSYTNYADSVKGRFTISRDNAKNSLYLQMNSLRAEDTAVYYCAR

SEQ ID NO: 120 - protein, 98 aa, synthetic construct
EVQLVESGGGLVKPGGSLRLSCAASGFTFSSYSMNWYRQAPGKEREGVSSISSSSSYIYYADSVKGRFTISRDNAKNSLYLQMNSLRAEDTAVYYCAR

SEQ ID NO: 121 - protein, 98 aa, synthetic construct
EVQLVESGGGLVQPGGSLRLSCAASGFTFSSYAMSWYRQAPGKEREGVSAISGSGGSTYYADSVKGRFTISRDNSKNTLYLQMNSLRAEDTAVYYCAK

SEQ ID NO: 122 - protein, 99 aa, synthetic construct
QLQLQESGPGLVKPSETLSLTCTVSGGSISSSSYYWGWYRQPPGKEREGIGSIYYSGSTYYNPSLKSRVTISVDTSKNQFSLKLSSVTAADTAVYYCAR

SEQ ID NO: 123 - protein, 98 aa, synthetic construct
EVQLVESGGGLVQPGGSLRLSCAASGFTFSSYWMHWYRQAPGKERVGVSRINSDGSSTSYADSVKGRFTISRDNAKNTLYLQMNSLRAEDTAVYYCAR

SEQ ID NO: 124 - DNA, 13 bp, synthetic construct
  source 1..13: mol_type = other DNA
ataacttcgtata

SEQ ID NO: 125 - DNA, 13 bp, synthetic construct
  source 1..13: mol_type = other DNA
ataacttcgtata

SEQ ID NO: 126 - DNA, 13 bp, synthetic construct
  source 1..13: mol_type = other DNA
ataacttcgtata

SEQ ID NO: 127 - DNA, 13 bp, synthetic construct
  source 1..13: mol_type = other DNA
ataacttcgtata

SEQ ID NO: 128 - DNA, 13 bp, synthetic construct
  source 1..13: mol_type = other DNA
ataacttcgtata

SEQ ID NO: 129 - DNA, 13 bp, synthetic construct
  source 1..13: mol_type = other DNA
ataacttcgtata

SEQ ID NO: 130 - DNA, 13 bp, synthetic construct
  source 1..13: mol_type = other DNA
ataacttcgtata

SEQ ID NO: 131 - DNA, 13 bp, synthetic construct
  source 1..13: mol_type = other DNA
ataacttcgtata

SEQ ID NO: 132 - DNA, 13 bp, synthetic construct
  source 1..13: mol_type = other DNA
ataacttcgtata

SEQ ID NO: 133 - DNA, 13 bp, synthetic construct
  source 1..13: mol_type = other DNA
ataacttcgtata

SEQ ID NO: 134 - DNA, 13 bp, synthetic construct
  source 1..13: mol_type = other DNA
ataacttcgtata

SEQ ID NO: 135 - DNA, 13 bp, synthetic construct
  source 1..13: mol_type = other DNA
taccgttcgtata

SEQ ID NO: 136 - DNA, 13 bp, synthetic construct
  source 1..13: mol_type = other DNA
taccgttcgtata

SEQ ID NO: 137 - DNA, 13 bp, synthetic construct
  source 1..13: mol_type = other DNA
ataacttcgtata

SEQ ID NO: 138 - DNA, 13 bp, synthetic construct
  source 1..13: mol_type = other DNA
ataacttcgtata

SEQ ID NO: 139 - DNA, 13 bp, synthetic construct
  source 1..13: mol_type = other DNA
ataacttcgtata

SEQ ID NO: 140 - DNA, 13 bp, synthetic construct
  source 1..13: mol_type = other DNA
ataacttcgtata

SEQ ID NO: 141 - DNA, 13 bp, synthetic construct
  source 1..13: mol_type = other DNA
ataacttcgtata

SEQ ID NO: 142-159 - skipped (18 entries)

SEQ ID NO: 160 - DNA, 13 bp, synthetic construct
  source 1..13: mol_type = other DNA
tatacgaagttat

SEQ ID NO: 161 - DNA, 13 bp, synthetic construct
  source 1..13: mol_type = other DNA
tatacgaagttat

SEQ ID NO: 162 - DNA, 13 bp, synthetic construct
  source 1..13: mol_type = other DNA
tatacgaagttat

SEQ ID NO: 163 - DNA, 13 bp, synthetic construct
  source 1..13: mol_type = other DNA
tatacgaagttat

SEQ ID NO: 164 - DNA, 13 bp, synthetic construct
  source 1..13: mol_type = other DNA
tatacgaagttat

SEQ ID NO: 165 - DNA, 13 bp, synthetic construct
  source 1..13: mol_type = other DNA
tatacgaagttat

SEQ ID NO: 166 - DNA, 13 bp, synthetic construct
  source 1..13: mol_type = other DNA
tatacgaagttat

SEQ ID NO: 167 - DNA, 13 bp, synthetic construct
  source 1..13: mol_type = other DNA
tatacgaagttat

SEQ ID NO: 168 - DNA, 13 bp, synthetic construct
  source 1..13: mol_type = other DNA
tatacgaagttat

SEQ ID NO: 169 - DNA, 13 bp, synthetic construct
  source 1..13: mol_type = other DNA
tatacgaagttat

SEQ ID NO: 170 - DNA, 13 bp, synthetic construct
  source 1..13: mol_type = other DNA
tatacgaacggta

SEQ ID NO: 171 - DNA, 13 bp, synthetic construct
  source 1..13: mol_type = other DNA
tatacgaagttat

SEQ ID NO: 172 - DNA, 13 bp, synthetic construct
  source 1..13: mol_type = other DNA
tatacgaacggta

SEQ ID NO: 173 - DNA, 13 bp, synthetic construct
  source 1..13: mol_type = other DNA
tatacgaagttat

SEQ ID NO: 174 - DNA, 13 bp, synthetic construct
  source 1..13: mol_type = other DNA
tatacgaagttat

SEQ ID NO: 175 - DNA, 13 bp, synthetic construct
  source 1..13: mol_type = other DNA
tatacgaagttat

SEQ ID NO: 176 - DNA, 13 bp, synthetic construct
  source 1..13: mol_type = other DNA
tatacgaagttat

SEQ ID NO: 177 - DNA, 13 bp, synthetic construct
  source 1..13: mol_type = other DNA
tatacgaagttat